(19) United States
(12) Patent Application Publication
(10) Pub. No.: US 2017/0299608 A1
(43) Pub. Date: Oct. 19, 2017

(54) COMPOSITIONS AND METHODS FOR MODULATING BODY WEIGHT

(71) Applicant: NGM Biopharmaceuticals, Inc., South San Francisco, CA (US)

(72) Inventors: Jer-Yuan HSU, San Bruno, CA (US); Yu CHEN, Foster City, CA (US); Suzanne Christine CRAWLEY, Brisbane, CA (US); Hui TIAN, Foster City, CA (US); Wenyan SHEN, Redwood City, CA (US); Jie TANG, Palo Alto, CA (US); Jared Martin HIGBEE, San Francisco, CA (US)

(21) Appl. No.: 15/449,841

(22) Filed: Mar. 3, 2017

Related U.S. Application Data
(60) Provisional application No. 62/304,141, filed on Mar. 4, 2016.

(51) Int. Cl. G01N 33/68; G06F 19/16; C07K 16/22; C07K 14/495; C07K 16/28; C07K 14/71; G01N 33/50

(52) U.S. Cl. CPC G01N 33/6854; C07K 14/71; G06F 19/16; G01N 33/5008; C07K 14/495; C07K 16/2863; C07K 16/22; G01N 2500/02; C07K 2317/76

(57) ABSTRACT

The present disclosure provides a protein complex, including a three-dimensional structure of the protein complex, that plays a role in regulation of body weight. In addition, the protein complex and components thereof, including three-dimensional structures thereof, find use in identifying agents that can be used to control body weight.

### CROSS-REFERENCE TO RELATED APPLICATIONS

This application claims the benefit of priority of U.S. Provisional Application No. 62/304,141, filed Mar. 4, 2016, the entire contents of which is incorporated herein by reference.

### INTRODUCTION

Weight loss is associated with a number of diseases and conditions. For example, involuntary body weight loss is associated with certain wasting diseases such as cachexia and/or may be associated with systemic inflammation or an acute inflammatory response. Cachexia, is typically characterized by loss of weight, muscle atrophy, fatigue, weakness and significant loss of appetite, can greatly contribute to morbidity of patients suffering from some chronic diseases (e.g., cancer, chronic renal disease, chronic obstructive pulmonary disease, AIDS, tuberculosis, chronic inflammatory disease, sepsis and other forms of systemic inflammation, muscle wasting, such as muscular dystrophy, and the eating disorder known as anorexia nervosa). For example, in late stage cancer, cachexia is common (occurring in most terminally ill cancer patients), and is responsible for about a quarter of all cancer-related deaths. Metabolic processes (e.g., that act directly on muscle, reducing its mass and/or formation) and reduced food intake (e.g., that leads to loss of fat and/or muscle) may drive development and/or progression of cachexia. Cachexia may progress through stages that have been designated precachexia, cachexia, and refractory cachexia.

Regulation of body weight is a complex multifactorial process and agents that can regulate body weight and control involuntary weight loss, including, for example, agents that can regulate body weight and control involuntary weight loss, as well as methods to identify such agents, are of great interest.

### SUMMARY

The present disclosure provides a protein complex that plays a role in regulation of body weight. The components of the protein complex of the present disclosure can be used to modulate body weight. In addition, the protein complex and components thereof find use in identifying agents that can be used to control body weight. Also provided herein are methods for treating and/or preventing involuntary body weight loss. In addition, methods for reducing GDF15 activity in subjects having increased GDF15 level or at risk of developing increased GDF15 level are also disclosed.

In certain embodiments, an isolated complex that includes a GDNF family receptor alpha like (GFRAL) protein; and a GDF15 protein is provided. The GFRAL protein may be present on a surface of a cell that is genetically modified to express GFRAL. In certain embodiments, the GFRAL protein is purified from a cell genetically modified to express GFRAL. The GFRAL protein may be immobilized on a support. In certain embodiments, the isolated complex may also include a RET protein.

In certain embodiments, at least one of GDF15, GFRAL and RET protein may be fused to a heterologous protein. The heterologous protein fused to GDF15, GFRAL and RET protein may be independently selected from the group consisting of Ig Fc, albumin, and maltose binding protein. In certain embodiments, the albumin fused to at least one of GDF15, GFRAL and RET protein may be human serum albumin.

In certain embodiments, at least one of the GDF15 protein and GFRAL protein may be detectably labeled.

In certain embodiments, the complex is a crystal. The crystal, in some aspects, has the atomic coordinates described herein. The crystal can have the cell unit dimensions of a=75.4 Å, b=88.8 Å, c=121.3 Å, and/or have a resolution of about 2.20 Å.

Also provided herein is a composition that includes an isolated GDF15 protein; and a recombinant cell genetically modified to express a GFRAL protein. In certain embodiments, the recombinant cell may be genetically modified to express RET.

In certain embodiments, the recombinant cell may include a reporter construct. The reporter construct may include a promoter sequence operably linked to a nucleic acid sequence encoding a reporter, wherein the promoter directs expression of the reporter upon activation of RET by binding of the GDF15 protein to GFRAL.

A method for identifying an agent that binds to an extracellular domain of a GFRAL protein is also disclosed. The method may include assaying for binding of a candidate agent to an extracellular domain of GFRAL, wherein a candidate agent that binds the GFRAL protein is identified as an agent that binds to a GFRAL protein, wherein binding of the candidate agent is compared to binding of a GDF15 protein to the extracellular domain of a GFRAL protein. In certain embodiments, the candidate agent binds to the extracellular domain of the GFRAL protein with an affinity similar to the GDF15 protein. In another embodiment, the method may include constructing a three-dimensional structure of a complex with a GDF15 protein defined by the atomic coordinates described herein; and employing the three-dimensional structure and a modeling method to identify a candidate agent that binds to the GFRAL protein; assaying the candidate agent for binding to the extracellular domain of the GFRAL protein; and comparing the binding of the candidate agent to the binding of the GDF15 protein to the extracellular domain of the GFRAL protein, wherein the candidate agent is identified as an agent that binds to the extracellular domain of the GFRAL protein when the candidate agent binds with an affinity similar to the GDF15 protein. In certain cases, GDF15 may be detectably labeled.

In exemplary methods, GFRAL may be immobilized on a support or expressed by a recombinant cell genetically modified to express GFRAL. In certain cases, the recombinant cell may be genetically modified to express RET.

In an additional embodiment, the recombinant cell may include a reporter construct comprising a promoter sequence operably linked to a nucleic acid sequence encoding a reporter, where the promoter directs expression of the reporter upon activation of RET, and where the method may include assaying for expression of the reporter, wherein increased expression of the reporter as compared to a negative control identifies the agent as an agent that binds to GFRAL and activates RET.

In a further additional embodiment, the extracellular domain of the GFRAL protein comprises one or more amino acid residues of a GFRAL domain associated with the interface between a GFRAL protein and a GDF15 protein. In this embodiment, the one or more amino acid residues of the GFRAL domain can correspond to the amino acid residues at the positions selected from the group consisting of GLY140, LEU148, ALA149, ALA146, VAL142, ASN145, VAL139, ALA135, GLU136, LEU152, LEU132, SER201, ALA204, LEU205, LYS153, ILE196, PRO197, and GLN200 of SEQ ID NO: 9. Alternatively or in addition, the extracellular domain of the GFRAL protein comprises one or more amino acid residues of a GFRAL domain associated with the interface between a GFRAL protein and a RET protein. In this embodiment, the one or more amino acid residues of the GFRAL domain can correspond to the amino acid residues at the positions selected from the group consisting of GLN246, ARG247, ARG250, LYS251, CYS252, ASP255, GLU256, ASN257, CYS258, ILE259, SER260, THR261, LEU262, THR297, and GLN298 SER299 of SEQ ID NO: 9.

A recombinant cell genetically modified to express a GFRAL protein and a RET protein is disclosed. In certain examples, at least one of the GFRAL protein or a RET protein may be fused to a heterologous protein. In some examples, GFRAL may be fused to a heterologous protein. In other cases, RET may be fused to a heterologous protein. In some cases, both GFRAL and RET may be fused to a heterologous protein which heterologous protein may be independently selected from the group consisting of Ig Fc, albumin, and maltose binding protein. In some embodiments, at least one of the GFRAL protein or a RET protein may be detectably labeled.

A method for identifying an agent that modulates binding of a GDF15 protein to a GFRAL protein is also provided. The method may include contacting a candidate agent with a recombinant cell genetically modified to express GFRAL, wherein the contacting is in the presence of the GDF15; and assaying a level of binding of the GDF15 protein to the GFRAL protein, wherein a change in the level of binding of the GDF15 protein to the GFRAL protein in the presence of the candidate agent as compared to a level of binding of the GDF15 protein to the GFRAL protein in absence of the candidate agent identifies the candidate agent as an agent that modulates binding of the GDF15 protein to the GFRAL protein. In another embodiment, the method includes constructing a three-dimensional structure of a complex with a GDF15 protein defined by the atomic coordinates described herein; employing the three-dimensional structure and a modeling method to identify a candidate agent that modulates binding of a GDF15 protein to a GFRAL protein; contacting the candidate agent with a recombinant cell genetically modified to express the GFRAL protein, wherein the contacting is in the presence of the GDF15 protein; and assaying a level of binding of the GDF15 protein to the GFRAL protein, wherein a change in the level of binding of the GDF15 protein to the GFRAL protein in the presence of the candidate agent as compared to a level of binding of the GDF15 protein to the GFRAL protein in absence of the candidate agent identifies the candidate agent as an agent that modulates binding of the GDF15 protein to the GFRAL protein. In certain cases, the recombinant cell may be genetically modified to express RET.

In certain embodiments, the recombinant cell may include a reporter construct that includes a promoter sequence operably linked to a nucleic acid sequence encoding a reporter, where the promoter directs expression of the reporter upon activation of RET, where the assaying includes assaying for expression of the reporter, where a change in expression of the reporter as compared to the expression in the absence of the agent identifies the agent as an agent that modulates binding of GDF15 to GFRAL.

In certain embodiments, the agent may inhibit binding of GDF15 to GFRAL and the agent is identified an antagonist of GDF15-GFRAL binding. In other embodiments, when the agent increases binding of GDF15 to GFRAL, the agent is identified as an agonist of GDF15-GFRAL binding.

In a further additional embodiment, the GFRAL protein expressed by the recombinant cell includes an extracellular domain of the GFRAL protein. Accordingly, in some embodiments, the extracellular domain of the GFRAL protein comprises one or more amino acid residues of a GFRAL domain associated with the interface between a GFRAL protein and a GDF15 protein. In this embodiment, the one or more amino acid residues of the GFRAL domain can correspond to the amino acid residues at the positions selected from the group consisting of GLY140, LEU148, ALA149, ALA146, VAL142, ASN145, VAL139, ALA135, GLU136, LEU152, LEU132, SER201, ALA204, LEU205, LYS153, ILE196, PRO197, and GLN200 of SEQ ID NO: 9. Alternatively or in addition, the extracellular domain of the GFRAL protein comprises one or more amino acid residues of a GFRAL domain associated with the interface between a GFRAL protein and a RET protein. In this embodiment, the one or more amino acid residues of the GFRAL domain can correspond to the amino acid residues at the positions selected from the group consisting of GLN246, ARG247, ARG250, LYS251, CYS252, ASP255, GLU256, ASN257, CYS258, ILE259, SER260, THR261, LEU262, THR297, and GLN298 SER299 of SEQ ID NO: 9.

A method for identifying an agent that modulates binding of a GFRAL protein to a RET protein is also provided. The method may include contacting a candidate agent with a recombinant cell genetically modified to express GFRAL and RET; and assaying a level of binding of GFRAL to RET; wherein a change in the level of binding of the GFRAL protein and the RET protein in the presence of the candidate agent as compared to a level of binding of the GFRAL protein and the RET protein in absence of the candidate agent identifies the candidate agent as an agent that modulates binding of the GFRAL protein to the RET protein. In another embodiment, the method includes constructing a three-dimensional structure of a complex with a GDF15 protein defined by the atomic coordinates described herein; employing the three-dimensional structure and a modeling method to identify a candidate agent that modulates binding of the GFRAL protein to the RET protein; contacting the candidate agent with a recombinant cell genetically modified to express the GFRAL protein and the RET protein; and assaying a level of binding of the GFRAL protein and the RET protein, wherein a change in the level of binding of the GFRAL protein and the RET protein in the presence of the candidate agent as compared to a level of binding of the GFRAL protein and the RET protein in absence of the candidate agent identifies the candidate agent as an agent that modulates binding of the GFRAL protein to the RET protein.

Additionally, in some embodiments, the GFRAL protein expressed by the recombinant cell comprises an extracellular domain of the GFRAL protein. In some aspects, the extracellular domain of the GFRAL protein comprises one or more amino acid residues of a GFRAL domain associated with the interface between a GFRAL protein and a RET protein. In this embodiment, the one or more amino acid residues of the GFRAL domain can correspond to the amino acid residues at the positions selected from the group consisting of GLN246, ARG247, ARG250, LYS251, CYS252, ASP255, GLU256, ASN257, CYS258, ILE259, SER260, THR261, LEU262, THR297, and GLN298 of SEQ ID NO: 9.

In certain embodiments, a method of treating involuntary body weight loss in a subject or preventing involuntary body weight loss in a subject at risk of developing involuntary body weight loss is disclosed. The method may include administering to the subject at least one of: i) an agent that binds an extracellular domain of a GFRAL protein; and ii) an extracellular domain of GFRAL (GFRAL-ECD), wherein the agent or GFRAL-ECD is administered in an amount effective to treat, or prevent onset of, involuntary body weight loss in the subject.

Also provided herein is a method of reducing a GDF15 protein activity in a subject having increased GDF15 protein activity or at risk of developing increased GDF15 protein activity. The method may include administering to the subject at least one of: i) an agent that binds an extracellular domain of a GFRAL protein; and ii) an extracellular domain of GFRAL (GFRAL-ECD), wherein the agent or GFRAL-ECD is administered in an amount effective to reduce GDF15 activity in the subject.

A method of treating cachexia in a subject, or preventing cachexia in a subject at risk of cachexia is also provided. The method may include administering to the subject at least one of: i) an agent that binds an extracellular domain of a GFRAL protein; and ii) a soluble extracellular domain of GFRAL (GFRAL-ECD), wherein the agent or GFRAL-ECD is administered in an amount effective to treat, or prevent onset of, cachexia in the subject.

In certain embodiments, when the GFRAL-ECD is administered, the GFRAL-ECD comprises one or more amino acid residues of a GFRAL domain associated with the interface between a GFRAL protein and a GDF15 protein. In this embodiment, the one or more amino acid residues of the GFRAL domain can correspond to the amino acid residues at the positions selected from the group consisting of GLY140, LEU148, ALA149, ALA146, VAL142, ASN145, VAL139, ALA135, GLU136, LEU152, LEU132, SER201, ALA204, LEU205, LYS153, ILE196, PRO197, and GLN200 of SEQ ID NO: 9. Alternatively or in addition, the GFRAL-ECD comprises one or more amino acid residues of a GFRAL domain associated with the interface between a GFRAL protein and a RET protein, wherein the one or more amino acid residues of the GFRAL domain correspond to the amino acid residues at the positions selected from the group consisting of GLN246, ARG247, ARG250, LYS251, CYS252, ASP255, GLU256, ASN257, CYS258, ILE259, SER260, THR261, LEU262, THR297, and GLN298 of SEQ ID NO: 9.

In certain embodiments, the agent may include a soluble GFRAL-ECD. In certain embodiments, the GFRAL-ECD may be fused to a heterologous protein. The heterologous protein may be selected from the group consisting of Ig Fc, albumin, and maltose binding protein. For example, the albumin may be human serum albumin.

In other embodiments, when the agent is administered, the agent may be an antibody that binds to an extracellular domain of GFRAL. The extracellular domain of the GFRAL protein that the antibody binds to, in some embodiments, comprises one or more amino acid residues of a GFRAL domain associated with the interface between a GFRAL protein and a GDF15 protein. In this embodiment, the one or more amino acid residues of the GFRAL domain can correspond to the amino acid residues at the positions selected from the group consisting of GLY140, LEU148, ALA149, ALA146, VAL142, ASN145, VAL139, ALA135, GLU136, LEU152, LEU132, SER201, ALA204, LEU205, LYS153, ILE196, PRO197, and GLN200 of SEQ ID NO: 9. Alternatively or in addition, the extracellular domain of the GFRAL protein that the antibody binds to comprises one or more amino acid residues of a GFRAL domain associated with the interface between a GFRAL protein and a RET protein. In this embodiment, the one or more amino acid residues of the GFRAL domain can correspond to the amino acid residues at the positions selected from the group consisting of GLN246, ARG247, ARG250, LYS251, CYS252, ASP255, GLU256, ASN257, CYS258, ILE259, SER260, THR261, LEU262, THR297, and GLN298 of SEQ ID NO: 9.

Also provided herein is a crystal comprising a GFRAL protein and a GDF15 protein. In some embodiments, the crystal diffracts x-ray radiation to produce a diffraction pattern representing the three-dimensional structure of the complex having approximately the following cell constants: a=75.4 Å, b=88.8 Å, c=121.3 Å, and space group P2₁. In some embodiments, the crystal diffracts x-ray radiations at a resolution of about 2.20 Å. The crystal can also include the GFRAL protein having the amino acid sequence of SEQ ID NO: 23, and/or the GDF15 protein in the form of a homodimer. The crystal can also have the atomic coordinates described herein. The crystal provided herein can be used in a screening assay for the identification of an antagonist of a GDF15 protein.

Also provided is a composition comprising the crystal provided herein.

Still further provided is a method for identifying a variant GFRAL protein with the ability to bind a GDF15 protein. The method may include constructing a three-dimensional structure of a complex comprising a GFRAL protein and a GDF15 protein defined by the atomic coordinates provided herein; employing the three-dimensional structure and a modeling method to identify a site for mutating the GFRAL protein and mutating the site to generate the variant GFRAL protein; producing the variant GFRAL protein; and assaying the variant GFRAL protein to determine its ability to bind the GDF15 protein.

In some embodiments, the site for mutating the GFRAL protein is located in a GFRAL domain associated with the interface between a GFRAL protein and a GDF15 protein. In this embodiment, the domain can comprise one or more amino acid residues selected from the group consisting of GLY140, LEU148, ALA149, ALA146, VAL142, ASN145, VAL139, ALA135, GLU136, LEU152, LEU132, SER201, ALA204, LEU205, LYS153, ILE196, PRO197, and GLN200 of SEQ ID NO 9.

In some embodiments, the site for mutating the GFRAL protein is at an amino acid corresponding to a position selected from the group consisting of GLY140, LEU148, ALA149, ALA146, VAL142, ASN145, VAL139, ALA135, GLU136, LEU152, LEU132, SER201, ALA204, LEU205, LYS153, ILE196, PRO197, and GLN200 of SEQ ID NO 9.

Still further provided is a method for identifying a variant GFRAL protein with the ability to bind a RET protein. The method may include constructing a three-dimensional structure of a complex comprising a GFRAL protein and a GDF15 protein defined by the atomic coordinates described herein; employing the three-dimensional structure and a modeling method to identify a site for mutating the GFRAL protein and mutating the site to generate the variant GFRAL protein; producing the variant GFRAL protein; and assaying the variant GFRAL protein to determine its ability to bind the RET protein.

In some embodiments, the site for mutating the GFRAL protein is located in a GFRAL domain associated with the interface between a GFRAL protein and a RET protein. In this embodiment, the domain can comprise one or more amino acid residues selected from the group consisting of GLN246, ARG247, ARG250, LYS251, CYS252, ASP255, GLU256, ASN257, CYS258, ILE259, SER260, THR261, LEU262, THR297, and GLN298 of SEQ ID NO 9.

In some embodiments, the site for mutating the GFRAL is at an amino acid corresponding to a position selected from the group consisting of GLN246, ARG247, ARG250, LYS251, CYS252, ASP255, GLU256, ASN257, CYS258, ILE259, SER260, THR261, LEU262, THR297, and GLN298 of SEQ ID NO 9.

Even still further provided is a method for identifying a variant GDF15 protein with the ability to bind a GFRAL protein. The method may include constructing a three-dimensional structure of a complex comprising a GFRAL protein and a GDF15 protein defined by the atomic coordinates described herein; employing the three-dimensional structure and a modeling method to identify a site for mutating the GDF15 protein and mutating the site to generate the variant GDF15 protein; producing the variant GDF15 protein; and assaying the variant GDF15 protein to determine its ability to bind the GFRAL protein.

In some embodiments, the site for mutating the GDF15 protein is located in a GDF15 domain associated with the interface between a GDF15 protein and a GFRAL protein. In this embodiment, the domain can comprise one or more amino acid residues selected from the group consisting of SER35, LEU34, THR94, GLY95, GLN40, VAL96, LEU98, PRO36, VAL87, LEU88, ILE89, ASP102, THR100, PRO85, and MET86 of SEQ ID NO: 6.

In some embodiments, the site for mutating the GDF15 protein is at an amino acid corresponding to a position selected from the group consisting of SER35, LEU34, THR94, GLY95, GLN40, VAL96, LEU98, PRO36, VAL87, LEU88, ILE89, ASP102, THR100, PRO85, and MET86 of SEQ ID NO: 6.

Also provided herein is a method for producing an agent that inhibits formation of a complex comprising a GFRAL protein and a GDF15 protein (GFRAL/GDF15 complex) or a complex comprising a GFRAL protein and a RET protein (GFRAL/RET complex). The method can include obtaining two or more 3-dimensional structures of a complex comprising a GFRAL protein and one of two or more agents (GFRAL/agent complex); comparing each of the 3-dimensional GFRAL/agent complex structures with a 3-dimensional structure of the GFRAL/GDF15 complex or with a 3-dimensional structure of a GFRAL/RET complex; selecting at least one of the two or more agents based on the structural similarity of the GFRAL/agent complex with the 3-dimensional structure of a GFRAL/GDF15 complex or with a 3-dimensional structure of a GFRAL/RET complex; and producing at least 1 g of the agent.

In some embodiments, the at least one agent is selected if the at least one agent binds to the GFRAL protein with the same or higher affinity as it binds to the GDF15 protein or the RET protein.

Additionally, the above method include a comparing step that includes comparing the amino acids of the GFRAL protein in the two or more GFRAL/agent complexes with the amino acids of the GFRAL protein in the GFRAL/GDF15 complex selected from the group consisting of GLY140, LEU148, ALA149, ALA146, VAL142, ASN145, VAL139, ALA135, GLU136, LEU152, LEU132, SER201, ALA204, LEU205, LYS153, ILE196, PRO197, and GLN200 of SEQ ID NO: 9. Alternatively or in addition, the comparing can include comparing the amino acids of the GFRAL protein in the two or more GFRAL/agent complexes with the amino acids of the GDF15 protein in the GFRAL/GDF15 complex selected from the group consisting of SER35, LEU34, THR94, GLY95, GLN40, VAL96, LEU98, PRO36, VAL87, LEU88, ILE89, ASP102, THR100, PRO85, and MET86 of SEQ ID NO: 6. Yet still further, alternatively or in addition, the comparing step can include comparing the amino acids of the GFRAL protein in the two or more GFRAL/agent complexes with the amino acids of the GFRAL protein in the GFRAL/RET complex selected from the group consisting of GLN246, ARG247, ARG250, LYS251, CYS252, ASP255, GLU256, ASN257, CYS258, ILE259, SER260, THR261, LEU262, THR297, and GLN298 of SEQ ID NO 9.

In some embodiments, the 3-dimensional structure of a GFRAL/GDF15 complex is defined by the atomic coordinates provided herein.

In some embodiments, the comparing includes employing a modeling program.

In some embodiments, the agent identified by the method is produced in a recombinant cell. In this embodiment, the amount of the agent that is produced is at least 10 g, at least 100 g, or at least 1,000 g.

### BRIEF DESCRIPTION OF THE DRAWINGS

FIGS. 1A, 1B, and 1C depict amino acid sequence of a human GDF15 protein (FIG. 1A; SEQ ID NO: 6), Fc-hGDF15 (FIG. 1B; SEQ ID NOS: 7 and 8) and HSA-hGDF15 (FIG. 1C; SEQ ID NO: 24).

FIGS. 2A-2D depict protein sequences of a human GFRAL protein (FIG. 2A; SEQ ID NO: 9), a mouse GFRAL protein (FIG. 2B; SEQ ID NO: 10), a rat GFRAL protein (FIG. 2C; SEQ ID NO: 11), and a monkey GFRAL protein (FIG. 2D; SEQ ID NO:12). FIG. 2E depicts alignment of GFRAL proteins from human, mouse, rat and monkey (SEQ ID NOS: 9-12).

FIGS. 3A-3H depict various RET9 and RET51 protein sequences (SEQ ID NOS: 13-20).

FIG. 4 illustrates a GFRAL-Fc protein sequence (SEQ ID NO: 21).

FIG. 5 depicts the specific binding of an Fc-GDF15 protein to a mouse GFRAL protein.

FIGS. 6A and 6B illustrate results from analysis of expression of GFRAL mRNA in mouse tissue.

FIGS. 7A and 7B depict the binding of ¹²⁵I-GDF15 to cells expressing a human GFRAL protein.

FIG. 8 illustrates cellular response mediated by GDF15 via GFRAL-RET receptor complex.

FIGS. 9A-9D illustrate cellular response mediated by a human GDF15 protein via GFRAL-RET receptor complex from four species (FIG. 9A: human, FIG. 9B: cynomolgus monkey, FIG. 9C: rat, FIG. 9D: mouse).

FIG. 10 shows that a GDF15 protein, a GFRAL-Fc protein, and the antibody 1M03 compete with ¹²⁵I-GDF15 for binding to GFRAL expressing cells.

FIG. 11 depicts the effect of inhibitors (GFRAL-Fc and 1M03 antibody) of GFRAL-GDF15 binding on the cellular response to GDF15.

FIG. 12 illustrates that anti-GFRAL ECD antibodies compete with a GDF15 protein for binding to a GFRAL ECD protein.

FIG. 13 illustrates that anti-GFRAL ECD antibodies inhibit GDF15-mediated GFRAL-RET receptor complex activation.

FIG. 14 shows that GFRAL interacts with RET independent of GDF15.

FIG. 15 shows an exemplary crystal of a complex having a GFRAL protein and a GDF15 protein.

FIG. 16 illustrates an exemplary GFRAL electron density map.

FIG. 17 shows an exemplary ribbon diagram of the a GFRAL/GDF15 dimer formed in an asymmetric GFRAL/GDF15 crystal unit. The GFRAL protein domains D2 and D3 are indicated as GFRAL D2 and GFRAL D3.

FIG. 18 shows an exemplary ribbon diagram of a dimer of GFRAL/GDF15 hetero-dimers formed in an asymmetric GFRAL/GDF15 crystal unit. The GFRAL protein domains D2 and D3 are indicated as GFRAL D2 and GFRAL D3.

FIGS. 19A-19B show different surface representations of a dimer of GFRAL/GDF15 hetero-dimers.

FIG. 20 illustrates GFRAL residues interacting with GDF15.

FIGS. 21A-21D illustrate a GFRAL/GDF15 interface. The GFRAL protein domains D2 and D3 are indicated as GFRAL D2 and GFRAL D3.

FIGS. 22A-22B show different aspects of a superposition of a GFRAL protein and GFRα1 depicted as ribbon diagrams.

FIGS. 23A-23D illustrate different aspects of the interaction of a GFRAL protein with a RET protein in a RET/GFRAL/GDF15 model.

FIGS. 24A-24B illustrate amino acids on the RET interface of a GFRAL protein.

FIG. 25 shows a sequence alignment between various GFRAL proteins. SEQ ID NOS: 9 and 31 to 40 are depicted.

FIG. 26 shows a sequence alignment between various GDF15 proteins. SEQ ID NOS: 25 and 41 to 51 are depicted.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “the protein” includes reference to one or more proteins, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a protein complex that plays a role in regulation of body weight. The components of the protein complex of the present disclosure can be used to modulate body weight. In addition, the protein complex and components thereof find use in identifying agents that can be used to control body weight. Also provided herein are methods for treating and/or preventing involuntary body weight loss. In addition, methods for reducing GDF15 activity in subjects having increased GDF15 activity or at risk of developing increased GDF15 activity are also disclosed.

The present disclosure also provides methods for crystallizing a GDF15 protein and a GFRAL protein, which is a previously unknown and newly identified receptor for GDF15. The present disclosure provides for the first time crystals of a GDF15 protein and a GFRAL protein. The crystals provided herein diffract x-rays with sufficiently high resolution to allow determination of the three-dimensional structure of the GDF15 ligand-GFRAL receptor complex, including atomic coordinates. The three-dimensional structure (e.g., including as provided on computer readable media) is useful for rational drug design of GDF15-related mimetics or GFRAL-related ligands, as well as agents that interfere with the interaction of a GDF15 protein with its receptor, a GFRAL protein, and/or interfere with the interaction of a GFRAL protein with a RET protein. Such agents include antibodies that bind to a GFRAL protein in a GFRAL domain and that compete for the binding of a GDF15 protein with the GFRAL protein, thereby blocking in whole or in part GDF15-GFRAL complex formation. Such agents also include antibodies that bind to a GFRAL protein in a GFRAL domain and that interfere with the binding of a RET protein with the GFRAL protein, thereby blocking in whole or in part the GDF15-mediated activation of the RET protein (e.g., cell signaling).

Accordingly, the present disclosure provides compositions and methods for modulating body weight, including compositions comprising the newly identified receptor for GDF15 and methods comprising its use. Provided herein for the first time is a crystallized GDF15 receptor (e.g., a GFRAL protein), and a crystal structure of a complex of a GDF15 receptor (e.g., a GFRAL protein) and a GDF15 protein, resolved at 2.2 Å. The novel atomic coordinates from such crystals are useful to construct three-dimensional structures which are, in turn, useful for molecular modeling and for identifying agents that bind to a GFRAL protein and/or a GDF15 protein. Such agents are useful for modulating body weight and/or for the treatment and/or prevention GDF15-mediated diseases, disorders, or conditions.

### Definitions

The terms “patient” or “subject” as used interchangeably herein in the context of therapy, refer to a human and non-human animal, as the recipient of a therapy or preventive care.

The terms “treat”, “treating”, treatment” and the like refer to a course of action (such as administering an agent, e.g., a polypeptide or a pharmaceutical composition comprising a polypeptide) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (i.e., arresting the development or further development of the disease, disorder or condition or clinical symptoms associated therewith) an active disease.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “prevent”, “preventing”, “prevention” and the like refer to a course of action (such as administering an agent, e.g., a polypeptide or a pharmaceutical composition comprising a polypeptide) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The term “in need of prevention” as used herein refers to a judgment made by a physician or other caregiver that a subject requires or will benefit from preventative care. This judgment is made based on a variety of factors that are in the realm of a physician's or caregiver's expertise.

The phrase “therapeutically effective amount” refers to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to a patient. The therapeutically effective amount can be ascertained by measuring relevant physiological effects. The therapeutically effective amount can be adjusted in connection with the dosing regimen and diagnostic analysis of the subject's condition and the like.

The phrase “in a sufficient amount to effect a change” means that there is a detectable difference between a level of an indicator measured before (e.g., a baseline level) and after administration of a particular therapy. Indicators include any objective parameter (e.g., body weight or food intake) or subjective parameter (e.g., a subject's feeling of well-being or appetite).

The term “involuntary body weight loss” refers to the unintended loss of body weight that is observed in many conditions such as cachexia, liver cirrhosis, hyperthyroidism, chronic kidney disease, Parkinson's disease, cancer, eating disorder, and sarcopenia.

The term “cachexia” refers to wasting syndrome that is marked with loss of weight (e.g., involuntary loss of weight), muscle atrophy, fatigue, weakness, loss of fat mass, loss of lean mass, increased muscle protein breakdown, insulaine resistance, and/or significant loss of appetite in someone who is not actively trying to lose weight. Cachexia can greatly contribute to morbidity of patients suffering from some chronic diseases (e.g., cancer, chronic renal disease, chronic obstructive pulmonary disease, AIDS, tuberculosis, chronic inflammatory diseases, sepsis and other forms of systemic inflammation, muscle wasting, such as muscular dystrophy, and the eating disorder known as anorexia nervosa).e.g. For example, in late stage cancer, cachexia is common (occurring in most terminally ill cancer patients), and is responsible for about a quarter of all cancer-related deaths. Metabolic processes (e.g., that act directly on muscle, reducing its mass and/or formation) and reduced food intake (e.g., that leads to loss of fat and/or muscle) may drive development and/or progression of cachexia. Cachexia may progress through stages that have been designated precachexia, cachexia, and refractory cachexia.

The term “activators” refers to agents that, for example, stimulate, increase, activate, facilitate, enhance activation, sensitize or up-regulate the function or activity of one or more agents, e.g., polypeptides used to treat or prevent a metabolic disorder. In addition, activators include agents that operate through the same mechanism of action as the polypeptides of the present invention (i.e., agents that modulate the same signaling pathway as the polypeptides in a manner analogous to that of the polypeptides) and are capable of eliciting a biological response comparable to (or greater than) that of the polypeptides. Examples of activators include agonists such as small molecule compounds.

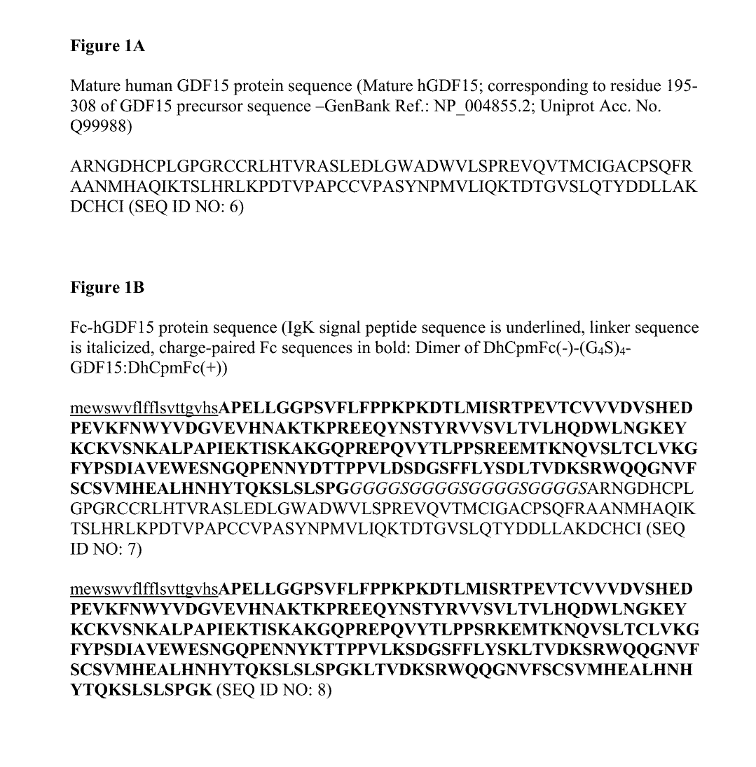

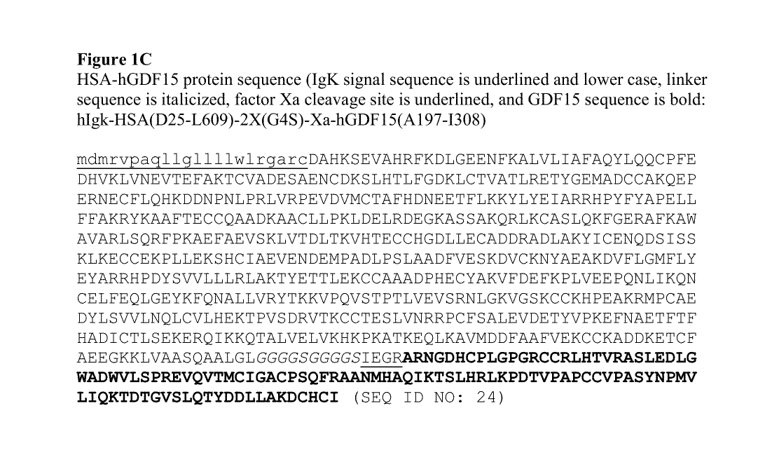

The term “native” or “wild type”, in reference to GDF15, refers to biologically active, naturally-occurring GDF15. The term includes the 112 amino acid human GDF15 mature sequence (FIG. 1A).

As used herein, “homologues” or “variants” refers to protein or DNA sequences that are similar based on their amino acid or nucleic acid sequences, respectively. Homologues or variants encompass naturally occurring DNA sequences and proteins encoded thereby and their isoforms. The homologues also include known allelic or splice variants of a protein/gene. Homologues and variants also encompass nucleic acid sequences that vary in one or more bases from a naturally-occurring DNA sequence but still translate into an amino acid sequence that correspond to the naturally-occurring protein due to degeneracy of the genetic code. Homologues and variants may also refer to those that differ from the naturally-occurring sequences by one or more conservative substitutions and/or tags and/or conjugates.

The terms “crystal”, and “crystallized” as used herein, refer to one or more proteins or fragments thereof that exist in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., ligand/receptor or antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ea., pp. 20 1-16, Oxford University Press, New York, N.Y., (1999).”

The terms “polypeptide” and “protein”, used interchangeably herein, refer to a polymeric form of amino acids, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

It will be appreciated that throughout this present disclosure reference is made to amino acids according to the single letter or three letter codes. For the reader's convenience, the single and three letter amino acid codes are provided below:

| | | | | | |
|---|---|---|---|---|---|
| G | Glycine | Gly | P | Proline | Pro |
| A | Alanine | Ala | V | Valine | Val |
| L | Leucine | Leu | I | Isoleucine | Ile |
| M | Methionine | Met | C | Cysteine | Cys |
| F | Phenylalanine | Phe | Y | Tyrosine | Tyr |
| W | Tryptophan | Trp | H | Histidine | His |
| K | Lysine | Lys | R | Arginine | Arg |
| Q | Glutamine | Gln | N | Asparagine | Asn |
| E | Glutamic Acid | Glu | D | Aspartic Acid | Asp |
| S | Serine | Ser | T | Threonine | Thr |

The terms “nucleic acid molecule” and “polynucleotide” are used interchangeably and refer to a polymeric form of nucleotides of any length, either deoxyribonucleotides or ribonucleotides, or analogs thereof. Non-limiting examples of polynucleotides include linear and circular nucleic acids, messenger RNA (mRNA), cDNA, recombinant polynucleotides, vectors, probes, and primers.

The term “heterologous” refers to two components that are defined by structures derived from different sources. For example, where “heterologous” is used in the context of a polypeptide, where the polypeptide includes operably linked amino acid sequences that can be derived from different polypeptides (e.g., a first component consisting of a tag peptide or protein and a second component derived from GFRAL polypeptide). Similarly, “heterologous” in the context of a polynucleotide encoding a chimeric polypeptide includes operably linked nucleic acid sequence that can be derived from different genes (e.g., a first component from a nucleic acid encoding a peptide according to an embodiment disclosed herein and a second component from a nucleic acid encoding a carrier polypeptide). Other exemplary “heterologous” nucleic acids include expression constructs in which a nucleic acid comprising a coding sequence is operably linked to a regulatory element (e.g., a promoter) that is from a genetic origin different from that of the coding sequence (e.g., to provide for expression in a host cell of interest, which may be of different genetic origin relative to the promoter, the coding sequence or both). For example, a T7 promoter operably linked to a polynucleotide encoding a GFRAL or RET polypeptide or domain thereof is said to be a heterologous nucleic acid. “Heterologous” in the context of recombinant cells can refer to the presence of a nucleic acid (or gene product, such as a polypeptide) that is of a different genetic origin than the host cell in which it is present.

The term “operably linked” refers to functional linkage between molecules to provide a desired function. For example, “operably linked” in the context of nucleic acids refers to a functional linkage between nucleic acids to provide a desired function such as transcription, translation, and the like, e.g., a functional linkage between a nucleic acid expression control sequence (such as a promoter or array of transcription factor binding sites) and a second polynucleotide, wherein the expression control sequence affects transcription and/or translation of the second polynucleotide. “Operably linked” in the context of a polypeptide refers to a functional linkage between amino acid sequences (e.g., of different domains) to provide for a described activity of the polypeptide.

As used herein in the context of the structure of a polypeptide, “N-terminus” and “C-terminus” refer to the extreme amino and carboxyl ends of the polypeptide, respectively, while “N-terminal” and “C-terminal” refer to relative positions in the amino acid sequence of the polypeptide toward the N-terminus and the C-term inus, respectively, and can include the residues at the N-terminus and C-terminus, respectively.

“Derived from” in the context of an amino acid sequence or polynucleotide sequence (e.g., an amino acid sequence “derived from” a GFRAL, RET, or GDF15 polypeptide) is meant to indicate that the polypeptide or nucleic acid has a sequence that is based on that of a reference polypeptide or nucleic acid (e.g., a naturally occurring GFRAL, RET, or GDF15 polypeptide or GFRAL, RET, or GDF15-encoding nucleic acid), and is not meant to be limiting as to the source or method in which the protein or nucleic acid is made.

“Isolated” refers to a protein of interest that, if naturally occurring, is in an environment different from that in which it may naturally occur. “Isolated” is meant to include proteins that are within samples that are substantially enriched for the protein of interest and/or in which the protein of interest is partially or substantially purified. Where the protein is not naturally occurring, “isolated” indicates the protein has been separated from an environment in which it was made by either synthetic or recombinant means.

“Enriched” means that a sample is non-naturally manipulated (e.g., by a scientist or a clinician) so that a protein of interest is present in a greater concentration (e.g., at least three-fold greater, at least 4-fold greater, at least 8-fold greater, at least 64-fold greater, or more) than the concentration of the protein in the starting sample, such as a biological sample (e.g., a sample in which the protein naturally occurs or in which it is present after administration), or in which the protein was made (e.g., as in a bacterial protein and the like).

“Substantially pure” indicates that an entity (e.g., polypeptide) makes up greater than about 50% of the total content of the composition (e.g., total protein of the composition) and typically, greater than about 60% of the total protein content. More typically, a “substantially pure” refers to compositions in which at least 75%, at least 85%, at least 90% or more of the total composition is the entity of interest (e.g., 95% of the total protein). Preferably, the protein will make up greater than about 90%, and more preferably, greater than about 95% of the total protein in the composition.

“Detectably labeled” in the context of a detectably labeled protein refers to a protein that has been modified by attachment of a detectable moiety. The detectable moiety may produce a signal directly or indirectly. Examples of a detectable moiety that produces a signal directly include a fluorescent molecule, a chemiluminescent molecule and a radioactive molecule. Detectable moieties that produce a signal indirectly include moieties that produce a signal upon exposure to detection reagents such as substrates, enzymes, or antibodies, etc. A detectable moiety that produces a signal directly can optionally be detected by indirect means such as by using a labeled antibody that binds to the moiety. The signal may be detectable by a radiation measuring device, e.g., a scintillation counter; a photodetector, e.g., a light microscope, a spectrophotometer, a fluorescent microscope, a fluorescent sample reader, or a florescence activated cell sorter, etc.

The term “endogenous” with reference to a gene, indicates that the gene is native to a cell, i.e., the gene is present at a particular locus in the genome of a non-modified cell. An endogenous gene may be a wild type gene present at that locus in a wild type cell (as found in nature). An endogenous protein is a protein expressed by an endogenous gene.

The term “construct” refers to a recombinant nucleic acid, generally recombinant DNA, that has been generated for the purpose of the expression of a specific nucleotide sequence(s), or is to be used in the construction of other recombinant nucleotide sequences. A construct might be present in a vector or in a genome.

The term “recombinant” refers to a polynucleotide or polypeptide that does not naturally occur in a host cell. A recombinant molecule may contain two or more naturally-occurring sequences that are linked together in a way that does not occur naturally. A recombinant cell contains a recombinant polynucleotide or polypeptide.

The term “coding sequence” refers to a nucleic acid sequence that once transcribed and translated produces a protein, for example, in vivo, when placed under the control of appropriate regulatory elements. A coding sequence as used herein may have a continuous ORF or might have an ORF interrupted by the presence of introns or non-coding sequences. In this embodiment, the non-coding sequences are spliced out from the pre-mRNA to produce a mature mRNA.

### Isolated Protein Complex

An isolated complex that includes a GDF15 protein and a GFRAL protein is provided. GDF15, also known as MIC-1 (macrophage inhibitory cytokine-1), PDF, PLAB, NAG-1, TGF-PL, and PTGFB, is a member of the transforming growth factor β (TGF-β) super-family. The inventors have discovered that GDF15 binds to GFRAL and mediates activation of GFRAL-Ret receptor complex. The proteins of the isolated complex find use in regulating body weight as well as identification of agents that modulate body weight.

“GDNF Family Receptor Alpha Like” (GFRAL) is also known as GRAL. As used herein, “GFRAL” refers to a protein having the amino acid sequence that is at least 65% identical to the amino acid sequence of SEQ ID NO: 1. SEQ ID NO: 1 is the sequence of mature human GFRAL that lacks the signal peptide:

(SEQ ID NO: 1)
qtnnctylreqclrdangckhawrvmedacndsdpgdpckmrnssycnlsi
qylvesnfqfkeclctddfyctvnkllgkkcinksdnykedkfkwnlttrs
hhgfkgmwsclevaeacvgdvvcnaqlasylkacsangnpcdlkqcqaair
ffyqnipfniaqmlafcdcaqsdipcqqskealhsktcavnmvppptclsv
irscqndelcrrhyrtfqskcwqrvtrkchedencistlskqdltcsgsdd
ckaayidilgtvlqvqctcrtitqseeslckifqhmlhrkscfnyptlsnv
kgmalytrkhankitltgfhspfngeviyaamcmtvtcgilllvmvklrts
risskardpssiqipgel

The amino acid sequence of a full-length precursor human GFRAL protein is provided below, which includes a signal peptide sequence (underlined and lowercase residues):

(SEQ ID NO: 9)
mivfiflamglsleneytsQTNNCTYLREQCLRDANGCKHAWRVMEDACN
DSDPGDPCKMRNSSYCNLSIQYLVESNFQFKECLCTDDFYCTVNKLLGKK
CINKSDNVKEDKFKWNLTTRSHHGFKGMWSCLEVAEACVGDVVCNAQLAS
YLKACSANGNPCDLKQCQAAIRFFYQNIPFNIAQMLAFCDCAQSDIPCQQ
SKEALHSKTCAVNMVPPPTCLSVIRSCQNDELCRRHYRTFQSKCWQRVTR
KCHEDENCISTLSKQDLTCSGSDDCKAAYIDILGTVLQVQCTCRTITQSE
ESLCKIFQHMLHRKSCFNYPTLSNVKGMALYTRKHANKITLTGFHSPFNG
EVIYAAMCMTVTCGILLLVMVKLRTSRISSKARDPSSIQIPGEL

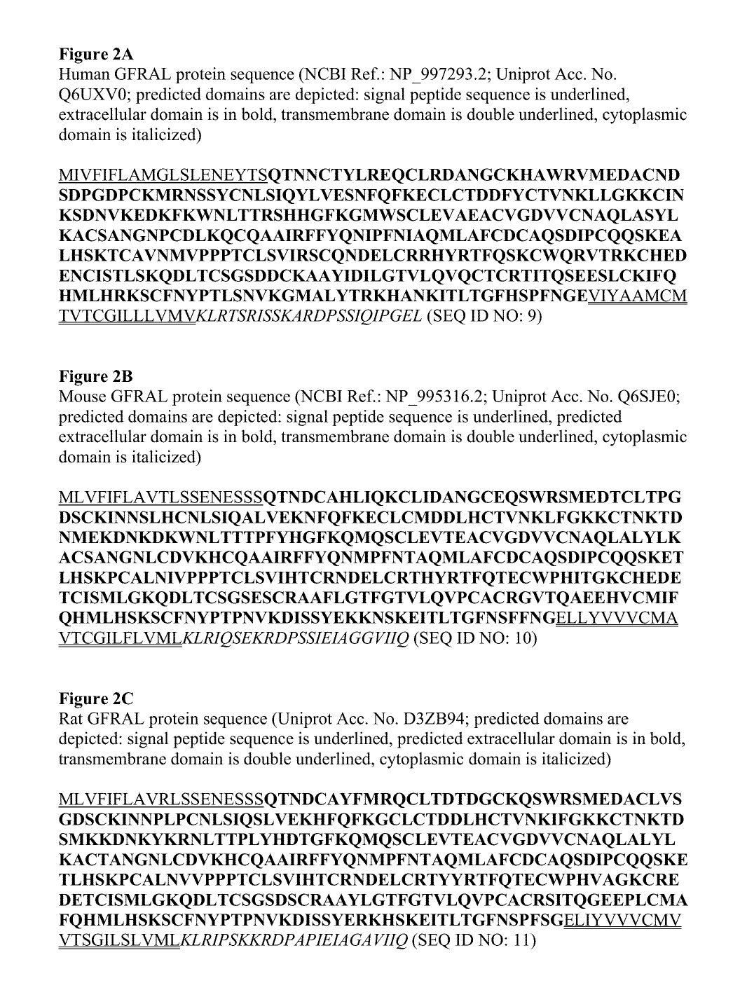

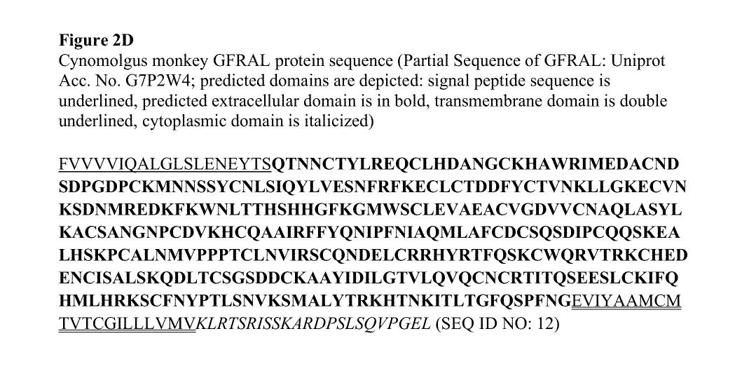

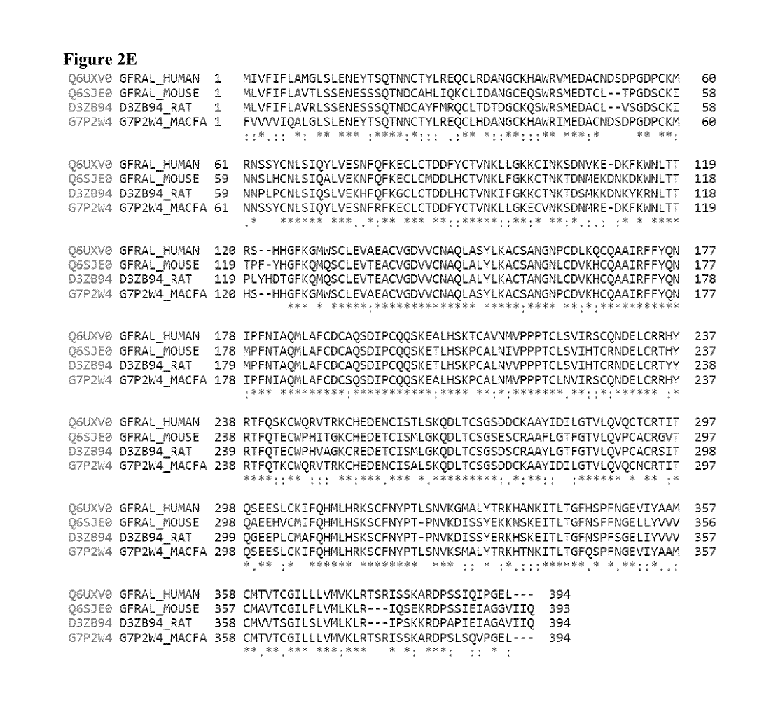

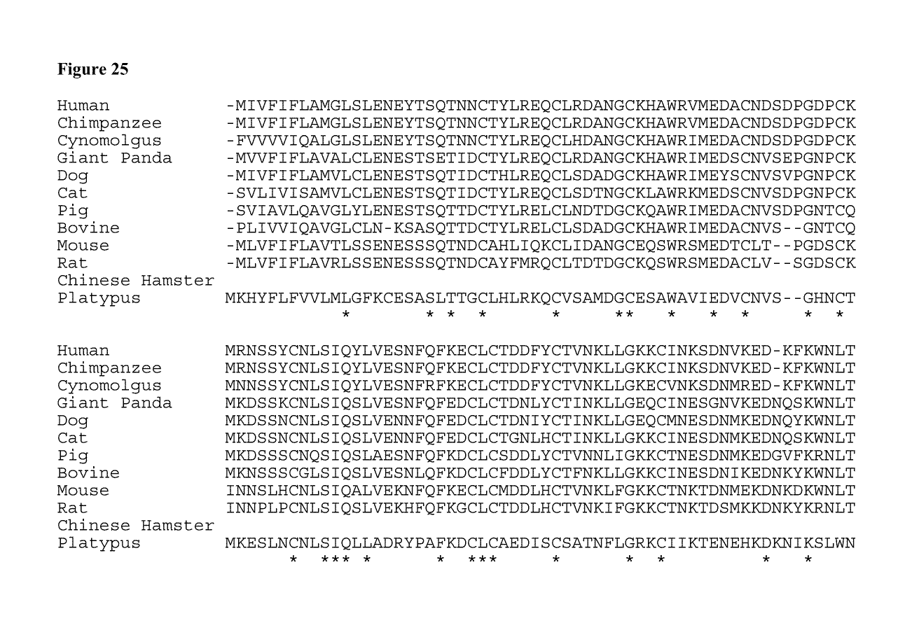

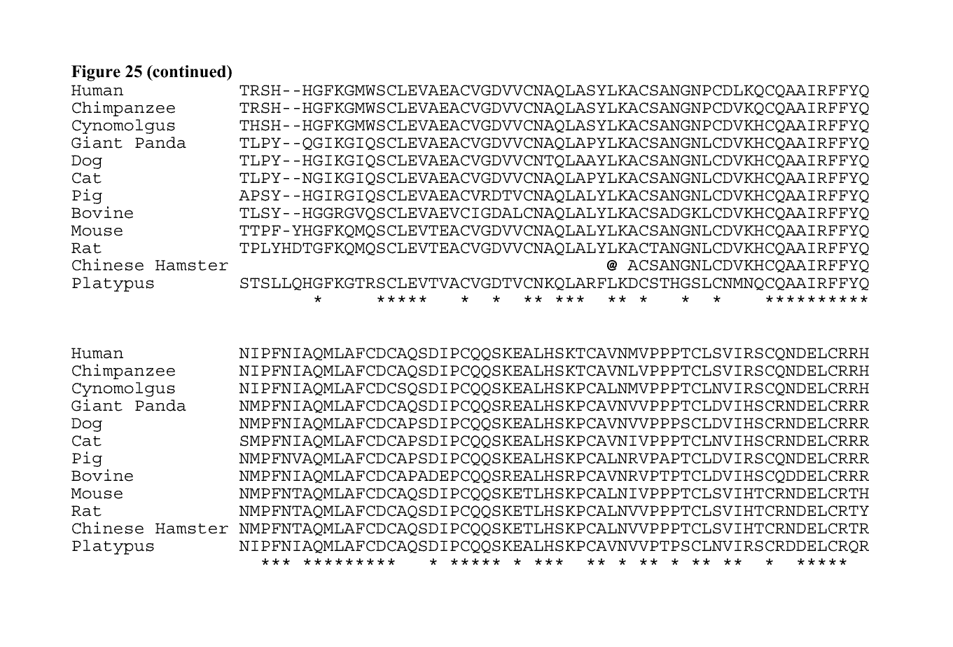

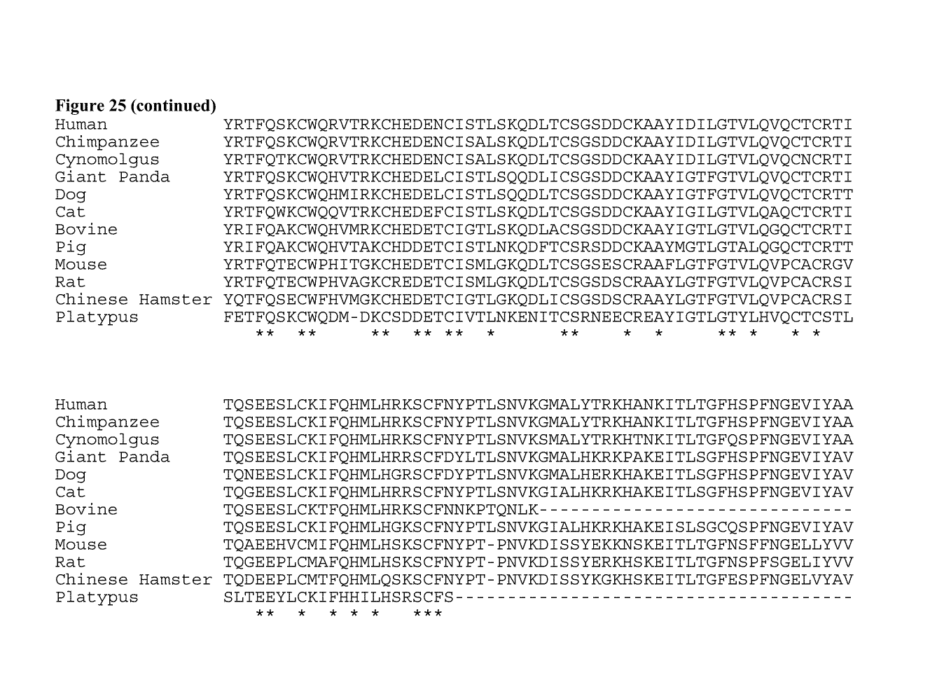

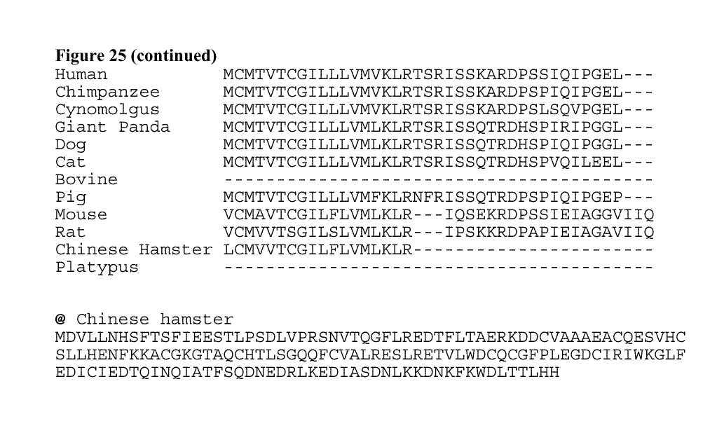

Accordingly, “GFRAL” as used herein encompasses human GFRAL and variants thereof, including but not limited to orthologs thereof, such as murine GFRAL, rat GFRAL, cyno GFRAL, and the like. Such sequences of GFRAL are depicted in FIG. 2. GFRAL is not TGFβ RII (Acc. Nos.: NM_001024847.2; NM_003242.5) or orthologs thereof. GFRAL is distinct from TGFβ RI (Acc. Nos.: NP_001124388.1; NP_004603.1) or orthologs thereof. In certain embodiments, GFRAL may be a protein having the amino acid sequence that is at least 55%, 60%, 65%, 70%, 75%, 80%, 85%, 90%, 95%, 96%, 97%, 98%, 99% or more identical to SEQ ID NO: 1. Such exemplary GFRAL proteins include chimpanzee (99%), cynomolgus monkey (92%), giant panda (82%), dog (81%), cat (80%), pig (77%), bovine (75%), mouse (70%), rat (70%), Chinese hamster (65%), and platypus (59%), as shown in FIG. 25

A GFRAL protein or GFRAL also refers to proteins that have one or more alteration in the amino acid residues (e.g., at locations that are not conserved across variants and/or species) while retaining the conserved domains and having a biological activity similar to the naturally-occurring GFRAL. GFRAL may be encoded by nucleic acid sequences that vary in one or more bases from a naturally-occurring DNA sequence but still translate into an amino acid sequence that corresponds to the a naturally-occurring protein due to degeneracy of the genetic code. GFRAL may also refer to those proteins that differ from the naturally-occurring sequences of GFRAL by one or more conservative substitutions and/or tags and/or conjugates.

Proteins of the present disclosure contain a contiguous amino acid residues of any length derived from GFRAL. A sufficient length of contiguous amino acid residues may vary depending on the specific naturally-occurring amino acid sequence from which the protein is derived. For example, the protein may be at least 100 amino acids to 150 amino acid residues in length, at least 150 amino acids to 200 amino acid residues in length, or at least 220 amino acids up to the full-length protein (e.g., 250 amino acids, 300 amino acids, 319 amino acids, 333 amino acids, 376 amino acids).

A protein containing an amino acid sequence that is substantially similar to the amino acid sequence of a GFRAL polypeptide includes a polypeptide comprising an amino acid sequence having at least about 70%, at least about 75%, at least about 80%, at least about 85%, at least about 90%, at least about 95%, at least about 98%, or at least about 99%, amino acid sequence identity to a contiguous stretch of from about 100 amino acids (aa) to about 150 aa, from about 150 aa to about 200 aa, from about 200 aa to about 250 aa, from about 250 aa to about 300, or from about 300 aa up to the full length of a naturally occurring GFRAL polypeptide. For example, a GFRAL polypeptide of the subject compositions and methods can comprise an amino acid sequence having at least about 71%, at least about 75%, at least about 80%, at least about 85%, at least about 90%, at least about 95%, at least about 98%, or at least about 99%, amino acid sequence identity to a contiguous stretch of from about 100 amino acids (aa) to about 150 aa, from about 150 aa to about 200 aa, from about 200 aa to about 250 aa, from about 250 aa to about 300, or from about 300 aa up to about 350 aa, from about 350 aa to about full length, of the GFRAL polypeptide amino acid sequence depicted in FIGS. 2A-2D.

The protein may lack at least 5, at least 10, up to at least 50 or more aa relative to a naturally-occurring full-length GFRAL polypeptide. For example, the protein may not contain the signal sequence based on the amino acid sequence of a naturally-occurring GFRAL polypeptide. The protein may also contain the same or similar post-translational modifications as a naturally-occurring GFRAL polypeptide or may not contain a post-translational modification. For example, the protein may have the same or similar glycosylation pattern as those of a naturally-occurring GFRAL polypeptide or may contain no glycosylation. In other embodiments, the GFRL protein may include mutations relative to the sequence of naturally-occurring GFRAL protein that introduce a glycosylation site at a location not present in the naturally-occurring GFRAL protein.

Many DNA and protein sequences of GFRAL are known in the art and certain sequences are discussed later below. Certain GFRAL protein sequences are depicted in FIGS. 2A-2E. FIG. 2E shows an alignment of the GFRAL sequences shown in FIGS. 2A-2D.

In certain embodiments, GFRAL may be expressed by a recombinant cell genetically modified to express a GFRAL protein on its cell surface. The cell may be present in a composition that includes an isolated GDF15 protein. In certain cases, the cell may additionally express RET—for example the cell may express RET endogenously without being genetically modified to include an exogenous sequence encoding RET. In other embodiments, the cell may not express detectable levels of RET and may be genetically modified to express RET from an exogenous sequence.

Also disclosed herein are fragments of GFRAL, such as GFRAL fragments that lack an intracellular domain present in native GFRAL, or the intracellular domain and the transmembrane domain present in native GFRAL, such as the native GFRAL depicted in FIGS. 2A-2D. As noted above, a fragment of GFRAL may also lack a signal sequence present in the native GFRAL and may or may not include a heterologous signal sequence. The fragment may lack the intracellular domain present in native GFRAL but include the transmembrane domain.

In certain embodiments, an isolated GFRAL-extracellular domain (GFRAL-ECD) polypeptide is provided. The GFRAL-ECD may be bound to a ligand such as GDF15 when present in the isolated protein complex of the present disclosure. The term “GFRAL-extracellular domain” (“GFRAL-ECD”) includes full-length GFRAL ECDs, GFRAL ECD fragments, and GFRAL ECD variants. As used herein, the term “GFRAL ECD” refers to a GFRAL polypeptide with or without a signal peptide that lacks the intracellular and transmembrane domains. In some embodiments, the GFRAL ECD refers to a protein having the amino acid sequence that is at least 70% identical to the amino acid sequence of human full-length GFRAL ECD having the amino acid sequence:

(SEQ ID NO: 2)
qtnnctylreqclrdangckhawrvmedacndsdpgdpckmrnssycnls
iqylvesnfqfkeclctddfyctvnkllgkkcinksdnvkedkfkwnltt
rshhgfkgmwsclevaeacvgdvvcnaqlasylkacsangnpcdlkqcqa
airffyqnipfniaqmlafcdcaqsdipcqqskealhsktcavnmvpppt
clsvirscqndelcrrhyrtfqskcwqrvtrkchedencistlskqdltc
sgsddckaayidilgtvlqvqctcrtitqseeslckifqhmlhrkscfny
ptlsnvkgmalytrkhankitltgfhspfnge

The term “full-length GFRAL ECD”, as used herein, refers to a GFRAL ECD that extends to the last amino acid of the extracellular domain, and may or may not include an N-terminal signal peptide. However, it is noted that “full-length GFRAL ECD” also encompasses GFRAL-ECD that are extended by 1, 2, 3, 4, 5, 6, 7, 8, 9, or 10 amino acids on the C-terminus to include amino acids residues of the transmembrane domain provided that the polypeptide is soluble. In other words, the GFRAL ECD lacks a sufficient length of a transmembrane domain such that it is not anchored into a cell membrane. The phrase “full-length GFRAL ECD” also encompasses GFRAL-ECD that are extended by 1, 2, 3, 4, 5, 6, 7, 8, 9, or 10 amino acids on the N-terminus to include amino acids residues of the signal peptide. In certain embodiments, GFRAL ECD fragment refers to a contiguous amino acid sequence that is at least 70%, 75%, 80%, 85%, 90%, 95%, 96%, 97%, 98%, or more identical to a contiguous amino acid sequence depicted in FIGS. 2A-2D and lacks at least 30, 33, 35, 40, 45, 50, or 55 amino acids or more at the C-terminus of the GFRAL sequences depicted in FIGS. 2A-2D.

GFRAL ECD is not ECD of TGFβ RII (Acc. Nos.: NM_001024847.2; NM_003242.5) or orthologs thereof. GFRAL ECD is distinct from ECD of TGFβ RI (Acc. Nos.: NP_001124388.1. NP_004603.1) or orthologs thereof. In certain embodiments, GFRAL ECD may be a protein having the amino acid sequence that is at least 70%, 75%, 80%, 85%, 90%, 95%, 96%, 97%, 98%, 99% or more identical to SEQ ID NO: 2.

As used herein, the term “GFRAL ECD fragment” refers to a GFRAL ECD having one or more residues deleted from the N and/or C terminus of the full-length ECD and that retains the ability to bind to GDF15. In some instances, the GFRAL ECD fragment may or may not include an N-terminal signal peptide. In some instances, the GFRAL ECD fragment is a human GFRAL ECD fragment that lacks 1, 5, 10, 15, 16, 17, 18, or 19 residues present at the N-terminus of the sequence:

(SEQ ID NO: 4)
mivfiflamglsleneytsqtnnctylreqclrdangckhawrvmedacn
dsdpgdpckmrnssycnlsiqylvesnfqfkeclctddfyctvnkllgkk
cinksdnvkedkfkwnlttrshhgfkgmwsclevaeacvgdvvcnaqlas
ylkacsangnpcdlkqcqaairffyqnipfniaqmlafcdcaqsdipcqq
skealhsktcavnmvppptclsvirscqndelcrrhyrtfqskcwqrvtr
kchedencistlskqdltcsgsddckaayidilgtvlqvqctcrtitqse
eslckifqhmlhrkscfnyptlsnvkgmalytrkhankitltgfhspfng
e

Another exemplary GFRAL ECD fragment comprises the following amino acid sequence, which corresponds to Q20 to C316 of a full-length human precursor GFRAL protein:

(SEQ ID NO: 22)
qtnnctylreqclrdangckhawrvmedacndsdpgdpckmrnssycnls
iqylvesnfqfkeclctddfyctvnkllgkkcinksdnvkedkfkwnltt
rshhgfkgmwsclevaeacvgdvvcnaqlasylkacsangnpcdlkqcqa
airffyqnipfniaqmlafcdcaqsdipcqqskealhsktcavnmvpppt
clsvirscqndelcrrhyrtfqskcwqrvtrkchedencistlskqdltc
sgsddckaayidilgtvlqvqctcrtitqseeslckifqhmlhrksc

Yet another exemplary GFRAL ECD fragment comprises the following amino acid sequence, which corresponds to W115 to E351 of a full-length human precursor GFRAL protein:

(SEQ ID NO: 23)
wnlttrshhgfkgmwsclevaeacvgdvvcnaqlasylkacsangnpcdl
kqcqaairffyqnipfniaqmlafcdcaqsdipcqqskealhsktcavnm
vppptclsvirscqndelcrrhyrtfqskcwqrvtrkchedencistlsk
qdltcsgsddckaayidilgtvlqvqctcrtitqseeslckifqhmlhrk
scfnyptlsnvkgmalytrkhankitltgfhspfnge.

The above exemplary GFRAL ECD fragment was used in the methods described in the Examples to produce a crystal of a complex comprising a GFRAL protein and a GDF15 protein.

Within the GFRAL ECD there are three separate domains—domain 1 (D1), domain 2 (D2) and domain 3 (D3). In some embodiments, the amino acid sequence demarcating D1 of the GFRAL ECD are residues Q20 to S130 of SEQ ID NO: 9. In some embodiments, the amino acid sequence demarcating D2 of the GFRAL ECD are residues C131 to C210 of SEQ ID NO: 9. In some embodiments, the amino acid sequence demarcating D3 of the GFRAL ECD are residues C220 to C316. Certain properties of GFRAL can be attributed to the activity and/or binding of these domains within the ECD. For example, as described herein, amino acid residues within D2 of the GFRAL ECD are identified as being core interaction interface amino acids and/or boundary interaction interface amino acids for GFRAL binding to GDF15. Likewise, as described herein, amino acid residues within D3 of the GFRAL ECD are identified as being core interaction interface amino acids and/or boundary interaction interface amino acids for GFRAL binding to RET.

The term “core interaction interface amino acid” or grammatical equivalent thereof refers to an amino acid residue of a given protein that has at least one atom within less or equal to 4.5 Å from an interacting protein (e.g., an amino acid on GFRAL that interacts with GDF15 or RET). A distance of 4.5 Å allows for atoms within a van der Waals radius plus a possible water-mediated hydrogen bond to form a bond with the interacting protein.

The term “boundary interaction interface amino acid” or grammatical equivalent thereof refers to an amino acid residue of a given protein that has at least one atom within less than or equal to 5 Å from a core interface amino acid on the given protein (e.g., an amino acid on GFRAL that is within 5 Å of a core interaction interface amino acid on GFRAL that interacts with GDF15 or RET). A distance of less than or equal to 5 Å allows proteins binding to residues less than 5 Å away from core interaction interface amino acids on a given protein to be within the van der Waals radius of an interacting protein.

As used herein, the term “GFRAL ECD variants” refers to GFRAL ECDs that contain amino acid additions, deletions, or substitutions and that remain capable of binding to GDF15. Such variants may be at least 80%, 85%, 90%, 92%, 95%, 97%, 98%, or 99% identical to the parent GFRAL ECD. The % identity of two polypeptides can be measured by a similarity score determined by comparing the amino acid sequences of the two polypeptides using an algorithm, such as, the Bestfit program with the default settings for determining similarity. Bestfit uses the local homology algorithm of Smith and Waterman, Advances in Applied Mathematics 2:482-489 (1981) to find the best segment of similarity between two sequences.

In certain embodiments, the GFRAL-ECD may include a soluble polypeptide that includes a contiguous amino acid sequence about 100-340 residues in length (for example, 100, 150, 200, 250, 300, 310, 320, 330, 333, or 335 residues long), that is at least 70%, 75%, 80%, 85%, 90%, 91%, 92%, 93%, 94%, 95%, 96%, 97%, 98%, 99% or 100% identical to the human soluble GFRAL-ECD:

(SEQ ID NO: 5)
qtnnctylreqclrdangckhawrvmedacndsdpgdpckmrnssycnls
iqylvesnfqfkeclctddfyctvnkllgkkcinksdnvkedkfkwnltt
rshhgfkgmwsclevaeacvgdvvcnaqlasylkacsangnpcdlkqcqa
airffyqnipfniaqmlafcdcaqsdipcqqskealhsktcavnmvpppt
clsvirscqndelcrrhyrtfqskcwqrvtrkchedencistlskqdltc
sgsddckaayidilgtvlqvqctcrtitqseeslckifqhmlhrkscfny
ptlsnvkgmalytrkhankitltgfhspfnge

In certain embodiments, a soluble GFRAL-ECD may be about 325, 329, 330, 331, 332, 335, 340 amino acids long and may be at least 75%, 80%, 85%, 90%, 95% or 99% identical to the human soluble GFRAL-ECD, mouse soluble GFRAL-ECD, or rat soluble GFRAL-ECD.

In certain cases, the GFRAL-extracellular domain may be expressed on the surface of a cell genetically modified to express the GFRAL-ECD with a transmembrane domain. In certain cases, the soluble GFRAL-ECD may be immobilized on a support. As noted herein, the polypeptides of the present disclosure may be fusion proteins that include the polypeptide conjugated to a heterologous protein sequence.

Suitable supports may have a variety of forms and compositions and may derive from naturally occurring materials, naturally occurring materials that have been synthetically modified, or synthetic materials. Examples of suitable materials include, but are not limited to, nitrocellulose, glasses, silicas, teflons, and metals (for example, gold, platinum, and the like). Suitable materials also include polymeric materials, including plastics (for example, polytetrafluoroethylene, polypropylene, polystyrene, polycarbonate, and blends thereof, and the like), polysaccharides such as agarose (e.g., that available commercially as Sepharose®, from Pharmacia) and dextran (e.g., those available commercially under the tradenames Sephadex® and Sephacyl®, also from Pharmacia), polyacrylamides, polystyrenes, polyvinyl alcohols, copolymers of hydroxyethyl methacrylate and methyl methacrylate, and the like.

Also contemplated herein is a composition that includes a RET protein and a GDF15 protein. In certain cases, such a composition may further include GFRAL. In certain cases, RET may be attached to a support or expressed on cell surface of a recombinant cell genetically modified to express RET. In certain cases, the composition may include a recombinant cell genetically modified to express RET; GDF15; and a carrier, such as a pharmaceutically acceptable carrier.

In certain embodiments, a composition may include GFRAL and RET. In certain cases, the composition may include a recombinant cell genetically modified to express RET and GFRAL; and a carrier, such as a pharmaceutically acceptable carrier.

In certain cases, the composition may include a recombinant cell genetically modified to express RET and GFRAL; GDF15; a carrier, such as a pharmaceutically acceptable carrier.

As used herein, “Ret” or “RET” refers to a protein having the amino acid sequence that is at least 75% identical, e.g., 77%, 79%, 80%, 85%, 90%, 95%, 96%, 97%, 98%, 99% or more identical to the amino acid sequence of SEQ ID NO: 3. RET is distinct from TGFβ RI and TGFβ RII. SEQ ID NO: 3 is the sequence of mature human RET9 that lacks a signal peptide:

(SEQ ID NO: 3)
KVALGLYFSRDAYWEKLYVDQAAGTPLLYVHALRDAPEEVPSFRLGQHL
YGTYRTRLHENNWICIQEDTGLLYLNRSLDHSSWEKLSVRNRGFPLLTVY
LKVFLSPTSLREGECQWPGCARVYFSFFNTSFPACSSLKPRELCFPETRP
SFRIRENRPPGTFHQFRLLPVQFLCPNISVAYRLLEGEGLPFRCAPDSLE
VSTRWALDREQREKYELVAVCTVHAGAREEVVMVPFPVTVYDEDDSAPTF
PAGVDTASAVVEFKRKEDTVVATLRVFDADVVPASGELVRRYTSTLLPGD
TWAQQTFRVEHWPNETSVQANGSFVRATVHDYRLVLNRNLSISENRTMQL
AVLVNDSDFQGPGAGVLLLHFNVSVLPVSLHLPSTYSLSVSRRARRFAQI
GKVCVENCQAFSGINVQYKLHSSGANCSTLGVVTSAEDTSGILFVNDTKA
LRRPKCAELHYMVVATDQQTSRQAQAQLLVTVEGSYVAEEAGCPLSCAVS
KRRLECEECGGLGSPTGRCEWRQGDGKGITRNFSTCSPSTKTCPDGHCDV
VETQDINICPQDCLRGSIVGGHEPGEPRGIKAGYGTCNCFPEEEKCFCEP
EDIQDPLCDELCRTVIAAAVLFSFIVSVLLSAFCIHCYHKFAHKPPISSA
EMTFRRPAQAFPVSYSSSGARRPSLDSMENQVSVDAFKILEDPKWEFPRK
NLVLGKTLGEGEFGKVVKATAFHLKGRAGYTTVAVKMLKENASPSELRDL
LSEFNVLKQVNHPHVIKLYGACSQDGPLLLIVEYAKYGSLRGFLRESRKV
GPGYLGSGGSRNSSSLDHPDERALTMGDLISFAWQISQGMQYLAEMKLVH
RDLAARNILVAEGRKMKISDFGLSRDVYEEDSYVKRSQGRIPVKWMAIES
LFDHIYTTQSDVWSFGVLLWEIVTLGGNPYPGIPPERLFNLLKTGHRMER
PDNCSEEMYRLMLQCWKQEPDKRPVFADISKDLEKMMVKRRDYLDLAAST
PSDSLIYDDGLSEEETPLVDCNNAPLPRALPSTWIENKLYGRISHAFTRF

The amino acid sequence of a full-length precursor human RET protein is provided below, which includes a signal peptide sequence (underlined and lowercase residues):

(SEQ ID NO: 26)
makatsgaaglrlllllllpllgKVALGLYFSRDAYWEKLYVDQAAGTPL
LYVHALRDAPEEVPSFRLGQHLYGTYRTRLHENNWICIQEDTGLLYLNRS
LDHSSWEKLSVRNRGFPLLTVYLKVFLSPTSLREGECQWPGCARVYFSFF
NTSFPACSSLKPRELCFPETRPSFRIRENRPPGTFHQFRLLPVQFLCPNI
SVAYRLLEGEGLPFRCAPDSLEVSTRWALDREQREKYELVAVCTVHAGAR
EEVVMVPFPVTVYDEDDSAPTFPAGVDTASAVVEFKRKEDTVVATLRVFD
ADVVPASGELVRRYTSTLLPGDTWAQQTFRVEHWPNETSVQANGSFVRAT
VHDYRLVLNRNLSISENRTMQLAVLVNDSDFQGPGAGVLLLHFNVSVLPV
SLHLPSTYSLSVSRRARRFAQIGKVCVENCQAFSGINVQYKLHSSGANCS
TLGVVTSAEDTSGILFVNDTKALRRPKCAELHYMVVATDQQTSRQAQAQL
LVTVEGSYVAEEAGCPLSCAVSKRRLECEECGGLGSPTGRCEWRQGDGKG
ITRNFSTCSPSTKTCPDGHCDVVETQDINICPQDCLRGSIVGGHEPGEPR
GIKAGYGTCNCFPEEEKCFCEPEDIQDPLCDELCRTVIAAAVLFSFIVSV
LLSAFCIHCYHKFAHKPPISSAEMTFRRPAQAFPVSYSSSGARRPSLDSM
ENQVSVDAFKILEDPKWEFPRKNLVLGKTLGEGEFGKVVKATAFHLKGRA
GYTTVAVKMLKENASPSELRDLLSEFNVLKQVNHPHVIKLYGACSQDGPL
LLIVEYAKYGSLRGFLRESRKVGPGYLGSGGSRNSSSLDHPDERALTMGD
LISFAWQISQGMQYLAEMKLVHRDLAARNILVAEGRKMKISDFGLSRDVY
EEDSYVKRSQGRIPVKWMAIESLFDHIYTTQSDVWSFGVLLWEIVTLGGN
PYPGIPPERLFNLLKTGHRMERPDNCSEEMYRLMLQCWKQEPDKRPVFAD
ISKDLEKMMVKRRDYLDLAASTPSDSLIYDDGLSEEETPLVDCNNAPLPR
ALPSTWIENKLYGRISHAFTRF

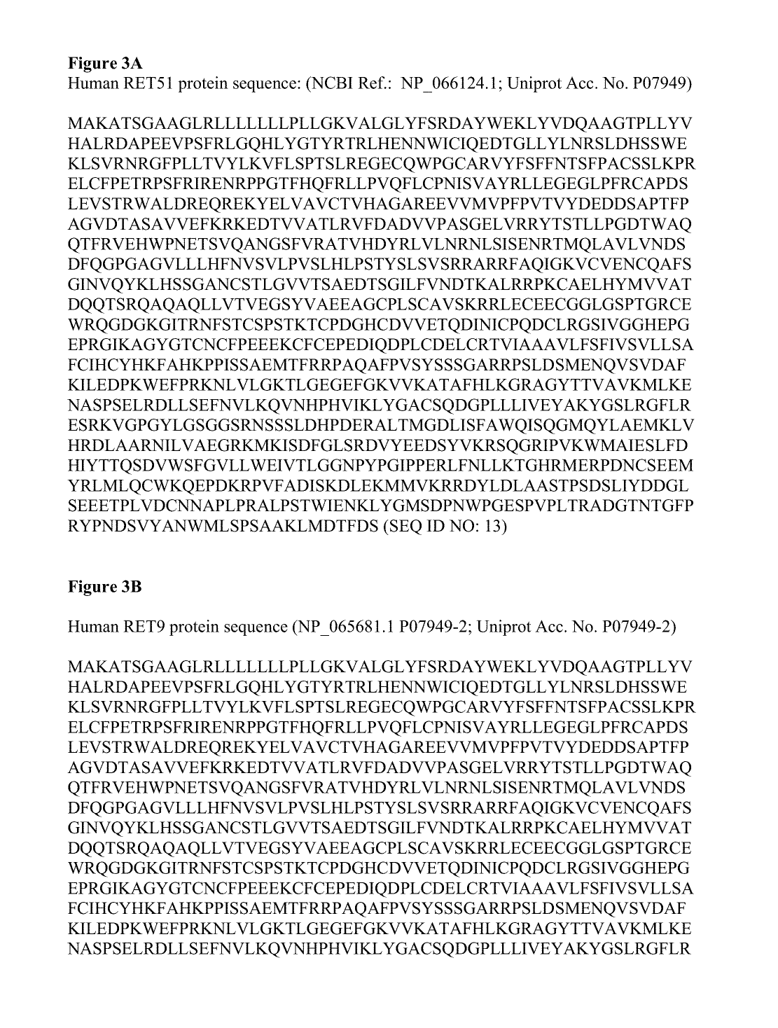

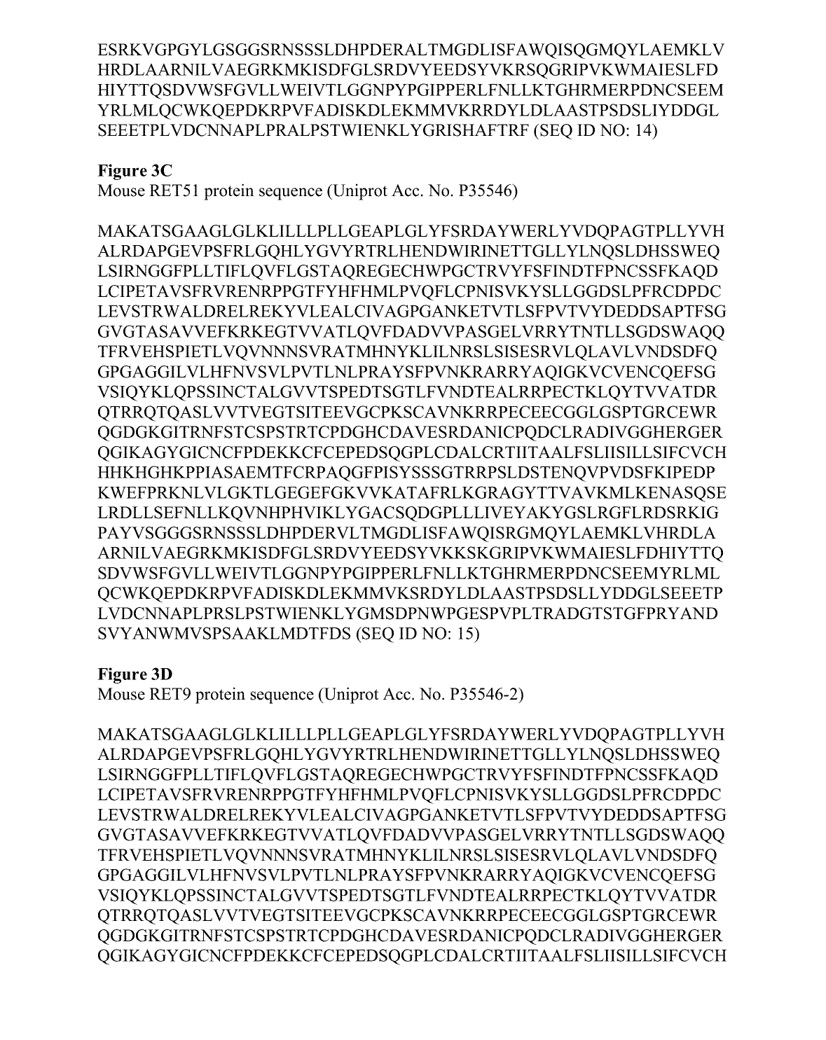

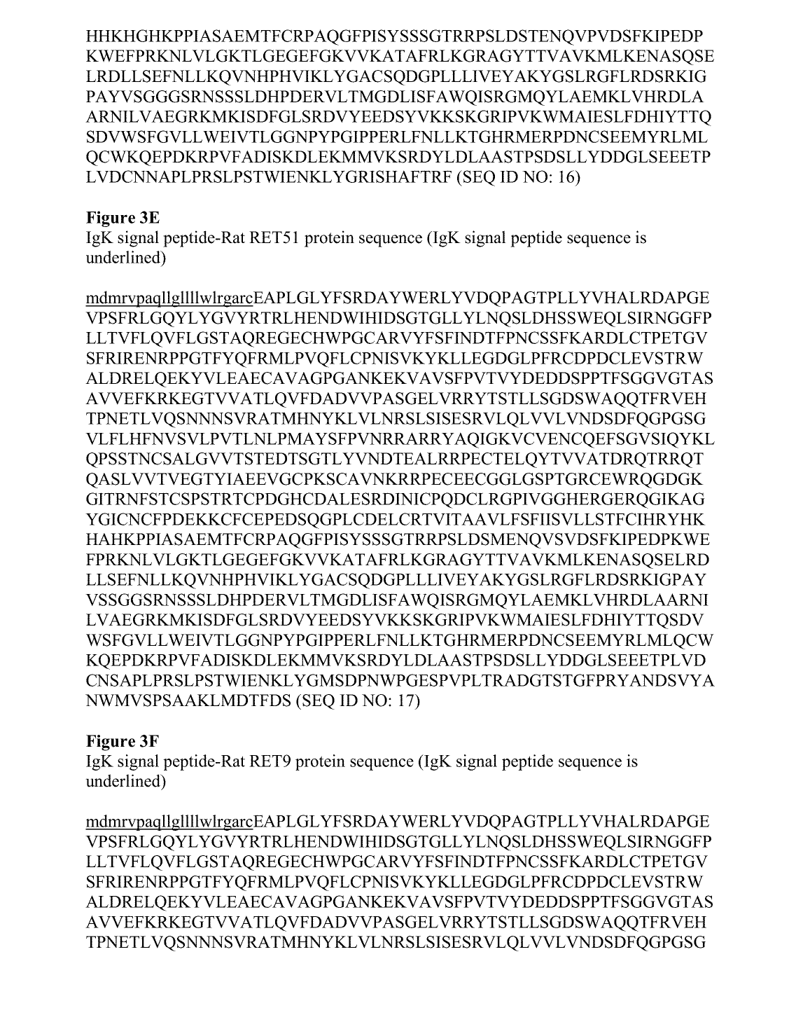

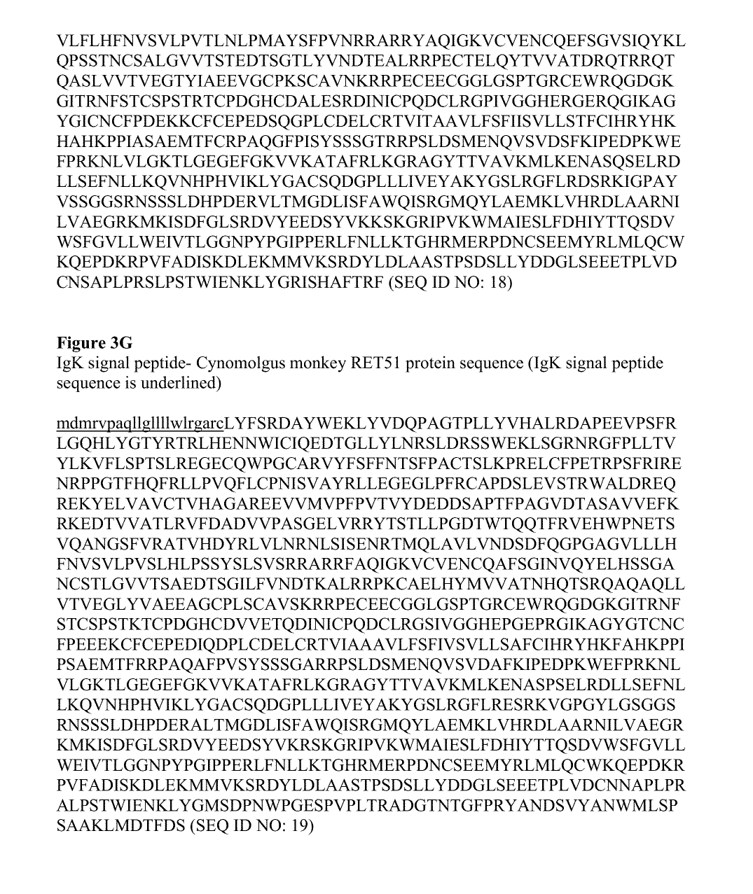

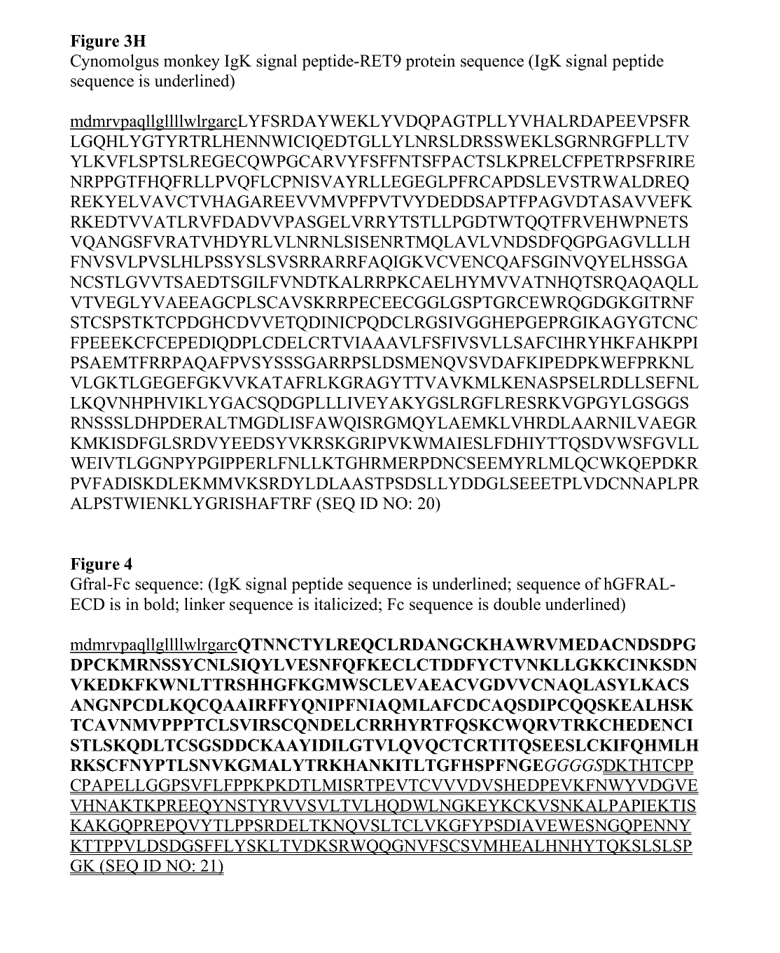

Accordingly, “RET” as used herein encompasses human RET and variants thereof, including but not limited to orthologs thereof, such as murine RET, cyno RET, and the like. Such sequences of RET are depicted in FIG. 3. In certain embodiments, RET may be a protein having the amino acid sequence that is at least 75%, 80%, 85%, 90%, 95%, 96%, 97%, 98%, 99% or more identical to SEQ ID NO: 3.

In certain embodiments, an isolated RET-extracellular domain (RET-ECD) polypeptide is provided. The RET-ECD may be bound to a ligand such as GFRAL when present in the isolated protein complex of the present disclosure. The term “RET-extracellular domain” (“RET-ECD”) includes full-length RET ECDs, RET ECD fragments, and RET ECD variants. As used herein, the term “RET ECD” refers to a RET polypeptide with or without a signal peptide that lacks the intracellular and transmembrane domains. In some embodiments, the RET ECD refers to a protein having the amino acid sequence that is at least 75% identical to the amino acid sequence of human full-length RET ECD having the amino acid sequence:

(SEQ ID NO: 27)
KVALGLYFSRDAYWEKLYVDQAAGTPLLYVHALRDAPEEVPSFRLGQHL
YGTYRTRLHENNWICIQEDTGLLYLNRSLDHSSWEKLSVRNRGFPLLTVY
LKVFLSPTSLREGECQWPGCARVYFSFFNTSFPACSSLKPRELCFPETRP
SFRIRENRPPGTFHQFRLLPVQFLCPNISVAYRLLEGEGLPFRCAPDSLE
VSTRWALDREQREKYELVAVCTVHAGAREEVVMVPFPVTVYDEDDSAPTF
PAGVDTASAVVEFKRKEDTVVATLRVFDADVVPASGELVRRYTSTLLPGD
TWAQQTFRVEHWPNETSVQANGSFVRATVHDYRLVLNRNLSISENRTMQL
AVLVNDSDFQGPGAGVLLLHFNVSVLPVSLHLPSTYSLSVSRRARRFAQI
GKVCVENCQAFSGINVQYKLHSSGANCSTLGVVTSAEDTSGILFVNDTKA
LRRPKCAELHYMVVATDQQTSRQAQAQLLVTVEGSYVAEEAGCPLSCAVS
KRRLECEECGGLGSPTGRCEWRQGDGKGITRNFSTCSPSTKTCPDGHCDV
VETQDINICPQDCLRGSIVGGHEPGEPRGIKAGYGTCNCFPEEEKCFCEP
EDIQDPLCDELCR

In another exemplary embodiment, the RET ECD refers to a protein having the amino acid sequence that is at least 75% identical to the amino acid sequence of human full-length RET ECD having the amino acid sequence:

(SEQ ID NO: 28)
LYFSRDAYWEKLYVDQAAGTPLLYVHALRDAPEEVPSFRLGQHLYGTY
RTRLHENNWICIQEDTGLLYLNRSLDHSSWEKLSVRNRGFPLLTVYLKVF
LSPTSLREGECQWPGCARVYFSFFNTSFPACSSLKPRELCFPETRPSFRI
RENRPPGTFHQFRLLPVQFLCPNISVAYRLLEGEGLPFRCAPDSLEVSTR
WALDREQREKYELVAVCTVHAGAREEVVMVPFPVTVYDEDDSAPTFPAGV
DTASAVVEFKRKEDTVVATLRVFDADVVPASGELVRRYTSTLLPGDTWAQ
QTFRVEHWPNETSVQANGSFVRATVHDYRLVLNRNLSISENRTMQLAVLV
NDSDFQGPGAGVLLLHFNVSVLPVSLHLPSTYSLSVSRRARRFAQIGKVC
VENCQAFSGINVQYKLHSSGANCSTLGVVTSAEDTSGILFVNDTKALRRP
KCAELHYMVVATDQQTSRQAQAQLLVTVEGSYVAEEAGCPLSCAVSKRRL
ECEECGGLGSPTGRCEWRQGDGKGITRNFSTCSPSTKTCPDGHCDVVETQ
DINICPQDCLRGSIVGGHEPGEPRGIKAGYGTCNCFPEEEKCFCEPEDIQ
DPLCDELCR

The term “full-length RET ECD”, as used herein, refers to a RET ECD that extends to the last amino acid of the extracellular domain, and may or may not include an N-terminal signal peptide. However, it is noted that “full-length RET ECD” also encompasses RET-ECD that are extended by 1, 2, 3, 4, 5, 6, 7, 8, 9, or 10 amino acids on the C-terminus to include amino acids residues of the transmembrane domain provided that the polypeptide is soluble. In other words, the RET ECD lacks a sufficient length of a transmembrane domain such that it is not anchored into a cell membrane. The phrase “full-length RET ECD” also encompasses RET-ECD that are extended by 1, 2, 3, 4, 5, 6, 7, 8, 9, or 10 amino acids on the N-terminus to include amino acids residues of the signal peptide. In certain embodiments, RET ECD fragment refers to a contiguous amino acid sequence that is at least 70%, 75%, 80%, 85%, 90%, 95%, 96%, 97%, 98%, 99% or more identical to a contiguous amino acid sequence depicted in FIGS. 3A-3H and lacks at least 30, 33, 35, 40, 45, 50, or 55 amino acids or more at the C-terminus of the RET sequences depicted in FIGS. 3A-3H.

As used herein, the term “RET ECD fragment” refers to a RET ECD having one or more residues deleted from the N and/or C terminus of the full-length ECD and that retains the ability to bind to GFRAL. In some instances, the RET ECD fragment may or may not include an N-terminal signal peptide. In some instances, the RET ECD fragment is a human RET ECD fragment that lacks 1, 5, 10, 15, 16, 17, 18, or 19 residues present at the N-terminus of the sequence:

(SEQ ID NO: 29)
LYFSRDAYWEKLYVDQAAGTPLLYVHALRDAPEEVPSFRLGQHLYGTY
RTRLHENNWICIQEDTGLLYLNRSLDHSSWEKLSVRNRGFPLLTVYLKVF
LSPTSLREGECQWPGCARVYFSFFNTSFPACSSLKPRELCFPETRPSFRI
RENRPPGTFHQFRLLPVQFLCPNISVAYRLLEGEGLPFRCAPDSLEVSTR
WALDREQREKYELVAVCTVHAGAREEVVMVPFPVTVYDEDDSAPTFPAGV
DTASAVVEFKRKEDTVVATLRVFDADVVPASGELVRRYTSTLLPGDTWAQ
QTFRVEHWPNETSVQANGSFVRATVHDYRLVLNRNLSISENRTMQLAVLV
NDSDFQGPGAGVLLLHFNVSVLPVSLHLPSTYSLSVSRRARRFAQIGKVC
VENCQAFSGINVQYKLHSSGANCSTLGVVTSAEDTSGILFVNDTKALRRP
KCAELHYMVVATDQQTSRQAQAQLLVTVEGSYVAEEAGCPLSCAVSKRRL
ECEECGGLGSPTGRCEWRQGDGKGITRNFSTCSPSTKTCPDGHCDVVETQ
DINICPQDCLRGSIVGGHEPGEPRGIKAGYGTCNCFPEEEKCFCEPEDIQ
DPLCDELCR

The above exemplary RET ECD fragment was used in the methods described in the Examples to produce a model of a complex comprising a RET protein, a GFRAL protein and a GDF15 protein.

In alternative embodiments of a RET ECD, the RET-ECD comprises a C64R, N75Q, N166Q, or C183S mutation in a RET ECD sequence of SEQ ID NO 27.

The proteins described in the method of the present disclosure include those containing contiguous amino acid sequences of any naturally-occurring GFRAL, as well as those having 1, 2, 3, 4, 5, 6, 7, 8, 9, 10, or more usually no more than 40, 30, 20, 10, or 5 amino acid substitutions, where the substitution is usually a conservative amino acid substitution. The phrase “conservative amino acid substitution” generally refers to substitution of amino acid residues within the following groups:

1) L, I, M, V, F;

2) R, K;

3) F, Y, H, W, R;

4) G, A, T, S;

5) Q, N; and

6) D, E.

Conservative amino acid substitutions in the context of a peptide or a protein disclosed herein are selected so as to preserve putative activity of the protein. Such presentation may be preserved by substituting with an amino acid having a side chain of similar acidity, basicity, charge, polarity, or size to the side chain of the amino acid being replaced. Guidance for substitutions, insertion, and deletion may be based on alignments of amino acid sequences of different variant proteins or proteins from different species. Residues that are semi-conserved (. or :) may tolerate changes that preserve charge, polarity, and/or size. See FIG. 2E for example.

The present disclosure provides any of the polypeptides described above. The protein may be isolated from a natural source, e.g., is in an environment other than its naturally-occurring environment. The subject protein may also be recombinantly made, e.g., in a genetically modified host cell (e.g., bacteria; yeast; insect; mammalian-murine, human cells; and the like), where the genetically modified host cell is genetically modified with a nucleic acid comprising a nucleotide sequence encoding the subject protein. The subject protein encompasses synthetic polypeptides, e.g., a subject synthetic polypeptide is synthesized chemically in a laboratory (e.g., by cell-free in vitro synthesis or chemical synthesis). Methods of productions are described in more detail below.

### Nucleic Acid and Protein Sequences

The subject polypeptide may be generated using recombinant techniques to manipulate nucleic acids of different GFRAL, RET, or GDF15 proteins to provide constructs encoding a protein of interest. It will be appreciated that, provided an amino acid sequence, the ordinarily skilled artisan will immediately recognize a variety of different nucleic acids encoding such amino acid sequence in view of the knowledge of the genetic code.

For production of subject proteins derived from naturally-occurring polypeptides, it is noted that nucleic acids encoding a variety of different polypeptides are known and available in the art. Nucleic acid (and amino acid sequences) for various GFRAL, RET, and GDF15 polypeptides are also provided as accession nos. GFRAL: i) Homo sapiens: amino acid sequence NP_997293.2; nucleotide sequence: NM_207410.2; ii) Mus musculus: amino acid sequence NP_995316.2; nucleotide sequence NM_205844; iii) Rattus norvegicus: amino acid sequence: NP_001178927.1; nucleotide sequence: NM_001191998.1; iv) Macaca fascicularis: amino acid sequence: G7P2W4; v) Gallus gallus: amino acid sequence XP_419904.4; nucleotide sequence XM_419904.4. RET: i) Homo sapiens RET51: amino acid sequence: NP_066124.1; nucleotide sequence: NM_020975.4; ii) Homo sapiens RET9: amino acid sequence: NP_065681.1; nucleotide sequence: NM_020630.4; iii) Mus musculus RET51: amino acid sequence: P35546; nucleotide sequence: NM_001080780.1; iv) Mus musculus RET9: amino acid sequence: P35546-2; nucleotide sequence: NM_001080780.1; v) Rattus norvegicus RET51: amino acid sequence: NP_036775.2 nucleotide sequence: NM_012643.2; vi) Rattus norvegicus RET9: amino acid sequence: NP_001103569.1; nucleotide sequence: NM_001110099.1; vii) Macaca fascicularis RET51: amino acid sequence: XP_005565094.1; nucleotide sequence: XM_005565037.1; viii) Macaca fascicularis RET9: amino acid sequence: XP_005565095.1; nucleotide sequence: XM_005565038.1. Exemplary GFRAL and RET amino acid sequences are depicted in FIGS. 2 and 3, respectively.

“Growth differentiation factor 15” or “GDF15,” also known in the art as MIC-1 (macrophage inhibitory cytokine-1), PDF (prostate differentiation factor), PLAB (placental bone morphogenetic protein), NAG-1 (non-steroidal anti-inflammatory drugs (NSAIDs) activated gene), TGF-PL, and PTGFB, is a member of the transforming growth factor β (TGF-β) super-family. GDF15, which is synthesized as a 62 kDa intracellular precursor protein that is subsequently cleaved by a furin-like protease, is secreted as a 25 kDa disulfide-linked protein (see, e.g., Fairlie et al., J. Leukoc. Biol 65:2-5 (1999)). GDF15 mRNA is seen in several tissues, including liver, kidney, pancreas, colon and placenta, and GDF15 expression in liver can be significantly up-regulated during injury of organs such as the liver, kidneys, heart and lungs.

The GDF15 precursor is a 308 amino acid polypeptide (NCBI Ref. Seq. NP_004855.2; GI:153792495) containing a 29 amino acid signal peptide, a 167 amino acid pro-domain, and a mature domain of 112 amino acids which is excised from the pro-domain by furin-like proteases.

An amino acid sequence of a precursor human GDF15 polypeptide is provided below:

(SEQ ID NO: 25)
MPGQELRTVNGSQMLLVLLVLSWLPHGGALSLAEASRASFPGPSELHS
EDSRFRELRKRYEDLLTRLRANQSWEDSNTDLVPAPAVRILTPEVRLGS
GGHLHLRISRAALPEGLPEASRLHRALFRLSPTASRSWDVTRPLRRQLS
LARPQAPALHLRLSPPPSQSDQLLAESSSARPQLELHLRPQAARGRRRA
RARNGDHCPLGPGRCCRLHTVRASLEDLGWADWVLSPREVQVTMCIG
ACPSQFRAANMHAQIKTSLHRLKPDTVPAPCCVPASYNPMVLIQKTDTG
VSLQTYDDLLAKDCHCI

The 308-amino acid GDF15 polypeptide is referred to as a “full-length” GDF15 polypeptide; a 112-amino acid GDF15 polypeptide (amino acids 197-308 of “full-length” GDF15) is a “mature” GDF15 polypeptide.

“GDF15” as used herein includes a protein having the amino acid sequence that is at least 65% identical to the amino acid sequence of SEQ ID NO: 6. An amino acid sequence of a mature human GDF15 polypeptide is provided below:

(SEQ ID NO: 6)
ARNGDHCPLGPGRCCRLHTVRASLEDLGWADWVLSPREVQVTMCIGA
CPSQFRAANMHAQIKTSLHRLKPDTVPAPCCVPASYNPMVLIQKTDTGV
SLQTYDDLLAKDCHCI

The above exemplary mature human GDF15 was used in the methods described in the Examples to produce a crystal of a complex comprising a GFRAL protein and a GDF15 protein.

Unless otherwise indicated, the term “GDF15” refers to the 112 amino acid mature human sequence. In addition, numerical references to particular GDF15 residues refer to the 112 amino acid mature sequence (i.e., residue 1 is Ala (A), and residue 112 is Ile (I); see SEQ ID NO: 6). Of note, while the GDF15 precursor amino acid sequence predicts three excision sites, resulting in three putative forms of “mature” human GDF15 (i.e., 110, 112 and 115 amino acids), the 112 amino acid mature sequence is accepted as being correct.

In some embodiments, a GDF15 protein is a homodimer (e.g., comprising two polypeptide chains each of SEQ ID NO: 6).

The GDF15 precursor is a 308 amino acid polypeptide (NCBI Ref. Seq. NP_004855.2) containing a 29 amino acid signal peptide, a 167 amino acid pro-domain, and a mature domain of 112 amino acids which is excised from the pro-domain by furin-like proteases. A 308-amino acid GDF15 polypeptide is referred to as a “full-length” GDF15 polypeptide; a 112-amino acid GDF15 polypeptide (see FIG. 1A) is a “mature” GDF15 polypeptide. Unless otherwise indicated, the term “GDF15” refers to the 112 amino acid mature sequence. In addition, numerical references to particular GDF15 residues refer to the 112 amino acid mature sequence (i.e., residue 1 is Ala (A), and residue 112 is Ile (I); see FIG. 1A).

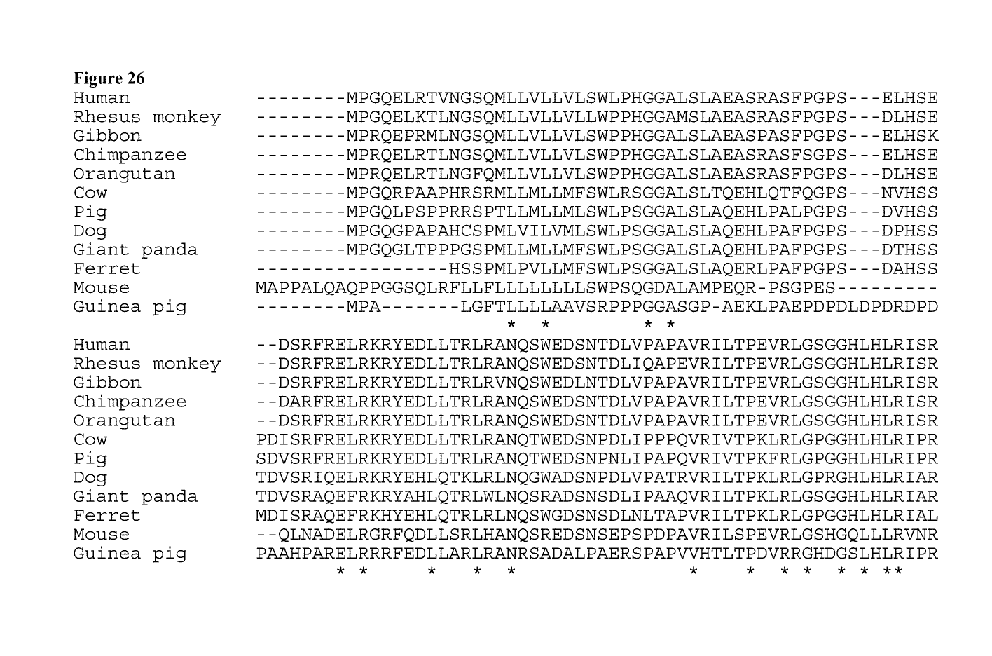

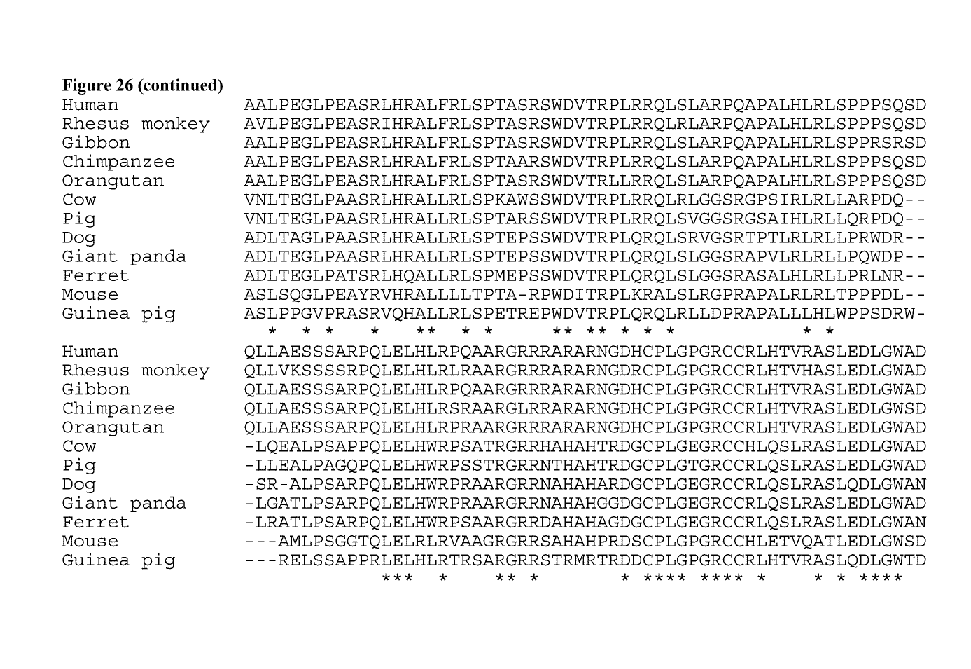

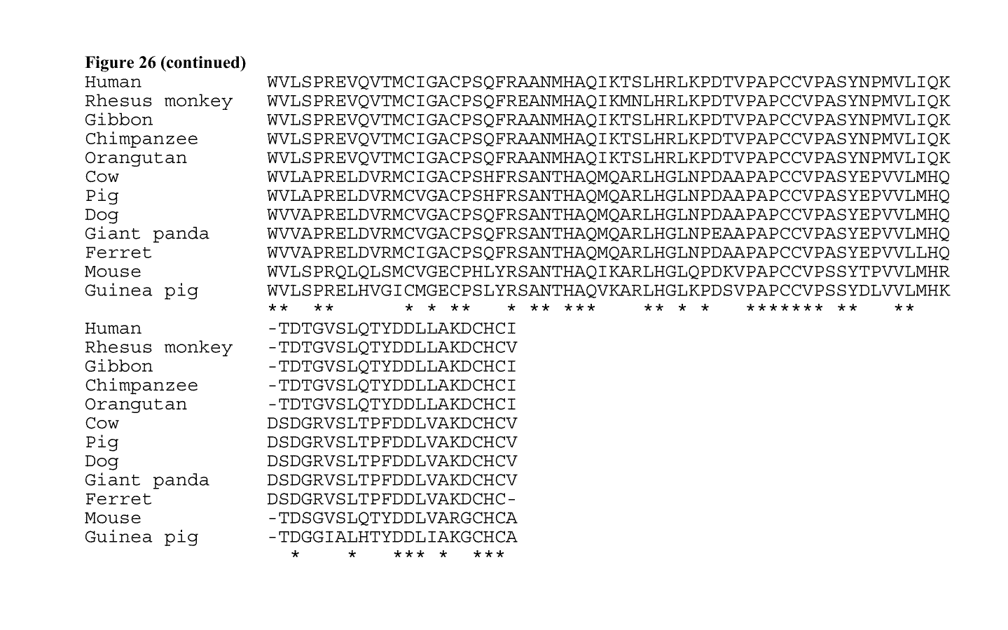

The scope of the present disclosure includes GDF15 orthologs, and modified forms thereof, from other mammalian species, and their use, including mouse (NP_035949), chimpanzee (XP_524157), orangutan (XP_002828972), Rhesus monkey (EHH29815), giant panda (XP_002912774), gibbon (XP_003275874), guinea pig (XP_003465238), ferret (AER98997), cow (NP_001193227), pig (NP_001167527) and dog (XP_541938). Such exemplary GDF15 proteins are shown in FIG. 26, which includes an alignment of the various exemplary GDF15 proteins. The mature form of human GDF15 has approximately 67% amino acid identity to the murine ortholog.

It will be appreciated that the nucleotide sequences encoding the protein may be modified so as to optimize the codon usage to facilitate expression in a host cell of interest (e.g., Escherichia coli, and the like). Methods for production of codon optimized sequences are known in the art.

### Protein Modifications

The proteins used in the present disclosure can be provided as proteins that are modified relative to the naturally-occurring protein. Purposes of the modifications may be to increase a property desirable in a protein formulated for therapy (e.g., serum half-life), to raise antibody for use in detection assays, and/or for protein purification, and the like.

One way to modify a subject protein is to conjugate (e.g., link) one or more additional elements at the N- and/or C-terminus of the protein, such as another protein (e.g., having an amino acid sequence heterologous to the subject protein) and/or a carrier molecule. Thus, an exemplary protein can be provided as fusion proteins with a polypeptide(s) derived from an immunoglobulin Fc polypeptide.

Conjugate modifications to proteins may result in a protein that retains the desired activity, while exploiting properties of the second molecule of the conjugate to impart and/or enhances certain properties (e.g., desirable for therapeutic uses). For example, the polypeptide may be conjugated to a molecule, e.g., to facilitate solubility, storage, half-life, reduction in immunogenicity, controlled release in tissue or other bodily location (e.g., blood or other particular organs, etc.).

Other features of a conjugated protein may include one where the conjugate reduces toxicity relative to an unconjugated protein. Another feature is that the conjugate may target a type of cell or organ more efficiently than an unconjugated material. The protein can optionally have attached a drug to further counter the causes or effects associated with disorders of metabolism (e.g., drug for muscle atrophy), and/or can optionally be modified to provide for improved pharmacokinetic profile (e.g., by PEGylation, hyperglycosylation, and the like).

Where a subject protein is a fusion protein comprising a GFRAL or GDF15 or RET polypeptide and a heterologous fusion partner polypeptide, a subject fusion protein can have a total length that is equal to the sum of the GFRAL or GDF15 or RET polypeptide and the heterologous fusion partner polypeptide and a linker, if present. Exemplary GDF15 fusion proteins are shown in FIG. 1B (Fc fusion) and FIG. 1C (human serum albumin fusion).

Any of the foregoing components and molecules used to modify the polypeptide sequences of the present disclosure may optionally be conjugated via a linker. Suitable linkers include “flexible linkers” which are generally of sufficient length to permit some movement between the modified polypeptide sequences and the linked components and molecules. The linker molecules can be about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1, 2, 3, 4, 5, 6, 7, 8, 9, 10, 10-20, 20-30, 30-50 amino acids.

Exemplary flexible linkers include glycine polymers (G)ₙ, glycine-alanine polymers, alanine-serine polymers, glycine-serine polymers (for example, (GₘSₒ)ₙ, (GSGGS), (SEQ ID NO: 52), (GₘSₒGₘ)ₙ, (GₘSₒGₘSₒGₘ)ₙ (SEQ ID NO: 53), (GSGGSₘ)ₙ (SEQ ID NO: 54), (GSGSₘG)ₙ (SEQ ID NO: 55) and (GGGSₘ)ₙ (SEQ ID NO: 56), and combinations thereof, where m, n, and o are each independently selected from an integer of at least 1 to 20, e.g., 1-18, 2-16, 3-14, 4-12, 5-10, 1, 2, 3, 4, 5, 6, 7, 8, 9, or 10), and other flexible linkers. Glycine and glycine-serine polymers are relatively unstructured, and therefore may serve as a neutral tether between components. Exemplary flexible linkers include, but are not limited to GGSG (SEQ ID NO: 57), GGSGG (SEQ ID NO: 58), GSGSG (SEQ ID NO: 59), GSGGG (SEQ ID NO: 60), GGGSG (SEQ ID NO: 61), and GSSSG (SEQ ID NO: 62).

Additional flexible linkers include glycine polymers (G)ₙ or glycine-serine polymers (e.g., (GS)ₙ, (GSGGS)ₙ (SEQ ID NO: 63), (GGGS)ₙ (SEQ ID NO: 64) and (GGGGS)ₙ (SEQ ID NO: 65), where n=1 to 50, for example, 1, 2, 3, 4, 5, 6, 7, 8, 9, 10, 10-20, 20-30, 30-50. Exemplary flexible linkers include, but are not limited to GGGS (SEQ ID NO: 66), GGGGS (SEQ ID NO: 67), GGSG (SEQ ID NO: 57), GGSGG (SEQ ID NO: 58), GSGSG (SEQ ID NO: 59), GSGGG (SEQ ID NO: 60), GGGSG (SEQ ID NO: 61), and GSSSG (SEQ ID NO: 62). A multimer (e.g., 1, 2, 3, 4, 5, 6, 7, 8, 9, 10, 10-20, 20-30, or 30-50) of these linker sequences may be linked together to provide flexible linkers that may be used to conjugate a heterologous amino acid sequence to the polypeptides disclosed herein. As described herein, the heterologous amino acid sequence may be a signal sequence and/or a fusion partner, such as, albumin, Fc sequence, and the like.

### Methods of Production

The proteins of the present disclosure can be produced by any suitable method, including recombinant and non-recombinant methods (e.g., chemical synthesis). Where a polypeptide is chemically synthesized, the synthesis may proceed via liquid-phase or solid-phase. Solid-phase synthesis (SPPS) allows the incorporation of unnatural amino acids and/or peptide/protein backbone modification. Various forms of SPPS, such as Fmoc and Boc, are available for synthesizing peptides of the present disclosure. Details of the chemical synthesis are known in the art (e.g., Ganesan A. 2006 Mini Rev. Med Chem. 6:3-10 and Camarero J A et al. 2005 Protein Pept Lett. 12:723-8). Briefly, small insoluble, porous beads are treated with functional units on which peptide chains are built. After repeated cycling of coupling/deprotection, the free N-terminal amine of a solid-phase attached is coupled to a single N-protected amino acid unit. This unit is then deprotected, revealing a new N-terminal amine to which a further amino acid may be attached. The peptide remains immobilized on the solid-phase and undergoes a filtration process before being cleaved off.

Where the protein is produced using recombinant techniques, the proteins may be produced as an intracellular protein or as an secreted protein, using any suitable construct and any suitable host cell, which can be a prokaryotic or eukaryotic cell, such as a bacterial (e.g., E. coli) or a yeast host cell, respectively.

Other examples of eukaryotic cells that may be used as host cells include insect cells, mammalian cells, and/or plant cells. Where mammalian host cells are used, the cells may include one or more of the following: human cells (e.g., HeLa, 293, H9 and Jurkat cells); mouse cells (e.g., NIH3T3, L cells, and C127 cells); primate cells (e.g., Cos 1, Cos 7 and CV1) and hamster cells (e.g., Chinese hamster ovary (CHO) cells).

A wide range of host-vector systems suitable for the expression of the subject protein may be employed according to standard procedures known in the art. See for example, Sambrook et al. 1989 Current Protocols in Molecular Biology Cold Spring Harbor Press, New York and Ausubel et al. 1995 Current Protocols in Molecular Biology, Eds. Wiley and Sons.

Methods for introduction of genetic material into host cells include, for example, transformation, electroporation, conjugation, calcium phosphate methods and the like. The method for transfer can be selected so as to provide for stable expression of the introduced GFRAL and/or RET-encoding nucleic acid. The polypeptide-encoding nucleic acid can be provided as an inheritable episomal element (e.g., plasmid) or can be genomically integrated. A variety of appropriate vectors for use in production of a polypeptide of interest are available commercially.

Vectors can provide for extrachromosomal maintenance in a host cell or can provide for integration into the host cell genome. The expression vector provides transcriptional and translational regulatory sequences, and may provide for inducible or constitutive expression, where the coding region is operably linked under the transcriptional control of the transcriptional initiation region, and a transcriptional and translational termination region. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7, CMV, and the like). In certain embodiments, the proteins of the present disclosure may be expressed from a nucleic acid construct in which a heterologous promoter is operably linked to a nucleic acid sequence encoding the protein.

Expression constructs generally have convenient restriction sites located near the promoter sequence to provide for the insertion of nucleic acid sequences encoding proteins of interest. A selectable marker operative in the expression host may be present to facilitate selection of cells containing the vector. In addition, the expression construct may include additional elements. For example, the expression vector may have one or two replication systems, thus allowing it to be maintained in organisms, for example in mammalian or insect cells for expression and in a prokaryotic host for cloning and amplification. In addition the expression construct may contain a selectable marker gene to allow the selection of transformed host cells. Selectable genes are well known in the art and will vary with the host cell used.

Isolation and purification of a protein can be accomplished according to methods known in the art. For example, a protein can be isolated from a lysate of cells genetically modified to express the protein constitutively and/or upon induction, or from a synthetic reaction mixture, by immunoaffinity purification, which generally involves contacting the sample with an anti-protein antibody, washing to remove non-specifically bound material, and eluting the specifically bound protein. The isolated protein can be further purified by dialysis and other methods normally employed in protein purification methods. In one embodiment, the protein may be isolated using metal chelate chromatography methods. Protein of the present disclosure may contain modifications to facilitate isolation, as discussed above.

The subject proteins may be prepared in substantially pure or isolated form (e.g., free from other polypeptides). The protein can be present in a composition that is enriched for the polypeptide relative to other components that may be present (e.g., other polypeptides or other host cell components). Purified protein may be provided such that the protein is present in a composition that is substantially free of other expressed proteins, e.g., less than 90%, usually less than 60% and more usually less than 50% of the composition is made up of other expressed proteins.

### Recombinant Cells

As noted above, a recombinant cell genetically modified to express a GFRAL protein is disclosed. As explained above the GFRAL protein expressed by the cell may be a full length GFRAL protein as depicted in FIGS. 1C-1E or a variant or a fragment thereof. For example, the GFRAL protein may lack an intracellular domain present in native GFRAL and/or may include a heterologous signal sequence. The recombinant cell may endogenously express RET and/or may be genetically modified to express RET.

In certain cases, the recombinant cell may include a reporter construct that includes a promoter sequence operably linked to a nucleic acid sequence encoding a reporter, wherein the promoter directs expression of the reporter upon activation of RET by binding of the GDF15 protein to GFRAL.

In certain embodiments, the recombinant cell may include a transcriptional activator such as an Elk protein or a functionally active fragment thereof which may be phosphorylated by the activated RET. The phosphorylated Elk can induce transcription of the reporter when bound to the promoter operably linked to the nucleic acid sequence encoding the reporter while Elk that is not phosphorylated is not capable of inducing transcription. The Elk protein may be fused to a DNA binding domain (DBD) of a heterologous protein, e.g., a GAL4DBD which specifically binds to a GAL4 upstream activating sequence (GAL4-UAS). In certain embodiments, the promoter sequence of the reporter construct may include a GAL4-UAS. In certain embodiments, activation of RET by binding of GFRAL to GDF15 may lead to activation of Elk via phosphorylation by activated RET. In certain embodiments, the phosphorylated Elk when bound to the GAL4-UAS via the GAL4DBD may mediate the transcription of the reporter.

A number of reporter constructs may be used for detecting RET activation. For example, the reporter sequence may encode a reporter protein that is directly or indirectly detectable. For example, the reporter may be a fluorescent protein, an enzyme, or a protein that may be detected using an antibody.

The recombinant cell may include a plasmid or a stably integrated nucleic acid that includes a promoter sequence that directs the expression of an Elk-GAL4 protein. The promoter may be a constitutive or an inducible promoter. In certain embodiments, in absence of RET activation, the Elk-GAL4 protein may not be significantly phosphorylated and may not activate transcription of a reporter operably connected to a GAL4-UAS promoter sequence.

A recombinant cell as disclosed herein may be used for identifying an agent that binds to the extracellular domain of GFRAL. In additional embodiments, a recombinant cell that expresses GFRAL and RET may be used to identify agents that bind to GFRAL and lead to activation of RET. In addition, a recombinant cell genetically modified to express GFRAL and RET may be used to identify agents that modulate the binding between GFRAL and RET. Methods for identifying such agents are discussed later below.

Also disclosed herein are compositions that include a recombinant cell as described herein and an isolated GDF15 protein. As explained further below, such compositions may be used in screening methods to identify modulators of GFRAL-GDF15 receptor-ligand complex.

### Methods of Screening

A method using the isolated protein complex described herein and/or the recombinant cell described herein for identifying agents that bind to an extracellular domain of GFRAL is disclosed.

Candidate agents of interest for screening include biologically active agents of numerous chemical classes, primarily organic molecules, although including in some instances, inorganic molecules, organometallic molecules, immunoglobulins, genetic sequences, etc. Also of interest are small organic molecules, which comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Compounds may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

A plurality of assays may be run in parallel with different concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent.

In certain embodiments, a method for identifying agents that bind to an extracellular domain of GFRAL is disclosed. The method may include assaying for binding of a candidate agent to an extracellular domain of GFRAL, where a candidate agent that binds GFRAL is identified as an agent that binds to GFRAL, where binding of the candidate agent is compared to binding of GDF15 to the extracellular domain of GFRAL.

In certain cases, a GFRAL or an ECD-containing fragment thereof may be expressed on the surface of a cell. In other cases, a GFRAL or an ECD-containing fragment thereof may be immobilized on a support. In other cases, a cell expressing GFRAL or an ECD-containing fragment thereof expressed on the cell surface may be immobilized on a support. The assay may include contacting the cell and/or the support with a candidate agent and determining whether the candidate agent is bound to the extracellular domain of GFRAL. Any standard technique for determining binding may be utilized. For example, the candidate agents may be labeled and retention of the label to the cell or solid support after washing to remove non-specific binders may indicate that the candidate agent binds to extracellular domain of GFRAL. As noted above, the binding may be compared to the binding of GDF15 under similar conditions, where a candidate agent that binds the extracellular domain of GFRAL with an affinity similar to GDF15 may be identified as a candidate agent.

In certain cases, the assay may include contacting a recombinant cell expressing GFRAL on the cell surface with a candidate agent, where the recombinant cell may be genetically modified to express RET. The recombinant cell may also include a reporter construct containing a promoter sequence operably linked to a nucleic acid sequence encoding a reporter, where the promoter directs expression of the reporter upon activation of RET, and where the method includes assaying for expression of the reporter, where increased expression of the reporter as compared to a negative control identifies the agent as an agent that binds to GFRAL and activates RET.

In certain cases, the expression of the reporter upon contacting the recombinant cell with a candidate agent may be compared to the expression of the reporter upon the contacting of the recombinant cell with GDF15 in a separate assay, which may be conducted in parallel to the screening method. In certain cases, a candidate agent that induces reporter expression at a level similar to that induced by GDF15 is identified as an agent that binds to GFRAL and activates RET.

Also provided herein is a method for identifying agents that modulate binding of GDF15 to GFRAL. In certain embodiments, the method may include contacting a candidate agent with a recombinant cell genetically modified to express GFRAL, where the contacting is in the presence of the GDF15; and assaying a level of binding of GDF15 to GFRAL; wherein a change in the level of binding of GDF15 to GFRAL in the presence of the agent as compared to a level of binding of GDF15 to GFRAL in absence of the agent identifies the agent as a modulator of GDF15 binding to GFRAL.

In certain embodiments, GDF15 may be detectably labeled and a decrease in the amount of label bound to the recombinant cell in the presence of a candidate agent may identify it as an agent that competes with GDF15 for binding to GFRAL. In alternate embodiments the candidate agent may be detectably labeled and the assay may include determining binding of the candidate agent to GFRAL in the presence of GDF15, which may be unlabeled.

In certain cases, the recombinant cell used in the screening in the presence of GDF15 may be genetically modified to express RET as noted above and include a reporter construct containing a promoter sequence operably linked to a nucleic acid sequence encoding a reporter, where the promoter directs expression of the reporter upon activation of RET, where the assaying comprises assaying for expression of the reporter, where a change in expression of the reporter as compared to the expression in absence of the agent identifies the agent as an agent that modulates binding of GDF15 to GFRAL.

In certain cases, the agent may inhibit binding of GDF15 to GFRAL and may be identified as an antagonist of GDF15-GFRAL binding. In other cases, the agent may increase binding of GDF15 to GFRAL and may be identified as an agonist of GDF15-GFRAL binding.

In certain embodiments, the agent may compete with GDF15 for binding to GFRAL. In certain cases, the agent when bound to GFRAL may lead to activation of RET and reporter expression.

As provided herein is an assay for identifying an agent that modulates the binding between GFRAL and RET. The assay may include contacting a recombinant cell genetically engineered to express GFRAL and RET on the cell surface with a candidate agent and assessing the binding between GFRAL and RET. In certain cases, the binding between GFRAL and RET may be increased or decreased in the presence of the agent as compared to a negative control, which may identify the agent as a modulator of GFRAL and RET binding. Binding between GFRA and RET may be assessed using a standard method for assessing protein-protein binding.

Exemplary methods for assessing protein-protein binding include immunoprecipitation, immunostaining, bioluminescence resonance energy transfer (BRET), fluorescence resonance energy transfer (FRET), TR-FRET (time-resolved-FRET) or by HTRF (homogeneous time resolved fluorescence).

As noted herein, one of more of the proteins, e.g., GFRAL, RET, and GDF15 may be conjugated to a heterologous sequence such as a tag (e.g., poly-Histidine tag, Glutathione S-transferase (GST) tag, FLAG tag, HA tag, Fc tag, HSA tag, and the like); a fluorescent protein (GFP, YFP, RFP, and the like), bioluminescent protein (e.g., luciferase).

In certain embodiments, binding between members of a binding pair (e.g., a pair of proteins or binding of an agent (non-protein agent) to a protein may be assessed using FRET or BRET. For example, one member of a binding pair may be conjugated to a first fluorophore (e.g., CFP) or a bioluminescent protein (e.g., luciferase) and the other member may be conjugated to a second fluorophore (e.g., YFP).

### Compositions

The present disclosure provides compositions comprising a subject protein, which may be administered to a subject in need of treatment or prevention of involuntary body weight loss or in need of reduction of GDF15 activity. In certain cases, the composition may include a polypeptide, such as, a GFRAL ECD, a GFRAL ECD fragment, a GFRAL ECD variant as described herein, or a combination thereof.

The polypeptides of the present disclosure may be in the form of compositions suitable for administration to a subject. In general, such compositions are “pharmaceutical compositions” comprising one or more polypeptides and one or more pharmaceutically acceptable or physiologically acceptable diluents, carriers or excipients. In certain embodiments, the polypeptides are present in a therapeutically effective amount. The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein. Furthermore, the pharmaceutical compositions may be used in combination with other therapeutically active agents or compounds (e.g., an appetite enhancing agent) in order to treat or prevent the diseases, disorders and conditions as contemplated by the present disclosure.

The pharmaceutical compositions typically comprise a therapeutically effective amount of at least one of the polypeptides contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that could be used in the pharmaceutical compositions and dosage forms. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-am inopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments. Any drug delivery apparatus may be used to deliver the polypeptides, including implants (e.g., implantable pumps) and catheter systems, both of which are well known to the skilled artisan. Depot injections, which are generally administered subcutaneously or intramuscularly, may also be utilized to release the polypeptides disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed including synthetic mono- or diglycerides. Moreover, fatty acids such as oleic acid find use in the preparation of injectables. Prolonged absorption of particular injectable formulations can be achieved by including an agent that delays absorption (e.g., aluminum monostearate or gelatin).

The pharmaceutical compositions containing the active ingredient (e.g., polypeptides of the present disclosure) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods of preparing liposomes are described in, for example, U.S. Pat. Nos. 4,235,871, 4,501,728, and 4,837,028. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally-occurring gums, for example, gum acacia or gum tragacanth; naturally-occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Formulations can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, may be employed.

The present disclosure contemplates the administration of the polypeptides in the form of suppositories for rectal administration of the drug. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The polypeptides contemplated by the present disclosure may be in the form of any other suitable pharmaceutical composition (e.g., sprays for nasal or inhalation use) currently known or developed in the future.

The concentration of a polypeptide or fragment thereof in a formulation can vary widely (e.g., from less than about 0.1%, usually at or at least about 2% to as much as 20% to 50% or more by weight) and will usually be selected primarily based on fluid volumes, viscosities, and subject-based factors in accordance with, for example, the particular mode of administration selected.

Contemplated herein is the use of Nano Precision Medical's depot delivery technology (Nano Precision Medical; Emeryville, Calif.). The technology utilizes a titania nanotube membrane that produces zero-order release rates of macromolecules, such as protein and peptide therapeutics. The biocompatible membrane is housed in a small, subcutaneous implant that provides long-term (e.g., up to one year), constant-rate delivery of therapeutic macromolecules. The technology is currently being evaluated for the delivery of GLP-1 agonists for the treatment of Type II diabetes. In certain embodiments, the polypeptide(s) disclosed herein may be a formulation with a membrane. For example, the polypeptide may be impregnated into the membrane or surrounded by the membrane. The membrane may be in shape of a disc, tube or sphere. In certain embodiments, the tube may be a nanotube or the sphere may be a nanosphere.

A subject pharmaceutical composition can include a GFRAL, GFRAL extracellular domain, or soluble GFRAL-ECD polypeptide, and a pharmaceutically acceptable excipient.

### Patient Populations

The present disclosure provides a method to treat a patient suffering from involuntary weight loss. An example of a suitable patient may be one who is diagnosed with a wasting disease or cachexia. Suitable patients include those suffering from liver cirrhosis, hyperthyroidism, chronic kidney disease, Parkinson's disease, cancer, eating disorder (e.g., anorexia nervosa), chronic inflammatory disease (e.g., rheumatoid arthritis), sepsis or other forms of systemic inflammation, chronic obstructive pulmonary disease, AIDS, tuberculosis, and muscle wasting, such as muscular dystrophy or multiple sclerosis), or sarcopenia.

The present disclosure also provides methods for preventing involuntary weight loss in a patient who may be at risk of involuntary weight loss due to a chronic disease, such as, liver cirrhosis, hyperthyroidism, chronic kidney disease, Parkinson's disease, cancer, eating disorder (e.g., anorexia nervosa), chronic inflammatory disease (e.g., rheumatoid arthritis), sepsis or other forms of systemic inflammation, chronic obstructive pulmonary disease, AIDS, tuberculosis, and muscle wasting, such as muscular dystrophy or multiple sclerosis), or sarcopenia. Such patients may include patients who have elevated levels of GDF15, are undergoing treatment for cancer, and the like.

The present disclosure provides a method to treat a patient suffering from cachexia. An example of a suitable patient may be one who is diagnosed with cachexia. The present disclosure also provides methods for preventing involuntary weight loss in a patient who may be at risk of involuntary weight loss due to onset of cachexia. Such patients include patients who have elevated levels of GDF15, have cancer, are undergoing treatment for cancer, have an eating disorder, and the like.

Also disclosed is a method for reducing GDF15 activity in a patient having elevated GDF15 activity. As used herein, “elevated GDF15 activity” refers to increased activity or amount of GDF15 in a biological fluid of a subject in comparison to a normal subject. A number of conditions are associated with increased GDF15 serum level, wherein the increased GDF15 results in a number of symptoms such as appetite loss, weight loss, and the like. Examples of conditions associated with increased GDF15 serum level include cancer, e.g., melanoma, gastric cancer, pancreatic cancer, prostate cancer; autoimmune diseases such as, arthritis and inflammation; cardiovascular diseases like atherosclerosis, heart failure, hypertension, myocardial infarction, chest pain, and cardiovascular events; metabolic diseases like anemia, cachexia, anorexia, kidney disease, and thalassemia, etc.

A patient having any of the above disorders may be a suitable candidate for receiving an agent that binds an extracellular domain of a GFRAL protein, or a fragment of GFRAL that includes a GFRAL extracellular domain, e.g., a soluble GFRAL ECD, or a combination of the agent and GFRAL fragment.

Administering the subject GFRAL protein fragments, such as, GFRAL ECD, GFRAL ECD fragment, and/or GFRAL ECD variant in such an individual may decrease or prevent one or more of the symptoms associated with the disorder. For example, administering the proteins of the present disclosure may increase body weight and/or appetite in a subject.

### Methods

The subject method involves administering the subject proteins to a patient who has involuntary body weight loss or is at risk of developing involuntary body weight loss. The subject methods include administering proteins disclosed herein to a subject who has elevated serum levels of GDF15. The methods of the present disclosure include administering at least one of: an agent that binds an extracellular domain of a GFRAL protein; and a fragment of GFRAL that includes a GFRAL extracellular domain, e.g., a soluble GFRAL-ECD.

In certain cases, the agent may be an anti-GFRAL antibody that competes with GDF15 for binding to extracellular domain of GFRAL. In certain cases, the agent may be an agent that binds to an extracellular domain of a GFRAL protein but does not activate RET. For example, the agent may be an anti-GFRAL antibody that competes with GDF15 for binding to extracellular domain of GFRAL but does not activate RET upon binding to GFRAL. Such an antibody may be generated by immunizing a laboratory animal with a GFRAL ECD and screening the generated antibodies in a GFRAL binding assay and/or GFRAL signaling assay as described herein. Such anti-GFRAL antibodies may be modified to generate chimeric or humanized antibodies by using standard methods.

In certain cases, the fragment of GFRAL administered to a patient who has cachexia or is at risk of developing cachexia may be a GFRAL ECD, a GFRAL ECD fragment, and/or a GFRAL ECD variant, as described herein.

Subjects having, suspected of having, or at risk of developing cachexia are contemplated for therapy described herein.

In the methods of the present disclosure, protein compositions described herein can be administered to a subject (e.g., a human patient) to, for example, achieve a target body weight and/or maintain body weight; achieve a target body mass index (BMI) and/or maintain a BMI; increase appetite; and the like. A normal human adult has a BMI in the range 18.5-24.9 Kg/m².

The subject treatment methods may increase body weight, BMI, muscle weight, and/or food intake in a patient by at least about 5%, e.g., 10%, 15%, 20%, 25%, 30%, 35%, 40%, 45%, 50% or more.

In certain cases, the agent may be an agent identified via a screening method for GFRAL binding agents, such as, the screening methods disclosed herein.

The methods relating to treatment or prevention of cachexia contemplated herein include, for example, use of protein described above for therapy/prevention alone or in combination with other types of therapy. The method involves administering to a subject the subject protein (e.g., subcutaneously, intradermally, or intravenously).

In some embodiments, the agent is administered to a patient experiencing loss of muscle mass, for example, loss of muscle mass associated with an underlying disease. Underlying diseases associated with cachexia include, but are not limited to, cancer, chronic renal disease, chronic obstructive pulmonary disease, AIDS, tuberculosis, chronic inflammatory diseases, sepsis and other forms of systemic inflammation, muscle wasting, such as muscular dystrophy, and the eating disorder known as anorexia nervosa. In some embodiments, the agent inhibits loss of lean mass (e.g., muscle mass) and or fat mass by at least 40%, 50%, 60%, 70%, 80%, 90%, 95%, 98%, 99%, or 100%.

In some embodiments, a loss of lean mass (e.g., muscle mass) is accompanied by a loss of fat mass. In some embodiments, the agent can inhibit loss of fat mass by at least 40%, 50%, 60%, 70%, 80%, 90%, 95%, 98%, 99%, or 100%.

In some embodiments, the agent is administered to a patient diagnosed with body weight loss (e.g., involuntary weight loss). In some embodiments, the agent can revert body weight loss (e.g., involuntary weight loss) by at least 2%, 5%, 10%, 15%, 20%, 25%, 30% or 35%.

In some embodiments, the agent is administered to a patient diagnosed with loss of organ mass, for example, loss of organ mass associated with an underlying disease. Underlying diseases associated with cachexia include, but are not limited to, cancer, chronic renal disease, chronic obstructive pulmonary disease, AIDS, tuberculosis, chronic inflammatory diseases, sepsis and other forms of systemic inflammation, muscle wasting, such as muscular dystrophy, and the eating disorder known as anorexia nervosa. In some embodiments, the agent can inhibit loss of organ mass by at least 40%, 50%, 60%, 70%, 80%, 90%, 95%, 98%, 99%, or 100%. In some embodiments, loss of organ mass is observed in heart, liver, kidney, and/or spleen. In some embodiments, the loss of organ mass in accompanied by a loss of muscle mass, a loss of fat mass and/or involuntary weight loss.

Sarcopenia, muscle wasting disorders and significant muscle weight loss can occur in the absence of cachexia, decreased appetite or body weight loss. In some embodiments, the agent can be used to treat a subject diagnosed with sarcopenia, a muscle wasting disorder and/or significant muscle weight loss, whether or not the subject has, or has been diagnosed with, cachexia or decreased appetite. Such a method comprises administering a therapeutically effective amount of one or more agents to a subject in need thereof.

In some embodiments, the agent is administered to a patient diagnosed with obesity. In some embodiments, the agent can inhibit weight gain or to reduce body weight by at least 5%, 10%, 15%, 20%, 25%, 30%, 35%, 40%, 45% or 50%. Use of the agent to treat obesity in a patient comprises administering to the patient a therapeutically effective amount of the agent.

### Routes of Administration

The present disclosure contemplates the administration of the disclosed polypeptides, and compositions thereof, in any appropriate manner. Suitable routes of administration include parenteral (e.g., intramuscular, intravenous, subcutaneous (e.g., injection or implant), intraperitoneal, intracisternal, intraarticular, intraperitoneal, intracerebral (intraparenchymal) and intracerebroventricular), oral, nasal, vaginal, sublingual, intraocular, rectal, topical (e.g., transdermal), sublingual and inhalation.

Depot injections, which are generally administered subcutaneously or intramuscularly, may also be utilized to release the polypeptides disclosed herein over a defined period of time. Depot injections are usually either solid- or oil-based and generally comprise at least one of the formulation components set forth herein. One of ordinary skill in the art is familiar with possible formulations and uses of depot injections.

Regarding antibodies, in an exemplary embodiment an antibody or antibody fragment of the present disclosure is stored at 10 mg/ml in sterile isotonic aqueous saline solution for injection at 4° C. and is diluted in either 100 ml or 200 ml 0.9% sodium chloride for injection prior to administration to the subject. The antibody is administered by intravenous infusion over the course of 1 hour at a dose of between 0.2 and 10 mg/kg. In other embodiments, the antibody is administered by intravenous infusion over a period of between 15 minutes and 2 hours. In still other embodiments, the administration procedure is via subcutaneous bolus injection.

The present disclosure contemplates methods wherein the polypeptide or an antibody or antibody fragment of the present disclosure is administered to a subject at least twice daily, at least once daily, at least once every 48 hours, at least once every 72 hours, at least once weekly, at least once every 2 weeks, at least once monthly, at least once every 2 months, or at least once every 3 months, or less frequently.

### Combination Therapy

Any of a wide variety of therapies directed to treating or preventing cachexia can be combined in a composition or therapeutic method with the subject proteins.

“Combination” as used herein is meant to include therapies that can be administered separately, e.g., formulated separately for separate administration (e.g., as may be provided in a kit), as well as for administration in a single formulation (i.e., “co-formulated”). Examples of agents that may be provided in a combination therapy include an agent that binds an extracellular domain of a GFRAL protein and competes with GDF15 for binding to the ECD of GFRAL, e.g., an antibody that competes with GDF15 for binding to ECD of GFRAL. An exemplary combination therapy may include administering an anti-GFRAL ECD antibody and a fragment of GFRAL that includes a GFRAL extracellular domain, e.g., a soluble GFRAL-ECD.

Where the GFRAL-ECD protein is administered in combination with one or more other therapies, the combination can be administered anywhere from simultaneously to up to 5 hours or more, e.g., 10 hours, 15 hours, 20 hours or more, prior to or after administration of a subject protein. In certain embodiments, a subject protein and other therapeutic intervention are administered or applied sequentially, e.g., where a subject protein is administered before or after another therapeutic treatment. In yet other embodiments, a subject protein and other therapy are administered simultaneously, e.g., where a subject protein and a second therapy are administered at the same time, e.g., when the second therapy is a drug it can be administered along with a subject protein as two separate formulations or combined into a single composition that is administered to the subject. Regardless of whether administered sequentially or simultaneously, as illustrated above, the treatments are considered to be administered together or in combination for purposes of the present disclosure.

Cytokines that are implicated in cachexia include Activin A and IL-6. Increased activin levels have been associated with cancer-associated cachexia and gonadal tumors. See, e.g., Marino et al. (2013) CYTOKINE & GROWTH FACTOR REV. 24:477-484. Activin A is a member of the TGF-beta family, and is a ligand of the activin type 2 receptor, ActRIIB. See, e.g., Zhou et al. (2010) CELL 142:531-543. Circulating levels of IL-6 have been shown to correlate with weight loss in cancer patients, as well as with reduced survival. See, e.g., Fearon et al. (2012) CELL METABOLISM 16: 153-166.

Accordingly, in some embodiments, one or more inhibitors of Activin-A or the Activin-A receptor, ActRIIB, IL-6 or the IL-6 receptor (IL-6R), can be administered in combination with (for example, administered at the same time as, administered before, or administered after) an agent that binds an extracellular domain of a GFRAL protein and/or competes with GDF15 for binding to the ECD of GFRAL (e.g., an antibody that competes with GDF15 for binding to ECD of GFRAL). Exemplary inhibitors of Activin A or ActRIIB, include, for example, an anti-Activin-A antibody or an antigen binding fragment thereof, an anti-ActRIIB antibody or an antigen binding fragment thereof, a small molecule inhibitor of Activin-A, a small molecule inhibitor of ActRIIB, and a ‘decoy’ receptor of ActRIIB, such as a soluble ActRIIB receptor and a fusion of the soluble ActRIIB receptor with an Fc molecule (ActRIIB-Fc). See, e.g., Zhou et al. (2010), supra. Suitable inhibitors of IL-6 or IL-6R, include an anti-IL-6 antibody or an antigen binding fragment thereof, an anti-IL-6R antibody or an antigen binding fragment thereof, a small molecule inhibitor of IL-6, a small molecule inhibitor of IL-6R, and a ‘decoy’ receptor of IL-6R, such as a soluble IL-6 receptor and a fusion of the soluble IL-6 receptor with an Fc molecule (IL6R-Fc). See, e.g., Enomoto et al. (2004) BIOCHEM. AND BIOPHYS. RES. COMM. 323: 1096-1 102; Argiles et al. (2011) EUR. J. PHARMACOL. 668:S81-S86; Tuca et al. (2013) ONCOLOGY/HEMATOLOGY 88:625-636. Suitable inhibitors of IL-6 or IL-6R can include, e.g., Tocilizumab (Actemra®, Hoffmann-LaRoche), a humanized anti-IL-6R monoclonal antibody approved for treatment of rheumatoid arthritis, and Sarilumab/REGN88 (Regeneron), a humanized anti-IL6R antibody in clinical development for treatment of rheumatoid arthritis; and Selumetinib/AZD6244 (AstraZeneca), an allosteric inhibitor of MEK, which has been shown to inhibit IL-6 production. Prado et al. (2012) BRITISH J. CANCER 106: 1583-1586.

TNFα and IL-1 are cytokines known to be involved in mediation of the proinflammatory response, which are also implicated in muscle depletion, anorexia and cachexia. Increased circulating levels of TNFα appear to inhibit myogenesis. TNFα, also known as “cachectin,” stimulates interleukin-1 secretion and is implicated in the induction of cachexia. IL-1 is a potent trigger of the acute-phase inflammatory response, and it has been shown that infusion of IL-1 can lead to marked weight loss and appetite loss. IL-1 has been shown to contribute to the initiation of cancer cachexia in mice bearing a murine colon-26 adenocarcinoma (Strassmann et al. (1993) J. IMMUNOL. 150:2341). See also, Mathys and Billiau (1997) NUTRITION 13:763-770; Fong et al. (1989) AM. J. PHYSIOL.—REGULATORY, INTEGRATIVE AND COMPARATIVE PHYSIOL., 256:R659-R665. Thus, TNFα inhibitors and IL-1 inhibitors that are used in the treatment of rheumatoid arthritis may also be useful in the treatment of cachexia.

Accordingly, in some embodiments, one or more inhibitors of TNFα or IL-1 can be administered in combination with (e.g., administered at the same time as, administered before, or administered after) an agent that binds an extracellular domain of a GFRAL protein and/or competes with GDF15 for binding to the ECD of GFRAL (e.g., an antibody that competes with GDF15 for binding to ECD of GFRAL). Suitable inhibitors of TNFα or IL-1 include an anti-TNFα antibody or an antigen binding fragment thereof, an anti-IL-1 antibody or an antigen binding fragment thereof, a small molecule inhibitor of TNFα or IL-1, and a ‘decoy’ receptor of TNFα or IL-1, such as a soluble TNFα or IL-1 receptor and a fusion of the soluble form of TNFα or IL-1 with an Fc molecule. Suitable inhibitors of TNFα include for example, etanercept (Enbrel®, Pfizer/Amgen), infliximab (Remicade®, Janssen Biotech), adalimumab (Humira®, Abbvie), golimumab (Simponi®, Johnson and Johnson/Merck), and certolizumab pegol (Cimzia®, UCB). Suitable IL-1 inhibitors include, e.g., Xilonix® antibody that targets IL-1 a (XBiotech), anikinra (Kinaret®, Amgen), canakinumab (Ilaris®, Novartis), and rilonacept (Arcalyst®, Regeneron). In certain embodiments, the TNFa inhibitor or IL-1 inhibitor, which is typically administered systemically for the treatment of rheumatoid arthritis may be administered locally and directly to the tumor site.

Myostatin, also known as GDF-8, is a member of the TGF-β family of peptides that is a negative regulator of muscle mass, as shown by increased muscle mass in myostatin deficient mammals. Myostatin is a ligand of the activin type 2 receptor, ActRIIB.

Accordingly, in some embodiments, one or more inhibitors of myostatin or its receptor may be administered in combination with (for example, administered at the same time as, administered before, or administered after) an agent that binds an extracellular domain of a GFRAL protein and/or competes with GDF15 for binding to the ECD of GFRAL (e.g., an antibody that competes with GDF15 for binding to ECD of GFRAL). Suitable inhibitors of myostatin or ActRIIB, include an anti-myostatin antibody or an antigen binding fragment thereof, an anti-ActRIIB antibody or an antigen binding fragment thereof, a small molecule inhibitor of myostatin, a small molecule inhibitor of ActRIIB, and a ‘decoy’ receptor of GDF-8, such as a soluble ActRIIB and a fusion of the soluble form of ActRIIB with an Fc molecule. See, e.g., Lokireddy et al. (2012) BIOCHEM. J. 446(I):23-26. Myostatin inhibitors that may be suitable for the present methods include REGN1033 (Regeneron); see Bauerlein et al. (2013) J. CACHEXIA SARCOPENIA MUSCLE: Abstracts of the 7^{th} Cachexia Conference, Kobe/Osaka, Japan, Dec. 9-11, 2013, Abstract 4-06; LY2495655 (Lilly), a humanized anti-myostatin antibody in clinical development by Eli Lilly; see also “A PHASE 2 STUDY OF LY2495655 IN PARTICIPANTS WITH PANCREATIC CANCER,” available on the world wide web at clinicaltrials.gov/ct2/NCT01505530; NML identifier: NCT01505530; ACE-031 (Acceleron Pharma); and stamulumab (Pfizer).

Agents such as Ghrelin or ghrelin mimetics, or other growth hormone secretagogues (GHS) which are able to activate the GHS receptor (GHS-Rla), also known as the ghrelin receptor, can be useful for increasing food intake and body weight in humans. See Guillory et al. (2013) in VITAMINS AND HORMONES vol. 92, chap. 3; and Steinman and DeBoer (2013) VITAMINS AND HORMONES vol. 92, chap. 8. Accordingly, in some embodiments, one or more Ghrelin or ghrelin mimetics, or other growth hormone secretagogues (GHS), can be administered in combination with (for example, administered at the same time as, administered before, or administered after) an agent that binds an extracellular domain of a GFRAL protein and/or competes with GDF15 for binding to the ECD of GFRAL (e.g., an antibody that competes with GDF15 for binding to ECD of GFRAL). Suitable ghrelin mimetics include anamorelin (Helsinn, Lugano, CH); see Temel et al. (2013) J. CACHEXIA SARCOPENIA MUSCLE: Abstracts of the 7^{th} Cachexia Conference, Kobe/Osaka, Japan, Dec. 9-11, 2013, Abstract 5-01. Other suitable GHS molecules can be identified, for example, using the growth hormone secretagogue receptor Ghrelin competition assay described in PCT Publication Nos. WO201 1/1 17254 and WO2012/1 13103.

Agonists of the androgen receptor, including small molecules and other selective androgen receptor modulators (SARMs) can be useful in treating cachexia and/or sarcopenia. See, e.g., Mohler et al. (2009) J. MED. CHEM. 52:3597-3617; Nagata et al. (2011) BIOORGANIC AND MED. CHEM. LETTERS 21: 1744-1747; and Chen et al. (2005) MOL. INTERV. 5: 173-188. Ideally, SARMs should act as full agonists, like testosterone, in anabolic target tissues, such as muscle and bone, but should demonstrate only partial or pure androgen receptor antagonistic activities on prostate tissue. See, e.g., Bovee et al. (2010) J. STEROID BIOCHEM. & MOL. BIOL. 118:85-92. Suitable SARMs can be identified, e.g., by use of the methods and assays described in Zhang et al. (2006) BIOORG. MED. CHEM. LETT. 16:5763-5766; and Zhang et al. (2007) BIOORG. MED. CHEM. LETT. 17:439-443.

Accordingly, in some embodiments, one or more androgen receptor agonists can be administered in combination with (for example, administered at the same time as, administered before, or administered after) an agent that binds an extracellular domain of a GFRAL protein and/or competes with GDF15 for binding to the ECD of GFRAL (e.g., an antibody that competes with GDF15 for binding to ECD of GFRAL). Suitable SARMs include, for example, GTx-024 (enobosarm, Ostarine®, GTx, Inc.), a SARM in phase II clinical development by GTx, Inc. See also, Dalton et al. (2011) J. CACHEXIA SARCOPENIA MUSCLE 2: 153-161. Other suitable SARMs include 2-(2,2,2)-trifluoroethyl-benzimidazoles (Ng et al. (2007) BIOORG. MED. CHEM. LETT. 17: 1784-1787) and JNJ-26146900 (Allan et al. (2007) J. STEROID BIOCHEM. & MOL. BIOL. 103:76-83).

β-adrenergic receptor blockers, or beta-blockers, have been studied for their effect on body weight in cachexia subjects, and have been associated with partial reversal of cachexia in patients with congestive heart failure. See, e.g., Hryniewicz et al. (2003) J. CARDIAC FAILURE 9:464-468. Beta-blocker MT-102 (PsiOxus Therapeutics, Ltd.) has been evaluated in a phase 2 clinical trial for subjects with cancer cachexia. See Coats et al. (2011) J. CACHEXIA SARCOPENIA MUSCLE 2:201-207. Accordingly, in some embodiments, one or more β-adrenergic receptor blockers, or beta-blockers, can be administered in combination with (for example, administered at the same time as, administered before, or administered after) an agent that binds an extracellular domain of a GFRAL protein and/or competes with GDF15 for binding to the ECD of GFRAL (e.g., an antibody that competes with GDF15 for binding to ECD of GFRAL).

Melanocortin receptor-knockout mice with a genetic defect in melanocortin signaling exhibit a phenotype opposite that of cachexia: increased appetite, increased lean body mass, and decreased metabolism. Thus, melanocortin antagonism has emerged as a potential treatment for cachexia associated with chronic disease (DeBoer and Marks (2006) TRENDS IN ENDOCRINOLOGY AND METABOLISM 17: 199-204).

Accordingly, in some embodiments, one or more inhibitors of a melanocortin peptide or a melanocortin receptor can be administered in combination (e.g., administered at the same time as, administered before, or administered after) with an agent that binds an extracellular domain of a GFRAL protein and/or competes with GDF15 for binding to the ECD of GFRAL (e.g., an antibody that competes with GDF15 for binding to ECD of GFRAL). Suitable inhibitors of melanocortins or melanocortin receptors include an anti-melanocortin peptide antibody or an antigen binding fragment thereof, an anti-melanocortin receptor antibody or an antigen binding fragment thereof, a small molecule inhibitor of a melanocortin peptide, a small molecule inhibitor of a melanocortin receptor, and a ‘decoy’ receptor of a melanocortin receptor, such as soluble melanocortin receptor and a fusion of a soluble melanocortin receptor with an Fc molecule. Suitable melacortin receptor inhibitors include, for example, the melanocortin receptor antagonist agouri-related peptide (AgRP(83-132)), which has been demonstrated to prevent cachexia-related symptoms in a mouse model of cancer-related cachexia (Joppa et al. (2007) PEPTIDES 28:636-642).

Anti-cancer agents, especially those that can cause cachexia and elevate GDF-15 levels, such as cisplatin, can be used in methods of the present disclosure in combination with (for example, administered at the same time as, administered before, or administered after) an agent that binds an extracellular domain of a GFRAL protein and/or competes with GDF15 for binding to the ECD of GFRAL (e.g., an antibody that competes with GDF15 for binding to ECD of GFRAL). Many cancer patients are weakened by harsh courses of radio- and/or chemotherapy, which can limit the ability of the patient to tolerate such therapies, and hence restrict the dosage regimen. Certain cancer agents themselves, such as fluorouracil, adriamycin, methotrexate and cisplatin, can contribute to cachexia, for example by inducing severe gastrointestinal complications. See, e.g., Inui (2002) CANCER J. FOR CLINICIANS 52:72-91. By the methods of the present disclosure, in which an anti-cancer agent is administered in combination with an anti-GDF-15 antibody of the disclosure, it is possible to decrease the incidence and/or severity of cachexia, and ultimately increase the maximum tolerated dose of such an anti-cancer agent. Accordingly, efficacy of treatment with anti-cancer agents that can cause cachexia can be improved by reducing the incidence of cachexia as a dose-limiting adverse effect, and by allowing administration of higher doses of a given anticancer agent.

Thus, provided herein are pharmaceutical compositions comprising an agent that binds an extracellular domain of a GFRAL protein and/or competes with GDF15 for binding to the ECD of GFRAL (e.g., an antibody that competes with GDF15 for binding to ECD of GFRAL) in combination with an agent selected from the group consisting of an inhibitor of Activin-A, an inhibitor of ActRIIB, an inhibitor of IL-6 or an inhibitor of IL-6R, a ghrelin, a ghrelin mimetic or a GHS-Rla agonist, a SARM, a TNFα inhibitor, an IL-la inhibitor, a myostatin inhibitor, a beta-blocker, a melanocortin peptide inhibitor, a melanocortin receptor inhibitor, and an anti-cancer agent. The present disclosure also includes methods of treating, preventing or minimizing cachexia and/or sarcopenia in a mammal comprising administering to a mammal in need thereof a pharmaceutical composition or compositions comprising an effective amount of an anti-GDF-15 antibody of the disclosure in combination with an effective amount of an inhibitor of Activin-A, an inhibitor of ActRIIB, an inhibitor of IL-6 or an inhibitor of IL-6R, a ghrelin, a ghrelin mimetic or a GHS-Rla agonist, a SARM, a TNFα inhibitor, an IL-la inhibitor, a myostatin inhibitor, a beta-blocker, a melanocortin peptide inhibitor, or a melanocortin receptor inhibitor.

In another aspect, provided herein is a method of inhibiting loss of muscle mass associated with an underlying disease comprising administering to a mammal in need thereof a pharmaceutical composition or compositions comprising an effective amount of an agent that binds an extracellular domain of a GFRAL protein and/or competes with GDF15 for binding to the ECD of GFRAL (e.g., an antibody that competes with GDF15 for binding to ECD of GFRAL) in combination with an effective amount of an inhibitor of Activin-A, an inhibitor of ActRIIB, an inhibitor of IL-6 or an inhibitor of IL-6R, a ghrelin, a ghrelin mimetic or a GHS-Rla agonist, a SARM, a TNFα inhibitor, an IL-lα inhibitor, a myostatin inhibitor, a beta-blocker, a melanocortin peptide inhibitor, or a melanocortin receptor inhibitor to prevent or reduce loss of muscle mass. The underlying disease can be selected from the group consisting of cancer, chronic heart failure, chronic kidney disease, chronic obstructive pulminary disease, AIDS, multiple sclerosis, rheumatoid arthritis, sepsis, and tuberculosis. Additionally, in some embodiments, the loss of muscle mass is accompanied by a loss of fat mass.

In another aspect, provided herein is a method of inhibiting or reducing involuntary weight loss in a mammal comprising administering to a mammal in need thereof a pharmaceutical composition or pharmaceutical compositions comprising an effective amount of an anti-GDF-15 antibody of the disclosure in combination with an effective amount of an inhibitor of Activin-A, an inhibitor of ActRIIB, an inhibitor of IL-6 or an inhibitor of IL-6R, a ghrelin, a ghrelin mimetic or a GHS-Rla agonist, a SARM, a TNFα inhibitor, a IL-lα inhibitor, a myostatin inhibitor, a beta-blocker, a melanocortin peptide inhibitor, or a melanocortin receptor inhibitor.

Certain anti-cancer agents, such as cisplatin, have one or more undesirable adverse effects that involve causing or increasing one or more syndromes such as cachexia, sarcopenia, muscle wasting, bone wasting or involuntary body weight loss. Accordingly, in another aspect, provided herein is a method of treating cancer, while preventing, minimizing or reducing the occurrence, frequency or severity of cachexia, sarcopenia, or muscle wasting, bone wasting or involuntary loss of body weight in a mammal, comprising administering to a mammal in need thereof a pharmaceutical composition comprising an effective amount of an agent that binds an extracellular domain of a GFRAL protein and/or competes with GDF15 for binding to the ECD of GFRAL (e.g., an antibody that competes with GDF15 for binding to ECD of GFRAL) in combination with one or more anti-cancer agents. In some embodiments, the method of treating cancer, while preventing, minimizing or reducing the occurrence, frequency or severity of cachexia, sarcopenia or muscle wasting, bone wasting or involuntary loss of body weight in a mammal, comprises administering to a mammal in need thereof a pharmaceutical composition comprising an effective amount of an agent that binds an extracellular domain of a GFRAL protein and/or competes with GDF15 for binding to the ECD of GFRAL (e.g., an antibody that competes with GDF15 for binding to ECD of GFRAL) in combination with one or more anti-cancer agents known to cause or increase the occurrence, frequency or severity of cachexia, sarcopenia, or muscle wasting, bone wasting or involuntary loss of body weight in a mammal.

### Dosages

In the methods, a therapeutically effective amount of a subject protein is administered to a subject in need thereof. For example, a subject protein causes the body weight to return to a normal level relative to a healthy individual when the subject protein is delivered to the bloodstream in an effective amount to a patient who previously did not have a normal body weight relative to a healthy individual prior to being treated. The amount administered varies depending upon the goal of the administration, the health and physical condition of the individual to be treated, age, the degree of resolution desired, the formulation of a subject protein, the activity of the subject proteins employed, the treating clinician's assessment of the medical situation, the condition of the subject, and the body weight of the subject, as well as the severity of cachexia, and other relevant factors. The size of the dose will also be determined by the existence, nature, and extent of any adverse side-effects that might accompany the administration of a particular protein.

It is expected that the amount will fall in a relatively broad range that can be determined through routine trials. For example, the amount of subject protein employed to restore body weight and/or appetite is not more than about the amount that could otherwise be irreversibly toxic to the subject (i.e., maximum tolerated dose). In other cases, the amount is around or even well below the toxic threshold, but still in an effective concentration range, or even as low as threshold dose.

Individual doses are typically not less than an amount required to produce a measurable effect on the subject, and may be determined based on the pharmacokinetics and pharmacology for absorption, distribution, metabolism, and excretion (“ADME”) of the subject protein or its by-products, and thus based on the disposition of the composition within the subject. This includes consideration of the route of administration as well as dosage amount, which can be adjusted for enteral (applied via digestive tract for systemic or local effects when retained in part of the digestive tract) or parenteral (applied by routes other than the digestive tract for systemic or local effects) applications. For instance, administration of a subject protein is typically via injection and often intravenous, intramuscular, or a combination thereof.

An effective dose (ED) is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors.

In some embodiments, the effective amount is the same as the calculated ED₅₀, and in certain embodiments the effective amount is an amount that is more than the calculated ED₅₀. In certain embodiments the effective amount is an amount that is less than the calculated ED₅₀.

An effective amount of a protein may also be an amount that is effective, when administered in one or more doses, to increase body weight of an individual by at least about 10%, at least about 20%, at least about 25%, at least about 30%, at least about 40%, at least about 50%, at least about 60%, at least about 70%, at least about 80%, or more than 80%, compared to body weight in the individual prior to the treatment.

Further examples of dose per administration may be at less than 10 μg, less than 2 μg, or less than 1 μg. Dose per administration may also be more than 50 μg, more than 100 μg, more than 300 μg up to 600 μg or more. An example of a range of dosage per weight is about 0.1 μg/kg to about 1 μg/kg, up to about 1 mg/kg or more. Effective amounts and dosage regimens can readily be determined empirically from assays, from safety and escalation and dose range trials, individual clinician-patient relationships, as well as in vitro and in vivo assays known in the art.

The term “unit dosage form,” as used herein, refers to physically discrete units suitable as unitary dosages for human and animal subjects, each unit containing a predetermined quantity of proteins of the present disclosure calculated in an amount sufficient to produce the desired effect in association with a pharmaceutically acceptable diluent, carrier or vehicle. The specifications for the novel unit dosage forms depend on the particular protein employed and the effect to be achieved, and the pharmacodynamics associated with each protein in the host.

In some embodiments, a therapeutically effective amount of a subject protein (e.g., an antibody that competes with GDF15 for binding to ECD of GFRAL) is in the range of 0.1 mg/kg to 100 mg/kg, 1 mg/kg to 100 mg/kg, 1 mg/kg to 10 mg/kg, or 2.0 mg/kg to 10 mg/kg. The amount administered can depend on variables such as the type and extent of disease or indication to be treated, the overall health of the patient, the in vivo potency of the antibody or fusion protein, the pharmaceutical formulation, the serum half-life of the antibody or fusion protein, and the route of administration. The initial dosage can be increased beyond the upper level in order to rapidly achieve the desired blood-level or tissue level. Alternatively, the initial dosage can be smaller than the optimum, and the dosage can be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody or fusion protein, and the disease being treated. Exemplary dosing frequencies are once per day, once per week and once every two weeks. In some embodiments, dosing is once every two weeks. A preferred route of administration is parenteral, e.g., intravenous infusion. Formulation of monoclonal antibody-based drugs and fusion protein-based drugs are within ordinary skill in the art. In some embodiments, the antibody or fusion protein is lyophilized, and then reconstituted in buffered saline, at the time of administration. The effective amount of a second active agent, for example, an anti-cancer agent or another agent described herein, will also follow the principles discussed hereinabove and will be chosen so as to elicit the required therapeutic benefit in the patient.

### Kits

Also provided by the present disclosure are kits for using the compositions disclosed herein and for practicing the methods, as described above. The kits may be provided for administration of the subject protein in a subject in need of treatment or prevention of cachexia. The kit can include one or more of the proteins disclosed herein, which may be provided in a sterile container, and can be provided in a formulation with a suitable pharmaceutically acceptable excipient for administration to a subject. The proteins can be provided with a formulation that is ready to be used as it is or can be reconstituted to have the desired concentrations. Where the proteins are provided to be reconstituted by a user, the kit may also provide buffers, pharmaceutically acceptable excipient, and the like, packaged separately from the subject protein. The proteins of the present kit may be formulated separately or in combination with other drugs.

In addition to above-mentioned components, the kits can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, the means for obtaining the instructions is recorded on a suitable substrate.

### EXAMPLES

The following examples are put forth so as to provide those of ordinary skill in the art with a complete disclosure and description of how to make and use the present invention, and are not intended to limit the scope of what the inventor(s) regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Materials and Methods

The following methods and materials were used in the Examples below.

Generation of HEK239-GFRAL Cell Line.

A plasmid containing human GFRAL was transfected into HEK293 cells using Lipofectamine 2000 (Life Technology). Two days after transfection, cells were incubated with DMEM containing 10% FBS and hygromycin (0.2 mg/mL) until colonies became visible. Clones were picked and evaluated for Fc-GDF15 binding using indirect immunofluorescence staining.

RT-PCR.

Tissues from C57/BL6 mice were harvested and total RNA was extracted by Trizol (Life Technology). 250 μg of total RNA was subjected to RT-qPCR analysis using Quantitect Multiplex RT-PCR kit (Qiagen). The PCR primer for mouse GFRAL was purchased from Life Technology (Catalog no. Mm 02344885-m1). The copy number of transcripts was calculated based on a standard curve generated using mouse GFRAL cDNA as the template.

In Situ Hybridization.

RNA in situ hybridization of mouse GFRAL was performed by Advanced Cell Diagnostics (Hayward, Calif.) using its RNASCOPE® Assay technology. Whole brains from C57BL/6 mice were dissected and fresh frozen in optimal cutting temperature (OCT) compound (VWR International). 20 microns cryosections were prepared, dried on slide for approximately 1 hr and stored at −20° C. until processing at Advanced Cell Diagnostics.

Immunofluorescence Staining of Mouse Brain Sections.

Whole brains from C57BL/6 mice were fixed in 4% paraformaldehyde:PBS overnight at 4° C. Tissues were washed in PBS and sectioned with a Vibratome. 50 μm sections were processed free floating in 2% BSA+0.2% Triton X-100:PBS solution through all blocking, antibody incubation, and wash steps. Sections were incubated overnight at 4° C. with primary antibodies: chicken anti-tyrosine hydroxylase (Ayes Labs), rabbit anti-Calcitonin Receptor (Thermo Scientific), and sheep anti-GFRAL antibody (R&D Systems). After washes, sections were incubated with secondary Alexa-conjugated antibodies (Invitrogen) overnight at 4° C. Tissue sections were brush-transferred and mounted on slides with Fluoromount-G (Southern Biotech). Epifluorescent images were obtained using a Leica DM 4000 B equipped with a Leica DFC 500 camera. Overlays and additional image processing were done using Image J software.

¹²⁵I-GDF15 binding to human GFRAL.

HEK293 or HEK293-human GFRAL cells were suspended in ice-cold binding buffer (Dulbecco's modified Eagle's medium containing 2 mg/ml BSA, and 25 mM HEPES, pH 7.4) and transferred to pre-wetted Multiscreen filter plates (96-well Dura PVDF 0.65 um Opaque; Millipore), which were kept on ice. Typically, 1×10⁵ of cells in 25 μl were used per well. Cells were incubated with 25 μl of binding buffer containing serial dilution of ¹²⁵I-GDF15 (2 fold serial dilution from 1000 pM to 0.1 pM final reaction concentration) with or without unlabeled GDF15 (500 nM final reaction concentration) for 2 hr at 4° C. Plates were then transferred to a vacuum filtration manifold (Pall Corporation) and supernatants were filtered. Each well was washed four times with 100 μl of ice cold binding buffer. Bound ¹²⁵I-GDF15 were measured by scintillation counting (150 μl/well of EcoLite, MP Biomedicals, Santa Anna, Calif.) using MicroBeta2 Plate Counter (Perkin Elmer). Bound (molecule/cell) was calculated according to a standard curve correlating CPM and molarity of ¹²⁵I-GDF15.

Expression and Purification of GFRAL-Fc.

Mature GFRAL-Fc proteins were secreted into the culture medium of HEK293 cells transiently transfected with a plasmid containing GFRAL-Fc. Recombinant GFRAL-Fc was purified using Protein A capturing followed by Phenyl hydrophobic interaction chromatography.

GDF15-Mediated Cellular Response.

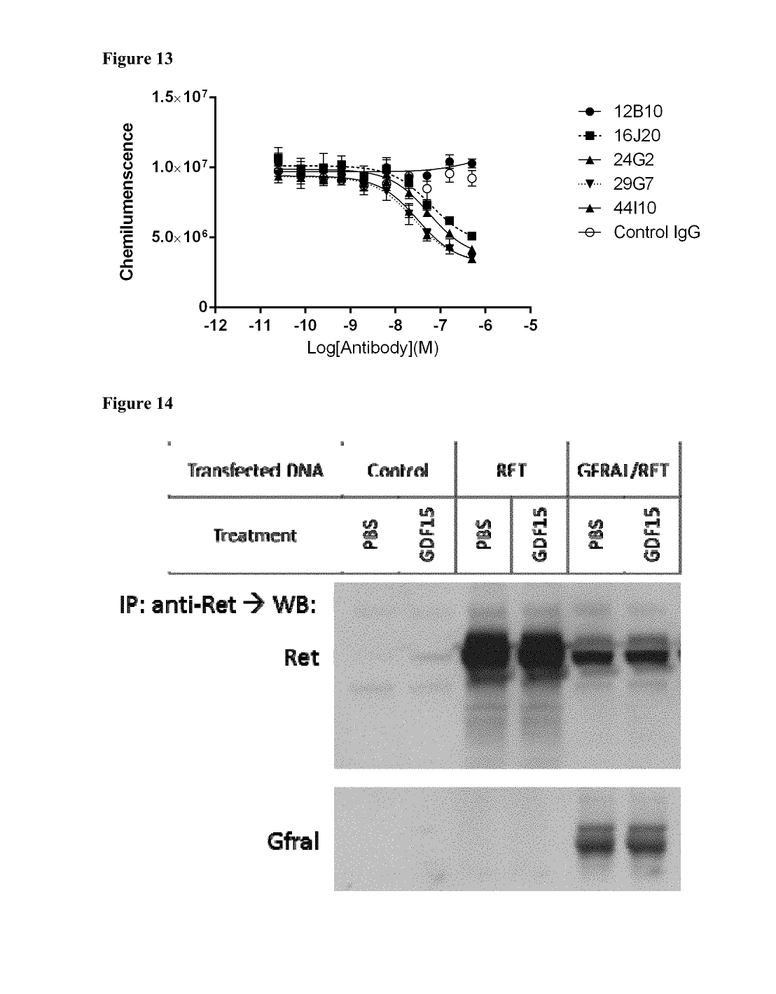

The PathDetect Trans-reporting System (Agilent Technology) was adapted. Briefly, HEK293T cells were co-transfected with the following plasmid pairs: pFA-Elk/pFR-Luc (trans-Activator/trans-Reporter; Agilent Technology) and GFRAL/RET9 expression constructs (from human, Cynomolgus monkey, rat or mouse) using Fugene 6 (Promega). Transfected cells were cultured in DMEM with 10% FBS at 37° C. overnight. Cells were treated with ligands, such as GDF15 or GDNF (Peprotech) in the presence or absence of inhibitors, such as GFRAL-Fc or an anti-GDF15 antibody (1M03) at 37° C. for 6 hr. In experiments where anti GFRAL antibodies were included (FIGS. 12 and 13), the cells were pre-treated with antibodies for an hour prior to incubation with GDF15. The reporter luciferase activities were determined using Bright Glo (Promega) as the substrate and detected by EnSpire Multimode Plate Reader (Perkin Elmer). Upon nonlinear regression curve fit (GraphPad Prism), the EC₅₀ for GDF15 activity toward human, cynomolgus monkey, rat and mouse receptors were 2.1 pM, 5.2 pM, 6.2 pM, and 1.5 pM, respectively.

GFRAL-Fc or an anti-GDF15 antibody (1M03) were added at following concentrations (nM): 100, 33.33, 11.11, 3.7, 1.23, 0.41, 0.14, 0.05, 0.02, and 0.01. Anti-GFRAL antibodies (12B10, 16J20, 24G2, 29G7, 44I10) were added at following concentrations (nM): 500, 166.67, 55.56, 18.52, 6.17, 2.06, 0.69, 0.23, 0.08, and 0.03. IC₅₀ for GFRAL-Fc and 1M03 for inhibiting 1 nM GDF15-mediated receptor activation were 9.5 nM and 11.7 nM, respectively. IC₅₀ for inhibiting 10 pM GDF15-mediated receptor activation for 16J20, 24G2, 29G7, and 44I10 were 61.1 nM, 55.9 nM, 25.7 nM, and 31.4 nM, according to nonlinear regression curve fit using GraphPad Prizm. Antibody 1M03 is a mouse monoclonal antibody that was generated using mature hGDF15 protein as the antigen.

Radio-Ligand Competition Binding.

HEK293-GFRAL cells were suspended in ice-cold binding buffer (Dulbecco's modified Eagle's medium containing 2 mg/ml BSA, and 25 mM HEPES, pH 7.4) and transferred to pre-wetted Multiscreen filter plates (96-well Dura PVDF 0.65 um Opaque; Millipore), which were kept on ice. Typically, 1×10⁵ of cells in 25 μl were used per well. 12.5 μl/well of binding buffer containing 0.6 nM ¹²⁵I-GDF15 was added. 12.5 μl/well of unlabeled inhibitors at varying concentrations were then added and incubated for 2 hrs at 4° C. Plates were then transferred to a vacuum filtration manifold (Pall Corporation) and supernatants were filtered. Each well was washed four times with 100 μl of ice cold binding buffer. Bound ¹²⁵I-GDF15 (CPM) were measured by scintillation counting (150 μl/well of EcoLite, MP Biomedicals, Santa Anna, Calif.) using MicroBeta2 Plate Counter (Perkin Elmer). The concentrations (in nM) of unlabeled inhibitors used in this experiment are as follows. GDF15 and GFRAL-Fc: 100, 25, 6.25, 1.56, 0.39, 0.098, 0.024, 0.006; 1M03: 1000, 250, 62.5, 15.63, 3.91, 0.98, 0.24, 0.06. IC₅₀ for inhibiting 0.15 nM of ¹²⁵I-GDF15 binding to HEK-293-GFRAL cells are 0.4 nM, 0.56 nM, and 0.66 nM for GDF15, GFRAL-Fc, and 1M03, respectively, according to nonlinear regression curve fit using GraphPad Prism.

ELISA-Based Competition Binding Assay.

An antibody for human Fc (Jackson ImmunoResearch) was coated onto 96-well Nunc Maxisorp plates (Fisher Scientific) at 1 μg/ml in PBS for overnight at 4 C. The wells were washed three times with washing buffer prepared from PBS-TWEEN tablets (EMD Chemicals). The wells were blocked by incubating with 1% BSA in PBS for 1 hr at RT. After washing, 1 μg/ml of GFRAL-Fc was added and incubated for 2 hr at RT. The wells were washed three times and dose titration of purified anti-GFRAL antibodies were added and incubated for 1 hr at RT. Biotinylated-GDF15 was then added to 100 ng/ml and incubated for 1 hr at RT. The wells were washed three times and incubated with HRP-conjugated streptavidin (1:5000 dilution, Life Technology) for 1 hr at RT. The wells were washed and treated with TMB (Life Technology) for 15 min at RT. OD450 was measured using SpectroMax plate reader (Molecular Devices). The antibodies were added at the following concentrations (nM): 62.5, 20.8, 6.9, 2.3, 0.8, 0.3, 0.09, 0.03. IC₅₀ for inhibiting 4 nM of biotinylated GDF15 binding to immobilized GFRAL-Fc are (in nM) 4.9, 0.6, 0.9, 0.8, and 0.4 for antibodies 12B10, 16J20, 24G2, 29G7, and 44I10, respectively.

GFRAL-RET Complex.

HEK293 cells were transfected with cDNA encoding RET51 (control) or with cDNAs encoding GFRAL and RET51 using Lipofectamine® 2000 (Life Technology) according to manufacturer's instructions. Two days after transfection, cells were starved in serum-free DMEM for 1 hour and then treated with 100 ng/ml of GDF15 for 15 minutes. Culture media were aspirated and the cells were lysed in ice-cold RIPA buffer (Sigma) containing protein phosphatase inhibitors cocktail I and II (Sigma), protease inhibitor mixture (Roche), NaF (1 mM) and NaVO₄ (1 mM). 1 mg equivalent of cell lysates were incubated with 20 μg of anti-RET51 antibody (Santa Cruz Biotechnology) for 45 min at 4° C. 100 μl of Protein G Dynabeads® (Life Technology) were added and incubated for 1.5 hr at 4° C. The beads were washed three times with PBS and the bound proteins were eluted with 60 μl of SDS-PAGE buffer. 15 μl of eluted lysates were subjected to SDS-PAGE followed by western blotting using anti-Ret51 antibody (Santa Cruz Biotechnology) or anti-GFRAL antibody (R&D Systems).

Anti-GFRAL Antibody Production.

Recombinant proteins containing GFRAL ECD were used as immunogens to immunize B6/129 mice. Hybridoma supernatants were screened for binding to GFRAL-expressing CHO cells using high content imaging system, Cell InSight (Thermo Scientific) or by binding to GFRAL-Fc using ELISA.

### Example 1: Identification of GDF15 Receptor

To identify a binding partner for mature human GDF15, a number of assays were performed. In order to identify a tissue that expresses a protein that binds to GDF15, ¹²⁵I-GDF15 was used to stain tissue sections from mouse and rats, including brain tissue sections. In addition, human biological fluid including urine, ascites fluid, and amniotic fluid was assayed for presence of GDF15 binding partner. FLAG tagged GDF15 (FLAG-GDF15) was incubated with human urine, ascites fluid, and amniotic fluid and proteins bound to the FLAG-GDF15 were analyzed by mass spectrometry. Proteins present in human urine, ascites fluid, and amniotic fluid were chromatographically fractionated followed by detection by binding to ¹²⁵I-GDF15.

Screening of cDNA libraries from mouse hypothalamus and human placenta and cDNA clones of membrane proteins for binding to GDF15 was also performed. A subset (37) of ˜4000 cDNA clones of membrane proteins were expressed in HEK293T cells and screened for binding to Fc-GDF15 (FIG. 1B; Dimer of DhCpmFc(−)-(G4S)4-GDF15:DhCpmFc(+) as disclosed in PCT/US2013/023465). Membrane protein cDNA expression constructs (0.08 μg/well) were reverse transfected in HEK-293T cells (ATCC) in 384-well poly-D-lysine coated plates (Greiner) using Lipofectamine 2000 (Invitrogen Corporation, Carlsbad, Calif.) according to manufacturer's instructions. Transfected cells were cultured for three days. Cells were washed with DMEM (no phenol red; Corning Cellgro) three times, and incubated with Fc-GDF15 (5 μg/ml) in the same medium at room temperature for 1.5 hours. An Alexa647-conjugated goat anti-human Fc antibody (Jackson Immunologicals) was added to 0.33 ng/mL and Hoescht33342 (6.7 ng/ml) (Sigma) was added to the cells and incubated for 40 minutes. Plates were washed three times with cold HBSS/M2+(Cellgro), and fixed for 5 minutes with 3.7% formalin in PBS. Fluorescence intensities were measured using Cell InSight (Thermo Scientific).

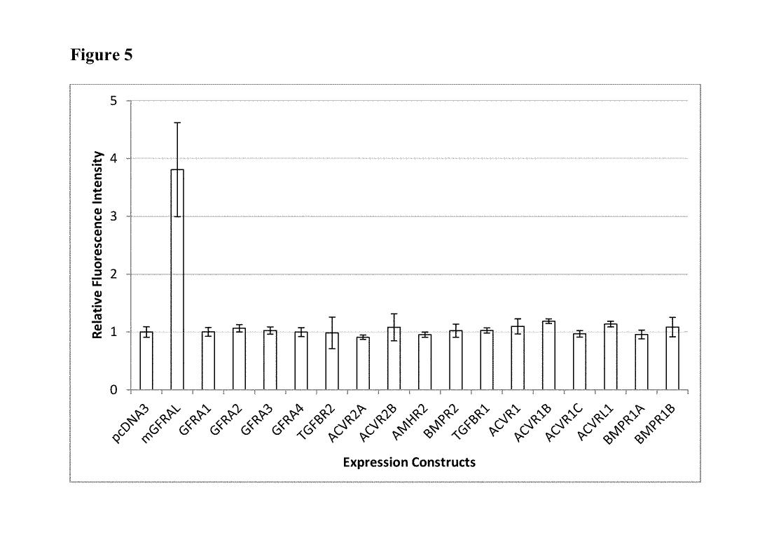

Mouse GFRAL was identified as a membrane protein that bound to Fc-GDF15. The subset of cDNA clones screened also included TGFβ family receptors and GDNF family receptors (GFRAs) which did not bind to GDF15. (FIG. 5).

### Example 2: GFRAL is Expressed in Brain Stem

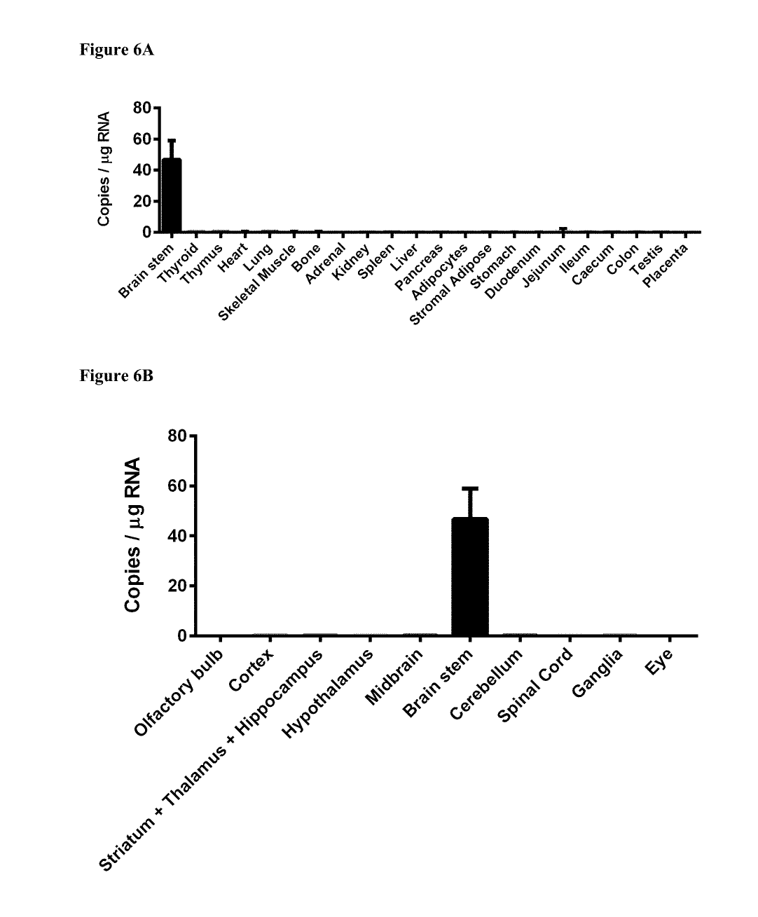

Tissues from mice were analyzed by quantitative RT-PCR to identify organs that express GFRAL. GFRAL RNA was almost exclusively expressed in brain stem (FIG. 6A). Tissue harvested from different regions of the central nervous system as well as eye was analyzed by RT-PCR and the expression of GFRAL in brain stem was confirmed (FIG. 6B).

Using RNA in situ hybridization, the expression of GFRAL mRNA in mouse brain sagittal sections was examined. Upon careful inspection, definitively positive staining of GFRAL was only observed in the area postrema region of brain stem. Thus, mouse GFRAL appears to be expressed exclusively in area postrema of brain stem.

Using indirect immunofluorescence staining, the expression of GFRAL protein in mouse brain sagittal sections was examined. Upon careful inspection, the definitively positive staining of GFRAL protein was only observed in the area postrema region of brain stem. Co-staining for GFRAL with neuronal markers including tyrosine hydroxylase (TH), calcitonin receptor (CT), and GLP-1 receptor was performed. GFRAL expression partially overlaps with TH- or GLP-1 receptor-expressing neurons but are excluded from CT positive neurons. These data indicate that GFRAL is expressed in a unique subset of neurons in area postrema.

### Example 3: GDF15 Binds to GFRAL at Sub-Nanomolar Affinity

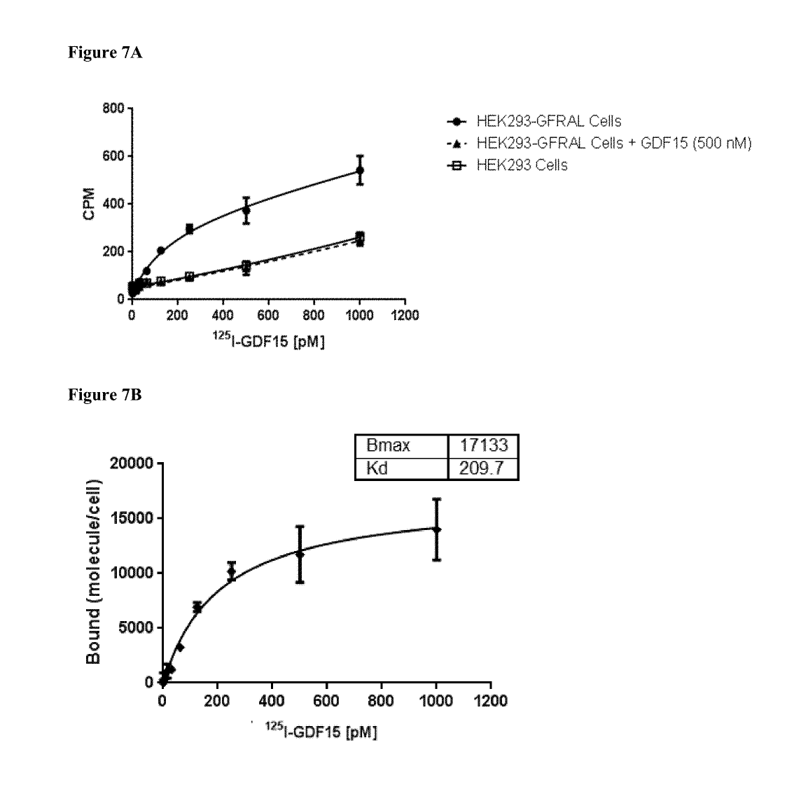

To determine the binding affinity of GDF15 to GFRAL, radio-labeled ¹²⁵I-GDF15 was used to bind HEK293 cells expressing human GFRAL (FIG. 2A) under equilibrium conditions. As shown in FIG. 7A, binding of ¹²⁵I-GDF15 to HEK293-GFRAL cells followed a typical ligand-receptor binding hyperbolic curve; in contrast, ¹²⁵I-GDF15 binding to control HEK293T cells is linear representing non-specific interaction. Excess amount of unlabeled GDF15 (500 nM) reduced the ¹²⁵I-GDF15-GFRAL binding to ‘non-specific’ background level. This further supported the specificity of ¹²⁵I-GDF15 and GFRAL interaction. The binding affinity of ¹²⁵I-GDF15-GFRAL is around 200 pM (FIG. 7B), which is also typical for high affinity ligand-receptor interaction.

### Example 4: GDF15 Stimulates Erk Signal Transduction Pathway Via GFRAL and RET

The extracellular domain of GFRAL shares sequence homology with GDNF family receptor alpha (GFRA) family receptors. GFRA receptors are anchored to cell surface by glycosylphosphatidylinositol(GPI). GFRA receptors, upon binding to their ligand, associate with receptor tyrosine kinase RET and mediate activation of RET. Activated RET becomes tyrosine phosphorylated, which induces an intracellular signaling cascade including phosphorylation of transcription factor ELK1.

To test the hypothesis that GDF15 mediates cellular response, such as, ELK1 phosphorylation, via GFRAL-RET receptor complex, cells expressing both hGFRAL and human RET9 (FIG. 3B) were assayed for response to GDF15. HEK293T cells were co-transfected with expression plasmids for hGFRAL and hRET9, together with a reporter system that includes a GAL4DBD-ELK1 fusion plasmid and a GAL4-UAS-Luciferase reporter gene (PathDetect Trans-Reporting Systems, Agilent Technology).

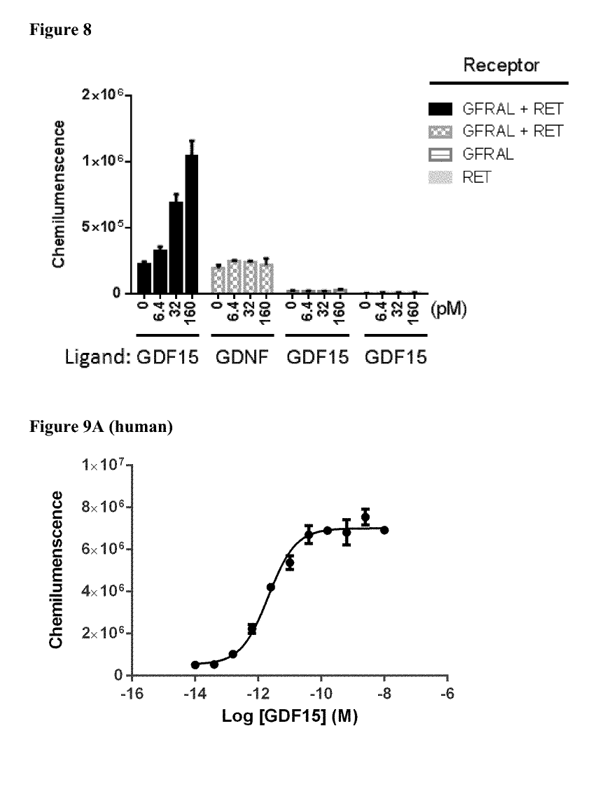

As shown in FIG. 8, GDF15 mediates expression of luciferase in a dose-dependent manner. In contrast, GDNF, which mediates RET activation by binding to GFRA1, does not activate GFRAL-RET. Both GFRAL and RET are required for GDF15-mediated cellular response as omitting either one of these receptors abrogated luciferase expression. This result supports the hypothesis that GDF15 exerts its biological function via the GFRAL-RET receptor system.

### Example 5: Human GDF15 Activates GFRAL-RET Receptor Systems from Human, Cynomolgus Monkey, Rat and Mouse

The conservation of GFRAL-RET receptor system in response to GDF15 treatment was examined using the reporter assay described in Example 4. GFRAL and RET9 receptors from human, cynomolgus monkey, rat and mouse were transfected into HEK293T cells and the reporter gene activation in response to human GDF15 was measured.

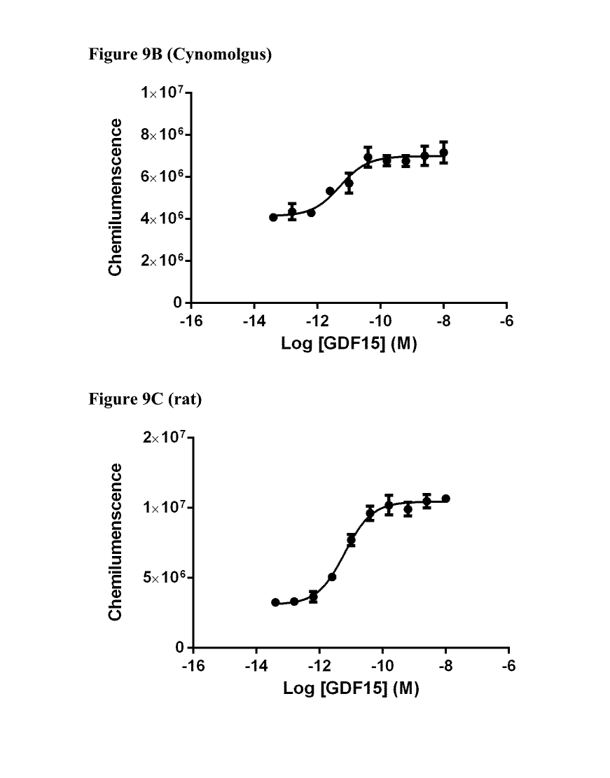

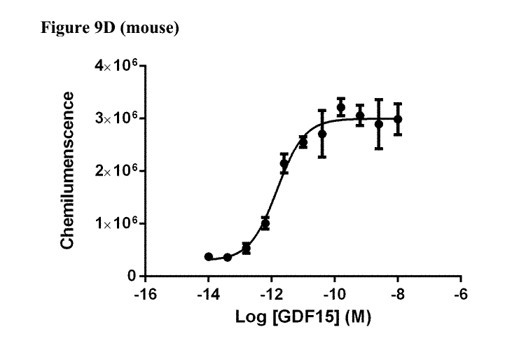

As shown in FIGS. 9A-9D, GDF15 dose-dependently mediated cellular response via the GFRAL-RET receptor complex from all four species tested. These data demonstrated that the GDF15/GFRAL-RET ligand/receptor system is conserved.

### Example 6: GFRAL-Fc and an Anti-GDF15 Antibody, 1M03, Inhibit Binding of GDF15 to GFRAL

Molecules that inhibit binding of GDF15 to GFRAL were identified using a competition binding experiment. HEK293-GFRAL cells bound to ¹²⁵I-GDF15 (0.15 nM) were incubated with unlabeled GDF15, GFRAL-Fc or antibody 1M03.

GFRAL-Fc includes the extracellular domain of hGFRAL. As shown in FIG. 4, GFRAL-Fc includes a heterologous signal peptide: IgK signal peptide which replaces the endogenous signal peptide (see FIG. 2A). GFRAL-Fc is expressed with the signal peptide which is cleaved upon secretion from the cells. GFRAL-Fc includes the extracellular domain of GFRAL but not the transmembrane domain or the intracellular domain of GFRAL.

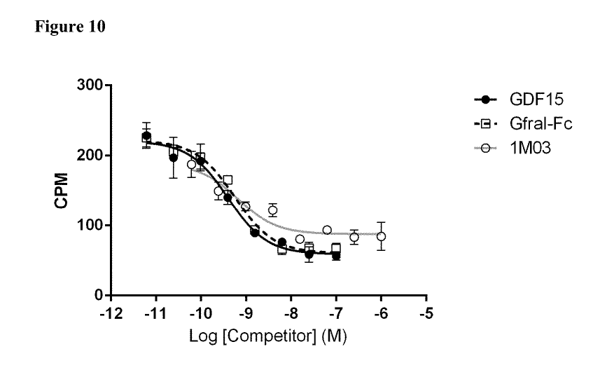

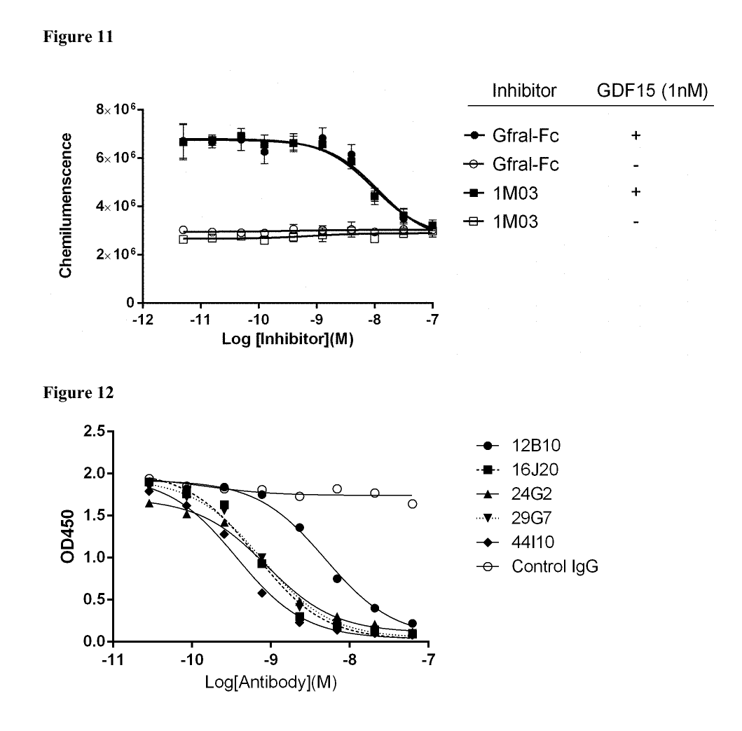

Unlabeled GDF15, GFRAL-Fc or antibody 1M03 inhibited ¹²⁵I-GDF15 (0.15 nM) binding to HEK293-GFRAL cells in dose-dependent manner (FIG. 10). The IC₅₀ for GDF15, GFRAL-Fc, and 1M03 were 0.4 nM, 0.56 nM, and 0.66 nM, respectively.

### Example 7: GFRAL-Fc and Anti-GDF15 Antibody Inhibit GDF15-Mediated Receptor Activation

Binding of GFRAL-Fc or an anti-GDF15 antibody (1M03) to GDF15 inhibits the GDF15 mediated activation of GFRAL-RET receptor complex. Effect of GFRAL-Fc and 1M03 on GDF15 mediated activation of GFRAL-RET receptor complex was examined using the reporter assay described in Example 4. GFRAL-Fc and 1M03 dose-dependently inhibited GDF15 (1 nM)-mediated reporter activation in HEK293T cells expressing GFRAL and RET (FIG. 11). The IC₅₀ for GFRAL-Fc and 1M03 were 9.5 nM and 11.7 nM, respectively.

### Example 8: Anti-GFRAL Antibodies Inhibit Binding of GDF15 to GFRAL

Anti-GFRAL antibodies were generated using GFRAL ECD as described in materials and methods. Several monoclonal anti-GFRAL antibodies were examined for their ability to interfere with GDF15 binding to GFRAL. GFRAL-Fc (FIG. 4) was immobilized on plates for conducting ELISA based binding assay as described in the materials and methods. As shown in FIG. 12, biotinylated-GDF15 (4 nM) binding to the GFRAL-Fc immobilized on plates was competed by these anti-GFRAL antibodies in a dose-dependent fashion. The IC₅₀ for anti-GFRAL ECD antibodies: 12B10, 16J20, 24G2, 29G7, and 44I10 were 4.9 nM, 0.6 nM, 0.9 nM, 0.8 nM, and 0.4 nM, respectively.

### Example 9: Anti-GFRAL Antibodies Inhibit GDF15-Mediated Receptor Activation

Effect of anti-GFRAL antibodies on the GDF15-mediated GFRAL-RET receptor complex activation was examined using the reporter assay described in Example 4. Four anti-GFRAL ECD antibodies, 16J20, 24G2, 29G7 and 44I10, dose-dependently inhibited GDF15 (10 pM)-mediated reporter activation in HEK293T cells expressing GFRAL and RET (FIG. 13). As illustrated by FIG. 12, the 12B10 antibody, which inhibits biotinylated-GDF15 binding to GFRAL-Fc less efficiently compared to the other anti-GFRAL ECD antibodies, failed to inhibit GDF15-mediated receptor activation.

### Example 10: GFRAL Interacts with Ret Independent of GDF15

To examine the interaction between GFRAL and RET, immunoprecipitation of RET was performed. HEK293 cells transfected with a plasmid encoding RET alone or co-transfected with plasmids encoding RET and GFRAL were treated with PBS only or 100 ng/ml of GDF15 for 15 minutes. Whole cell lysates from the treated cells were subjected to immunoprecipitation using an anti-Ret51 antibody followed by western blot analysis.

As shown in FIG. 14, GFRAL when co-expressed with RET on HEK293 cells, was pulled down by an anti-Ret antibody. This interaction is independent of GDF15 since equivalent GFRAL was pulled down from GFRAL/RET-expressing cells treated with GDF15 or with PBS. This result indicated that GFRAL-Ret complex is formed on the cell surface independent of GDF15.

### Example 11: Complex Formation and Crystallization of a GFRAL/GDF15 Complex

A complex of a GFRAL protein and a GDF15 protein was made by mixing 1.2 molar excess of a GFRAL (W115-E351) protein with 1 molar GDF15 protein subunit (0.5 molar GDF15, which is a homodimer of two GDF15 subunits linked by a pair of disulfide bonds). The complex was purified by size exclusion chromatography to remove excess GFRAL. The GFRAL/GDF15 complex was crystallized by mixing 1 μL protein at 5 mg/ml with 0.5 μL reservoir solution and 0.5 μL seed in a crystallization drop, with the reservoir solution containing 1.0 mL of 0.1 M Bis-Tris pH 6.0, 1.5 M (NH₄)₂SO₄ and 10% ethylene glycol. The seed crystals were obtained from a crystallization condition including a reservoir solution of 0.1 M Bis-Tris pH 6.0 and 1.5 M (NH₄)₂SO₄. The crystallization setup was kept at room temperature in Rigaku 24 well clover leaf plate. The crystallization drop showed small needle crystals after three days of incubation.

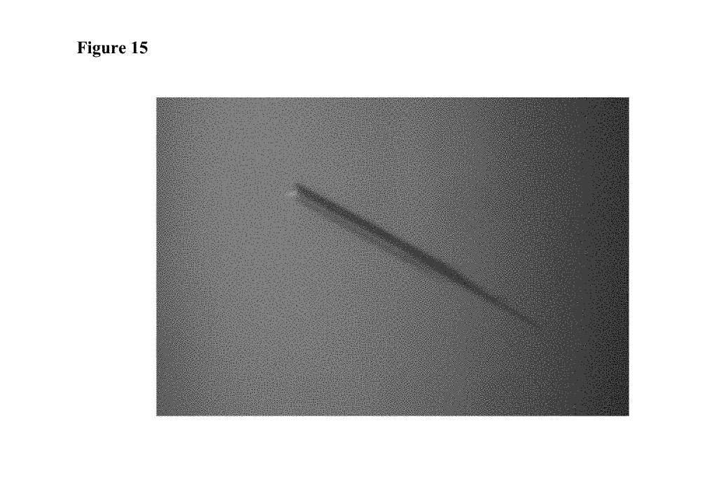

An exemplary small needle crystal of a comples of a GFRAL protein and a GDF15 protein is shown in FIG. 15.

The molecular model was not available for GFRAL, hence NaBr soaking was used to determine crystal phasing. A GFRAL/GDF15 crystal obtained as described above was soaked with 0.5 M NaBr and 0.75 M NaBr containing reservoir solution. After 30 minutes, 0.5 M NaBr soaked crystals were in good condition, whereas 0.75 M NaBr soaking yielded cracked crystals. Crystals from both soaks and un-soaked crystals were mounted with 30% EG as a cryo-protectant.

The model described herein provides the first structural information for a GFRAL protein and the binding of a GFRAL protein to a GDF15 protein.

### Example 12: Data Collection and Structure Determination

GFRAL/GDF15 complex crystals were obtained and harvested from a 0.1 M Bis-Tris pH 6.0, 1.5 M (NH₄)₂SO₄ and 10% ethylene glycol reservoir condition as soaked and unsoaked crystals from 0.5 M and 0.7 M NaBr soaks. The crystals were treated with the mother liquor supplemented with 20% ethylene glycol as cryoprotectant and flash-frozen in liquid nitrogen. These crystals were then examined for x-ray diffraction at the synchrotron beamline IMCA-CAT, Advanced Photon Source, Argonne National Lab. The crystal diffracted up to 2.28-2.20 Å resolution.

X-ray diffraction statistics for exemplary GFRAL/GDF15 complex crystals are shown in Table 1.

TABLE 1

| Data collection statistics | Crystal I | |
|---|---|---|
| Wave length | 0.9786 Å | |
| Space group | P2₁ | |
| Unit cell (Å) | a = 75.352 | |
| | b = 88.768 | |
| | c = 121.293 | |
| Resolution (Å)^{†} | 50-2.20 | |
| | (2.28-2.20) | |
| Number of measurements | 118,710 | |
| Number of unique reflections | 20,379 | |
| R_{sym} (%)^{†} | 0.09 | (0.58) |
| Completeness (%)^{†} | 97.4 | (85.4) |
| I/σ ^{†} | 18.9 | (2.2) |
| Redundancy^{†} | 5.8 | (4.9) |

| | |
|---|---|
| Molecules in the A.U. | 1 GFRAL |
| | 1 GDF15 |

^{†}The parenthesis is for the highest resolution shell in Å.

Molecular replacement of GFRAL/GDF15 was performed by using the scaled dataset with a previously solved GFRAL/GDF15 complex at 3.2 Å resolutions as a starting model and the rigid body refinement (See Vagin, A. A., et al., (2004) “REFMAC5 dictionary: Organization of prior chemical knowledge and guidelines for its use.” Acta Crystallogr. D 60:2284-2295) and initial positional refinement was completed in REFMAC5 as implemented in CCP4. Several rounds of model rebuilding resulted in structures of the GFRAL/GDF15 complex.

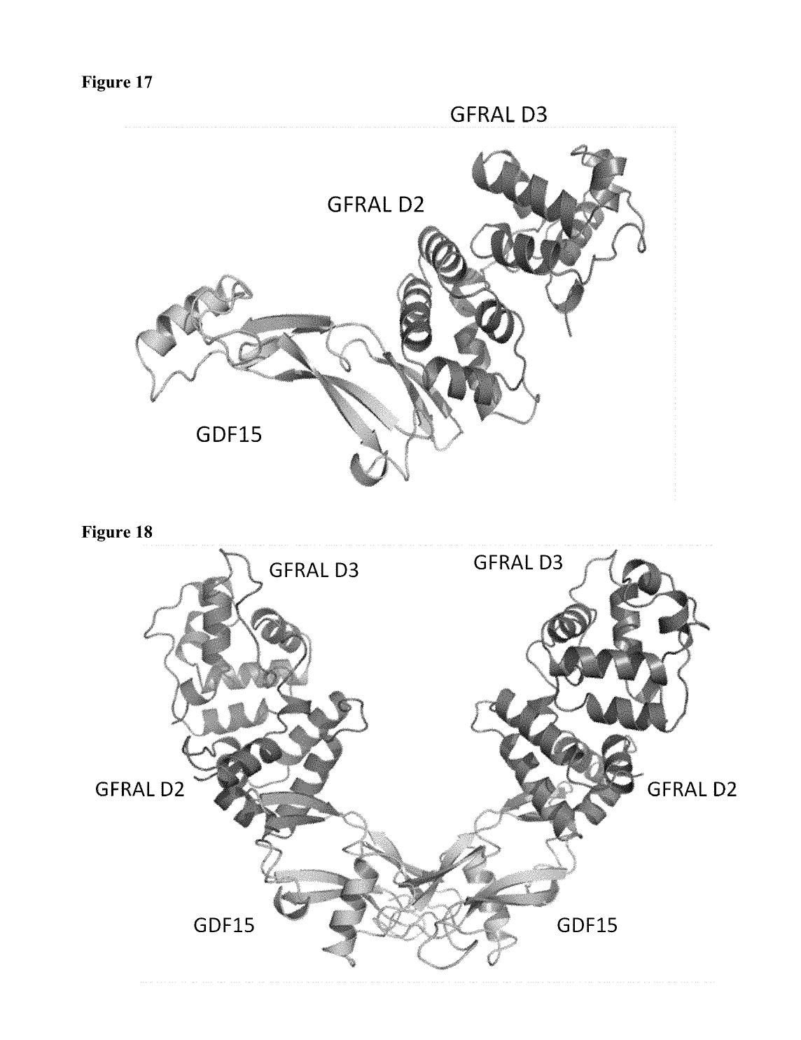

Exemplary structures of the a comples of a GFRAL protein and a GDF15 protein are shown, for example, in FIGS. 17-24B.

Inspection of the initial electron density maps showed unambiguous density for GFRAL and GDF15. After rigid body refinement, several rounds of model building and restrained refinement were performed using COOT (See Emsley, P. and Cowtan, K. (2004) “COOT: model-building tools for molecular graphics.” Acta Crystallogr. D 60:2126-2132). After placement of the solvent molecules final refinement was completed.

The atomic coordinates from the x-ray diffraction patterns are found in Table 6.

Refinement statistics for exemplary crystals are shown in Table 2.

TABLE 2

| Refinement Statistics | |
|---|---|
| Refinement Range (Å) | 35.82-2.20 |
| R_{cryst} (%) | 20.1 |
| R_{free} (%) | 26.2 |
| Molecules GDF15, GFRAL; | 1.1; 132 |
| Water molecules | |
| Bond lengths (Å) | 0.019 |
| Bond angles (°) | 1.943 |
| Average B-factors (Å²) | Overall |
| Main chain atoms | 43.9, 57.6 |
| (GDF15, GFRAL) | |
| Side chain atoms | 51.2, 66.8 |
| (GDF15, GFRAL) | |
| Water molecules | 56.7 |
| Ramachandran Plot (%) | Overall |
| Favored | 96.3 |
| Allowed | 2.7 |
| Disallowed | 1.0 |

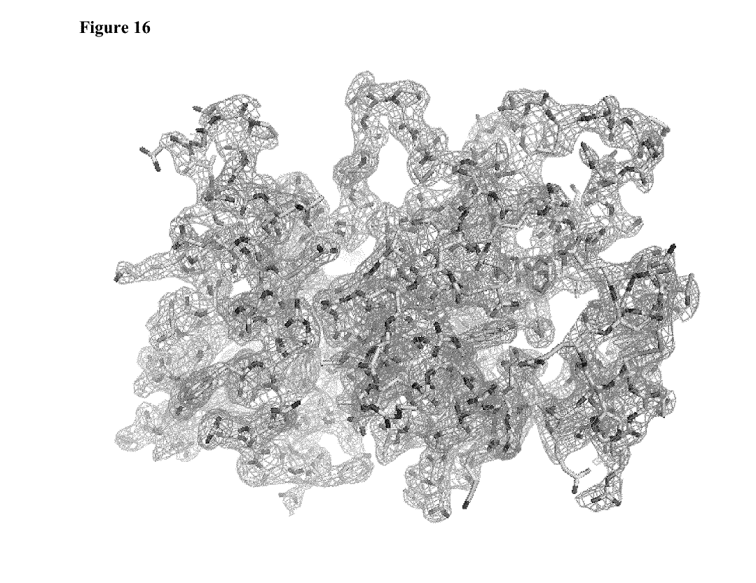

The clear electron density for GFRAL in an exemplary GFRAL/GDF15 complex crystal is illustrated in FIG. 16. FIG. 16 shows an electron density map (2fo-fc) for the GFRAL molecule calculated with 2.20 Å resolution data and contoured at the 1σ level. The GFRAL residues are clearly visible.

### Example 13: Crystal Structure of GFRAL/GDF15 Complex

The crystal structure of a complex of a GFRAL protein and a GDF15 protein was determined.

Core interaction interface amino acids were determined as being the amino acid residues (on a protein such as GFRAL) with at least one atom less than or equal to 4.5 Å from the GFRAL interacting proteins (such as GDF15). 4.5 Å was chosen as the core region cutoff distance to allow for atoms within a van der Waals radius plus a possible water-mediated hydrogen bond.

Boundary interaction interface amino acids were determined as the amino acid residues (on a protein such as GFRAL) with at least one atom less than or equal to 5 Å from core interaction interface amino acids on GFRAL that interact with GFRAL interacting proteins (such as GDF15). Less than or equal to 5 Å was chosen as the boundary region cutoff distance because proteins binding to residues less than 5 Å away from core interaction interface amino acids on GFRAL will be within the van der Waals radius of GFRAL interacting proteins.

Amino acids that met these distance criteria were calculated with the Molecular Operating Environment (MOE) program from CCG (Chemical Computing Group).

FIG. 17 shows an exemplary illustration of a heterodimeric GFRAL/GDF15 complex, as found in the asymmetric unit of a GFRAL/GDF15 protein crystal. The dimeric molecule GDF15 has one intermolecular disulfide link, which was found to be weak due to radiation damage. One side of a GDF15 molecule can form a dimer in the asymmetric unit. FIG. 18 shows an exemplary dimeric arrangement of the GFRAL/GDF15 hetero dimers in a GFRAL/GDF15 crystal.

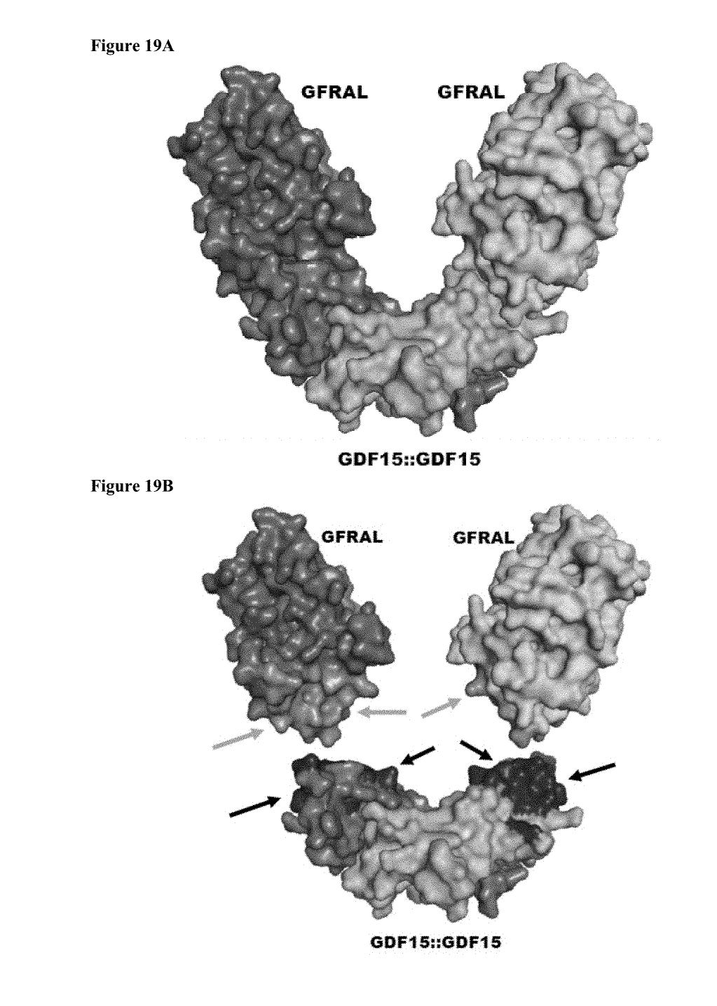

FIGS. 19A-19B illustrate the extent of the protein-protein contacts on a GFRAL-GDF15 interface. The contact region on GFRAL is indicated by light gray arrows; the contact region on GDF15 is indicated by the black arrows.

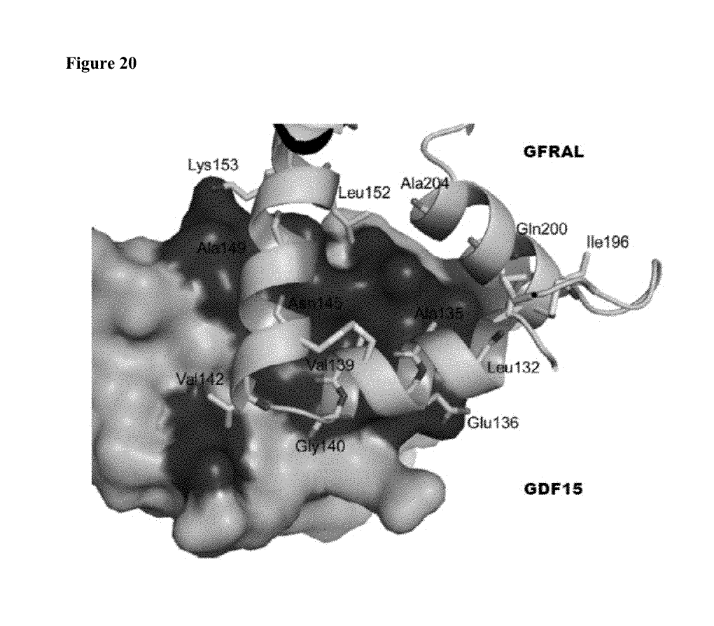

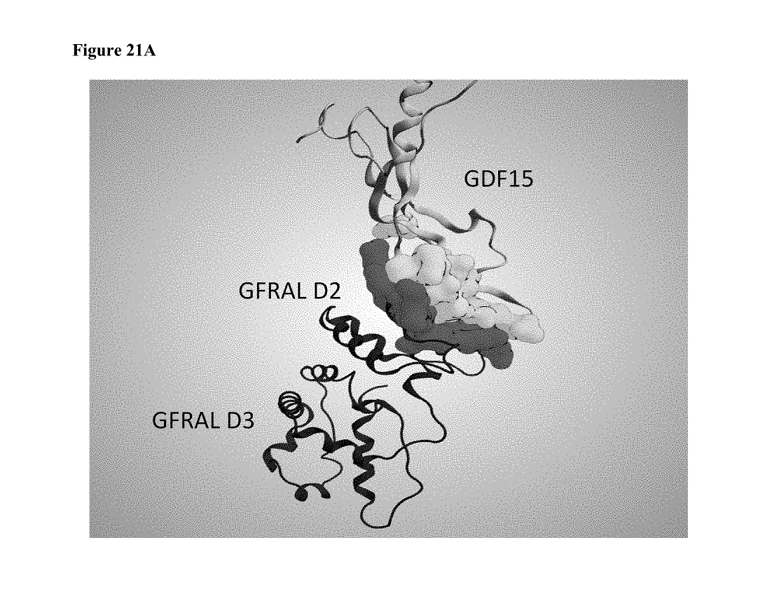

FIG. 20 shows that three α-helices of GFRAL are involved in a GFRAL/GDF15 interface. Multiple disulfide bridges appear to stabilize the structural arrangement of the three GFRAL α-helices.

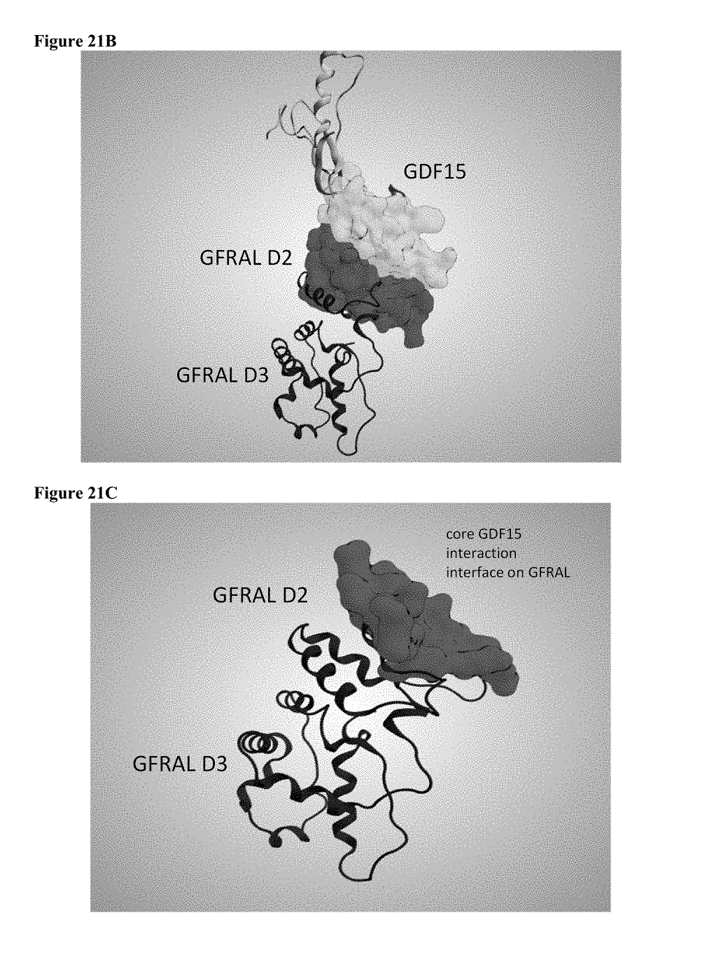

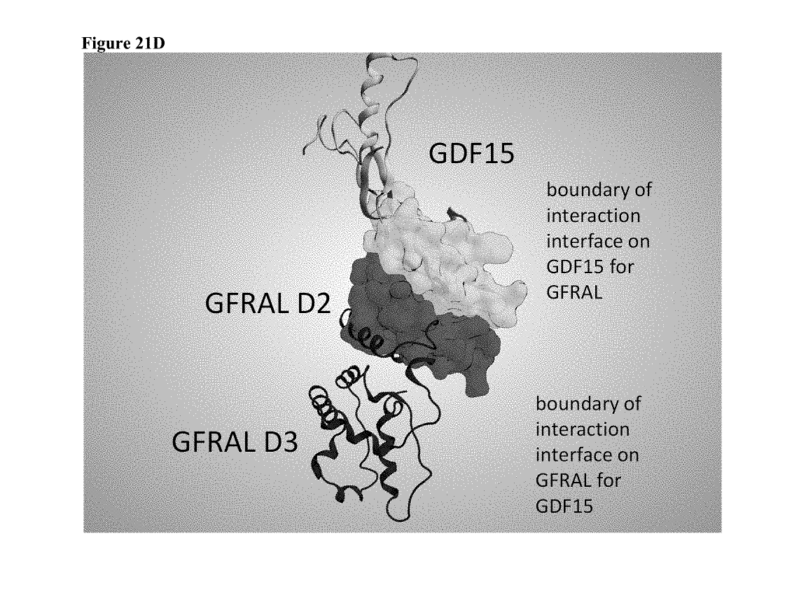

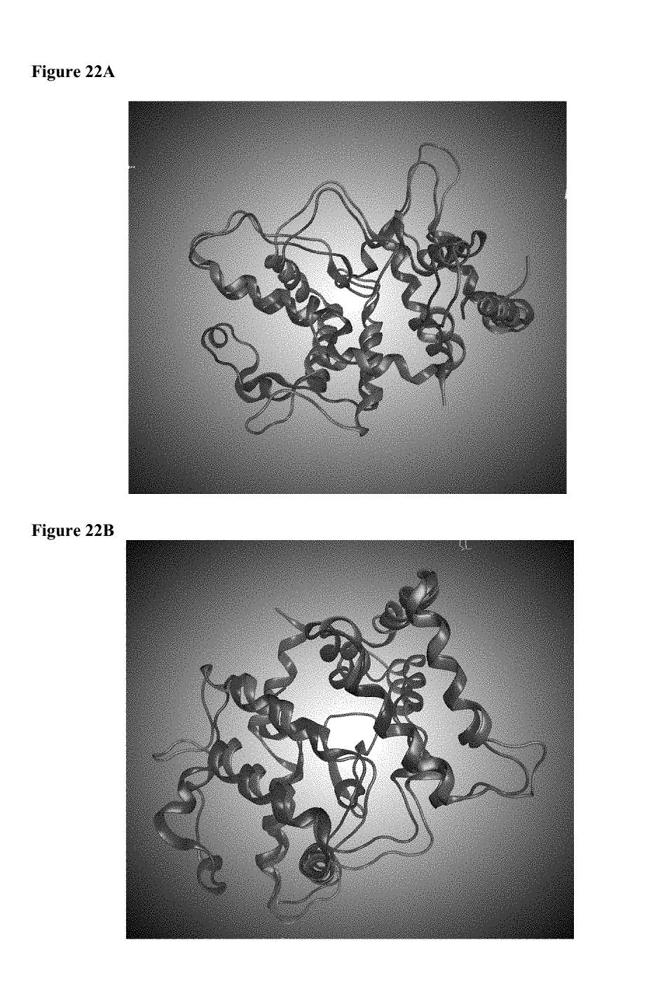

FIGS. 21A-21D illustrate different aspects of a GFRAL/GDF15 interface and the core and boundary amino acid residues of a GFRAL protein and a GDF15 protein involved in forming a GFRAL/GDF15 interface. The GFRAL protein and the GDF15 protein are depicted as ribbon diagrams with residues in the GFRAL/GDF15 interface shown in a space-filled surface representation. FIGS. 21A-21C show core interaction interface amino acids of the GFRAL protein and the GDF15 protein. FIG. 21D shows boundary interaction interface amino acids.

The amino acid sequence of a full-length precursor human GFRAL protein is shown below:

```
GFRAL sequences
SEQ ID NO: 9
                10 20 30 40
MIVFIFLAMG LSLENEYTSQ TNNCTYLREQ CLRDANGCKH
                50 60 70 80
AWRVMEDACN DSDPGDPCKM RNSSYCNLSI QYLVESNFQF
                90 100 110 120
KECLCTDDFY CTVNKLLGKK CINKSDNVKE DKFRWNLTTR
              130 140 150 160
SHHGFKGMWS CLEVAEACVG DVVCNAQLAS YLKACSANGN
              170 180 190 200
PCDLKQCQAA IRFFYQNIPF NIAQMLAFCD CAQSDIPCQQ
              210 220 230 240
SKEALHSKTC AVNMVPPPTC LSVIRSCQND ELCRRHYRTF
              250 260 270 280
QSKCWQRVTR KCHEDENCIS TLSKQDLTCS GSDDCKAAYI
              290 300 310 320
DILGTVLQVQ CTCRTITQSE ESLCKIFQHM LHRKSCFNYP
              330 340 350 360
TLSNVKGMAL YTRKHANKIT LTGFHSPFNG EVIYAAMCMT
              370 380 390
VTCGILLLVM VKLRTSRISS KARDPSSIQI PGEL
```

GFRAL amino acids at the interface of the GFRAL/GDF15 complex are shown in Table 3.

TABLE 3
GFRAL Residues Binding GDF15*

| Core interaction interface | Boundary interaction |
|---|---|
| amino acids | interface amino acids |
| GLY140 | SER156 |
| LEU148 | GLN147 |
| ALA149 | LEU148 |
| ALA146 | ALA149 |
| VAL142 | SER150 |
| ASN145 | TYR151 |
| VAL139 | LEU152 |
| ALA135 | LYS153 |
| GLU136 | ALA154 |
| LEU152 | CYS155 |
| LEU132 | PHE174 |
| SER201 | TYR175 |
| ALA204 | GLU136 |
| LEU205 | ALA137 |
| LYS153 | CYS138 |
| ILE196 | VAL139 |
| PRO197 | GLY140 |
| GLN200 | ASP141 |
| | VAL142 |
| | VAL143 |
| | CYS144 |
| | ASN145 |
| | ALA146 |
| | LEU186 |
| | CYS189 |
| | CYS191 |
| | ALA192 |
| | GLN193 |
| | SER194 |
| | ASP195 |
| | ILE196 |
| | PRO197 |
| | CYS198 |
| | GLN199 |
| | GLN200 |
| | SER201 |
| | LYS202 |
| | GLU203 |
| | ALA204 |
| | LEU205 |
| | HIS206 |
| | SER207 |
| | SER130 |
| | CYS131 |
| | LEU132 |
| | GLU133 |
| | VAL134 |
| | ALA135 |

*GFRAL amino acid numbering according to SEQ ID NO: 9

The amino acid sequence of mature human GDF15 is shown below:

(SEQ ID NO: 6)
ARNGDHCPLG PGRCCRLHTV RASLEDLGWA DWVLSPREVQ
VTMCIGACPS QFRAANMHAQ IKTSLHRLKP DTVPAPCCVP
ASYNPMVLIQ KTDTGVSLQT YDDLLAKDCH CI

GDF15 residues at the interface of the GFRAL/GDF15 complex are shown in Table 4.

TABLE 4
Residues on GDF15 that bind to GFRAL*

| Core interaction interface | Boundary interaction |
|---|---|
| amino acids | interface amino acids |
| SER35 | SER35 |
| LEU34 | VAL33 |
| THR94 | LEU34 |
| GLY95 | ASP93 |
| GLN40 | THR94 |
| VAL96 | GLY95 |
| LEU98 | VAL39 |
| PRO36 | GLN40 |
| VAL87 | ARG37 |
| LEU88 | GLU38 |
| ILE89 | VAL96 |
| ASP102 | SER97 |
| THR100 | LEU98 |
| PRO85 | PRO36 |
| MET86 | VAL87 |
| | LEU88 |
| | ILE89 |
| | VAL41 |
| | GLN90 |
| | LYS91 |
| | THR92 |
| | THR42 |
| | LEU104 |
| | LEU105 |
| | TYR101 |
| | ASP102 |
| | ASP103 |
| | GLN99 |
| | THR100 |
| | LEU24 |
| | TRP32 |
| | TRP29 |
| | ARG21 |
| | THR19 |
| | TYR83 |
| | ASN84 |
| | PRO85 |
| | MET86 |
| | VAL20 |

*GDF15 amino acid numbering according to SEQ ID NO: 6

### Example 14: Model of GFRAL/RET/GDF15 Complex

The RET/GFRα1/GDNF ternary complex described by Goodman et al. (2014) CELL REPORTS 8, 1894-1904 (PDB 4UX8) was used as a template to build a model of the complex of GFRAL/GDF15/RET (from GFRAL/GDF15 structure, see, e.g., Examples 11-13). The RET/GFRα1/GDNF template resulted from an electron microscopy reconstruction of a reconstituted mammalian RET(ECD)-GDNF-GFRα1 ternary complex (Goodman et al., supra).

To compare the structural similarity of the GFRAL/GDF15 crystal structure from Example 13 and the structure of GFRα1/GDNF in the RET/GFRα1/GDNF template, the GFRAL structure in GFRAL/GDF15 crystal was superposed with GFRα1 in GFRα1/GDNF/RET model (PDB 4UX8) using MOE from CCG. The high quality of the superposition, and therefore the structural similarity of the GFRAL/GFRα1 and GFRAL/GDF15 complexes was demonstrated by an RMSD of GFRAL/GFRα1 backbone residues of 2.21 Å. This ternary complex model, including the GFRAL/GDF15 structure and the RET structure, was used to map the interactions between GFRAL and RET.

FIGS. 22A-B illustrate exemplary aspects of the superposition of GFRAL and GFRα1 in 4XU8. RMSD of backbone residues was 2.21 Å.

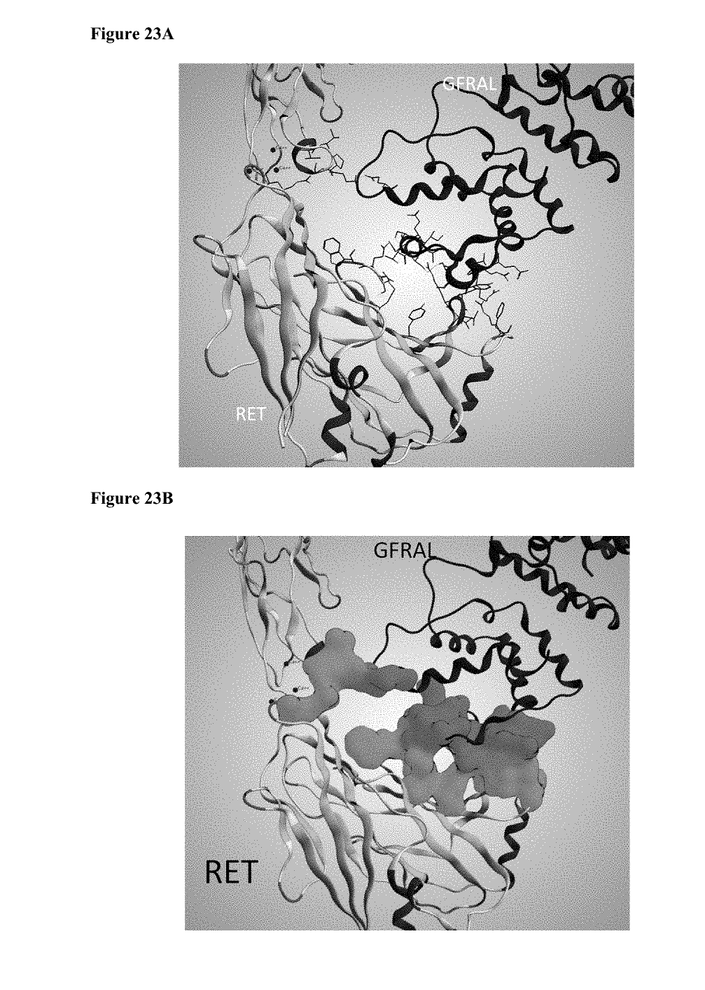

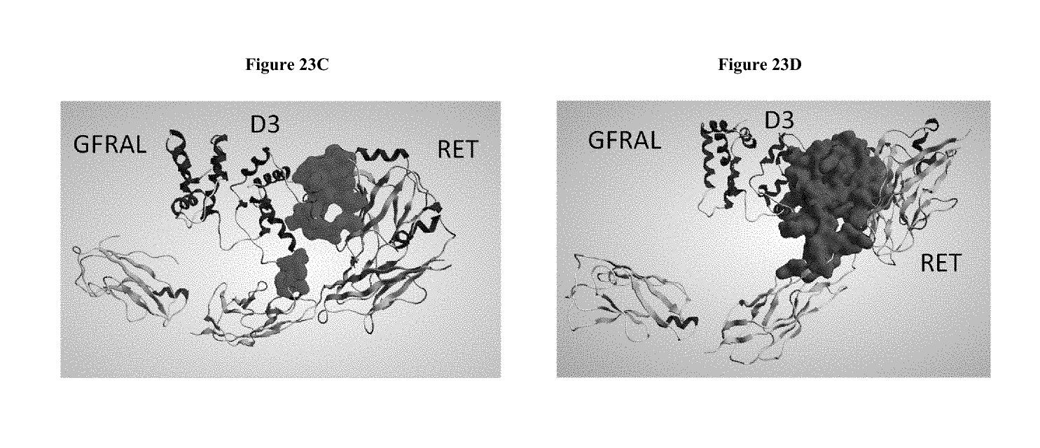

FIGS. 23A-D illustrate exemplary aspects of the interaction of a GFRAL protein with a RET protein in a RET/GFRAL/GDF15 model. In FIG. 23A, interacting GFRAL and GDF15 residues at the GFRAL/GDF15 interface as modeled are represented by stick models. In FIG. 23B, the RET-interacting residues on GFRAL are depicted in a space filled surface model. In FIG. 23C, the space filled surface model of the core interaction residues are highlighted on GFRAL and RET. In FIG. 23D, the space filled surface model of the boundary interaction residues are highlighted on GFRAL and RET.

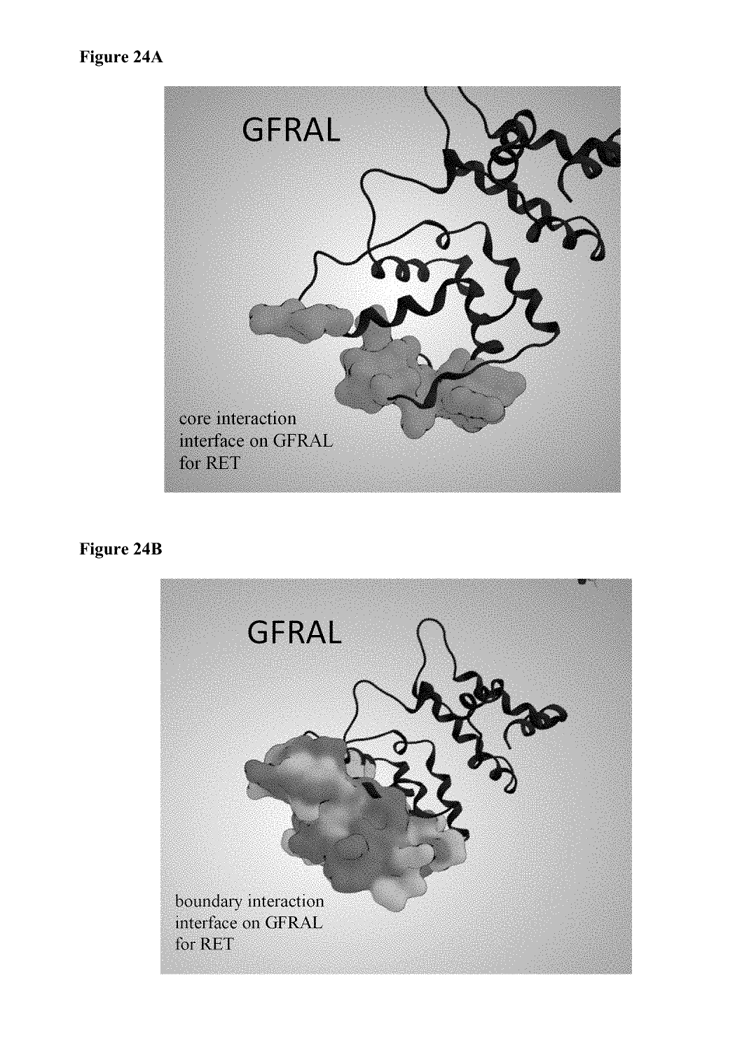

FIGS. 24A-B illustrate the core and boundary amino acid residues on a GFRAL protein identified in space filled surface models at the modeled RET interface. In FIG. 24A, core residues on GFRAL as modeled are shown in a darker grey in a space-filled surface model. In FIG. 24B, boundary residues on GFRAL as modeled are shown in a lighter grey in a space filled surface model.

Based on this modeling, a number of GFRAL residues were identified for interaction with RET residues, as shown in Table 5 Å. Additionally, a number of RET residues were identified for interaction with GFRAL residues, as shown in Table 5B.

TABLE 5A
Residues on GFRAL that bind to RET in
RET/GFRAL/GDF15 Model

| Core interaction | Boundary interaction |
|---|---|
| interface amino acids | interface amino acids |
| GLN246 | ILE224 |
| ARG247 | ARG225 |
| ARG250 | GLN241 |
| LYS251 | SER242 |
| CYS252 | LYS243 |
| ASP255 | CYS244 |
| GLU256 | TRP245 |
| ASN257 | GLN246 |
| CYS258 | ARG247 |
| ILE259 | VAL248 |
| SER260 | THR249 |
| THR261 | ARG250 |
| LEU262 | LYS251 |
| THR297 | CYS252 |
| GLN298 | HIS253 |
| SER299 | GLU254 |
| | ASP255 |
| | GLU256 |
| | ASN257 |
| | CYS258 |
| | ILE259 |
| | SER260 |
| | THR261 |
| | LEU262 |
| | SER263 |
| | LYS264 |
| | GLN265 |
| | ASP266 |
| | LEU267 |
| | THR268 |
| | THR295 |
| | ILE296 |
| | THR297 |
| | GLN298 |
| | SER299 |
| | GLU300 |
| | GLU301 |
| | SER302 |
| | LEU303 |
| | ILE306 |
| | PHE307 |
| | MET310 |

TABLE 5B
Residues on RET that bind to GFRAL in
RET/GFRAL/GDF15 Model

| Core interaction | Boundary interaction |
|---|---|
| interface amino acids | interface amino acids |
| GLY74 | ASP34 |
| THR75 | ALA35 |
| TYR76 | TYR36 |
| ARG77 | HIS71 |
| THR78 | TYR73 |
| ASN113 | LEU72 |
| ARG114 | GLY74 |
| PHE116 | TYR76 |
| TYR122 | THR75 |
| GLN138 | ARG77 |
| ARG144 | THR78 |
| PRO305 | ARG79 |
| ALA306 | LEU80 |
| LEU310 | LEU109 |
| | SER110 |
| | VAL111 |
| | ARG112 |
| | ASN113 |
| | GLY115 |
| | ARG114 |
| | PHE116 |
| | PRO117 |
| | LEU118 |
| | THR120 |
| | VAL121 |
| | TYR122 |
| | LEU123 |
| | LYS124 |
| | CYS137 |
| | GLN138 |
| | TRP139 |
| | PRO140 |
| | GLY141 |
| | CYS142 |
| | ALA143 |
| | ARG144 |
| | VAL145 |
| | TYR146 |
| | PHE147 |
| | ARG231 |
| | ASP264 |
| | ASP300 |
| | VAL303 |
| | VAL304 |
| | PRO305 |
| | ALA306 |
| | SER307 |
| | GLY308 |
| | GLU309 |
| | LEU310 |
| | ARG312 |
| | VAL311 |
| | ASN336 |

TABLE 6

| 1 | 2 | 3 | 4 | 5 | 6 | 7 | 8 | 9 | 10 | 11 | 12 | 13 |
|---|---|---|---|---|---|---|---|---|---|---|---|---|
| ATOM | 1 | N | ASP | A | 5 | −29.647 | −38.53 | 22.338 | 1 | 107.89 | A | N |
| ATOM | 2 | CA | ASP | A | 5 | −29.096 | −38.791 | 20.97 | 1 | 108.54 | A | C |
| ATOM | 3 | CB | ASP | A | 5 | −29.843 | −39.971 | 20.331 | 1 | 100.53 | A | C |
| ATOM | 4 | CG | ASP | A | 5 | −29.07 | −40.64 | 19.218 | 1 | 86.72 | A | C |
| ATOM | 5 | OD1 | ASP | A | 5 | −27.842 | −40.461 | 19.118 | 1 | 79.65 | A | O |
| ATOM | 6 | OD2 | ASP | A | 5 | −29.702 | −41.388 | 18.449 | 1 | 91.44 | A | O |
| ATOM | 7 | C | ASP | A | 5 | −29.229 | −37.55 | 20.075 | 1 | 107.46 | A | C |
| ATOM | 8 | O | ASP | A | 5 | −30.341 | −37.026 | 19.939 | 1 | 108.4 | A | O |
| ATOM | 9 | N | HIS | A | 6 | −28.127 | −37.121 | 19.436 | 1 | 105.39 | A | N |
| ATOM | 10 | CA | HIS | A | 6 | −28.13 | −35.922 | 18.544 | 1 | 100.79 | A | C |
| ATOM | 11 | CB | HIS | A | 6 | −26.77 | −35.188 | 18.564 | 1 | 109.43 | A | C |
| ATOM | 12 | CG | HIS | A | 6 | −25.631 | −35.935 | 17.932 | 1 | 118.36 | A | C |
| ATOM | 13 | ND1 | HIS | A | 6 | −25.266 | −35.76 | 16.614 | 1 | 123.98 | A | N |
| ATOM | 14 | CE1 | HIS | A | 6 | −24.212 | −36.512 | 16.344 | 1 | 124.11 | A | C |
| ATOM | 15 | NE2 | HIS | A | 6 | −23.865 | −37.151 | 17.448 | 1 | 122.91 | A | N |
| ATOM | 16 | CD2 | HIS | A | 6 | −24.727 | −36.798 | 18.458 | 1 | 121.3 | A | C |
| ATOM | 17 | C | HIS | A | 6 | −28.657 | −36.133 | 17.092 | 1 | 91.62 | A | C |
| ATOM | 18 | O | HIS | A | 6 | −28.298 | −35.402 | 16.164 | 1 | 93.2 | A | O |
| ATOM | 19 | N | CYS | A | 7 | −29.508 | −37.146 | 16.935 | 1 | 75.73 | A | N |
| ATOM | 20 | CA | CYS | A | 7 | −30.401 | −37.322 | 15.804 | 1 | 63.79 | A | C |
| ATOM | 21 | CB | CYS | A | 7 | −30.888 | −38.78 | 15.796 | 1 | 52.38 | A | C |
| ATOM | 22 | SG | CYS | A | 7 | −32.172 | −39.202 | 14.618 | 1 | 54.69 | A | S |
| ATOM | 23 | C | CYS | A | 7 | −31.587 | −36.369 | 15.975 | 1 | 61.67 | A | C |
| ATOM | 24 | O | CYS | A | 7 | −32.353 | −36.517 | 16.912 | 1 | 65.41 | A | O |
| ATOM | 25 | N | PRO | A | 8 | −31.763 | −35.403 | 15.061 | 1 | 68.82 | A | N |
| ATOM | 26 | CA | PRO | A | 8 | −32.972 | −34.563 | 15.074 | 1 | 69.64 | A | C |
| ATOM | 27 | CB | PRO | A | 8 | −32.921 | −33.847 | 13.718 | 1 | 69.48 | A | C |
| ATOM | 28 | CG | PRO | A | 8 | −31.478 | −33.831 | 13.327 | 1 | 70.01 | A | C |
| ATOM | 29 | CD | PRO | A | 8 | −30.866 | −35.064 | 13.935 | 1 | 72.51 | A | C |
| ATOM | 30 | C | PRO | A | 8 | −34.292 | −35.346 | 15.187 | 1 | 74.75 | A | C |
| ATOM | 31 | O | PRO | A | 8 | −35.176 | −34.932 | 15.91 | 1 | 78.12 | A | O |
| ATOM | 32 | N | LEU | A | 9 | −34.397 | −36.49 | 14.514 | 1 | 72.61 | A | N |
| ATOM | 33 | CA | LEU | A | 9 | −35.657 | −37.222 | 14.415 | 1 | 69.49 | A | C |
| ATOM | 34 | CB | LEU | A | 9 | −35.634 | −38.147 | 13.197 | 1 | 71.26 | A | C |
| ATOM | 35 | CG | LEU | A | 9 | −35.228 | −37.563 | 11.837 | 1 | 71.78 | A | C |
| ATOM | 36 | CD1 | LEU | A | 9 | −35.448 | −38.642 | 10.801 | 1 | 73.76 | A | C |
| ATOM | 37 | CD2 | LEU | A | 9 | −35.975 | −36.288 | 11.443 | 1 | 74.5 | A | C |
| ATOM | 38 | C | LEU | A | 9 | −36.021 | −38.065 | 15.64 | 1 | 71.64 | A | C |
| ATOM | 39 | O | LEU | A | 9 | −37.008 | −38.819 | 15.594 | 1 | 79.87 | A | O |
| ATOM | 40 | N | GLY | A | 10 | −35.255 | −37.948 | 16.722 | 1 | 61.99 | A | N |
| ATOM | 41 | CA | GLY | A | 10 | −35.484 | −38.736 | 17.919 | 1 | 65.08 | A | C |
| ATOM | 42 | C | GLY | A | 10 | −34.43 | −39.824 | 18.05 | 1 | 71.72 | A | C |
| ATOM | 43 | O | GLY | A | 10 | −34.006 | −40.383 | 17.046 | 1 | 67.95 | A | O |
| ATOM | 44 | N | PRO | A | 11 | −33.986 | −40.123 | 19.287 | 1 | 75.85 | A | N |
| ATOM | 45 | CA | PRO | A | 11 | −33.041 | −41.212 | 19.551 | 1 | 76.1 | A | C |
| ATOM | 46 | CB | PRO | A | 11 | −33.286 | −41.519 | 21.033 | 1 | 75.37 | A | C |
| ATOM | 47 | CG | PRO | A | 11 | −33.644 | −40.19 | 21.618 | 1 | 76.52 | A | C |
| ATOM | 48 | CD | PRO | A | 11 | −34.217 | −39.33 | 20.512 | 1 | 77.29 | A | C |
| ATOM | 49 | C | PRO | A | 11 | −33.175 | −42.48 | 18.698 | 1 | 74.27 | A | C |
| ATOM | 50 | O | PRO | A | 11 | −34.303 | −42.961 | 18.476 | 1 | 62.1 | A | O |
| ATOM | 51 | N | GLY | A | 12 | −32.012 | −42.952 | 18.198 | 1 | 74.34 | A | N |
| ATOM | 52 | CA | GLY | A | 12 | −31.853 | −44.216 | 17.445 | 1 | 62.04 | A | C |
| ATOM | 53 | C | GLY | A | 12 | −32.325 | −44.234 | 16.004 | 1 | 56.96 | A | C |
| ATOM | 54 | O | GLY | A | 12 | −32.174 | −45.249 | 15.332 | 1 | 74.7 | A | O |
| ATOM | 55 | N | ARG | A | 13 | −32.861 | −43.123 | 15.509 | 1 | 55.39 | A | N |
| ATOM | 56 | CA | ARG | A | 13 | −33.578 | −43.082 | 14.222 | 1 | 59.08 | A | C |
| ATOM | 57 | CB | ARG | A | 13 | −34.705 | −42.046 | 14.281 | 1 | 69.86 | A | C |
| ATOM | 58 | CG | ARG | A | 13 | −35.749 | −42.317 | 15.362 | 1 | 78.39 | A | C |
| ATOM | 59 | CD | ARG | A | 13 | −36.853 | −43.206 | 14.862 | 1 | 79.36 | A | C |
| ATOM | 60 | NE | ARG | A | 13 | −38.026 | −42.449 | 14.423 | 1 | 95.71 | A | N |
| ATOM | 61 | CZ | ARG | A | 13 | −39.004 | −42.009 | 15.222 | 1 | 103.01 | A | C |
| ATOM | 62 | NH1 | ARG | A | 13 | −40.025 | −41.344 | 14.686 | 1 | 107.76 | A | N |
| ATOM | 63 | NH2 | ARG | A | 13 | −38.99 | −42.228 | 16.545 | 1 | 98.59 | A | N |
| ATOM | 64 | C | ARG | A | 13 | −32.711 | −42.712 | 13.051 | 1 | 52.1 | A | C |
| ATOM | 65 | O | ARG | A | 13 | −33.095 | −42.929 | 11.889 | 1 | 55.9 | A | O |
| ATOM | 66 | N | CYS | A | 14 | −31.573 | −42.098 | 13.339 | 1 | 48.83 | A | N |
| ATOM | 67 | CA | CYS | A | 14 | −30.707 | −41.597 | 12.295 | 1 | 48.1 | A | C |
| ATOM | 68 | CB | CYS | A | 14 | −30.112 | −40.231 | 12.685 | 1 | 50.76 | A | C |
| ATOM | 69 | SG | CYS | A | 14 | −31.304 | −38.85 | 12.8 | 1 | 55.94 | A | S |
| ATOM | 70 | C | CYS | A | 14 | −29.594 | −42.605 | 11.955 | 1 | 45.31 | A | C |
| ATOM | 71 | O | CYS | A | 14 | −29.34 | −43.585 | 12.673 | 1 | 42.95 | A | O |
| ATOM | 72 | N | CYS | A | 15 | −28.966 | −42.34 | 10.818 | 1 | 46.46 | A | N |
| ATOM | 73 | CA | CYS | A | 15 | −27.908 | −43.165 | 10.243 | 1 | 42.86 | A | C |
| ATOM | 74 | CB | CYS | A | 15 | −27.242 | −42.39 | 9.123 | 1 | 43.42 | A | C |
| ATOM | 75 | SG | CYS | A | 15 | −25.83 | −43.198 | 8.361 | 1 | 44.21 | A | S |
| ATOM | 76 | C | CYS | A | 15 | −26.855 | −43.551 | 11.279 | 1 | 42.46 | A | C |
| ATOM | 77 | O | CYS | A | 15 | −26.213 | −42.685 | 11.849 | 1 | 39.72 | A | O |
| ATOM | 78 | N | ARG | A | 16 | −26.71 | −44.849 | 11.506 | 1 | 36.74 | A | N |
| ATOM | 79 | CA | ARG | A | 16 | −25.828 | −45.376 | 12.521 | 1 | 39.41 | A | C |
| ATOM | 80 | CB | ARG | A | 16 | −26.413 | −45.146 | 13.931 | 1 | 43.83 | A | C |
| ATOM | 81 | CG | ARG | A | 16 | −27.496 | −46.106 | 14.375 | 1 | 50.15 | A | C |
| ATOM | 82 | CD | ARG | A | 16 | −28.045 | −45.724 | 15.741 | 1 | 58.55 | A | C |
| ATOM | 83 | NE | ARG | A | 16 | −29.3 | −46.406 | 16.072 | 1 | 68.87 | A | N |
| ATOM | 84 | CZ | ARG | A | 16 | −29.421 | −47.682 | 16.466 | 1 | 77.43 | A | C |
| ATOM | 85 | NH1 | ARG | A | 16 | −30.623 | −48.158 | 16.75 | 1 | 80.99 | A | N |
| ATOM | 86 | NH2 | ARG | A | 16 | −28.38 | −48.509 | 16.547 | 1 | 79.65 | A | N |
| ATOM | 87 | C | ARG | A | 16 | −25.569 | −46.866 | 12.296 | 1 | 36.24 | A | C |
| ATOM | 88 | O | ARG | A | 16 | −26.187 | −47.468 | 11.467 | 1 | 37.37 | A | O |
| ATOM | 89 | N | LEU | A | 17 | −24.699 | −47.466 | 13.086 | 1 | 38.89 | A | N |
| ATOM | 90 | CA | LEU | A | 17 | −24.268 | −48.86 | 12.886 | 1 | 37.13 | A | C |
| ATOM | 91 | CB | LEU | A | 17 | −22.905 | −49.073 | 13.565 | 1 | 41.23 | A | C |
| ATOM | 92 | CG | LEU | A | 17 | −22.1 | −50.275 | 13.12 | 1 | 43.12 | A | C |
| ATOM | 93 | CD1 | LEU | A | 17 | −21.629 | −50.095 | 11.676 | 1 | 38.64 | A | C |
| ATOM | 94 | CD2 | LEU | A | 17 | −20.902 | −50.433 | 14.054 | 1 | 44.92 | A | C |
| ATOM | 95 | C | LEU | A | 17 | −25.227 | −49.813 | 13.53 | 1 | 41.04 | A | C |
| ATOM | 96 | O | LEU | A | 17 | −25.53 | −49.65 | 14.711 | 1 | 41.05 | A | O |
| ATOM | 97 | N | HIS | A | 18 | −25.674 | −50.823 | 12.775 | 1 | 43.98 | A | N |
| ATOM | 98 | CA | HIS | A | 18 | −26.506 | −51.928 | 13.269 | 1 | 43.71 | A | C |
| ATOM | 99 | CB | HIS | A | 18 | −27.856 | −51.935 | 12.514 | 1 | 46.48 | A | C |
| ATOM | 100 | CG | HIS | A | 18 | −28.736 | −50.775 | 12.849 | 1 | 58.05 | A | C |
| ATOM | 101 | ND1 | HIS | A | 18 | −28.472 | −49.489 | 12.411 | 1 | 60.73 | A | N |
| ATOM | 102 | CE1 | HIS | A | 18 | −29.391 | −48.673 | 12.886 | 1 | 59.23 | A | C |
| ATOM | 103 | NE2 | HIS | A | 18 | −30.252 | −49.384 | 13.593 | 1 | 64.69 | A | N |
| ATOM | 104 | CD2 | HIS | A | 18 | −29.86 | −50.698 | 13.599 | 1 | 56.48 | A | C |
| ATOM | 105 | C | HIS | A | 18 | −25.802 | −53.298 | 13.027 | 1 | 42.3 | A | C |
| ATOM | 106 | O | HIS | A | 18 | −25.005 | −53.445 | 12.115 | 1 | 36.07 | A | O |
| ATOM | 107 | N | THR | A | 19 | −26.154 | −54.295 | 13.818 | 1 | 39.75 | A | N |
| ATOM | 108 | CA | THR | A | 19 | −25.625 | −55.642 | 13.686 | 1 | 41.06 | A | C |
| ATOM | 109 | CB | THR | A | 19 | −25.013 | −56.059 | 15.015 | 1 | 41.9 | A | C |
| ATOM | 110 | OG1 | THR | A | 19 | −24.086 | −55.048 | 15.4 | 1 | 43.26 | A | O |
| ATOM | 111 | CG2 | THR | A | 19 | −24.291 | −57.393 | 14.911 | 1 | 40.13 | A | C |
| ATOM | 112 | C | THR | A | 19 | −26.743 | −56.596 | 13.279 | 1 | 41.71 | A | C |
| ATOM | 113 | O | THR | A | 19 | −27.785 | −56.584 | 13.906 | 1 | 38.18 | A | O |
| ATOM | 114 | N | VAL | A | 20 | −26.536 | −57.396 | 12.227 | 1 | 40.53 | A | N |
| ATOM | 115 | CA | VAL | A | 20 | −27.484 | −58.429 | 11.817 | 1 | 39.22 | A | C |
| ATOM | 116 | CB | VAL | A | 20 | −28.044 | −58.13 | 10.41 | 1 | 43.74 | A | C |
| ATOM | 117 | CG1 | VAL | A | 20 | −28.987 | −59.232 | 9.948 | 1 | 43.53 | A | C |
| ATOM | 118 | CG2 | VAL | A | 20 | −28.75 | −56.786 | 10.382 | 1 | 46.49 | A | C |
| ATOM | 119 | C | VAL | A | 20 | −26.786 | −59.777 | 11.741 | 1 | 40.46 | A | C |
| ATOM | 120 | O | VAL | A | 20 | −25.85 | −59.956 | 10.93 | 1 | 38.14 | A | O |
| ATOM | 121 | N | ARG | A | 21 | −27.228 | −60.745 | 12.54 | 1 | 39.82 | A | N |
| ATOM | 122 | CA | ARG | A | 21 | −26.586 | −62.066 | 12.535 | 1 | 43.73 | A | C |
| ATOM | 123 | CB | ARG | A | 21 | −26.987 | −62.963 | 13.685 | 1 | 45.08 | A | C |
| ATOM | 124 | CG | ARG | A | 21 | −26.669 | −62.384 | 15.052 | 1 | 58.75 | A | C |
| ATOM | 125 | CD | ARG | A | 21 | −27.049 | −63.345 | 16.206 | 1 | 66.8 | A | C |
| ATOM | 126 | NE | ARG | A | 21 | −25.873 | −63.877 | 16.923 | 1 | 74.5 | A | N |
| ATOM | 127 | CZ | ARG | A | 21 | −25.32 | −63.361 | 18.032 | 1 | 75.89 | A | C |
| ATOM | 128 | NH1 | ARG | A | 21 | −25.811 | −62.277 | 18.632 | 1 | 76.6 | A | N |
| ATOM | 129 | NH2 | ARG | A | 21 | −24.247 | −63.946 | 18.561 | 1 | 80.23 | A | N |
| ATOM | 130 | C | ARG | A | 21 | −27.001 | −62.704 | 11.276 | 1 | 39.92 | A | C |
| ATOM | 131 | O | ARG | A | 21 | −28.143 | −62.657 | 10.956 | 1 | 43.31 | A | O |
| ATOM | 132 | N | ALA | A | 22 | −26.076 | −63.254 | 10.524 | 1 | 39.79 | A | N |
| ATOM | 133 | CA | ALA | A | 22 | −26.421 | −64.017 | 9.337 | 1 | 38.46 | A | C |
| ATOM | 134 | CB | ALA | A | 22 | −26.149 | −63.193 | 8.114 | 1 | 39.22 | A | C |
| ATOM | 135 | C | ALA | A | 22 | −25.592 | −65.311 | 9.271 | 1 | 39.48 | A | C |
| ATOM | 136 | O | ALA | A | 22 | −24.409 | −65.296 | 9.527 | 1 | 41.3 | A | O |
| ATOM | 137 | N | SER | A | 23 | −26.222 | −66.403 | 8.881 | 1 | 37.08 | A | N |
| ATOM | 138 | CA | SER | A | 23 | −25.546 | −67.652 | 8.678 | 1 | 38.93 | A | C |
| ATOM | 139 | CB | SER | A | 23 | −26.551 | −68.796 | 8.803 | 1 | 38.61 | A | C |
| ATOM | 140 | OG | SER | A | 23 | −27.502 | −68.72 | 7.741 | 1 | 39.78 | A | O |
| ATOM | 141 | C | SER | A | 23 | −25.018 | −67.648 | 7.254 | 1 | 40.87 | A | C |
| ATOM | 142 | O | SER | A | 23 | −25.426 | −66.809 | 6.41 | 1 | 35.36 | A | O |
| ATOM | 143 | N | LEU | A | 24 | −24.175 | −68.635 | 6.98 | 1 | 36.42 | A | N |
| ATOM | 144 | CA | LEU | A | 24 | −23.583 | −68.795 | 5.666 | 1 | 42.13 | A | C |
| ATOM | 145 | CB | LEU | A | 24 | −22.538 | −69.956 | 5.614 | 1 | 44.12 | A | C |
| ATOM | 146 | CG | LEU | A | 24 | −21.269 | −69.826 | 6.452 | 1 | 43.49 | A | C |
| ATOM | 147 | CD1 | LEU | A | 24 | −20.374 | −71.041 | 6.264 | 1 | 42.41 | A | C |
| ATOM | 148 | CD2 | LEU | A | 24 | −20.542 | −68.532 | 6.109 | 1 | 43.21 | A | C |
| ATOM | 149 | C | LEU | A | 24 | −24.665 | −69.034 | 4.628 | 1 | 39.9 | A | C |
| ATOM | 150 | O | LEU | A | 24 | −24.559 | −68.556 | 3.508 | 1 | 38.41 | A | O |
| ATOM | 151 | N | GLU | A | 25 | −25.706 | −69.751 | 5.006 | 1 | 43.34 | A | N |
| ATOM | 152 | CA | GLU | A | 25 | −26.795 | −70.044 | 4.065 | 1 | 50.43 | A | C |
| ATOM | 153 | CB | GLU | A | 25 | −27.66 | −71.293 | 4.443 | 1 | 52.95 | A | C |
| ATOM | 154 | CG | GLU | A | 25 | −27.505 | −71.876 | 5.867 | 1 | 69.02 | A | C |
| ATOM | 155 | CD | GLU | A | 25 | −26.088 | −72.383 | 6.28 | 1 | 71.32 | A | C |
| ATOM | 156 | OE1 | GLU | A | 25 | −25.555 | −73.368 | 5.684 | 1 | 76.67 | A | O |
| ATOM | 157 | OE2 | GLU | A | 25 | −25.522 | −71.804 | 7.251 | 1 | 58.26 | A | O |
| ATOM | 158 | C | GLU | A | 25 | −27.595 | −68.747 | 3.781 | 1 | 42.68 | A | C |
| ATOM | 159 | O | GLU | A | 25 | −27.94 | −68.492 | 2.642 | 1 | 40.98 | A | O |
| ATOM | 160 | N | ASP | A | 26 | −27.79 | −67.883 | 4.772 | 1 | 40.77 | A | N |
| ATOM | 161 | CA | ASP | A | 26 | −28.409 | −66.564 | 4.532 | 1 | 37.13 | A | C |
| ATOM | 162 | CB | ASP | A | 26 | −28.502 | −65.741 | 5.82 | 1 | 43.25 | A | C |
| ATOM | 163 | CG | ASP | A | 26 | −29.419 | −66.366 | 6.876 | 1 | 45.1 | A | C |
| ATOM | 164 | OD1 | ASP | A | 26 | −30.338 | −67.123 | 6.493 | 1 | 46.96 | A | O |
| ATOM | 165 | OD2 | ASP | A | 26 | −29.221 | −66.103 | 8.091 | 1 | 46.84 | A | O |
| ATOM | 166 | C | ASP | A | 26 | −27.591 | −65.768 | 3.51 | 1 | 44.61 | A | C |
| ATOM | 167 | O | ASP | A | 26 | −28.128 | −65.041 | 2.694 | 1 | 44.99 | A | O |
| ATOM | 168 | N | LEU | A | 27 | −26.271 | −65.913 | 3.549 | 1 | 45.46 | A | N |
| ATOM | 169 | CA | LEU | A | 27 | −25.417 | −65.13 | 2.683 | 1 | 41.01 | A | C |
| ATOM | 170 | CB | LEU | A | 27 | −24.07 | −64.903 | 3.363 | 1 | 38.39 | A | C |
| ATOM | 171 | CG | LEU | A | 27 | −24.124 | −64.067 | 4.623 | 1 | 39.65 | A | C |
| ATOM | 172 | CD1 | LEU | A | 27 | −22.738 | −63.929 | 5.228 | 1 | 41.65 | A | C |
| ATOM | 173 | CD2 | LEU | A | 27 | −24.69 | −62.687 | 4.363 | 1 | 40.96 | A | C |
| ATOM | 174 | C | LEU | A | 27 | −25.224 | −65.789 | 1.34 | 1 | 40.76 | A | C |
| ATOM | 175 | O | LEU | A | 27 | −24.78 | −65.126 | 0.43 | 1 | 38.17 | A | O |
| ATOM | 176 | N | GLY | A | 28 | −25.542 | −67.081 | 1.23 | 1 | 39.95 | A | N |
| ATOM | 177 | CA | GLY | A | 28 | −25.301 | −67.873 | 0.016 | 1 | 36.14 | A | C |
| ATOM | 178 | C | GLY | A | 28 | −23.887 | −68.377 | −0.086 | 1 | 35.77 | A | C |
| ATOM | 179 | O | GLY | A | 28 | −23.379 | −68.63 | −1.17 | 1 | 36.04 | A | O |
| ATOM | 180 | N | TRP | A | 29 | −23.212 | −68.489 | 1.047 | 1 | 36.89 | A | N |
| ATOM | 181 | CA | TRP | A | 29 | −21.766 | −68.788 | 1.074 | 1 | 38.59 | A | C |
| ATOM | 182 | CB | TRP | A | 29 | −21.065 | −67.84 | 2.052 | 1 | 34.32 | A | C |
| ATOM | 183 | CG | TRP | A | 29 | −20.968 | −66.45 | 1.653 | 1 | 35.3 | A | C |
| ATOM | 184 | CD1 | TRP | A | 29 | −21.518 | −65.847 | 0.56 | 1 | 34.09 | A | C |
| ATOM | 185 | NE1 | TRP | A | 29 | −21.241 | −64.507 | 0.58 | 1 | 32.16 | A | N |
| ATOM | 186 | CE2 | TRP | A | 29 | −20.499 | −64.214 | 1.676 | 1 | 33.46 | A | C |
| ATOM | 187 | CD2 | TRP | A | 29 | −20.334 | −65.416 | 2.402 | 1 | 35 | A | C |
| ATOM | 188 | CE3 | TRP | A | 29 | −19.65 | −65.388 | 3.626 | 1 | 34.78 | A | C |
| ATOM | 189 | CZ3 | TRP | A | 29 | −19.138 | −64.173 | 4.08 | 1 | 36.79 | A | C |
| ATOM | 190 | CH2 | TRP | A | 29 | −19.282 | −62.979 | 3.303 | 1 | 36.42 | A | C |
| ATOM | 191 | CZ2 | TRP | A | 29 | −19.973 | −62.989 | 2.105 | 1 | 38.78 | A | C |
| ATOM | 192 | C | TRP | A | 29 | −21.418 | −70.246 | 1.515 | 1 | 42.81 | A | C |
| ATOM | 193 | O | TRP | A | 29 | −20.244 | −70.622 | 1.459 | 1 | 41.1 | A | O |
| ATOM | 194 | N | ALA | A | 30 | −22.419 | −71.034 | 1.93 | 1 | 41.02 | A | N |
| ATOM | 195 | CA | ALA | A | 30 | −22.202 | −72.329 | 2.636 | 1 | 46.07 | A | C |
| ATOM | 196 | CB | ALA | A | 30 | −23.524 | −72.952 | 3.074 | 1 | 49.03 | A | C |
| ATOM | 197 | C | ALA | A | 30 | −21.364 | −73.34 | 1.877 | 1 | 41.75 | A | C |
| ATOM | 198 | O | ALA | A | 30 | −20.495 | −73.957 | 2.445 | 1 | 41.25 | A | O |
| ATOM | 199 | N | ASP | A | 31 | −21.544 | −73.421 | 0.578 | 1 | 43.65 | A | N |
| ATOM | 200 | CA | ASP | A | 31 | −20.684 | −74.273 | −0.237 | 1 | 43.75 | A | C |
| ATOM | 201 | CB | ASP | A | 31 | −21.273 | −74.376 | −1.648 | 1 | 51.71 | A | C |
| ATOM | 202 | CG | ASP | A | 31 | −22.617 | −75.125 | −1.683 | 1 | 53.98 | A | C |
| ATOM | 203 | OD1 | ASP | A | 31 | −23.042 | −75.654 | −0.655 | 1 | 57.9 | A | O |
| ATOM | 204 | OD2 | ASP | A | 31 | −23.236 | −75.217 | −2.758 | 1 | 58.38 | A | O |
| ATOM | 205 | C | ASP | A | 31 | −19.238 | −73.836 | −0.349 | 1 | 44.79 | A | C |
| ATOM | 206 | O | ASP | A | 31 | −18.398 | −74.633 | −0.735 | 1 | 46.95 | A | O |
| ATOM | 207 | N | TRP | A | 32 | −18.932 | −72.569 | −0.075 | 1 | 43.94 | A | N |
| ATOM | 208 | CA | TRP | A | 32 | −17.621 | −72.011 | −0.415 | 1 | 41.75 | A | C |
| ATOM | 209 | CB | TRP | A | 32 | −17.825 | −70.798 | −1.339 | 1 | 47.2 | A | C |
| ATOM | 210 | CG | TRP | A | 32 | −18.905 | −71.052 | −2.344 | 1 | 51.48 | A | C |
| ATOM | 211 | CD1 | TRP | A | 32 | −20.181 | −70.502 | −2.398 | 1 | 49.12 | A | C |
| ATOM | 212 | NE1 | TRP | A | 32 | −20.868 | −71.014 | −3.457 | 1 | 47.47 | A | N |
| ATOM | 213 | CE2 | TRP | A | 32 | −20.059 | −71.934 | −4.085 | 1 | 54.26 | A | C |
| ATOM | 214 | CD2 | TRP | A | 32 | −18.811 | −71.958 | −3.407 | 1 | 47.6 | A | C |
| ATOM | 215 | CE3 | TRP | A | 32 | −17.793 | −72.809 | −3.852 | 1 | 46.91 | A | C |
| ATOM | 216 | CZ3 | TRP | A | 32 | −18.021 | −73.588 | −4.949 | 1 | 53.12 | A | C |
| ATOM | 217 | CH2 | TRP | A | 32 | −19.274 | −73.557 | −5.619 | 1 | 58.43 | A | C |
| ATOM | 218 | CZ2 | TRP | A | 32 | −20.302 | −72.72 | −5.205 | 1 | 55.67 | A | C |
| ATOM | 219 | C | TRP | A | 32 | −16.794 | −71.573 | 0.809 | 1 | 42.61 | A | C |
| ATOM | 220 | O | TRP | A | 32 | −15.616 | −71.214 | 0.651 | 1 | 40.43 | A | O |
| ATOM | 221 | N | VAL | A | 33 | −17.423 | −71.512 | 1.985 | 1 | 38.54 | A | N |
| ATOM | 222 | CA | VAL | A | 33 | −16.808 | −70.938 | 3.167 | 1 | 41.98 | A | C |
| ATOM | 223 | CB | VAL | A | 33 | −17.417 | −69.558 | 3.522 | 1 | 38.54 | A | C |
| ATOM | 224 | CG1 | VAL | A | 33 | −16.89 | −69.04 | 4.862 | 1 | 43.37 | A | C |
| ATOM | 225 | CG2 | VAL | A | 33 | −17.113 | −68.583 | 2.414 | 1 | 38.78 | A | C |
| ATOM | 226 | C | VAL | A | 33 | −16.989 | −71.944 | 4.294 | 1 | 42.01 | A | C |
| ATOM | 227 | O | VAL | A | 33 | −18.085 | −72.397 | 4.562 | 1 | 41.04 | A | O |
| ATOM | 228 | N | LEU | A | 34 | −15.895 | −72.274 | 4.95 | 1 | 42.86 | A | N |
| ATOM | 229 | CA | LEU | A | 34 | −15.911 | −73.198 | 6.077 | 1 | 42.93 | A | C |
| ATOM | 230 | CB | LEU | A | 34 | −14.572 | −73.959 | 6.099 | 1 | 43.29 | A | C |
| ATOM | 231 | CG | LEU | A | 34 | −14.42 | −74.961 | 7.265 | 1 | 54.42 | A | C |
| ATOM | 232 | CD1 | LEU | A | 34 | −15.301 | −76.192 | 7.034 | 1 | 53.19 | A | C |
| ATOM | 233 | CD2 | LEU | A | 34 | −12.954 | −75.33 | 7.518 | 1 | 55.56 | A | C |
| ATOM | 234 | C | LEU | A | 34 | −16.178 | −72.433 | 7.413 | 1 | 40.42 | A | C |
| ATOM | 235 | O | LEU | A | 34 | −16.79 | −72.928 | 8.313 | 1 | 40.55 | A | O |
| ATOM | 236 | N | SER | A | 35 | −15.717 | −71.206 | 7.506 | 1 | 37.52 | A | N |
| ATOM | 237 | CA | SER | A | 35 | −15.749 | −70.47 | 8.724 | 1 | 39.64 | A | C |
| ATOM | 238 | CB | SER | A | 35 | −14.534 | −70.926 | 9.567 | 1 | 42.83 | A | C |
| ATOM | 239 | OG | SER | A | 35 | −14.601 | −70.373 | 10.865 | 1 | 44.11 | A | O |
| ATOM | 240 | C | SER | A | 35 | −15.632 | −68.968 | 8.441 | 1 | 32.13 | A | C |
| ATOM | 241 | O | SER | A | 35 | −14.844 | −68.604 | 7.605 | 1 | 35.53 | A | O |
| ATOM | 242 | N | PRO | A | 36 | −16.332 | −68.092 | 9.151 | 1 | 36.3 | A | N |
| ATOM | 243 | CA | PRO | A | 36 | −17.211 | −68.423 | 10.271 | 1 | 38.14 | A | C |
| ATOM | 244 | CB | PRO | A | 36 | −17.351 | −67.077 | 11.001 | 1 | 39.49 | A | C |
| ATOM | 245 | CG | PRO | A | 36 | −17.254 | −66.055 | 9.941 | 1 | 36.45 | A | C |
| ATOM | 246 | CD | PRO | A | 36 | −16.395 | −66.645 | 8.826 | 1 | 37.44 | A | C |
| ATOM | 247 | C | PRO | A | 36 | −18.572 | −68.885 | 9.874 | 1 | 38.93 | A | C |
| ATOM | 248 | O | PRO | A | 36 | −19.075 | −68.468 | 8.862 | 1 | 42.7 | A | O |
| ATOM | 249 | N | ARG | A | 37 | −19.219 | −69.679 | 10.711 | 1 | 44 | A | N |
| ATOM | 250 | CA | ARG | A | 37 | −20.531 | −70.215 | 10.356 | 1 | 45.51 | A | C |
| ATOM | 251 | CB | ARG | A | 37 | −20.889 | −71.414 | 11.228 | 1 | 45.67 | A | C |
| ATOM | 252 | CG | ARG | A | 37 | −20.025 | −72.651 | 11.007 | 1 | 55.76 | A | C |
| ATOM | 253 | CD | ARG | A | 37 | −20.525 | −73.774 | 11.919 | 1 | 69.94 | A | C |
| ATOM | 254 | NE | ARG | A | 37 | −19.478 | −74.724 | 12.336 | 1 | 81.13 | A | N |
| ATOM | 255 | CZ | ARG | A | 37 | −19.415 | −75.37 | 13.514 | 1 | 84.71 | A | C |
| ATOM | 256 | NH1 | ARG | A | 37 | −20.321 | −75.178 | 14.478 | 1 | 90.98 | A | N |
| ATOM | 257 | NH2 | ARG | A | 37 | −18.413 | −76.218 | 13.746 | 1 | 83.41 | A | N |
| ATOM | 258 | C | ARG | A | 37 | −21.634 | −69.177 | 10.456 | 1 | 39.03 | A | C |
| ATOM | 259 | O | ARG | A | 37 | −22.654 | −69.31 | 9.83 | 1 | 43.54 | A | O |
| ATOM | 260 | N | GLU | A | 38 | −21.441 | −68.194 | 11.31 | 1 | 41.18 | A | N |
| ATOM | 261 | CA | GLU | A | 38 | −22.372 | −67.098 | 11.516 | 1 | 48.45 | A | C |
| ATOM | 262 | CB | GLU | A | 38 | −22.904 | −67.113 | 12.964 | 1 | 54.37 | A | C |
| ATOM | 263 | CG | GLU | A | 38 | −24.42 | −67.26 | 13.185 | 1 | 69.9 | A | C |
| ATOM | 264 | CD | GLU | A | 38 | −24.922 | −66.513 | 14.439 | 1 | 71.65 | A | C |
| ATOM | 265 | OE1 | GLU | A | 38 | −26.123 | −66.145 | 14.507 | 1 | 69.26 | A | O |
| ATOM | 266 | OE2 | GLU | A | 38 | −24.102 | −66.26 | 15.356 | 1 | 72.37 | A | O |
| ATOM | 267 | C | GLU | A | 38 | −21.525 | −65.854 | 11.361 | 1 | 41.02 | A | C |
| ATOM | 268 | O | GLU | A | 38 | −20.428 | −65.807 | 11.896 | 1 | 43.5 | A | O |
| ATOM | 269 | N | VAL | A | 39 | −22.047 | −64.84 | 10.698 | 1 | 37.85 | A | N |
| ATOM | 270 | CA | VAL | A | 39 | −21.364 | −63.565 | 10.606 | 1 | 37.99 | A | C |
| ATOM | 271 | CB | VAL | A | 39 | −21.206 | −63.09 | 9.151 | 1 | 36.17 | A | C |
| ATOM | 272 | CG1 | VAL | A | 39 | −20.492 | −61.741 | 9.133 | 1 | 37.19 | A | C |
| ATOM | 273 | CG2 | VAL | A | 39 | −20.386 | −64.095 | 8.369 | 1 | 40.75 | A | C |
| ATOM | 274 | C | VAL | A | 39 | −22.178 | −62.531 | 11.305 | 1 | 36.27 | A | C |
| ATOM | 275 | O | VAL | A | 39 | −23.343 | −62.441 | 11.06 | 1 | 38.57 | A | O |
| ATOM | 276 | N | GLN | A | 40 | −21.547 | −61.722 | 12.132 | 1 | 37.54 | A | N |
| ATOM | 277 | CA | GLN | A | 40 | −22.208 | −60.605 | 12.778 | 1 | 40.96 | A | C |
| ATOM | 278 | CB | GLN | A | 40 | −21.657 | −60.366 | 14.166 | 1 | 47.52 | A | C |
| ATOM | 279 | CG | GLN | A | 40 | −22.697 | −60.446 | 15.259 | 1 | 65.12 | A | C |
| ATOM | 280 | CD | GLN | A | 40 | −22.643 | −61.756 | 15.981 | 1 | 68.79 | A | C |
| ATOM | 281 | OE1 | GLN | A | 40 | −23.299 | −62.703 | 15.59 | 1 | 81.84 | A | O |
| ATOM | 282 | NE2 | GLN | A | 40 | −21.858 | −61.815 | 17.051 | 1 | 75.03 | A | N |
| ATOM | 283 | C | GLN | A | 40 | −21.992 | −59.42 | 11.869 | 1 | 37.94 | A | C |
| ATOM | 284 | O | GLN | A | 40 | −20.99 | −58.716 | 11.959 | 1 | 35.73 | A | O |
| ATOM | 285 | N | VAL | A | 41 | −22.914 | −59.243 | 10.928 | 1 | 34.72 | A | N |
| ATOM | 286 | CA | VAL | A | 41 | −22.721 | −58.218 | 9.912 | 1 | 35.63 | A | C |
| ATOM | 287 | CB | VAL | A | 41 | −23.597 | −58.441 | 8.684 | 1 | 34.34 | A | C |
| ATOM | 288 | CG1 | VAL | A | 41 | −23.267 | −57.41 | 7.617 | 1 | 36.96 | A | C |
| ATOM | 289 | CG2 | VAL | A | 41 | −23.377 | −59.838 | 8.113 | 1 | 32.62 | A | C |
| ATOM | 290 | C | VAL | A | 41 | −22.985 | −56.838 | 10.494 | 1 | 34.63 | A | C |
| ATOM | 291 | O | VAL | A | 41 | −24.095 | −56.515 | 10.869 | 1 | 37.67 | A | O |
| ATOM | 292 | N | THR | A | 42 | −21.974 | −56.005 | 10.559 | 1 | 37.79 | A | N |
| ATOM | 293 | CA | THR | A | 42 | −22.193 | −54.597 | 10.968 | 1 | 34.49 | A | C |
| ATOM | 294 | CB | THR | A | 42 | −21.026 | −54.051 | 11.75 | 1 | 32.47 | A | C |
| ATOM | 295 | OG1 | THR | A | 42 | −19.795 | −54.381 | 11.08 | 1 | 34.31 | A | O |
| ATOM | 296 | CG2 | THR | A | 42 | −21.043 | −54.664 | 13.112 | 1 | 31.77 | A | C |
| ATOM | 297 | C | THR | A | 42 | −22.507 | −53.783 | 9.735 | 1 | 33.3 | A | C |
| ATOM | 298 | O | THR | A | 42 | −21.801 | −53.902 | 8.748 | 1 | 37.94 | A | O |
| ATOM | 299 | N | MET | A | 43 | −23.623 | −53.043 | 9.756 | 1 | 34.82 | A | N |
| ATOM | 300 | CA | MET | A | 43 | −24.056 | −52.241 | 8.614 | 1 | 35.4 | A | C |
| ATOM | 301 | CB | MET | A | 43 | −25.006 | −53.026 | 7.713 | 1 | 38.76 | A | C |
| ATOM | 302 | CG | MET | A | 43 | −26.285 | −53.481 | 8.392 | 1 | 46.61 | A | C |
| ATOM | 303 | SD | MET | A | 43 | −27.435 | −54.37 | 7.308 | 1 | 52.38 | A | S |
| ATOM | 304 | CE | MET | A | 43 | −26.48 | −55.75 | 6.691 | 1 | 44.11 | A | C |
| ATOM | 305 | C | MET | A | 43 | −24.656 | −50.914 | 9.025 | 1 | 36.95 | A | C |
| ATOM | 306 | O | MET | A | 43 | −25.013 | −50.707 | 10.196 | 1 | 38.3 | A | O |
| ATOM | 307 | N | CYS | A | 44 | −24.737 | −50.004 | 8.057 | 1 | 34.89 | A | N |
| ATOM | 308 | CA | CYS | A | 44 | −25.214 | −48.623 | 8.334 | 1 | 39.21 | A | C |
| ATOM | 309 | CB | CYS | A | 44 | −24.336 | −47.65 | 7.546 | 1 | 35.48 | A | C |
| ATOM | 310 | SG | CYS | A | 44 | −22.622 | −47.734 | 8.14 | 1 | 39.94 | A | S |
| ATOM | 311 | C | CYS | A | 44 | −26.695 | −48.463 | 7.953 | 1 | 34.27 | A | C |
| ATOM | 312 | O | CYS | A | 44 | −27.022 | −48.657 | 6.838 | 1 | 39.73 | A | O |
| ATOM | 313 | N | ILE | A | 45 | −27.569 | −48.138 | 8.883 | 1 | 36.41 | A | N |
| ATOM | 314 | CA | ILE | A | 45 | −29.031 | −48.012 | 8.613 | 1 | 37.02 | A | C |
| ATOM | 315 | CB | ILE | A | 45 | −29.831 | −49.215 | 9.153 | 1 | 40.52 | A | C |
| ATOM | 316 | CG1 | ILE | A | 45 | −29.318 | −50.557 | 8.589 | 1 | 43.65 | A | C |
| ATOM | 317 | CD1 | ILE | A | 45 | −29.762 | −51.759 | 9.419 | 1 | 45.06 | A | C |
| ATOM | 318 | CG2 | ILE | A | 45 | −31.302 | −49.133 | 8.768 | 1 | 43.05 | A | C |
| ATOM | 319 | C | ILE | A | 45 | −29.526 | −46.794 | 9.367 | 1 | 38.64 | A | C |
| ATOM | 320 | O | ILE | A | 45 | −29.083 | −46.541 | 10.488 | 1 | 37.35 | A | O |
| ATOM | 321 | N | GLY | A | 46 | −30.435 | −46.053 | 8.742 | 1 | 39.36 | A | N |
| ATOM | 322 | CA | GLY | A | 46 | −31.108 | −44.93 | 9.368 | 1 | 39.55 | A | C |
| ATOM | 323 | C | GLY | A | 46 | −31.153 | −43.703 | 8.512 | 1 | 39.12 | A | C |
| ATOM | 324 | O | GLY | A | 46 | −30.482 | −43.611 | 7.503 | 1 | 41.18 | A | O |
| ATOM | 325 | N | ALA | A | 47 | −31.938 | −42.728 | 8.95 | 1 | 46.44 | A | N |
| ATOM | 326 | CA | ALA | A | 47 | −32.175 | −41.514 | 8.169 | 1 | 44.38 | A | C |
| ATOM | 327 | CB | ALA | A | 47 | −33.432 | −40.816 | 8.651 | 1 | 48.42 | A | C |
| ATOM | 328 | C | ALA | A | 47 | −31.026 | −40.555 | 8.247 | 1 | 38.66 | A | C |
| ATOM | 329 | O | ALA | A | 47 | −30.28 | −40.518 | 9.216 | 1 | 39.82 | A | O |
| ATOM | 330 | N | CYS | A | 48 | −30.944 | −39.764 | 7.195 | 1 | 37.09 | A | N |
| ATOM | 331 | CA | CYS | A | 48 | −29.936 | −38.785 | 6.949 | 1 | 38.96 | A | C |
| ATOM | 332 | CB | CYS | A | 48 | −29.142 | −39.169 | 5.687 | 1 | 41.01 | A | C |
| ATOM | 333 | SG | CYS | A | 48 | −27.897 | −40.466 | 5.982 | 1 | 42.74 | A | S |
| ATOM | 334 | C | CYS | A | 48 | −30.603 | −37.404 | 6.778 | 1 | 36.79 | A | C |
| ATOM | 335 | O | CYS | A | 48 | −30.596 | −36.846 | 5.695 | 1 | 36.33 | A | O |
| ATOM | 336 | N | PRO | A | 49 | −31.169 | −36.851 | 7.856 | 1 | 35.37 | A | N |
| ATOM | 337 | CA | PRO | A | 49 | −31.684 | −35.459 | 7.77 | 1 | 39.45 | A | C |
| ATOM | 338 | CB | PRO | A | 49 | −32.381 | −35.257 | 9.129 | 1 | 37.94 | A | C |
| ATOM | 339 | CG | PRO | A | 49 | −31.678 | −36.199 | 10.063 | 1 | 37.45 | A | C |
| ATOM | 340 | CD | PRO | A | 49 | −31.229 | −37.38 | 9.234 | 1 | 36.05 | A | C |
| ATOM | 341 | C | PRO | A | 49 | −30.565 | −34.434 | 7.575 | 1 | 42.3 | A | C |
| ATOM | 342 | O | PRO | A | 49 | −29.411 | −34.759 | 7.839 | 1 | 45.9 | A | O |
| ATOM | 343 | N | SER | A | 50 | −30.916 | −33.209 | 7.159 | 1 | 42.24 | A | N |
| ATOM | 344 | CA | SER | A | 50 | −29.959 | −32.123 | 6.9 | 1 | 46.61 | A | C |
| ATOM | 345 | CB | SER | A | 50 | −30.675 | −30.775 | 6.741 | 1 | 48.12 | A | C |
| ATOM | 346 | OG | SER | A | 50 | −31.835 | −30.977 | 5.98 | 1 | 62.75 | A | O |
| ATOM | 347 | C | SER | A | 50 | −28.936 | −31.917 | 7.995 | 1 | 53.33 | A | C |
| ATOM | 348 | O | SER | A | 50 | −29.292 | −31.632 | 9.153 | 1 | 54.99 | A | O |
| ATOM | 349 | N | GLN | A | 51 | −27.671 | −32.031 | 7.608 | 1 | 52.54 | A | N |
| ATOM | 350 | CA | GLN | A | 51 | −26.517 | −31.74 | 8.455 | 1 | 49.77 | A | C |
| ATOM | 351 | CB | GLN | A | 51 | −26.632 | −30.362 | 9.115 | 1 | 52.68 | A | C |
| ATOM | 352 | CG | GLN | A | 51 | −26.89 | −29.266 | 8.087 | 1 | 56.89 | A | C |
| ATOM | 353 | CD | GLN | A | 51 | −27.364 | −28.036 | 8.766 | 1 | 62.12 | A | C |
| ATOM | 354 | OE1 | GLN | A | 51 | −28.561 | −27.843 | 8.96 | 1 | 86.28 | A | O |
| ATOM | 355 | NE2 | GLN | A | 51 | −26.444 | −27.24 | 9.216 | 1 | 59.61 | A | N |
| ATOM | 356 | C | GLN | A | 51 | −26.223 | −32.822 | 9.475 | 1 | 47.08 | A | C |
| ATOM | 357 | O | GLN | A | 51 | −25.374 | −32.638 | 10.346 | 1 | 47.4 | A | O |
| ATOM | 358 | N | PHE | A | 52 | −26.89 | −33.957 | 9.367 | 1 | 40.38 | A | N |
| ATOM | 359 | CA | PHE | A | 52 | −26.507 | −35.068 | 10.2 | 1 | 40.29 | A | C |
| ATOM | 360 | CB | PHE | A | 52 | −27.701 | −35.945 | 10.485 | 1 | 43.08 | A | C |
| ATOM | 361 | CG | PHE | A | 52 | −27.436 | −36.975 | 11.516 | 1 | 41.37 | A | C |
| ATOM | 362 | CD1 | PHE | A | 52 | −27.457 | −36.633 | 12.861 | 1 | 41.31 | A | C |
| ATOM | 363 | CE1 | PHE | A | 52 | −27.182 | −37.607 | 13.829 | 1 | 43.74 | A | C |
| ATOM | 364 | CZ | PHE | A | 52 | −26.872 | −38.914 | 13.435 | 1 | 41.63 | A | C |
| ATOM | 365 | CE2 | PHE | A | 52 | −26.856 | −39.257 | 12.085 | 1 | 39.25 | A | C |
| ATOM | 366 | CD2 | PHE | A | 52 | −27.116 | −38.289 | 11.134 | 1 | 41.38 | A | C |
| ATOM | 367 | C | PHE | A | 52 | −25.404 | −35.847 | 9.481 | 1 | 37.08 | A | C |
| ATOM | 368 | O | PHE | A | 52 | −25.621 | −36.336 | 8.345 | 1 | 36.06 | A | O |
| ATOM | 369 | N | ARG | A | 53 | −24.223 | −35.882 | 10.102 | 1 | 34.44 | A | N |
| ATOM | 370 | CA | ARG | A | 53 | −23.075 | −36.678 | 9.672 | 1 | 35.8 | A | C |
| ATOM | 371 | CB | ARG | A | 53 | −23.319 | −38.126 | 10.038 | 1 | 42.17 | A | C |
| ATOM | 372 | CG | ARG | A | 53 | −23.292 | −38.43 | 11.519 | 1 | 45.29 | A | C |
| ATOM | 373 | CD | ARG | A | 53 | −23.184 | −39.955 | 11.665 | 1 | 51.24 | A | C |
| ATOM | 374 | NE | ARG | A | 53 | −23.299 | −40.402 | 13.053 | 1 | 58.4 | A | N |
| ATOM | 375 | CZ | ARG | A | 53 | −23.311 | −41.672 | 13.462 | 1 | 57.44 | A | C |
| ATOM | 376 | NH1 | ARG | A | 53 | −23.214 | −42.657 | 12.601 | 1 | 60.06 | A | N |
| ATOM | 377 | NH2 | ARG | A | 53 | −23.418 | −41.948 | 14.75 | 1 | 64.66 | A | N |
| ATOM | 378 | C | ARG | A | 53 | −22.786 | −36.613 | 8.169 | 1 | 39.19 | A | C |
| ATOM | 379 | O | ARG | A | 53 | −22.564 | −37.643 | 7.489 | 1 | 35.45 | A | O |
| ATOM | 380 | N | ALA | A | 54 | −22.824 | −35.403 | 7.618 | 1 | 38.41 | A | N |
| ATOM | 381 | CA | ALA | A | 54 | −22.53 | −35.228 | 6.217 | 1 | 35.12 | A | C |
| ATOM | 382 | CB | ALA | A | 54 | −22.646 | −33.747 | 5.831 | 1 | 37.96 | A | C |
| ATOM | 383 | C | ALA | A | 54 | −21.116 | −35.763 | 5.987 | 1 | 39.88 | A | C |
| ATOM | 384 | O | ALA | A | 54 | −20.209 | −35.404 | 6.714 | 1 | 44.71 | A | O |
| ATOM | 385 | N | ALA | A | 55 | −20.931 | −36.651 | 5.012 | 1 | 39.44 | A | N |
| ATOM | 386 | CA | ALA | A | 55 | −19.632 | −37.292 | 4.805 | 1 | 36.66 | A | C |
| ATOM | 387 | CB | ALA | A | 55 | −19.747 | −38.392 | 3.783 | 1 | 37.03 | A | C |
| ATOM | 388 | C | ALA | A | 55 | −18.561 | −36.342 | 4.367 | 1 | 37.25 | A | C |
| ATOM | 389 | O | ALA | A | 55 | −17.442 | −36.553 | 4.669 | 1 | 41.75 | A | O |
| ATOM | 390 | N | ASN | A | 56 | −18.909 | −35.316 | 3.614 | 1 | 37.93 | A | N |
| ATOM | 391 | CA | ASN | A | 56 | −17.955 | −34.532 | 2.884 | 1 | 37.51 | A | C |
| ATOM | 392 | CB | ASN | A | 56 | −17.235 | −35.407 | 1.814 | 1 | 37.5 | A | C |
| ATOM | 393 | CG | ASN | A | 56 | −18.18 | −36.097 | 0.82 | 1 | 37.2 | A | C |
| ATOM | 394 | OD1 | ASN | A | 56 | −19.364 | −35.74 | 0.653 | 1 | 38.76 | A | O |
| ATOM | 395 | ND2 | ASN | A | 56 | −17.634 | −37.076 | 0.116 | 1 | 32.65 | A | N |
| ATOM | 396 | C | ASN | A | 56 | −18.705 | −33.375 | 2.267 | 1 | 39.91 | A | C |
| ATOM | 397 | O | ASN | A | 56 | −19.894 | −33.177 | 2.568 | 1 | 39.53 | A | O |
| ATOM | 398 | N | MET | A | 57 | −18.051 | −32.624 | 1.391 | 1 | 41.37 | A | N |
| ATOM | 399 | CA | MET | A | 57 | −18.684 | −31.453 | 0.794 | 1 | 43.15 | A | C |
| ATOM | 400 | CB | MET | A | 57 | −17.664 | −30.466 | 0.221 | 1 | 50.27 | A | C |
| ATOM | 401 | CG | MET | A | 57 | −17.193 | −29.531 | 1.319 | 1 | 56.71 | A | C |
| ATOM | 402 | SD | MET | A | 57 | −16.075 | −28.235 | 0.815 | 1 | 69.21 | A | S |
| ATOM | 403 | CE | MET | A | 57 | −16.975 | −27.396 | −0.49 | 1 | 64.21 | A | C |
| ATOM | 404 | C | MET | A | 57 | −19.695 | −31.808 | −0.245 | 1 | 41.13 | A | C |
| ATOM | 405 | O | MET | A | 57 | −20.689 | −31.109 | −0.362 | 1 | 41.62 | A | O |
| ATOM | 406 | N | HIS | A | 58 | −19.476 | −32.897 | −0.978 | 1 | 34.31 | A | N |
| ATOM | 407 | CA | HIS | A | 58 | −20.508 | −33.392 | −1.854 | 1 | 33.98 | A | C |
| ATOM | 408 | CB | HIS | A | 58 | −20.057 | −34.69 | −2.501 | 1 | 35.61 | A | C |
| ATOM | 409 | CG | HIS | A | 58 | −21.001 | −35.203 | −3.535 | 1 | 34.01 | A | C |
| ATOM | 410 | ND1 | HIS | A | 58 | −21.068 | −34.684 | −4.802 | 1 | 37.93 | A | N |
| ATOM | 411 | CE1 | HIS | A | 58 | −21.982 | −35.338 | −5.504 | 1 | 33.69 | A | C |
| ATOM | 412 | NE2 | HIS | A | 58 | −22.502 | −36.266 | −4.729 | 1 | 33.89 | A | N |
| ATOM | 413 | CD2 | HIS | A | 58 | −21.933 | −36.183 | −3.483 | 1 | 34.79 | A | C |
| ATOM | 414 | C | HIS | A | 58 | −21.818 | −33.624 | −1.086 | 1 | 31.95 | A | C |
| ATOM | 415 | O | HIS | A | 58 | −22.865 | −33.318 | −1.556 | 1 | 32.21 | A | O |
| ATOM | 416 | N | ALA | A | 59 | −21.735 | −34.196 | 0.099 | 1 | 35.12 | A | N |
| ATOM | 417 | CA | ALA | A | 59 | −22.898 | −34.458 | 0.885 | 1 | 35.66 | A | C |
| ATOM | 418 | CB | ALA | A | 59 | −22.532 | −35.212 | 2.152 | 1 | 36.66 | A | C |
| ATOM | 419 | C | ALA | A | 59 | −23.611 | −33.129 | 1.203 | 1 | 36.32 | A | C |
| ATOM | 420 | O | ALA | A | 59 | −24.859 | −33.056 | 1.12 | 1 | 34.81 | A | O |
| ATOM | 421 | N | GLN | A | 60 | −22.839 | −32.109 | 1.572 | 1 | 33.39 | A | N |
| ATOM | 422 | CA | GLN | A | 60 | −23.408 | −30.766 | 1.887 | 1 | 37.53 | A | C |
| ATOM | 423 | CB | GLN | A | 60 | −22.386 | −29.831 | 2.48 | 1 | 37.43 | A | C |
| ATOM | 424 | CG | GLN | A | 60 | −21.955 | −30.368 | 3.838 | 1 | 46.78 | A | C |
| ATOM | 425 | CD | GLN | A | 60 | −20.831 | −29.594 | 4.484 | 1 | 51.97 | A | C |
| ATOM | 426 | OE1 | GLN | A | 60 | −19.91 | −29.14 | 3.816 | 1 | 50.51 | A | O |
| ATOM | 427 | NE2 | GLN | A | 60 | −20.899 | −29.461 | 5.815 | 1 | 56.35 | A | N |
| ATOM | 428 | C | GLN | A | 60 | −24.098 | −30.067 | 0.739 | 1 | 37.04 | A | C |
| ATOM | 429 | O | GLN | A | 60 | −25.091 | −29.397 | 0.95 | 1 | 36.44 | A | O |
| ATOM | 430 | N | ILE | A | 61 | −23.576 | −30.249 | −0.461 | 1 | 35.1 | A | N |
| ATOM | 431 | CA | ILE | A | 61 | −24.193 | −29.73 | −1.643 | 1 | 34.26 | A | C |
| ATOM | 432 | CB | ILE | A | 61 | −23.194 | −29.843 | −2.787 | 1 | 35.08 | A | C |
| ATOM | 433 | CG1 | ILE | A | 61 | −22.096 | −28.803 | −2.632 | 1 | 38.47 | A | C |
| ATOM | 434 | CD1 | ILE | A | 61 | −20.825 | −29.178 | −3.387 | 1 | 43.35 | A | C |
| ATOM | 435 | CG2 | ILE | A | 61 | −23.853 | −29.666 | −4.136 | 1 | 37.38 | A | C |
| ATOM | 436 | C | ILE | A | 61 | −25.492 | −30.482 | −1.965 | 1 | 35.36 | A | C |
| ATOM | 437 | O | ILE | A | 61 | −26.462 | −29.893 | −2.428 | 1 | 35.29 | A | O |
| ATOM | 438 | N | LYS | A | 62 | −25.481 | −31.804 | −1.796 | 1 | 39.17 | A | N |
| ATOM | 439 | CA | LYS | A | 62 | −26.612 | −32.649 | −2.153 | 1 | 38.21 | A | C |
| ATOM | 440 | CB | LYS | A | 62 | −26.232 | −34.108 | −2 | 1 | 48.13 | A | C |
| ATOM | 441 | CG | LYS | A | 62 | −27.126 | −35.129 | −2.692 | 1 | 53.06 | A | C |
| ATOM | 442 | CD | LYS | A | 62 | −26.361 | −36.422 | −2.887 | 1 | 55.44 | A | C |
| ATOM | 443 | CE | LYS | A | 62 | −27.208 | −37.53 | −3.448 | 1 | 58.35 | A | C |
| ATOM | 444 | NZ | LYS | A | 62 | −27.571 | −38.549 | −2.391 | 1 | 65.81 | A | N |
| ATOM | 445 | C | LYS | A | 62 | −27.796 | −32.318 | −1.249 | 1 | 36.2 | A | C |
| ATOM | 446 | O | LYS | A | 62 | −28.911 | −32.264 | −1.691 | 1 | 38.08 | A | O |
| ATOM | 447 | N | THR | A | 63 | −27.529 | −32.097 | 0.022 | 1 | 33.98 | A | N |
| ATOM | 448 | CA | THR | A | 63 | −28.513 | −31.592 | 0.943 | 1 | 35.2 | A | C |
| ATOM | 449 | CB | THR | A | 63 | −27.937 | −31.498 | 2.349 | 1 | 35.65 | A | C |
| ATOM | 450 | OG1 | THR | A | 63 | −27.875 | −32.823 | 2.864 | 1 | 36.24 | A | O |
| ATOM | 451 | CG2 | THR | A | 63 | −28.807 | −30.681 | 3.312 | 1 | 34.62 | A | C |
| ATOM | 452 | C | THR | A | 63 | −29.077 | −30.256 | 0.539 | 1 | 38.25 | A | C |
| ATOM | 453 | O | THR | A | 63 | −30.29 | −30.088 | 0.558 | 1 | 38.9 | A | O |
| ATOM | 454 | N | SER | A | 64 | −28.226 | −29.305 | 0.182 | 1 | 38.7 | A | N |
| ATOM | 455 | CA | SER | A | 64 | −28.742 | −28.008 | −0.252 | 1 | 39.6 | A | C |
| ATOM | 456 | CB | SER | A | 64 | −27.625 | −27.051 | −0.609 | 1 | 38.63 | A | C |
| ATOM | 457 | OG | SER | A | 64 | −26.894 | −26.695 | 0.558 | 1 | 37.5 | A | O |
| ATOM | 458 | C | SER | A | 64 | −29.667 | −28.175 | −1.436 | 1 | 38.99 | A | C |
| ATOM | 459 | O | SER | A | 64 | −30.772 | −27.621 | −1.447 | 1 | 37.82 | A | O |
| ATOM | 460 | N | LEU | A | 65 | −29.202 | −28.931 | −2.425 | 1 | 39.4 | A | N |
| ATOM | 461 | CA | LEU | A | 65 | −29.982 | −29.149 | −3.648 | 1 | 38.08 | A | C |
| ATOM | 462 | CB | LEU | A | 65 | −29.134 | −29.691 | −4.775 | 1 | 32.49 | A | C |
| ATOM | 463 | CG | LEU | A | 65 | −28.031 | −28.734 | −5.222 | 1 | 39.3 | A | C |
| ATOM | 464 | CD1 | LEU | A | 65 | −27.191 | −29.373 | −6.326 | 1 | 37.33 | A | C |
| ATOM | 465 | CD2 | LEU | A | 65 | −28.496 | −27.313 | −5.631 | 1 | 39.44 | A | C |
| ATOM | 466 | C | LEU | A | 65 | −31.213 | −30.025 | −3.484 | 1 | 39.79 | A | C |
| ATOM | 467 | O | LEU | A | 65 | −32.151 | −29.854 | −4.21 | 1 | 40.29 | A | O |
| ATOM | 468 | N | HIS | A | 66 | −31.165 | −30.987 | −2.573 | 1 | 40.28 | A | N |
| ATOM | 469 | CA | HIS | A | 66 | −32.305 | −31.756 | −2.236 | 1 | 41.11 | A | C |
| ATOM | 470 | CB | HIS | A | 66 | −31.892 | −32.932 | −1.368 | 1 | 38.52 | A | C |
| ATOM | 471 | CG | HIS | A | 66 | −33.03 | −33.595 | −0.691 | 1 | 36.78 | A | C |
| ATOM | 472 | ND1 | HIS | A | 66 | −33.983 | −34.332 | −1.371 | 1 | 35.49 | A | N |
| ATOM | 473 | CE1 | HIS | A | 66 | −34.854 | −34.808 | −0.5 | 1 | 35.19 | A | C |
| ATOM | 474 | NE2 | HIS | A | 66 | −34.495 | −34.407 | 0.717 | 1 | 39 | A | N |
| ATOM | 475 | CD2 | HIS | A | 66 | −33.369 | −33.64 | 0.621 | 1 | 38.73 | A | C |
| ATOM | 476 | C | HIS | A | 66 | −33.372 | −30.871 | −1.553 | 1 | 40.57 | A | C |
| ATOM | 477 | O | HIS | A | 66 | −34.535 | −30.962 | −1.913 | 1 | 38.81 | A | O |
| ATOM | 478 | N | ARG | A | 67 | −32.988 | −29.995 | −0.631 | 1 | 37.94 | A | N |
| ATOM | 479 | CA | ARG | A | 67 | −33.98 | −29.11 | 0.013 | 1 | 39.23 | A | C |
| ATOM | 480 | CB | ARG | A | 67 | −33.488 | −28.412 | 1.292 | 1 | 40.88 | A | C |
| ATOM | 481 | CG | ARG | A | 67 | −32.936 | −29.376 | 2.349 | 1 | 44.02 | A | C |
| ATOM | 482 | CD | ARG | A | 67 | −32.318 | −28.645 | 3.556 | 1 | 44.22 | A | C |
| ATOM | 483 | NE | ARG | A | 67 | −33.39 | −27.899 | 4.185 | 1 | 47.25 | A | N |
| ATOM | 484 | CZ | ARG | A | 67 | −34.349 | −28.42 | 4.963 | 1 | 48.97 | A | C |
| ATOM | 485 | NH1 | ARG | A | 67 | −35.311 | −27.638 | 5.409 | 1 | 51.18 | A | N |
| ATOM | 486 | NH2 | ARG | A | 67 | −34.363 | −29.703 | 5.307 | 1 | 51.87 | A | N |
| ATOM | 487 | C | ARG | A | 67 | −34.55 | −28.112 | −0.969 | 1 | 42.98 | A | C |
| ATOM | 488 | O | ARG | A | 67 | −35.708 | −27.758 | −0.894 | 1 | 46.54 | A | O |
| ATOM | 489 | N | LEU | A | 68 | −33.775 | −27.732 | −1.951 | 1 | 44.17 | A | N |
| ATOM | 490 | CA | LEU | A | 68 | −34.229 | −26.797 | −2.94 | 1 | 40.05 | A | C |
| ATOM | 491 | CB | LEU | A | 68 | −32.992 | −26.111 | −3.507 | 1 | 47.84 | A | C |
| ATOM | 492 | CG | LEU | A | 68 | −33.154 | −24.932 | −4.439 | 1 | 48.82 | A | C |
| ATOM | 493 | CD1 | LEU | A | 68 | −33.711 | −23.771 | −3.65 | 1 | 50.19 | A | C |
| ATOM | 494 | CD2 | LEU | A | 68 | −31.812 | −24.59 | −5.068 | 1 | 52.69 | A | C |
| ATOM | 495 | C | LEU | A | 68 | −35.028 | −27.448 | −4.049 | 1 | 43.1 | A | C |
| ATOM | 496 | O | LEU | A | 68 | −35.855 | −26.795 | −4.67 | 1 | 45.11 | A | O |
| ATOM | 497 | N | LYS | A | 69 | −34.772 | −28.719 | −4.333 | 1 | 40.81 | A | N |
| ATOM | 498 | CA | LYS | A | 69 | −35.273 | −29.374 | −5.545 | 1 | 41.64 | A | C |
| ATOM | 499 | CB | LYS | A | 69 | −34.215 | −29.239 | −6.628 | 1 | 50.28 | A | C |
| ATOM | 500 | CG | LYS | A | 69 | −34.568 | −28.277 | −7.717 | 1 | 61.5 | A | C |
| ATOM | 501 | CD | LYS | A | 69 | −33.354 | −27.88 | −8.542 | 1 | 67.94 | A | C |
| ATOM | 502 | CE | LYS | A | 69 | −32.875 | −26.499 | −8.138 | 1 | 79.13 | A | C |
| ATOM | 503 | NZ | LYS | A | 69 | −32.569 | −25.7 | −9.353 | 1 | 77.8 | A | N |
| ATOM | 504 | C | LYS | A | 69 | −35.575 | −30.845 | −5.256 | 1 | 39.68 | A | C |
| ATOM | 505 | O | LYS | A | 69 | −34.951 | −31.746 | −5.824 | 1 | 40.69 | A | O |
| ATOM | 506 | N | PRO | A | 70 | −36.525 | −31.097 | −4.343 | 1 | 39.23 | A | N |
| ATOM | 507 | CA | PRO | A | 70 | −36.724 | −32.425 | −3.747 | 1 | 41.06 | A | C |
| ATOM | 508 | CB | PRO | A | 70 | −37.749 | −32.18 | −2.655 | 1 | 40.27 | A | C |
| ATOM | 509 | CG | PRO | A | 70 | −38.426 | −30.937 | −3.064 | 1 | 40.89 | A | C |
| ATOM | 510 | CD | PRO | A | 70 | −37.432 | −30.103 | −3.783 | 1 | 40.01 | A | C |
| ATOM | 511 | C | PRO | A | 70 | −37.175 | −33.527 | −4.695 | 1 | 40.12 | A | C |
| ATOM | 512 | O | PRO | A | 70 | −36.947 | −34.684 | −4.402 | 1 | 40.59 | A | O |
| ATOM | 513 | N | ASP | A | 71 | −37.792 | −33.179 | −5.812 | 1 | 43.12 | A | N |
| ATOM | 514 | CA | ASP | A | 71 | −38.161 | −34.182 | −6.814 | 1 | 48.15 | A | C |
| ATOM | 515 | CB | ASP | A | 71 | −39.373 | −33.695 | −7.628 | 1 | 45.93 | A | C |
| ATOM | 516 | CG | ASP | A | 71 | −40.614 | −33.537 | −6.772 | 1 | 47.79 | A | C |
| ATOM | 517 | OD1 | ASP | A | 71 | −40.649 | −34.141 | −5.7 | 1 | 45.88 | A | O |
| ATOM | 518 | OD2 | ASP | A | 71 | −41.525 | −32.762 | −7.134 | 1 | 48.23 | A | O |
| ATOM | 519 | C | ASP | A | 71 | −37.029 | −34.56 | −7.773 | 1 | 49.46 | A | C |
| ATOM | 520 | O | ASP | A | 71 | −37.096 | −35.595 | −8.429 | 1 | 50.83 | A | O |
| ATOM | 521 | N | THR | A | 72 | −36.03 | −33.709 | −7.895 | 1 | 48.85 | A | N |
| ATOM | 522 | CA | THR | A | 72 | −34.875 | −33.984 | −8.754 | 1 | 48.71 | A | C |
| ATOM | 523 | CB | THR | A | 72 | −34.318 | −32.69 | −9.347 | 1 | 49.41 | A | C |
| ATOM | 524 | OG1 | THR | A | 72 | −35.374 | −31.844 | −9.8 | 1 | 57 | A | O |
| ATOM | 525 | CG2 | THR | A | 72 | −33.364 | −32.995 | −10.489 | 1 | 57.6 | A | C |
| ATOM | 526 | C | THR | A | 72 | −33.722 | −34.606 | −7.939 | 1 | 47.52 | A | C |
| ATOM | 527 | O | THR | A | 72 | −33.076 | −35.488 | −8.417 | 1 | 45.82 | A | O |
| ATOM | 528 | N | VAL | A | 73 | −33.445 | −34.081 | −6.738 | 1 | 44.05 | A | N |
| ATOM | 529 | CA | VAL | A | 73 | −32.256 | −34.445 | −5.994 | 1 | 45.16 | A | C |
| ATOM | 530 | CB | VAL | A | 73 | −31.427 | −33.205 | −5.632 | 1 | 45.49 | A | C |
| ATOM | 531 | CG1 | VAL | A | 73 | −30.18 | −33.613 | −4.884 | 1 | 50.8 | A | C |
| ATOM | 532 | CG2 | VAL | A | 73 | −31.041 | −32.473 | −6.89 | 1 | 45.69 | A | C |
| ATOM | 533 | C | VAL | A | 73 | −32.681 | −35.201 | −4.744 | 1 | 40.58 | A | C |
| ATOM | 534 | O | VAL | A | 73 | −33.359 | −34.643 | −3.899 | 1 | 37.48 | A | O |
| ATOM | 535 | N | PRO | A | 74 | −32.318 | −36.485 | −4.641 | 1 | 41.21 | A | N |
| ATOM | 536 | CA | PRO | A | 74 | −32.77 | −37.241 | −3.483 | 1 | 43.58 | A | C |
| ATOM | 537 | CB | PRO | A | 74 | −32.476 | −38.68 | −3.889 | 1 | 43.61 | A | C |
| ATOM | 538 | CG | PRO | A | 74 | −31.285 | −38.561 | −4.748 | 1 | 45.06 | A | C |
| ATOM | 539 | CD | PRO | A | 74 | −31.459 | −37.296 | −5.525 | 1 | 44.93 | A | C |
| ATOM | 540 | C | PRO | A | 74 | −32.004 | −36.859 | −2.225 | 1 | 46.17 | A | C |
| ATOM | 541 | O | PRO | A | 74 | −30.96 | −36.217 | −2.299 | 1 | 42.53 | A | O |
| ATOM | 542 | N | ALA | A | 75 | −32.543 | −37.255 | −1.079 | 1 | 53.66 | A | N |
| ATOM | 543 | CA | ALA | A | 75 | −31.86 | −37.107 | 0.21 | 1 | 54.13 | A | C |
| ATOM | 544 | CB | ALA | A | 75 | −32.794 | −37.547 | 1.306 | 1 | 56.52 | A | C |
| ATOM | 545 | C | ALA | A | 75 | −30.567 | −37.96 | 0.308 | 1 | 54.78 | A | C |
| ATOM | 546 | O | ALA | A | 75 | −30.447 | −38.994 | −0.359 | 1 | 42.71 | A | O |
| ATOM | 547 | N | PRO | A | 76 | −29.625 | −37.558 | 1.185 | 1 | 55.5 | A | N |
| ATOM | 548 | CA | PRO | A | 76 | −28.405 | −38.361 | 1.337 | 1 | 52.04 | A | C |
| ATOM | 549 | CB | PRO | A | 76 | −27.612 | −37.549 | 2.369 | 1 | 58.18 | A | C |
| ATOM | 550 | CG | PRO | A | 76 | −27.977 | −36.117 | 2.055 | 1 | 53.52 | A | C |
| ATOM | 551 | CD | PRO | A | 76 | −29.464 | −36.238 | 1.846 | 1 | 57.77 | A | C |
| ATOM | 552 | C | PRO | A | 76 | −28.67 | −39.784 | 1.781 | 1 | 43.89 | A | C |
| ATOM | 553 | O | PRO | A | 76 | −29.721 | −40.039 | 2.341 | 1 | 46.97 | A | O |
| ATOM | 554 | N | CYS | A | 77 | −27.782 | −40.714 | 1.462 | 1 | 42.22 | A | N |
| ATOM | 555 | CA | CYS | A | 77 | −27.911 | −42.091 | 1.964 | 1 | 43.58 | A | C |
| ATOM | 556 | CB | CYS | A | 77 | −27.806 | −43.152 | 0.853 | 1 | 48.49 | A | C |
| ATOM | 557 | SG | CYS | A | 77 | −27.618 | −42.727 | −0.912 | 1 | 77.89 | A | S |
| ATOM | 558 | C | CYS | A | 77 | −26.92 | −42.465 | 3.103 | 1 | 36.1 | A | C |
| ATOM | 559 | O | CYS | A | 77 | −25.782 | −42.028 | 3.125 | 1 | 36.88 | A | O |
| ATOM | 560 | N | CYS | A | 78 | −27.36 | −43.337 | 4.002 | 1 | 34.84 | A | N |
| ATOM | 561 | CA | CYS | A | 78 | −26.552 | −43.854 | 5.123 | 1 | 35.57 | A | C |
| ATOM | 562 | CB | CYS | A | 78 | −27.464 | −44.44 | 6.23 | 1 | 33.58 | A | C |
| ATOM | 563 | SG | CYS | A | 78 | −26.603 | −44.959 | 7.735 | 1 | 40.5 | A | S |
| ATOM | 564 | C | CYS | A | 78 | −25.622 | −44.961 | 4.586 | 1 | 36.81 | A | C |
| ATOM | 565 | O | CYS | A | 78 | −26.116 | −45.997 | 4.183 | 1 | 37.76 | A | O |
| ATOM | 566 | N | VAL | A | 79 | −24.3 | −44.707 | 4.567 | 1 | 37.99 | A | N |
| ATOM | 567 | CA | VAL | A | 79 | −23.276 | −45.643 | 4.063 | 1 | 39.76 | A | C |
| ATOM | 568 | CB | VAL | A | 79 | −22.747 | −45.25 | 2.67 | 1 | 43 | A | C |
| ATOM | 569 | CG1 | VAL | A | 79 | −23.909 | −45.236 | 1.693 | 1 | 45.91 | A | C |
| ATOM | 570 | CG2 | VAL | A | 79 | −22.047 | −43.893 | 2.688 | 1 | 46.23 | A | C |
| ATOM | 571 | C | VAL | A | 79 | −22.095 | −45.759 | 5.018 | 1 | 35.34 | A | C |
| ATOM | 572 | O | VAL | A | 79 | −21.941 | −44.929 | 5.902 | 1 | 36.25 | A | O |
| ATOM | 573 | N | PRO | A | 80 | −21.273 | −46.808 | 4.852 | 1 | 35.99 | A | N |
| ATOM | 574 | CA | PRO | A | 80 | −20.101 | −46.948 | 5.709 | 1 | 37.36 | A | C |
| ATOM | 575 | CB | PRO | A | 80 | −19.546 | −48.356 | 5.331 | 1 | 34.42 | A | C |
| ATOM | 576 | CG | PRO | A | 80 | −20.733 | −49.092 | 4.855 | 1 | 34.43 | A | C |
| ATOM | 577 | CD | PRO | A | 80 | −21.459 | −48.048 | 4.047 | 1 | 37.91 | A | C |
| ATOM | 578 | C | PRO | A | 80 | −19.084 | −45.888 | 5.402 | 1 | 35.53 | A | C |
| ATOM | 579 | O | PRO | A | 80 | −18.785 | −45.661 | 4.253 | 1 | 34.05 | A | O |
| ATOM | 580 | N | ALA | A | 81 | −18.575 | −45.256 | 6.445 | 1 | 39.9 | A | N |
| ATOM | 581 | CA | ALA | A | 81 | −17.469 | −44.302 | 6.339 | 1 | 38.67 | A | C |
| ATOM | 582 | CB | ALA | A | 81 | −17.547 | −43.327 | 7.507 | 1 | 39.8 | A | C |
| ATOM | 583 | C | ALA | A | 81 | −16.132 | −45.013 | 6.37 | 1 | 35.89 | A | C |
| ATOM | 584 | O | ALA | A | 81 | −15.161 | −44.485 | 5.922 | 1 | 36.72 | A | O |
| ATOM | 585 | N | SER | A | 82 | −16.087 | −46.19 | 6.971 | 1 | 37.5 | A | N |
| ATOM | 586 | CA | SER | A | 82 | −14.881 | −47.004 | 7.04 | 1 | 36.94 | A | C |
| ATOM | 587 | CB | SER | A | 82 | −13.964 | −46.46 | 8.146 | 1 | 35.17 | A | C |
| ATOM | 588 | OG | SER | A | 82 | −14.627 | −46.61 | 9.397 | 1 | 36.11 | A | O |
| ATOM | 589 | C | SER | A | 82 | −15.316 | −48.443 | 7.374 | 1 | 33.43 | A | C |
| ATOM | 590 | O | SER | A | 82 | −16.449 | −48.656 | 7.747 | 1 | 34.74 | A | O |
| ATOM | 591 | N | TYR | A | 83 | −14.387 | −49.388 | 7.315 | 1 | 33.39 | A | N |
| ATOM | 592 | CA | TYR | A | 83 | −14.643 | −50.823 | 7.566 | 1 | 34.64 | A | C |
| ATOM | 593 | CB | TYR | A | 83 | −14.474 | −51.58 | 6.238 | 1 | 36.17 | A | C |
| ATOM | 594 | CG | TYR | A | 83 | −15.488 | −51.216 | 5.175 | 1 | 33.18 | A | C |
| ATOM | 595 | CD1 | TYR | A | 83 | −15.272 | −50.121 | 4.32 | 1 | 30.47 | A | C |
| ATOM | 596 | CE1 | TYR | A | 83 | −16.191 | −49.766 | 3.336 | 1 | 29.01 | A | C |
| ATOM | 597 | CZ | TYR | A | 83 | −17.382 | −50.505 | 3.24 | 1 | 30.48 | A | C |
| ATOM | 598 | OH | TYR | A | 83 | −18.303 | −50.194 | 2.304 | 1 | 31.23 | A | O |
| ATOM | 599 | CE2 | TYR | A | 83 | −17.621 | −51.593 | 4.081 | 1 | 32.56 | A | C |
| ATOM | 600 | CD2 | TYR | A | 83 | −16.673 | −51.949 | 5.04 | 1 | 32.34 | A | C |
| ATOM | 601 | C | TYR | A | 83 | −13.732 | −51.489 | 8.6 | 1 | 33.76 | A | C |
| ATOM | 602 | O | TYR | A | 83 | −12.627 | −51.052 | 8.837 | 1 | 38.91 | A | O |
| ATOM | 603 | N | ASN | A | 84 | −14.191 | −52.569 | 9.204 | 1 | 37.63 | A | N |
| ATOM | 604 | CA | ASN | A | 84 | −13.311 | −53.443 | 10.02 | 1 | 37.16 | A | C |
| ATOM | 605 | CB | ASN | A | 84 | −14.054 | −53.941 | 11.257 | 1 | 37.76 | A | C |
| ATOM | 606 | CG | ASN | A | 84 | −14.164 | −52.887 | 12.323 | 1 | 39.1 | A | C |
| ATOM | 607 | OD1 | ASN | A | 84 | −13.195 | −52.226 | 12.631 | 1 | 43.1 | A | O |
| ATOM | 608 | ND2 | ASN | A | 84 | −15.345 | −52.722 | 12.884 | 1 | 43 | A | N |
| ATOM | 609 | C | ASN | A | 84 | −12.941 | −54.646 | 9.2 | 1 | 35.3 | A | C |
| ATOM | 610 | O | ASN | A | 84 | −13.823 | −55.322 | 8.674 | 1 | 40.42 | A | O |
| ATOM | 611 | N | PRO | A | 85 | −11.662 | −54.955 | 9.084 | 1 | 34.85 | A | N |
| ATOM | 612 | CA | PRO | A | 85 | −11.325 | −56.188 | 8.354 | 1 | 36.3 | A | C |
| ATOM | 613 | CB | PRO | A | 85 | −9.79 | −56.228 | 8.378 | 1 | 39.95 | A | C |
| ATOM | 614 | CG | PRO | A | 85 | −9.347 | −55.204 | 9.372 | 1 | 41.33 | A | C |
| ATOM | 615 | CD | PRO | A | 85 | −10.481 | −54.223 | 9.568 | 1 | 40.25 | A | C |
| ATOM | 616 | C | PRO | A | 85 | −11.834 | −57.488 | 8.988 | 1 | 37.32 | A | C |
| ATOM | 617 | O | PRO | A | 85 | −11.908 | −57.613 | 10.22 | 1 | 40.5 | A | O |
| ATOM | 618 | N | MET | A | 86 | −12.075 | −58.479 | 8.15 | 1 | 31.09 | A | N |
| ATOM | 619 | CA | MET | A | 86 | −12.52 | −59.771 | 8.622 | 1 | 33.25 | A | C |
| ATOM | 620 | CB | MET | A | 86 | −14.038 | −59.903 | 8.394 | 1 | 35.78 | A | C |
| ATOM | 621 | CG | MET | A | 86 | −14.678 | −61.094 | 9.082 | 1 | 44.89 | A | C |
| ATOM | 622 | SD | MET | A | 86 | −16.413 | −61.283 | 8.594 | 1 | 45.5 | A | S |
| ATOM | 623 | CE | MET | A | 86 | −16.65 | −63.013 | 8.871 | 1 | 51.89 | A | C |
| ATOM | 624 | C | MET | A | 86 | −11.845 | −60.885 | 7.859 | 1 | 34.02 | A | C |
| ATOM | 625 | O | MET | A | 86 | −11.617 | −60.777 | 6.647 | 1 | 34.69 | A | O |
| ATOM | 626 | N | VAL | A | 87 | −11.65 | −62.001 | 8.539 | 1 | 32.65 | A | N |
| ATOM | 627 | CA | VAL | A | 87 | −11.083 | −63.185 | 7.933 | 1 | 33.25 | A | C |
| ATOM | 628 | CB | VAL | A | 87 | −10.057 | −63.863 | 8.875 | 1 | 35.01 | A | C |
| ATOM | 629 | CG1 | VAL | A | 87 | −9.347 | −65.021 | 8.185 | 1 | 35.09 | A | C |
| ATOM | 630 | CG2 | VAL | A | 87 | −9.054 | −62.861 | 9.401 | 1 | 37.43 | A | C |
| ATOM | 631 | C | VAL | A | 87 | −12.178 | −64.202 | 7.647 | 1 | 34.02 | A | C |
| ATOM | 632 | O | VAL | A | 87 | −13.132 | −64.357 | 8.431 | 1 | 32.57 | A | O |
| ATOM | 633 | N | LEU | A | 88 | −12.021 | −64.903 | 6.521 | 1 | 31.52 | A | N |
| ATOM | 634 | CA | LEU | A | 88 | −12.879 | −65.992 | 6.115 | 1 | 35.17 | A | C |
| ATOM | 635 | CB | LEU | A | 88 | −13.573 | −65.644 | 4.764 | 1 | 37.86 | A | C |
| ATOM | 636 | CG | LEU | A | 88 | −14.977 | −65.088 | 4.707 | 1 | 42.6 | A | C |
| ATOM | 637 | CD1 | LEU | A | 88 | −15.223 | −64.077 | 5.807 | 1 | 52.34 | A | C |
| ATOM | 638 | CD2 | LEU | A | 88 | −15.29 | −64.472 | 3.361 | 1 | 44.16 | A | C |
| ATOM | 639 | C | LEU | A | 88 | −11.963 | −67.182 | 5.882 | 1 | 35.12 | A | C |
| ATOM | 640 | O | LEU | A | 88 | −10.914 | −66.995 | 5.325 | 1 | 37.27 | A | O |
| ATOM | 641 | N | ILE | A | 89 | −12.375 | −68.396 | 6.234 | 1 | 37.58 | A | N |
| ATOM | 642 | CA | ILE | A | 89 | −11.708 | −69.594 | 5.696 | 1 | 35.03 | A | C |
| ATOM | 643 | CB | ILE | A | 89 | −11.548 | −70.684 | 6.755 | 1 | 35.1 | A | C |
| ATOM | 644 | CG1 | ILE | A | 89 | −10.824 | −70.115 | 7.995 | 1 | 36.24 | A | C |
| ATOM | 645 | CD1 | ILE | A | 89 | −10.73 | −71.104 | 9.15 | 1 | 39.26 | A | C |
| ATOM | 646 | CG2 | ILE | A | 89 | −10.78 | −71.885 | 6.191 | 1 | 38.2 | A | C |
| ATOM | 647 | C | ILE | A | 89 | −12.501 | −70.11 | 4.487 | 1 | 37.35 | A | C |
| ATOM | 648 | O | ILE | A | 89 | −13.569 | −70.645 | 4.649 | 1 | 35.18 | A | O |
| ATOM | 649 | N | GLN | A | 90 | −11.927 | −69.958 | 3.293 | 1 | 40.15 | A | N |
| ATOM | 650 | CA | GLN | A | 90 | −12.546 | −70.392 | 2.029 | 1 | 45.36 | A | C |
| ATOM | 651 | CB | GLN | A | 90 | −12.12 | −69.503 | 0.865 | 1 | 44.4 | A | C |
| ATOM | 652 | CG | GLN | A | 90 | −12.434 | −68.052 | 1.115 | 1 | 50.18 | A | C |
| ATOM | 653 | CD | GLN | A | 90 | −11.86 | −67.108 | 0.074 | 1 | 56.38 | A | C |
| ATOM | 654 | OE1 | GLN | A | 90 | −11.639 | −65.932 | 0.368 | 1 | 61.3 | A | O |
| ATOM | 655 | NE2 | GLN | A | 90 | −11.654 | −67.592 | −1.141 | 1 | 52.84 | A | N |
| ATOM | 656 | C | GLN | A | 90 | −12.107 | −71.799 | 1.67 | 1 | 47.78 | A | C |
| ATOM | 657 | O | GLN | A | 90 | −10.916 | −72.126 | 1.767 | 1 | 46.24 | A | O |
| ATOM | 658 | N | LYS | A | 91 | −13.072 | −72.6 | 1.241 | 1 | 44.79 | A | N |
| ATOM | 659 | CA | LYS | A | 91 | −12.834 | −73.921 | 0.62 | 1 | 48.76 | A | C |
| ATOM | 660 | CB | LYS | A | 91 | −14.143 | −74.683 | 0.567 | 1 | 45.67 | A | C |
| ATOM | 661 | CG | LYS | A | 91 | −14.845 | −74.835 | 1.91 | 1 | 48.89 | A | C |
| ATOM | 662 | CD | LYS | A | 91 | −16.159 | −75.583 | 1.732 | 1 | 52.05 | A | C |
| ATOM | 663 | CE | LYS | A | 91 | −16.971 | −75.654 | 3.007 | 1 | 54.44 | A | C |
| ATOM | 664 | NZ | LYS | A | 91 | −18.386 | −76.047 | 2.686 | 1 | 56.7 | A | N |
| ATOM | 665 | C | LYS | A | 91 | −12.346 | −73.755 | −0.808 | 1 | 51.56 | A | C |
| ATOM | 666 | O | LYS | A | 91 | −13.037 | −73.144 | −1.608 | 1 | 60.01 | A | O |
| ATOM | 667 | N | THR | A | 92 | −11.156 | −74.271 | −1.124 | 1 | 61.45 | A | N |
| ATOM | 668 | CA | THR | A | 92 | −10.583 | −74.235 | −2.483 | 1 | 55.7 | A | C |
| ATOM | 669 | CB | THR | A | 92 | −9.159 | −73.682 | −2.471 | 1 | 57.04 | A | C |
| ATOM | 670 | OG1 | THR | A | 92 | −8.351 | −74.533 | −1.657 | 1 | 61.11 | A | O |
| ATOM | 671 | CG2 | THR | A | 92 | −9.112 | −72.265 | −1.92 | 1 | 60.2 | A | C |
| ATOM | 672 | C | THR | A | 92 | −10.5 | −75.639 | −3.082 | 1 | 60.57 | A | C |
| ATOM | 673 | O | THR | A | 92 | −10.833 | −76.612 | −2.424 | 1 | 58.72 | A | O |
| ATOM | 674 | N | ASP | A | 93 | −10.057 | −75.716 | −4.34 | 1 | 73.89 | A | N |
| ATOM | 675 | CA | ASP | A | 93 | −9.779 | −76.992 | −5.048 | 1 | 82.2 | A | C |
| ATOM | 676 | CB | ASP | A | 93 | −9.252 | −76.741 | −6.486 | 1 | 85.53 | A | C |
| ATOM | 677 | CG | ASP | A | 93 | −10.326 | −76.19 | −7.435 | 1 | 96.89 | A | C |
| ATOM | 678 | OD1 | ASP | A | 93 | −11.515 | −76.589 | −7.333 | 1 | 97.88 | A | O |
| ATOM | 679 | OD2 | ASP | A | 93 | −9.975 | −75.354 | −8.295 | 1 | 94.75 | A | O |
| ATOM | 680 | C | ASP | A | 93 | −8.759 | −77.854 | −4.312 | 1 | 77.33 | A | C |
| ATOM | 681 | O | ASP | A | 93 | −8.96 | −79.057 | −4.159 | 1 | 68.5 | A | O |
| ATOM | 682 | N | THR | A | 94 | −7.677 | −77.216 | −3.869 | 1 | 70.86 | A | N |
| ATOM | 683 | CA | THR | A | 94 | −6.571 | −77.881 | −3.175 | 1 | 74.25 | A | C |
| ATOM | 684 | CB | THR | A | 94 | −5.271 | −77.099 | −3.422 | 1 | 82.12 | A | C |
| ATOM | 685 | OG1 | THR | A | 94 | −5.446 | −75.743 | −2.995 | 1 | 82.46 | A | O |
| ATOM | 686 | CG2 | THR | A | 94 | −4.906 | −77.112 | −4.91 | 1 | 84.53 | A | C |
| ATOM | 687 | C | THR | A | 94 | −6.719 | −78.058 | −1.646 | 1 | 69.71 | A | C |
| ATOM | 688 | O | THR | A | 94 | −5.967 | −78.817 | −1.04 | 1 | 72.97 | A | O |
| ATOM | 689 | N | GLY | A | 95 | −7.661 | −77.352 | −1.025 | 1 | 62.53 | A | N |
| ATOM | 690 | CA | GLY | A | 95 | −7.866 | −77.445 | 0.413 | 1 | 61.21 | A | C |
| ATOM | 691 | C | GLY | A | 95 | −8.607 | −76.256 | 1.004 | 1 | 56.35 | A | C |
| ATOM | 692 | O | GLY | A | 95 | −9.831 | −76.193 | 0.949 | 1 | 58.04 | A | O |
| ATOM | 693 | N | VAL | A | 96 | −7.86 | −75.291 | 1.523 | 1 | 51.53 | A | N |
| ATOM | 694 | CA | VAL | A | 96 | −8.442 | −74.184 | 2.251 | 1 | 48.99 | A | C |
| ATOM | 695 | CB | VAL | A | 96 | −8.608 | −74.656 | 3.712 | 1 | 52.06 | A | C |
| ATOM | 696 | CG1 | VAL | A | 96 | −7.85 | −73.814 | 4.717 | 1 | 56.47 | A | C |
| ATOM | 697 | CG2 | VAL | A | 96 | −10.08 | −74.834 | 4.046 | 1 | 50.44 | A | C |
| ATOM | 698 | C | VAL | A | 96 | −7.557 | −72.976 | 2.047 | 1 | 45.61 | A | C |
| ATOM | 699 | O | VAL | A | 96 | −6.402 | −73.137 | 1.752 | 1 | 50.46 | A | O |
| ATOM | 700 | N | SER | A | 97 | −8.103 | −71.783 | 2.23 | 1 | 45.6 | A | N |
| ATOM | 701 | CA | SER | A | 97 | −7.351 | −70.536 | 2.21 | 1 | 41.88 | A | C |
| ATOM | 702 | CB | SER | A | 97 | −7.371 | −69.967 | 0.817 | 1 | 43.35 | A | C |
| ATOM | 703 | OG | SER | A | 97 | −6.792 | −68.678 | 0.749 | 1 | 49.24 | A | O |
| ATOM | 704 | C | SER | A | 97 | −7.972 | −69.501 | 3.158 | 1 | 49.62 | A | C |
| ATOM | 705 | O | SER | A | 97 | −9.213 | −69.299 | 3.151 | 1 | 50.27 | A | O |
| ATOM | 706 | N | LEU | A | 98 | −7.115 | −68.838 | 3.947 | 1 | 44.05 | A | N |
| ATOM | 707 | CA | LEU | A | 98 | −7.541 | −67.82 | 4.91 | 1 | 47.74 | A | C |
| ATOM | 708 | CB | LEU | A | 98 | −6.685 | −67.827 | 6.188 | 1 | 46.42 | A | C |
| ATOM | 709 | CG | LEU | A | 98 | −7.099 | −68.798 | 7.268 | 1 | 44.95 | A | C |
| ATOM | 710 | CD1 | LEU | A | 98 | −7.051 | −70.203 | 6.74 | 1 | 47.84 | A | C |
| ATOM | 711 | CD2 | LEU | A | 98 | −6.177 | −68.658 | 8.453 | 1 | 50.85 | A | C |
| ATOM | 712 | C | LEU | A | 98 | −7.369 | −66.474 | 4.271 | 1 | 44.62 | A | C |
| ATOM | 713 | O | LEU | A | 98 | −6.269 | −66.099 | 3.968 | 1 | 43.48 | A | O |
| ATOM | 714 | N | GLN | A | 99 | −8.445 | −65.751 | 4.063 | 1 | 42.45 | A | N |
| ATOM | 715 | CA | GLN | A | 99 | −8.349 | −64.472 | 3.384 | 1 | 43.16 | A | C |
| ATOM | 716 | CB | GLN | A | 99 | −9.135 | −64.513 | 2.072 | 1 | 48.52 | A | C |
| ATOM | 717 | CG | GLN | A | 99 | −8.516 | −65.359 | 0.956 | 1 | 56.02 | A | C |
| ATOM | 718 | CD | GLN | A | 99 | −7.094 | −64.952 | 0.631 | 1 | 59.86 | A | C |
| ATOM | 719 | OE1 | GLN | A | 99 | −6.253 | −65.809 | 0.371 | 1 | 62.92 | A | O |
| ATOM | 720 | NE2 | GLN | A | 99 | −6.8 | −63.644 | 0.715 | 1 | 64.5 | A | N |
| ATOM | 721 | C | GLN | A | 99 | −8.934 | −63.394 | 4.24 | 1 | 35.27 | A | C |
| ATOM | 722 | O | GLN | A | 99 | −9.959 | −63.578 | 4.87 | 1 | 35.18 | A | O |
| ATOM | 723 | N | THR | A | 100 | −8.272 | −62.264 | 4.23 | 1 | 35.32 | A | N |
| ATOM | 724 | CA | THR | A | 100 | −8.701 | −61.07 | 4.899 | 1 | 37.73 | A | C |
| ATOM | 725 | CB | THR | A | 100 | −7.511 | −60.345 | 5.513 | 1 | 41.27 | A | C |
| ATOM | 726 | OG1 | THR | A | 100 | −6.964 | −61.182 | 6.535 | 1 | 44.05 | A | O |
| ATOM | 727 | CG2 | THR | A | 100 | −7.954 | −59.086 | 6.223 | 1 | 50.63 | A | C |
| ATOM | 728 | C | THR | A | 100 | −9.425 | −60.218 | 3.899 | 1 | 36.68 | A | C |
| ATOM | 729 | O | THR | A | 100 | −8.91 | −60.005 | 2.828 | 1 | 36.68 | A | O |
| ATOM | 730 | N | TYR | A | 101 | −10.636 | −59.783 | 4.241 | 1 | 35.31 | A | N |
| ATOM | 731 | CA | TYR | A | 101 | −11.435 | −58.821 | 3.434 | 1 | 35.19 | A | C |
| ATOM | 732 | CB | TYR | A | 101 | −12.82 | −59.356 | 3.201 | 1 | 34.38 | A | C |
| ATOM | 733 | CG | TYR | A | 101 | −12.796 | −60.542 | 2.302 | 1 | 33.66 | A | C |
| ATOM | 734 | CD1 | TYR | A | 101 | −12.567 | −61.787 | 2.79 | 1 | 33.9 | A | C |
| ATOM | 735 | CE1 | TYR | A | 101 | −12.498 | −62.885 | 1.938 | 1 | 38.86 | A | C |
| ATOM | 736 | CZ | TYR | A | 101 | −12.695 | −62.705 | 0.583 | 1 | 42.24 | A | C |
| ATOM | 737 | OH | TYR | A | 101 | −12.612 | −63.748 | −0.28 | 1 | 46.33 | A | O |
| ATOM | 738 | CE2 | TYR | A | 101 | −12.939 | −61.46 | 0.081 | 1 | 38.59 | A | C |
| ATOM | 739 | CD2 | TYR | A | 101 | −12.987 | −60.389 | 0.942 | 1 | 38.04 | A | C |
| ATOM | 740 | C | TYR | A | 101 | −11.546 | −57.51 | 4.157 | 1 | 34.93 | A | C |
| ATOM | 741 | O | TYR | A | 101 | −11.833 | −57.524 | 5.342 | 1 | 36.99 | A | O |
| ATOM | 742 | N | ASP | A | 102 | −11.28 | −56.394 | 3.469 | 1 | 35.42 | A | N |
| ATOM | 743 | CA | ASP | A | 102 | −11.309 | −55.056 | 4.083 | 1 | 44.08 | A | C |
| ATOM | 744 | CB | ASP | A | 102 | −10.285 | −54.092 | 3.443 | 1 | 47.73 | A | C |
| ATOM | 745 | CG | ASP | A | 102 | −8.833 | −54.368 | 3.851 | 1 | 56.95 | A | C |
| ATOM | 746 | OD1 | ASP | A | 102 | −8.565 | −55.129 | 4.808 | 1 | 59.13 | A | O |
| ATOM | 747 | OD2 | ASP | A | 102 | −7.941 | −53.808 | 3.165 | 1 | 62.44 | A | O |
| ATOM | 748 | C | ASP | A | 102 | −12.665 | −54.387 | 3.967 | 1 | 43.71 | A | C |
| ATOM | 749 | O | ASP | A | 102 | −12.932 | −53.492 | 4.722 | 1 | 57.62 | A | O |
| ATOM | 750 | N | ASP | A | 103 | −13.51 | −54.851 | 3.055 | 1 | 37.58 | A | N |
| ATOM | 751 | CA | ASP | A | 103 | −14.717 | −54.149 | 2.588 | 1 | 40.21 | A | C |
| ATOM | 752 | CB | ASP | A | 103 | −14.653 | −54.118 | 1.038 | 1 | 43.79 | A | C |
| ATOM | 753 | CG | ASP | A | 103 | −14.615 | −55.577 | 0.42 | 1 | 47.95 | A | C |
| ATOM | 754 | OD1 | ASP | A | 103 | −14.001 | −56.506 | 1.048 | 1 | 41.48 | A | O |
| ATOM | 755 | OD2 | ASP | A | 103 | −15.22 | −55.798 | −0.658 | 1 | 52.61 | A | O |
| ATOM | 756 | C | ASP | A | 103 | −15.992 | −54.906 | 2.989 | 1 | 38.24 | A | C |
| ATOM | 757 | O | ASP | A | 103 | −16.92 | −55.039 | 2.188 | 1 | 39.02 | A | O |
| ATOM | 758 | N | LEU | A | 104 | −16.037 | −55.455 | 4.182 | 1 | 34.45 | A | N |
| ATOM | 759 | CA | LEU | A | 104 | −17.081 | −56.434 | 4.498 | 1 | 35.78 | A | C |
| ATOM | 760 | CB | LEU | A | 104 | −16.418 | −57.775 | 4.674 | 1 | 37.65 | A | C |
| ATOM | 761 | CG | LEU | A | 104 | −17.274 | −59.02 | 4.644 | 1 | 37.69 | A | C |
| ATOM | 762 | CD1 | LEU | A | 104 | −16.37 | −60.252 | 4.463 | 1 | 39.36 | A | C |
| ATOM | 763 | CD2 | LEU | A | 104 | −18.128 | −59.136 | 5.874 | 1 | 38.07 | A | C |
| ATOM | 764 | C | LEU | A | 104 | −17.921 | −56.073 | 5.692 | 1 | 35.62 | A | C |
| ATOM | 765 | O | LEU | A | 104 | −19.132 | −56.143 | 5.616 | 1 | 35.02 | A | O |
| ATOM | 766 | N | LEU | A | 105 | −17.29 | −55.696 | 6.802 | 1 | 36.57 | A | N |
| ATOM | 767 | CA | LEU | A | 105 | −17.989 | −55.289 | 7.996 | 1 | 31.36 | A | C |
| ATOM | 768 | CB | LEU | A | 105 | −17.311 | −55.899 | 9.201 | 1 | 32.04 | A | C |
| ATOM | 769 | CG | LEU | A | 105 | −17.294 | −57.43 | 9.315 | 1 | 35.48 | A | C |
| ATOM | 770 | CD1 | LEU | A | 105 | −16.486 | −57.82 | 10.554 | 1 | 34.34 | A | C |
| ATOM | 771 | CD2 | LEU | A | 105 | −18.708 | −57.945 | 9.447 | 1 | 35.44 | A | C |
| ATOM | 772 | C | LEU | A | 105 | −17.857 | −53.804 | 8.121 | 1 | 34.05 | A | C |
| ATOM | 773 | O | LEU | A | 105 | −16.747 | −53.296 | 8.14 | 1 | 32.83 | A | O |
| ATOM | 774 | N | ALA | A | 106 | −18.959 | −53.081 | 8.251 | 1 | 35.04 | A | N |
| ATOM | 775 | CA | ALA | A | 106 | −18.844 | −51.613 | 8.434 | 1 | 34.16 | A | C |
| ATOM | 776 | CB | ALA | A | 106 | −20.169 | −50.935 | 8.195 | 1 | 33.44 | A | C |
| ATOM | 777 | C | ALA | A | 106 | −18.375 | −51.311 | 9.832 | 1 | 33.98 | A | C |
| ATOM | 778 | O | ALA | A | 106 | −18.742 | −51.974 | 10.762 | 1 | 32.86 | A | O |
| ATOM | 779 | N | LYS | A | 107 | −17.593 | −50.266 | 9.962 | 1 | 36.29 | A | N |
| ATOM | 780 | CA | LYS | A | 107 | −17.06 | −49.822 | 11.233 | 1 | 37.95 | A | C |
| ATOM | 781 | CB | LYS | A | 107 | −15.594 | −49.484 | 11.021 | 1 | 39.49 | A | C |
| ATOM | 782 | CG | LYS | A | 107 | −14.85 | −48.81 | 12.127 | 1 | 42.53 | A | C |
| ATOM | 783 | CD | LYS | A | 107 | −13.355 | −48.953 | 11.812 | 1 | 49.03 | A | C |
| ATOM | 784 | CE | LYS | A | 107 | −12.46 | −47.988 | 12.538 | 1 | 50.92 | A | C |
| ATOM | 785 | NZ | LYS | A | 107 | −12.987 | −47.709 | 13.887 | 1 | 54.27 | A | N |
| ATOM | 786 | C | LYS | A | 107 | −17.781 | −48.611 | 11.74 | 1 | 37.57 | A | C |
| ATOM | 787 | O | LYS | A | 107 | −18.04 | −48.527 | 12.888 | 1 | 38.51 | A | O |
| ATOM | 788 | N | ASP | A | 108 | −18.008 | −47.62 | 10.896 | 1 | 41.51 | A | N |
| ATOM | 789 | CA | ASP | A | 108 | −18.862 | −46.475 | 11.252 | 1 | 45.33 | A | C |
| ATOM | 790 | CB | ASP | A | 108 | −18.094 | −45.376 | 12.012 | 1 | 50.5 | A | C |
| ATOM | 791 | CG | ASP | A | 108 | −16.78 | −45.073 | 11.416 | 1 | 58.08 | A | C |
| ATOM | 792 | OD1 | ASP | A | 108 | −16.681 | −45.084 | 10.184 | 1 | 61.77 | A | O |
| ATOM | 793 | OD2 | ASP | A | 108 | −15.818 | −44.825 | 12.185 | 1 | 74.18 | A | O |
| ATOM | 794 | C | ASP | A | 108 | −19.594 | −45.96 | 10.014 | 1 | 40.03 | A | C |
| ATOM | 795 | O | ASP | A | 108 | −19.451 | −46.518 | 8.937 | 1 | 41.17 | A | O |
| ATOM | 796 | N | CYS | A | 109 | −20.432 | −44.951 | 10.199 | 1 | 37.48 | A | N |
| ATOM | 797 | CA | CYS | A | 109 | −21.428 | −44.585 | 9.225 | 1 | 38.68 | A | C |
| ATOM | 798 | CB | CYS | A | 109 | −22.756 | −45.06 | 9.783 | 1 | 38.13 | A | C |
| ATOM | 799 | SG | CYS | A | 109 | −22.768 | −46.857 | 10.032 | 1 | 43.3 | A | S |
| ATOM | 800 | C | CYS | A | 109 | −21.442 | −43.074 | 8.967 | 1 | 38.48 | A | C |
| ATOM | 801 | O | CYS | A | 109 | −21.252 | −42.28 | 9.876 | 1 | 39.92 | A | O |
| ATOM | 802 | N | HIS | A | 110 | −21.698 | −42.666 | 7.728 | 1 | 37.77 | A | N |
| ATOM | 803 | CA | HIS | A | 110 | −22.021 | −41.261 | 7.481 | 1 | 35.38 | A | C |
| ATOM | 804 | CB | HIS | A | 110 | −20.747 | −40.415 | 7.336 | 1 | 38.02 | A | C |
| ATOM | 805 | CG | HIS | A | 110 | −19.832 | −40.852 | 6.243 | 1 | 39.63 | A | C |
| ATOM | 806 | ND1 | HIS | A | 110 | −20.213 | −41.687 | 5.21 | 1 | 47.3 | A | N |
| ATOM | 807 | CE1 | HIS | A | 110 | −19.214 | −41.819 | 4.356 | 1 | 41.43 | A | C |
| ATOM | 808 | NE2 | HIS | A | 110 | −18.198 | −41.106 | 4.804 | 1 | 37.91 | A | N |
| ATOM | 809 | CD2 | HIS | A | 110 | −18.559 | −40.49 | 5.976 | 1 | 39.91 | A | C |
| ATOM | 810 | C | HIS | A | 110 | −22.995 | −41.104 | 6.34 | 1 | 37.11 | A | C |
| ATOM | 811 | O | HIS | A | 110 | −23.454 | −42.125 | 5.766 | 1 | 32.86 | A | O |
| ATOM | 812 | N | CYS | A | 111 | −23.386 | −39.85 | 6.088 | 1 | 35.59 | A | N |
| ATOM | 813 | CA | CYS | A | 111 | −24.429 | −39.543 | 5.119 | 1 | 37.83 | A | C |
| ATOM | 814 | CB | CYS | A | 111 | −25.502 | −38.652 | 5.763 | 1 | 39.42 | A | C |
| ATOM | 815 | SG | CYS | A | 111 | −26.387 | −39.509 | 7.076 | 1 | 40.31 | A | S |
| ATOM | 816 | C | CYS | A | 111 | −23.868 | −38.905 | 3.862 | 1 | 39.56 | A | C |
| ATOM | 817 | O | CYS | A | 111 | −23.048 | −37.948 | 3.929 | 1 | 43.88 | A | O |
| ATOM | 818 | N | ILE | A | 112 | −24.306 | −39.408 | 2.706 | 1 | 39.08 | A | N |
| ATOM | 819 | CA | ILE | A | 112 | −23.852 | −38.835 | 1.419 | 1 | 42.88 | A | C |
| ATOM | 820 | CB | ILE | A | 112 | −22.51 | −39.437 | 0.965 | 1 | 43.81 | A | C |
| ATOM | 821 | CG1 | ILE | A | 112 | −22.105 | −38.811 | −0.375 | 1 | 43.24 | A | C |
| ATOM | 822 | CD1 | ILE | A | 112 | −20.626 | −38.782 | −0.534 | 1 | 42.73 | A | C |
| ATOM | 823 | CG2 | ILE | A | 112 | −22.545 | −40.946 | 0.826 | 1 | 41.93 | A | C |
| ATOM | 824 | C | ILE | A | 112 | −24.891 | −38.817 | 0.28 | 1 | 47.04 | A | C |
| ATOM | 825 | O | ILE | A | 112 | −25.482 | −39.859 | −0.044 | 1 | 58.35 | A | O |
| TER | 826 | ILE | A | 112 | | | | | | | | |
| ATOM | 827 | N | TRP | B | 129 | −4.147 | −80.481 | 18.979 | 1 | 84.64 | B | N |
| ATOM | 828 | CA | TRP | B | 129 | −4.734 | −79.241 | 18.366 | 1 | 77.53 | B | C |
| ATOM | 829 | CB | TRP | B | 129 | −4.199 | −77.995 | 19.083 | 1 | 89 | B | C |
| ATOM | 830 | CG | TRP | B | 129 | −4.63 | −77.832 | 20.53 | 1 | 102.56 | B | C |
| ATOM | 831 | CD1 | TRP | B | 129 | −5.858 | −78.141 | 21.078 | 1 | 100.87 | B | C |
| ATOM | 832 | NE1 | TRP | B | 129 | −5.872 | −77.821 | 22.418 | 1 | 102.38 | B | N |
| ATOM | 833 | CE2 | TRP | B | 129 | −4.651 | −77.293 | 22.767 | 1 | 111.5 | B | C |
| ATOM | 834 | CD2 | TRP | B | 129 | −3.844 | −77.272 | 21.598 | 1 | 107.85 | B | C |
| ATOM | 835 | CE3 | TRP | B | 129 | −2.532 | −76.763 | 21.685 | 1 | 103.13 | B | C |
| ATOM | 836 | CZ3 | TRP | B | 129 | −2.07 | −76.285 | 22.93 | 1 | 99.55 | B | C |
| ATOM | 837 | CH2 | TRP | B | 129 | −2.898 | −76.314 | 24.075 | 1 | 100.69 | B | C |
| ATOM | 838 | CZ2 | TRP | B | 129 | −4.185 | −76.811 | 24.017 | 1 | 107.52 | B | C |
| ATOM | 839 | C | TRP | B | 129 | −4.397 | −79.133 | 16.875 | 1 | 65.87 | B | C |
| ATOM | 840 | O | TRP | B | 129 | −3.269 | −78.774 | 16.529 | 1 | 64.3 | B | O |
| ATOM | 841 | N | SER | B | 130 | −5.344 | −79.465 | 15.992 | 1 | 63.64 | B | N |
| ATOM | 842 | CA | SER | B | 130 | −5.178 | −79.177 | 14.547 | 1 | 58.11 | B | C |
| ATOM | 843 | CB | SER | B | 130 | −6.328 | −79.697 | 13.703 | 1 | 58.52 | B | C |
| ATOM | 844 | OG | SER | B | 130 | −6.075 | −79.414 | 12.331 | 1 | 60.85 | B | O |
| ATOM | 845 | C | SER | B | 130 | −5.154 | −77.682 | 14.352 | 1 | 55.08 | B | C |
| ATOM | 846 | O | SER | B | 130 | −6.012 | −76.99 | 14.899 | 1 | 52.08 | B | O |
| ATOM | 847 | N | CYS | B | 131 | −4.157 | −77.201 | 13.604 | 1 | 57.32 | B | N |
| ATOM | 848 | CA | CYS | B | 131 | −4.119 | −75.827 | 13.142 | 1 | 55.51 | B | C |
| ATOM | 849 | CB | CYS | B | 131 | −2.87 | −75.569 | 12.302 | 1 | 55.66 | B | C |
| ATOM | 850 | SG | CYS | B | 131 | −1.325 | −75.591 | 13.285 | 1 | 55.06 | B | S |
| ATOM | 851 | C | CYS | B | 131 | −5.423 | −75.371 | 12.419 | 1 | 54 | B | C |
| ATOM | 852 | O | CYS | B | 131 | −5.867 | −74.248 | 12.639 | 1 | 60.47 | B | O |
| ATOM | 853 | N | LEU | B | 132 | −6.059 | −76.256 | 11.656 | 1 | 49.93 | B | N |
| ATOM | 854 | CA | LEU | B | 132 | −7.361 | −75.99 | 11.002 | 1 | 51.9 | B | C |
| ATOM | 855 | CB | LEU | B | 132 | −7.81 | −77.213 | 10.166 | 1 | 54.66 | B | C |
| ATOM | 856 | CG | LEU | B | 132 | −9.125 | −77.075 | 9.371 | 1 | 54.16 | B | C |
| ATOM | 857 | CD1 | LEU | B | 132 | −9.138 | −75.769 | 8.598 | 1 | 51.69 | B | C |
| ATOM | 858 | CD2 | LEU | B | 132 | −9.333 | −78.236 | 8.411 | 1 | 55.71 | B | C |
| ATOM | 859 | C | LEU | B | 132 | −8.456 | −75.675 | 12.001 | 1 | 48.94 | B | C |
| ATOM | 860 | O | LEU | B | 132 | −9.108 | −74.66 | 11.905 | 1 | 48.74 | B | O |
| ATOM | 861 | N | GLU | B | 133 | −8.629 | −76.56 | 12.969 | 1 | 48.27 | B | N |
| ATOM | 862 | CA | GLU | B | 133 | −9.638 | −76.419 | 14.011 | 1 | 47.85 | B | C |
| ATOM | 863 | CB | GLU | B | 133 | −9.66 | −77.647 | 14.911 | 1 | 51.5 | B | C |
| ATOM | 864 | CG | GLU | B | 133 | −10.194 | −78.864 | 14.177 | 1 | 61.81 | B | C |
| ATOM | 865 | CD | GLU | B | 133 | −10.22 | −80.105 | 15.042 | 1 | 71.01 | B | C |
| ATOM | 866 | OE1 | GLU | B | 133 | −9.224 | −80.331 | 15.763 | 1 | 80.29 | B | O |
| ATOM | 867 | OE2 | GLU | B | 133 | −11.226 | −80.851 | 14.991 | 1 | 72.66 | B | O |
| ATOM | 868 | C | GLU | B | 133 | −9.456 | −75.192 | 14.864 | 1 | 49.99 | B | C |
| ATOM | 869 | O | GLU | B | 133 | −10.436 | −74.56 | 15.216 | 1 | 51.03 | B | O |
| ATOM | 870 | N | VAL | B | 134 | −8.21 | −74.833 | 15.164 | 1 | 48.56 | B | N |
| ATOM | 871 | CA | VAL | B | 134 | −7.93 | −73.601 | 15.904 | 1 | 47.66 | B | C |
| ATOM | 872 | CB | VAL | B | 134 | −6.451 | −73.543 | 16.333 | 1 | 54.4 | B | C |
| ATOM | 873 | CG1 | VAL | B | 134 | −6.113 | −72.197 | 16.959 | 1 | 56.48 | B | C |
| ATOM | 874 | CG2 | VAL | B | 134 | −6.134 | −74.67 | 17.308 | 1 | 59.73 | B | C |
| ATOM | 875 | C | VAL | B | 134 | −8.239 | −72.408 | 15.01 | 1 | 42.81 | B | C |
| ATOM | 876 | O | VAL | B | 134 | −8.775 | −71.4 | 15.464 | 1 | 40.31 | B | O |
| ATOM | 877 | N | ALA | B | 135 | −7.858 | −72.523 | 13.737 | 1 | 42.75 | B | N |
| ATOM | 878 | CA | ALA | B | 135 | −8.168 | −71.5 | 12.765 | 1 | 42.24 | B | C |
| ATOM | 879 | CB | ALA | B | 135 | −7.554 | −71.8 | 11.422 | 1 | 40.45 | B | C |
| ATOM | 880 | C | ALA | B | 135 | −9.703 | −71.284 | 12.695 | 1 | 41.09 | B | C |
| ATOM | 881 | O | ALA | B | 135 | −10.156 | −70.13 | 12.843 | 1 | 40.83 | B | O |
| ATOM | 882 | N | GLU | B | 136 | −10.478 | −72.37 | 12.648 | 1 | 42.57 | B | N |
| ATOM | 883 | CA | GLU | B | 136 | −11.985 | −72.298 | 12.641 | 1 | 42.83 | B | C |
| ATOM | 884 | CB | GLU | B | 136 | −12.624 | −73.662 | 12.479 | 1 | 40.96 | B | C |
| ATOM | 885 | CG | GLU | B | 136 | −12.383 | −74.292 | 11.106 | 1 | 52.45 | B | C |
| ATOM | 886 | CD | GLU | B | 136 | −13.049 | −75.661 | 10.956 | 1 | 60.06 | B | C |
| ATOM | 887 | OE1 | GLU | B | 136 | −12.448 | −76.703 | 11.322 | 1 | 66.51 | B | O |
| ATOM | 888 | OE2 | GLU | B | 136 | −14.199 | −75.691 | 10.478 | 1 | 64.3 | B | O |
| ATOM | 889 | C | GLU | B | 136 | −12.561 | −71.659 | 13.884 | 1 | 45.4 | B | C |
| ATOM | 890 | O | GLU | B | 136 | −13.49 | −70.865 | 13.781 | 1 | 52.26 | B | O |
| ATOM | 891 | N | ALA | B | 137 | −11.995 | −71.99 | 15.048 | 1 | 43.05 | B | N |
| ATOM | 892 | CA | ALA | B | 137 | −12.444 | −71.417 | 16.32 | 1 | 44.49 | B | C |
| ATOM | 893 | CB | ALA | B | 137 | −11.861 | −72.175 | 17.518 | 1 | 44.93 | B | C |
| ATOM | 894 | C | ALA | B | 137 | −12.087 | −69.942 | 16.413 | 1 | 40.96 | B | C |
| ATOM | 895 | O | ALA | B | 137 | −12.895 | −69.19 | 16.88 | 1 | 39.85 | B | O |
| ATOM | 896 | N | CYS | B | 138 | −10.89 | −69.548 | 15.965 | 1 | 34.81 | B | N |
| ATOM | 897 | CA | CYS | B | 138 | −10.475 | −68.161 | 16.037 | 1 | 38.43 | B | C |
| ATOM | 898 | CB | CYS | B | 138 | −8.98 | −68.003 | 15.735 | 1 | 38.72 | B | C |
| ATOM | 899 | SG | CYS | B | 138 | −8.441 | −66.271 | 15.755 | 1 | 50.88 | B | S |
| ATOM | 900 | C | CYS | B | 138 | −11.305 | −67.331 | 15.067 | 1 | 39.96 | B | C |
| ATOM | 901 | O | CYS | B | 138 | −11.791 | −66.223 | 15.393 | 1 | 38.84 | B | O |
| ATOM | 902 | N | VAL | B | 139 | −11.473 | −67.868 | 13.863 | 1 | 43.03 | B | N |
| ATOM | 903 | CA | VAL | B | 139 | −12.191 | −67.133 | 12.803 | 1 | 41.3 | B | C |
| ATOM | 904 | CB | VAL | B | 139 | −12.015 | −67.824 | 11.433 | 1 | 43.29 | B | C |
| ATOM | 905 | CG1 | VAL | B | 139 | −12.952 | −67.208 | 10.375 | 1 | 44.13 | B | C |
| ATOM | 906 | CG2 | VAL | B | 139 | −10.583 | −67.688 | 10.973 | 1 | 40.84 | B | C |
| ATOM | 907 | C | VAL | B | 139 | −13.661 | −66.99 | 13.15 | 1 | 37.13 | B | C |
| ATOM | 908 | O | VAL | B | 139 | −14.273 | −65.958 | 12.867 | 1 | 34.89 | B | O |
| ATOM | 909 | N | GLY | B | 140 | −14.202 | −68.043 | 13.781 | 1 | 37.64 | B | N |
| ATOM | 910 | CA | GLY | B | 140 | −15.569 | −68.095 | 14.289 | 1 | 37.29 | B | C |
| ATOM | 911 | C | GLY | B | 140 | −15.94 | −67.152 | 15.417 | 1 | 41.77 | B | C |
| ATOM | 912 | O | GLY | B | 140 | −17.11 | −66.964 | 15.646 | 1 | 44.72 | B | O |
| ATOM | 913 | N | ASP | B | 141 | −14.952 | −66.554 | 16.08 | 1 | 39.39 | B | N |
| ATOM | 914 | CA | ASP | B | 141 | −15.128 | −65.668 | 17.185 | 1 | 37.52 | B | C |
| ATOM | 915 | CB | ASP | B | 141 | −14.055 | −66.037 | 18.226 | 1 | 39.33 | B | C |
| ATOM | 916 | CG | ASP | B | 141 | −14.16 | −65.181 | 19.494 | 1 | 47.58 | B | C |
| ATOM | 917 | OD1 | ASP | B | 141 | −15.21 | −65.284 | 20.177 | 1 | 51.89 | B | O |
| ATOM | 918 | OD2 | ASP | B | 141 | −13.232 | −64.391 | 19.787 | 1 | 49.26 | B | O |
| ATOM | 919 | C | ASP | B | 141 | −14.968 | −64.182 | 16.781 | 1 | 38.08 | B | C |
| ATOM | 920 | O | ASP | B | 141 | −14.081 | −63.828 | 16.014 | 1 | 39.23 | B | O |
| ATOM | 921 | N | VAL | B | 142 | −15.781 | −63.295 | 17.323 | 1 | 38.68 | B | N |
| ATOM | 922 | CA | VAL | B | 142 | −15.784 | −61.887 | 16.878 | 1 | 37.8 | B | C |
| ATOM | 923 | CB | VAL | B | 142 | −16.869 | −61.061 | 17.565 | 1 | 40.3 | B | C |
| ATOM | 924 | CG1 | VAL | B | 142 | −16.685 | −59.562 | 17.344 | 1 | 38.98 | B | C |
| ATOM | 925 | CG2 | VAL | B | 142 | −18.219 | −61.451 | 16.997 | 1 | 45.37 | B | C |
| ATOM | 926 | C | VAL | B | 142 | −14.439 | −61.221 | 17.098 | 1 | 37.65 | B | C |
| ATOM | 927 | O | VAL | B | 142 | −13.864 | −60.696 | 16.163 | 1 | 34.4 | B | O |
| ATOM | 928 | N | VAL | B | 143 | −13.932 | −61.289 | 18.314 | 1 | 35.35 | B | N |
| ATOM | 929 | CA | VAL | B | 143 | −12.681 | −60.624 | 18.682 | 1 | 39.43 | B | C |
| ATOM | 930 | CB | VAL | B | 143 | −12.564 | −60.47 | 20.239 | 1 | 42.03 | B | C |
| ATOM | 931 | CG1 | VAL | B | 143 | −11.13 | −60.189 | 20.709 | 1 | 37.07 | B | C |
| ATOM | 932 | CG2 | VAL | B | 143 | −13.479 | −59.341 | 20.706 | 1 | 42.47 | B | C |
| ATOM | 933 | C | VAL | B | 143 | −11.429 | −61.299 | 18.098 | 1 | 38.55 | B | C |
| ATOM | 934 | O | VAL | B | 143 | −10.522 | −60.608 | 17.673 | 1 | 39.69 | B | O |
| ATOM | 935 | N | CYS | B | 144 | −11.377 | −62.629 | 18.106 | 1 | 33.67 | B | N |
| ATOM | 936 | CA | CYS | B | 144 | −10.221 | −63.31 | 17.624 | 1 | 39.77 | B | C |
| ATOM | 937 | CB | CYS | B | 144 | −10.261 | −64.822 | 17.963 | 1 | 42.41 | B | C |
| ATOM | 938 | SG | CYS | B | 144 | −8.653 | −65.673 | 17.776 | 1 | 50.74 | B | S |
| ATOM | 939 | C | CYS | B | 144 | −10.07 | −63.076 | 16.118 | 1 | 37.13 | B | C |
| ATOM | 940 | O | CYS | B | 144 | −8.938 | −62.823 | 15.662 | 1 | 35.09 | B | O |
| ATOM | 941 | N | ASN | B | 145 | −11.204 | −63.16 | 15.393 | 1 | 34.92 | B | N |
| ATOM | 942 | CA | ASN | B | 145 | −11.285 | −62.835 | 13.958 | 1 | 32.26 | B | C |
| ATOM | 943 | CB | ASN | B | 145 | −12.708 | −62.881 | 13.429 | 1 | 35.06 | B | C |
| ATOM | 944 | CG | ASN | B | 145 | −12.764 | −62.816 | 11.914 | 1 | 35.15 | B | C |
| ATOM | 945 | OD1 | ASN | B | 145 | −12.267 | −61.857 | 11.308 | 1 | 34.04 | B | O |
| ATOM | 946 | ND2 | ASN | B | 145 | −13.297 | −63.86 | 11.288 | 1 | 31.36 | B | N |
| ATOM | 947 | C | ASN | B | 145 | −10.685 | −61.475 | 13.68 | 1 | 35.76 | B | C |
| ATOM | 948 | O | ASN | B | 145 | −9.768 | −61.347 | 12.851 | 1 | 32.04 | B | O |
| ATOM | 949 | N | ALA | B | 146 | −11.16 | −60.466 | 14.405 | 1 | 36.14 | B | N |
| ATOM | 950 | CA | ALA | B | 146 | −10.621 | −59.095 | 14.249 | 1 | 36.78 | B | C |
| ATOM | 951 | CB | ALA | B | 146 | −11.378 | −58.096 | 15.114 | 1 | 34.28 | B | C |
| ATOM | 952 | C | ALA | B | 146 | −9.127 | −59.014 | 14.524 | 1 | 38.43 | B | C |
| ATOM | 953 | O | ALA | B | 146 | −8.387 | −58.379 | 13.74 | 1 | 38.21 | B | O |
| ATOM | 954 | N | GLN | B | 147 | −8.659 | −59.667 | 15.596 | 1 | 34.9 | B | N |
| ATOM | 955 | CA | GLN | B | 147 | −7.246 | −59.573 | 15.903 | 1 | 37.1 | B | C |
| ATOM | 956 | CB | GLN | B | 147 | −6.93 | −60.007 | 17.326 | 1 | 40.1 | B | C |
| ATOM | 957 | CG | GLN | B | 147 | −7.554 | −59.137 | 18.405 | 1 | 42.04 | B | C |
| ATOM | 958 | CD | GLN | B | 147 | −7.049 | −57.705 | 18.445 | 1 | 45.64 | B | C |
| ATOM | 959 | OE1 | GLN | B | 147 | −6.134 | −57.31 | 17.745 | 1 | 46.97 | B | O |
| ATOM | 960 | NE2 | GLN | B | 147 | −7.674 | −56.916 | 19.281 | 1 | 51.25 | B | N |
| ATOM | 961 | C | GLN | B | 147 | −6.409 | −60.383 | 14.928 | 1 | 37.32 | B | C |
| ATOM | 962 | O | GLN | B | 147 | −5.284 | −59.991 | 14.573 | 1 | 36.6 | B | O |
| ATOM | 963 | N | LEU | B | 148 | −6.938 | −61.517 | 14.506 | 1 | 36.07 | B | N |
| ATOM | 964 | CA | LEU | B | 148 | −6.256 | −62.287 | 13.514 | 1 | 38.39 | B | C |
| ATOM | 965 | CB | LEU | B | 148 | −6.992 | −63.607 | 13.259 | 1 | 36.05 | B | C |
| ATOM | 966 | CG | LEU | B | 148 | −6.35 | −64.506 | 12.213 | 1 | 40.43 | B | C |
| ATOM | 967 | CD1 | LEU | B | 148 | −5.011 | −65.041 | 12.703 | 1 | 45.08 | B | C |
| ATOM | 968 | CD2 | LEU | B | 148 | −7.25 | −65.689 | 11.951 | 1 | 41.59 | B | C |
| ATOM | 969 | C | LEU | B | 148 | −6.13 | −61.433 | 12.218 | 1 | 40.4 | B | C |
| ATOM | 970 | O | LEU | B | 148 | −5.074 | −61.397 | 11.592 | 1 | 39.66 | B | O |
| ATOM | 971 | N | ALA | B | 149 | −7.186 | −60.727 | 11.827 | 1 | 35.23 | B | N |
| ATOM | 972 | CA | ALA | B | 149 | −7.104 | −59.95 | 10.581 | 1 | 36.99 | B | C |
| ATOM | 973 | CB | ALA | B | 149 | −8.418 | −59.276 | 10.238 | 1 | 34.78 | B | C |
| ATOM | 974 | C | ALA | B | 149 | −5.985 | −58.927 | 10.607 | 1 | 39.14 | B | C |
| ATOM | 975 | O | ALA | B | 149 | −5.286 | −58.786 | 9.607 | 1 | 39.83 | B | O |
| ATOM | 976 | N | SER | B | 150 | −5.764 | −58.254 | 11.729 | 1 | 39.56 | B | N |
| ATOM | 977 | CA | SER | B | 150 | −4.718 | −57.23 | 11.767 | 1 | 43.78 | B | C |
| ATOM | 978 | CB | SER | B | 150 | −4.693 | −56.499 | 13.079 | 1 | 46.9 | B | C |
| ATOM | 979 | OG | SER | B | 150 | −5.887 | −55.815 | 13.239 | 1 | 46.21 | B | O |
| ATOM | 980 | C | SER | B | 150 | −3.356 | −57.83 | 11.57 | 1 | 48.49 | B | C |
| ATOM | 981 | O | SER | B | 150 | −2.488 | −57.249 | 10.906 | 1 | 47.09 | B | O |
| ATOM | 982 | N | TYR | B | 151 | −3.192 | −59.011 | 12.133 | 1 | 46.28 | B | N |
| ATOM | 983 | CA | TYR | B | 151 | −1.93 | −59.708 | 12.073 | 1 | 46.04 | B | C |
| ATOM | 984 | CB | TYR | B | 151 | −1.856 | −60.733 | 13.26 | 1 | 49.6 | B | C |
| ATOM | 985 | CG | TYR | B | 151 | −1.175 | −62.018 | 12.973 | 1 | 53.58 | B | C |
| ATOM | 986 | CD1 | TYR | B | 151 | 0.071 | −62.021 | 12.392 | 1 | 54.29 | B | C |
| ATOM | 987 | CE1 | TYR | B | 151 | 0.721 | −63.186 | 12.1 | 1 | 61.09 | B | C |
| ATOM | 988 | CZ | TYR | B | 151 | 0.147 | −64.385 | 12.393 | 1 | 67.39 | B | C |
| ATOM | 989 | OH | TYR | B | 151 | 0.89 | −65.488 | 12.062 | 1 | 80.4 | B | O |
| ATOM | 990 | CE2 | TYR | B | 151 | −1.104 | −64.438 | 13.002 | 1 | 67.14 | B | C |
| ATOM | 991 | CD2 | TYR | B | 151 | −1.755 | −63.24 | 13.298 | 1 | 64.93 | B | C |
| ATOM | 992 | C | TYR | B | 151 | −1.706 | −60.285 | 10.647 | 1 | 42.07 | B | C |
| ATOM | 993 | O | TYR | B | 151 | −0.64 | −60.114 | 10.059 | 1 | 39.45 | B | O |
| ATOM | 994 | N | LEU | B | 152 | −2.676 | −60.995 | 10.1 | 1 | 40.01 | B | N |
| ATOM | 995 | CA | LEU | B | 152 | −2.539 | −61.511 | 8.729 | 1 | 38.13 | B | C |
| ATOM | 996 | CB | LEU | B | 152 | −3.781 | −62.263 | 8.298 | 1 | 38.49 | B | C |
| ATOM | 997 | CG | LEU | B | 152 | −4.1 | −63.552 | 9.033 | 1 | 40.64 | B | C |
| ATOM | 998 | CD1 | LEU | B | 152 | −5.257 | −64.28 | 8.379 | 1 | 38.96 | B | C |
| ATOM | 999 | CD2 | LEU | B | 152 | −2.868 | −64.438 | 9.041 | 1 | 42.98 | B | C |
| ATOM | 1000 | C | LEU | B | 152 | −2.23 | −60.426 | 7.703 | 1 | 39.75 | B | C |
| ATOM | 1001 | O | LEU | B | 152 | −1.408 | −60.62 | 6.794 | 1 | 40.43 | B | O |
| ATOM | 1002 | N | LYS | B | 153 | −2.843 | −59.274 | 7.876 | 1 | 41.28 | B | N |
| ATOM | 1003 | CA | LYS | B | 153 | −2.566 | −58.147 | 7.002 | 1 | 48.39 | B | C |
| ATOM | 1004 | CB | LYS | B | 153 | −3.53 | −56.971 | 7.209 | 1 | 47.96 | B | C |
| ATOM | 1005 | CG | LYS | B | 153 | −4.831 | −57.21 | 6.477 | 1 | 56.5 | B | C |
| ATOM | 1006 | CD | LYS | B | 153 | −5.938 | −56.24 | 6.878 | 1 | 62.78 | B | C |
| ATOM | 1007 | CE | LYS | B | 153 | −5.721 | −54.869 | 6.253 | 1 | 63.98 | B | C |
| ATOM | 1008 | NZ | LYS | B | 153 | −6.228 | −53.841 | 7.196 | 1 | 71.29 | B | N |
| ATOM | 1009 | C | LYS | B | 153 | −1.145 | −57.686 | 7.168 | 1 | 46.97 | B | C |
| ATOM | 1010 | O | LYS | B | 153 | −0.434 | −57.612 | 6.206 | 1 | 50.41 | B | O |
| ATOM | 1011 | N | ALA | B | 154 | −0.724 | −57.393 | 8.384 | 1 | 46.25 | B | N |
| ATOM | 1012 | CA | ALA | B | 154 | 0.582 | −56.813 | 8.582 | 1 | 49.44 | B | C |
| ATOM | 1013 | CB | ALA | B | 154 | 0.766 | −56.419 | 10.041 | 1 | 54.7 | B | C |
| ATOM | 1014 | C | ALA | B | 154 | 1.709 | −57.732 | 8.126 | 1 | 47.24 | B | C |
| ATOM | 1015 | O | ALA | B | 154 | 2.796 | −57.276 | 7.876 | 1 | 57.26 | B | O |
| ATOM | 1016 | N | CYS | B | 155 | 1.443 | −59.019 | 8.031 | 1 | 46.82 | B | N |
| ATOM | 1017 | CA | CYS | B | 155 | 2.431 | −60.008 | 7.692 | 1 | 47.64 | B | C |
| ATOM | 1018 | CB | CYS | B | 155 | 2.364 | −61.138 | 8.74 | 1 | 45.94 | B | C |
| ATOM | 1019 | SG | CYS | B | 155 | 2.85 | −60.645 | 10.43 | 1 | 45.42 | B | S |
| ATOM | 1020 | C | CYS | B | 155 | 2.244 | −60.616 | 6.291 | 1 | 53.03 | B | C |
| ATOM | 1021 | O | CYS | B | 155 | 2.713 | −61.719 | 6.043 | 1 | 56.8 | B | O |
| ATOM | 1022 | N | SER | B | 156 | 1.552 | −59.922 | 5.388 | 1 | 57.31 | B | N |
| ATOM | 1023 | CA | SER | B | 156 | 1.301 | −60.43 | 4.04 | 1 | 63.29 | B | C |
| ATOM | 1024 | CB | SER | B | 156 | −0.152 | −60.156 | 3.637 | 1 | 67.38 | B | C |
| ATOM | 1025 | OG | SER | B | 156 | −0.468 | −58.756 | 3.747 | 1 | 64.8 | B | O |
| ATOM | 1026 | C | SER | B | 156 | 2.235 | −59.713 | 3.068 | 1 | 68.43 | B | C |
| ATOM | 1027 | O | SER | B | 156 | 2.737 | −58.624 | 3.372 | 1 | 62.04 | B | O |
| ATOM | 1028 | N | ALA | B | 157 | 2.414 | −60.306 | 1.887 | 1 | 86.18 | B | N |
| ATOM | 1029 | CA | ALA | B | 157 | 3.248 | −59.737 | 0.794 | 1 | 97.02 | B | C |
| ATOM | 1030 | CB | ALA | B | 157 | 3.403 | −60.769 | −0.34 | 1 | 98.84 | B | C |
| ATOM | 1031 | C | ALA | B | 157 | 2.78 | −58.339 | 0.246 | 1 | 98.22 | B | C |
| ATOM | 1032 | O | ALA | B | 157 | 1.895 | −57.713 | 0.828 | 1 | 89.22 | B | O |
| ATOM | 1033 | N | ASN | B | 158 | 3.355 | −57.881 | −0.879 | 1 | 112.93 | B | N |
| ATOM | 1034 | CA | ASN | B | 158 | 3.349 | −56.449 | −1.304 | 1 | 120.5 | B | C |
| ATOM | 1035 | CB | ASN | B | 158 | 1.909 | −55.873 | −1.456 | 1 | 127.93 | B | C |
| ATOM | 1036 | CG | ASN | B | 158 | 1.648 | −54.641 | −0.595 | 1 | 137.85 | B | C |
| ATOM | 1037 | OD1 | ASN | B | 158 | 1.613 | −53.517 | −1.1 | 1 | 141.42 | B | O |
| ATOM | 1038 | ND2 | ASN | B | 158 | 1.469 | −54.849 | 0.71 | 1 | 144.72 | B | N |
| ATOM | 1039 | C | ASN | B | 158 | 4.334 | −55.557 | −0.473 | 1 | 125.83 | B | C |
| ATOM | 1040 | O | ASN | B | 158 | 4.32 | −54.326 | −0.576 | 1 | 130.25 | B | O |
| ATOM | 1041 | N | GLY | B | 159 | 5.226 | −56.199 | 0.288 | 1 | 123.24 | B | N |
| ATOM | 1042 | CA | GLY | B | 159 | 6.186 | −55.526 | 1.17 | 1 | 114.52 | B | C |
| ATOM | 1043 | C | GLY | B | 159 | 7.37 | −54.789 | 0.558 | 1 | 116.39 | B | C |
| ATOM | 1044 | O | GLY | B | 159 | 7.513 | −53.6 | 0.827 | 1 | 135.54 | B | O |
| ATOM | 1045 | N | ASN | B | 160 | 8.259 | −55.435 | −0.213 | 1 | 111.89 | B | N |
| ATOM | 1046 | CA | ASN | B | 160 | 8.264 | −56.869 | −0.528 | 1 | 101.95 | B | C |
| ATOM | 1047 | CB | ASN | B | 160 | 7.967 | −57.091 | −2.019 | 1 | 103.89 | B | C |
| ATOM | 1048 | CG | ASN | B | 160 | 6.588 | −57.712 | −2.278 | 1 | 109.29 | B | C |
| ATOM | 1049 | OD1 | ASN | B | 160 | 6.226 | −58.733 | −1.695 | 1 | 98.47 | B | O |
| ATOM | 1050 | ND2 | ASN | B | 160 | 5.829 | −57.114 | −3.191 | 1 | 114.85 | B | N |
| ATOM | 1051 | C | ASN | B | 160 | 9.592 | −57.562 | −0.105 | 1 | 99.55 | B | C |
| ATOM | 1052 | O | ASN | B | 160 | 10.662 | −56.955 | −0.265 | 1 | 79 | B | O |
| ATOM | 1053 | N | PRO | B | 161 | 9.542 | −58.818 | 0.412 | 1 | 104.41 | B | N |
| ATOM | 1054 | CA | PRO | B | 161 | 8.305 | −59.642 | 0.495 | 1 | 85.67 | B | C |
| ATOM | 1055 | CB | PRO | B | 161 | 8.786 | −60.981 | 1.084 | 1 | 86.49 | B | C |
| ATOM | 1056 | CG | PRO | B | 161 | 10.089 | −60.684 | 1.767 | 1 | 91.58 | B | C |
| ATOM | 1057 | CD | PRO | B | 161 | 10.573 | −59.298 | 1.363 | 1 | 96.4 | B | C |
| ATOM | 1058 | C | PRO | B | 161 | 7.234 | −58.985 | 1.365 | 1 | 76.44 | B | C |
| ATOM | 1059 | O | PRO | B | 161 | 6.175 | −58.663 | 0.86 | 1 | 84.26 | B | O |
| ATOM | 1060 | N | CYS | B | 162 | 7.534 | −58.728 | 2.632 | 1 | 65.64 | B | N |
| ATOM | 1061 | CA | CYS | B | 162 | 6.619 | −58.032 | 3.561 | 1 | 68.76 | B | C |
| ATOM | 1062 | CB | CYS | B | 162 | 6.107 | −59.01 | 4.615 | 1 | 71.14 | B | C |
| ATOM | 1063 | SG | CYS | B | 162 | 7.474 | −59.86 | 5.454 | 1 | 75.18 | B | S |
| ATOM | 1064 | C | CYS | B | 162 | 7.446 | −56.972 | 4.248 | 1 | 56.83 | B | C |
| ATOM | 1065 | O | CYS | B | 162 | 8.643 | −56.925 | 4.024 | 1 | 65.14 | B | O |
| ATOM | 1066 | N | ASP | B | 163 | 6.816 | −56.154 | 5.081 | 1 | 52.73 | B | N |
| ATOM | 1067 | CA | ASP | B | 163 | 7.507 | −55.25 | 5.991 | 1 | 52.92 | B | C |
| ATOM | 1068 | CB | ASP | B | 163 | 6.694 | −53.957 | 6.138 | 1 | 57.11 | B | C |
| ATOM | 1069 | CG | ASP | B | 163 | 7.26 | −52.991 | 7.214 | 1 | 63.98 | B | C |
| ATOM | 1070 | OD1 | ASP | B | 163 | 8.132 | −53.372 | 8.023 | 1 | 63.77 | B | O |
| ATOM | 1071 | OD2 | ASP | B | 163 | 6.783 | −51.843 | 7.278 | 1 | 71.07 | B | O |
| ATOM | 1072 | C | ASP | B | 163 | 7.681 | −55.975 | 7.331 | 1 | 52.76 | B | C |
| ATOM | 1073 | O | ASP | B | 163 | 6.73 | −56.049 | 8.104 | 1 | 60.97 | B | O |
| ATOM | 1074 | N | LEU | B | 164 | 8.886 | −56.485 | 7.604 | 1 | 49.99 | B | N |
| ATOM | 1075 | CA | LEU | B | 164 | 9.164 | −57.335 | 8.781 | 1 | 48.89 | B | C |
| ATOM | 1076 | CB | LEU | B | 164 | 10.579 | −57.903 | 8.758 | 1 | 51.78 | B | C |
| ATOM | 1077 | CG | LEU | B | 164 | 10.975 | −58.873 | 9.9 | 1 | 57.32 | B | C |
| ATOM | 1078 | CD1 | LEU | B | 164 | 10.026 | −60.056 | 10.052 | 1 | 60 | B | C |
| ATOM | 1079 | CD2 | LEU | B | 164 | 12.366 | −59.411 | 9.637 | 1 | 57.6 | B | C |
| ATOM | 1080 | C | LEU | B | 164 | 8.964 | −56.695 | 10.135 | 1 | 47.44 | B | C |
| ATOM | 1081 | O | LEU | B | 164 | 8.403 | −57.324 | 11.024 | 1 | 46.96 | B | O |
| ATOM | 1082 | N | LYS | B | 165 | 9.428 | −55.462 | 10.295 | 1 | 50.52 | B | N |
| ATOM | 1083 | CA | LYS | B | 165 | 9.207 | −54.699 | 11.523 | 1 | 53.94 | B | C |
| ATOM | 1084 | CB | LYS | B | 165 | 9.846 | −53.311 | 11.43 | 1 | 58.73 | B | C |
| ATOM | 1085 | CG | LYS | B | 165 | 11.348 | −53.373 | 11.223 | 1 | 73.59 | B | C |
| ATOM | 1086 | CD | LYS | B | 165 | 12.156 | −52.406 | 12.079 | 1 | 81.38 | B | C |
| ATOM | 1087 | CE | LYS | B | 165 | 13.636 | −52.775 | 12.011 | 1 | 86.48 | B | C |
| ATOM | 1088 | NZ | LYS | B | 165 | 14.476 | −51.82 | 12.776 | 1 | 91.26 | B | N |
| ATOM | 1089 | C | LYS | B | 165 | 7.715 | −54.565 | 11.84 | 1 | 51.6 | B | C |
| ATOM | 1090 | O | LYS | B | 165 | 7.308 | −54.751 | 12.98 | 1 | 49.6 | B | O |
| ATOM | 1091 | N | GLN | B | 166 | 6.923 | −54.264 | 10.807 | 1 | 49.41 | B | N |
| ATOM | 1092 | CA | GLN | B | 166 | 5.471 | −54.157 | 10.925 | 1 | 52.87 | B | C |
| ATOM | 1093 | CB | GLN | B | 166 | 4.879 | −53.605 | 9.611 | 1 | 63.88 | B | C |
| ATOM | 1094 | CG | GLN | B | 166 | 3.385 | −53.873 | 9.411 | 1 | 73.01 | B | C |
| ATOM | 1095 | CD | GLN | B | 166 | 2.679 | −52.905 | 8.465 | 1 | 77.98 | B | C |
| ATOM | 1096 | OE1 | GLN | B | 166 | 3.31 | −52.144 | 7.712 | 1 | 69.61 | B | O |
| ATOM | 1097 | NE2 | GLN | B | 166 | 1.339 | −52.935 | 8.508 | 1 | 76.38 | B | N |
| ATOM | 1098 | C | GLN | B | 166 | 4.834 | −55.5 | 11.287 | 1 | 44.34 | B | C |
| ATOM | 1099 | O | GLN | B | 166 | 3.994 | −55.572 | 12.175 | 1 | 48.1 | B | O |
| ATOM | 1100 | N | CYS | B | 167 | 5.246 | −56.562 | 10.611 | 1 | 41.5 | B | N |
| ATOM | 1101 | CA | CYS | B | 167 | 4.703 | −57.903 | 10.875 | 1 | 40.85 | B | C |
| ATOM | 1102 | CB | CYS | B | 167 | 5.342 | −58.935 | 9.975 | 1 | 39.5 | B | C |
| ATOM | 1103 | SG | CYS | B | 167 | 4.926 | −60.658 | 10.33 | 1 | 44.1 | B | S |
| ATOM | 1104 | C | CYS | B | 167 | 4.966 | −58.275 | 12.323 | 1 | 39.76 | B | C |
| ATOM | 1105 | O | CYS | B | 167 | 4.077 | −58.714 | 13.026 | 1 | 40.44 | B | O |
| ATOM | 1106 | N | GLN | B | 168 | 6.174 | −58.012 | 12.777 | 1 | 39.45 | B | N |
| ATOM | 1107 | CA | GLN | B | 168 | 6.591 | −58.372 | 14.159 | 1 | 40.53 | B | C |
| ATOM | 1108 | CB | GLN | B | 168 | 8.108 | −58.213 | 14.287 | 1 | 42.35 | B | C |
| ATOM | 1109 | CG | GLN | B | 168 | 8.862 | −59.354 | 13.6 | 1 | 46.29 | B | C |
| ATOM | 1110 | CD | GLN | B | 168 | 10.388 | −59.207 | 13.697 | 1 | 49.83 | B | C |
| ATOM | 1111 | OE1 | GLN | B | 168 | 10.916 | −58.115 | 13.648 | 1 | 45.58 | B | O |
| ATOM | 1112 | NE2 | GLN | B | 168 | 11.087 | −60.325 | 13.832 | 1 | 52.47 | B | N |
| ATOM | 1113 | C | GLN | B | 168 | 5.842 | −57.608 | 15.237 | 1 | 40.56 | B | C |
| ATOM | 1114 | O | GLN | B | 168 | 5.483 | −58.154 | 16.255 | 1 | 37.84 | B | O |
| ATOM | 1115 | N | ALA | B | 169 | 5.609 | −56.33 | 15.014 | 1 | 45.74 | B | N |
| ATOM | 1116 | CA | ALA | B | 169 | 4.838 | −55.519 | 15.954 | 1 | 49.57 | B | C |
| ATOM | 1117 | CB | ALA | B | 169 | 4.899 | −54.041 | 15.54 | 1 | 46.23 | B | C |
| ATOM | 1118 | C | ALA | B | 169 | 3.381 | −56.026 | 16.009 | 1 | 48.45 | B | C |
| ATOM | 1119 | O | ALA | B | 169 | 2.778 | −56.15 | 17.077 | 1 | 47.76 | B | O |
| ATOM | 1120 | N | ALA | B | 170 | 2.826 | −56.349 | 14.849 | 1 | 47.07 | B | N |
| ATOM | 1121 | CA | ALA | B | 170 | 1.456 | −56.866 | 14.808 | 1 | 45.45 | B | C |
| ATOM | 1122 | CB | ALA | B | 170 | 0.939 | −56.897 | 13.369 | 1 | 45.27 | B | C |
| ATOM | 1123 | C | ALA | B | 170 | 1.332 | −58.251 | 15.472 | 1 | 41.61 | B | C |
| ATOM | 1124 | O | ALA | B | 170 | 0.3 | −58.568 | 16.016 | 1 | 43.57 | B | O |
| ATOM | 1125 | N | ILE | B | 171 | 2.387 | −59.06 | 15.442 | 1 | 44.84 | B | N |
| ATOM | 1126 | CA | ILE | B | 171 | 2.39 | −60.353 | 16.121 | 1 | 40.1 | B | C |
| ATOM | 1127 | CB | ILE | B | 171 | 3.542 | −61.247 | 15.66 | 1 | 41.01 | B | C |
| ATOM | 1128 | CG1 | ILE | B | 171 | 3.37 | −61.579 | 14.167 | 1 | 44.02 | B | C |
| ATOM | 1129 | CD1 | ILE | B | 171 | 4.553 | −62.333 | 13.553 | 1 | 46.95 | B | C |
| ATOM | 1130 | CG2 | ILE | B | 171 | 3.518 | −62.613 | 16.393 | 1 | 45.1 | B | C |
| ATOM | 1131 | C | ILE | B | 171 | 2.397 | −60.188 | 17.626 | 1 | 40.08 | B | C |
| ATOM | 1132 | O | ILE | B | 171 | 1.657 | −60.845 | 18.329 | 1 | 40.04 | B | O |
| ATOM | 1133 | N | ARG | B | 172 | 3.227 | −59.291 | 18.107 | 1 | 45.58 | B | N |
| ATOM | 1134 | CA | ARG | B | 172 | 3.257 | −58.93 | 19.523 | 1 | 46.46 | B | C |
| ATOM | 1135 | CB | ARG | B | 172 | 4.37 | −57.909 | 19.791 | 1 | 46.22 | B | C |
| ATOM | 1136 | CG | ARG | B | 172 | 5.772 | −58.491 | 19.73 | 1 | 49.52 | B | C |
| ATOM | 1137 | CD | ARG | B | 172 | 6.868 | −57.504 | 20.198 | 1 | 46.28 | B | C |
| ATOM | 1138 | NE | ARG | B | 172 | 7.624 | −57.171 | 19.007 | 1 | 58.43 | B | N |
| ATOM | 1139 | CZ | ARG | B | 172 | 7.71 | −55.975 | 18.421 | 1 | 58.61 | B | C |
| ATOM | 1140 | NH1 | ARG | B | 172 | 7.203 | −54.883 | 18.958 | 1 | 53.14 | B | N |
| ATOM | 1141 | NH2 | ARG | B | 172 | 8.369 | −55.878 | 17.286 | 1 | 62.38 | B | N |
| ATOM | 1142 | C | ARG | B | 172 | 1.908 | −58.379 | 19.996 | 1 | 49.77 | B | C |
| ATOM | 1143 | O | ARG | B | 172 | 1.4 | −58.762 | 21.049 | 1 | 53.25 | B | O |
| ATOM | 1144 | N | PHE | B | 173 | 1.306 | −57.501 | 19.207 | 1 | 49.75 | B | N |
| ATOM | 1145 | CA | PHE | B | 173 | 0.027 | −56.945 | 19.586 | 1 | 49.26 | B | C |
| ATOM | 1146 | CB | PHE | B | 173 | −0.359 | −55.824 | 18.613 | 1 | 52.98 | B | C |
| ATOM | 1147 | CG | PHE | B | 173 | −1.567 | −55.052 | 19.032 | 1 | 52.42 | B | C |
| ATOM | 1148 | CD1 | PHE | B | 173 | −2.831 | −55.46 | 18.652 | 1 | 49.14 | B | C |
| ATOM | 1149 | CE1 | PHE | B | 173 | −3.944 | −54.743 | 19.059 | 1 | 51.98 | B | C |
| ATOM | 1150 | CZ | PHE | B | 173 | −3.799 | −53.612 | 19.846 | 1 | 52.24 | B | C |
| ATOM | 1151 | CE2 | PHE | B | 173 | −2.539 | −53.201 | 20.242 | 1 | 47.25 | B | C |
| ATOM | 1152 | CD2 | PHE | B | 173 | −1.435 | −53.908 | 19.818 | 1 | 54.59 | B | C |
| ATOM | 1153 | C | PHE | B | 173 | −1.029 | −58.05 | 19.63 | 1 | 50.95 | B | C |
| ATOM | 1154 | O | PHE | B | 173 | −1.83 | −58.124 | 20.566 | 1 | 49.81 | B | O |
| ATOM | 1155 | N | PHE | B | 174 | −1.028 | −58.918 | 18.622 | 1 | 42.7 | B | N |
| ATOM | 1156 | CA | PHE | B | 174 | −1.977 | −60.032 | 18.602 | 1 | 42.57 | B | C |
| ATOM | 1157 | CB | PHE | B | 174 | −1.761 | −60.892 | 17.359 | 1 | 41.06 | B | C |
| ATOM | 1158 | CG | PHE | B | 174 | −2.638 | −62.078 | 17.27 | 1 | 41.99 | B | C |
| ATOM | 1159 | CD1 | PHE | B | 174 | −3.917 | −61.963 | 16.813 | 1 | 46.31 | B | C |
| ATOM | 1160 | CE1 | PHE | B | 174 | −4.734 | −63.074 | 16.682 | 1 | 48.07 | B | C |
| ATOM | 1161 | CZ | PHE | B | 174 | −4.26 | −64.328 | 16.998 | 1 | 51.45 | B | C |
| ATOM | 1162 | CE2 | PHE | B | 174 | −2.965 | −64.456 | 17.457 | 1 | 50.82 | B | C |
| ATOM | 1163 | CD2 | PHE | B | 174 | −2.166 | −63.334 | 17.59 | 1 | 49.07 | B | C |
| ATOM | 1164 | C | PHE | B | 174 | −1.889 | −60.869 | 19.876 | 1 | 45.11 | B | C |
| ATOM | 1165 | O | PHE | B | 174 | −2.911 | −61.1 | 20.531 | 1 | 45.28 | B | O |
| ATOM | 1166 | N | TYR | B | 175 | −0.689 | −61.298 | 20.266 | 1 | 45.39 | B | N |
| ATOM | 1167 | CA | TYR | B | 175 | −0.614 | −62.219 | 21.415 | 1 | 48.53 | B | C |
| ATOM | 1168 | CB | TYR | B | 175 | 0.685 | −63.002 | 21.406 | 1 | 43.19 | B | C |
| ATOM | 1169 | CG | TYR | B | 175 | 0.639 | −64.146 | 20.427 | 1 | 43.94 | B | C |
| ATOM | 1170 | CD1 | TYR | B | 175 | −0.117 | −65.27 | 20.7 | 1 | 41.93 | B | C |
| ATOM | 1171 | CE1 | TYR | B | 175 | −0.173 | −66.325 | 19.845 | 1 | 39.55 | B | C |
| ATOM | 1172 | CZ | TYR | B | 175 | 0.553 | −66.303 | 18.689 | 1 | 44.93 | B | C |
| ATOM | 1173 | OH | TYR | B | 175 | 0.48 | −67.389 | 17.823 | 1 | 48.57 | B | O |
| ATOM | 1174 | CE2 | TYR | B | 175 | 1.328 | −65.197 | 18.381 | 1 | 46.09 | B | C |
| ATOM | 1175 | CD2 | TYR | B | 175 | 1.361 | −64.12 | 19.238 | 1 | 42.3 | B | C |
| ATOM | 1176 | C | TYR | B | 175 | −0.865 | −61.542 | 22.782 | 1 | 55.58 | B | C |
| ATOM | 1177 | O | TYR | B | 175 | −1.227 | −62.218 | 23.729 | 1 | 60.65 | B | O |
| ATOM | 1178 | N | GLN | B | 176 | −0.71 | −60.222 | 22.865 | 1 | 53.67 | B | N |
| ATOM | 1179 | CA | GLN | B | 176 | −1.148 | −59.478 | 24.02 | 1 | 60.16 | B | C |
| ATOM | 1180 | CB | GLN | B | 176 | −0.25 | −58.264 | 24.197 | 1 | 64.26 | B | C |
| ATOM | 1181 | CG | GLN | B | 176 | 1.172 | −58.593 | 24.614 | 1 | 67.62 | B | C |
| ATOM | 1182 | CD | GLN | B | 176 | 2.143 | −57.554 | 24.12 | 1 | 74.94 | B | C |
| ATOM | 1183 | OE1 | GLN | B | 176 | 1.767 | −56.411 | 23.838 | 1 | 83.26 | B | O |
| ATOM | 1184 | NE2 | GLN | B | 176 | 3.4 | −57.943 | 23.988 | 1 | 86.57 | B | N |
| ATOM | 1185 | C | GLN | B | 176 | −2.604 | −58.98 | 23.96 | 1 | 63.93 | B | C |
| ATOM | 1186 | O | GLN | B | 176 | −2.98 | −58.178 | 24.794 | 1 | 71.6 | B | O |
| ATOM | 1187 | N | ASN | B | 177 | −3.406 | −59.384 | 22.979 | 1 | 56.98 | B | N |
| ATOM | 1188 | CA | ASN | B | 177 | −4.779 | −58.898 | 22.873 | 1 | 52.82 | B | C |
| ATOM | 1189 | CB | ASN | B | 177 | −4.868 | −57.792 | 21.842 | 1 | 55.87 | B | C |
| ATOM | 1190 | CG | ASN | B | 177 | −4.333 | −56.495 | 22.362 | 1 | 54.56 | B | C |
| ATOM | 1191 | OD1 | ASN | B | 177 | −3.257 | −56.07 | 22.009 | 1 | 55.98 | B | O |
| ATOM | 1192 | ND2 | ASN | B | 177 | −5.089 | −55.869 | 23.215 | 1 | 56.45 | B | N |
| ATOM | 1193 | C | ASN | B | 177 | −5.729 | −60.017 | 22.547 | 1 | 52.45 | B | C |
| ATOM | 1194 | O | ASN | B | 177 | −6.773 | −59.815 | 21.909 | 1 | 54.86 | B | O |
| ATOM | 1195 | N | ILE | B | 178 | −5.384 | −61.195 | 23.05 | 1 | 50.55 | B | N |
| ATOM | 1196 | CA | ILE | B | 178 | −6.178 | −62.383 | 22.855 | 1 | 50.34 | B | C |
| ATOM | 1197 | CB | ILE | B | 178 | −5.699 | −63.131 | 21.563 | 1 | 59.17 | B | C |
| ATOM | 1198 | CG1 | ILE | B | 178 | −6.877 | −63.424 | 20.675 | 1 | 60.02 | B | C |
| ATOM | 1199 | CD1 | ILE | B | 178 | −7.267 | −62.175 | 19.944 | 1 | 64.99 | B | C |
| ATOM | 1200 | CG2 | ILE | B | 178 | −4.806 | −64.353 | 21.765 | 1 | 57.65 | B | C |
| ATOM | 1201 | C | ILE | B | 178 | −5.975 | −63.125 | 24.156 | 1 | 53.58 | B | C |
| ATOM | 1202 | O | ILE | B | 178 | −4.874 | −63.089 | 24.698 | 1 | 52.87 | B | O |
| ATOM | 1203 | N | PRO | B | 179 | −7.026 | −63.767 | 24.682 | 1 | 59.37 | B | N |
| ATOM | 1204 | CA | PRO | B | 179 | −6.821 | −64.495 | 25.925 | 1 | 65.12 | B | C |
| ATOM | 1205 | CB | PRO | B | 179 | −8.194 | −65.133 | 26.192 | 1 | 67.72 | B | C |
| ATOM | 1206 | CG | PRO | B | 179 | −9.159 | −64.453 | 25.279 | 1 | 66.95 | B | C |
| ATOM | 1207 | CD | PRO | B | 179 | −8.357 | −64.026 | 24.098 | 1 | 62.5 | B | C |
| ATOM | 1208 | C | PRO | B | 179 | −5.756 | −65.575 | 25.765 | 1 | 65.21 | B | C |
| ATOM | 1209 | O | PRO | B | 179 | −5.638 | −66.136 | 24.674 | 1 | 62.76 | B | O |
| ATOM | 1210 | N | PHE | B | 180 | −5 | −65.832 | 26.837 | 1 | 64.23 | B | N |
| ATOM | 1211 | CA | PHE | B | 180 | −3.96 | −66.882 | 26.892 | 1 | 62.82 | B | C |
| ATOM | 1212 | CB | PHE | B | 180 | −3.332 | −66.947 | 28.31 | 1 | 64.6 | B | C |
| ATOM | 1213 | CG | PHE | B | 180 | −2.349 | −68.082 | 28.503 | 1 | 69.56 | B | C |
| ATOM | 1214 | CD1 | PHE | B | 180 | −2.788 | −69.356 | 28.897 | 1 | 73.32 | B | C |
| ATOM | 1215 | CE1 | PHE | B | 180 | −1.886 | −70.411 | 29.063 | 1 | 75.21 | B | C |
| ATOM | 1216 | CZ | PHE | B | 180 | −0.526 | −70.212 | 28.834 | 1 | 75.02 | B | C |
| ATOM | 1217 | CE2 | PHE | B | 180 | −0.076 | −68.959 | 28.435 | 1 | 76.14 | B | C |
| ATOM | 1218 | CD2 | PHE | B | 180 | −0.983 | −67.899 | 28.274 | 1 | 73.14 | B | C |
| ATOM | 1219 | C | PHE | B | 180 | −4.453 | −68.28 | 26.456 | 1 | 65.97 | B | C |
| ATOM | 1220 | O | PHE | B | 180 | −3.693 | −69.053 | 25.854 | 1 | 68.73 | B | O |
| ATOM | 1221 | N | ASN | B | 181 | −5.7 | −68.622 | 26.762 | 1 | 70.01 | B | N |
| ATOM | 1222 | CA | ASN | B | 181 | −6.215 | −69.967 | 26.411 | 1 | 73.87 | B | C |
| ATOM | 1223 | CB | ASN | B | 181 | −7.587 | −70.346 | 27.071 | 1 | 78.98 | B | C |
| ATOM | 1224 | CG | ASN | B | 181 | −8.382 | −69.139 | 27.641 | 1 | 89.31 | B | C |
| ATOM | 1225 | OD1 | ASN | B | 181 | −7.947 | −68.476 | 28.589 | 1 | 82.44 | B | O |
| ATOM | 1226 | ND2 | ASN | B | 181 | −9.575 | −68.892 | 27.096 | 1 | 97.28 | B | N |
| ATOM | 1227 | C | ASN | B | 181 | −6.235 | −70.144 | 24.888 | 1 | 69.59 | B | C |
| ATOM | 1228 | O | ASN | B | 181 | −5.875 | −71.198 | 24.364 | 1 | 68.28 | B | O |
| ATOM | 1229 | N | ILE | B | 182 | −6.59 | −69.077 | 24.186 | 1 | 63.15 | B | N |
| ATOM | 1230 | CA | ILE | B | 182 | −6.667 | −69.083 | 22.715 | 1 | 63.74 | B | C |
| ATOM | 1231 | CB | ILE | B | 182 | −7.604 | −67.938 | 22.193 | 1 | 66.17 | B | C |
| ATOM | 1232 | CG1 | ILE | B | 182 | −9.065 | −68.196 | 22.601 | 1 | 74.9 | B | C |
| ATOM | 1233 | CD1 | ILE | B | 182 | −9.438 | −67.767 | 24.007 | 1 | 75.65 | B | C |
| ATOM | 1234 | CG2 | ILE | B | 182 | −7.537 | −67.809 | 20.687 | 1 | 61.77 | B | C |
| ATOM | 1235 | C | ILE | B | 182 | −5.253 | −68.947 | 22.141 | 1 | 53.94 | B | C |
| ATOM | 1236 | O | ILE | B | 182 | −4.877 | −69.634 | 21.175 | 1 | 53.75 | B | O |
| ATOM | 1237 | N | ALA | B | 183 | −4.484 | −68.037 | 22.733 | 1 | 48.16 | B | N |
| ATOM | 1238 | CA | ALA | B | 183 | −3.119 | −67.823 | 22.336 | 1 | 48.24 | B | C |
| ATOM | 1239 | CB | ALA | B | 183 | −2.456 | −66.811 | 23.228 | 1 | 44.6 | B | C |
| ATOM | 1240 | C | ALA | B | 183 | −2.328 | −69.122 | 22.299 | 1 | 48.09 | B | C |
| ATOM | 1241 | O | ALA | B | 183 | −1.649 | −69.39 | 21.326 | 1 | 47.73 | B | O |
| ATOM | 1242 | N | GLN | B | 184 | −2.461 | −69.957 | 23.315 | 1 | 55.71 | B | N |
| ATOM | 1243 | CA | GLN | B | 184 | −1.652 | −71.185 | 23.372 | 1 | 55.12 | B | C |
| ATOM | 1244 | CB | GLN | B | 184 | −1.578 | −71.785 | 24.781 | 1 | 57.57 | B | C |
| ATOM | 1245 | CG | GLN | B | 184 | −2.764 | −72.607 | 25.229 | 1 | 64.02 | B | C |
| ATOM | 1246 | CD | GLN | B | 184 | −2.686 | −72.912 | 26.716 | 1 | 86.43 | B | C |
| ATOM | 1247 | OE1 | GLN | B | 184 | −1.591 | −73.071 | 27.276 | 1 | 89.95 | B | O |
| ATOM | 1248 | NE2 | GLN | B | 184 | −3.843 | −72.986 | 27.369 | 1 | 94.41 | B | N |
| ATOM | 1249 | C | GLN | B | 184 | −2.115 | −72.203 | 22.366 | 1 | 45.84 | B | C |
| ATOM | 1250 | O | GLN | B | 184 | −1.292 | −72.925 | 21.832 | 1 | 49.16 | B | O |
| ATOM | 1251 | N | MET | B | 185 | −3.409 | −72.264 | 22.088 | 1 | 47.29 | B | N |
| ATOM | 1252 | CA | MET | B | 185 | −3.885 | −73.175 | 21.033 | 1 | 53.23 | B | C |
| ATOM | 1253 | CB | MET | B | 185 | −5.39 | −73.234 | 20.991 | 1 | 60.76 | B | C |
| ATOM | 1254 | CG | MET | B | 185 | −5.959 | −73.874 | 22.24 | 1 | 65.64 | B | C |
| ATOM | 1255 | SD | MET | B | 185 | −7.73 | −74.059 | 22.101 | 1 | 71.68 | B | S |
| ATOM | 1256 | CE | MET | B | 185 | −8.356 | −72.459 | 22.587 | 1 | 70.06 | B | C |
| ATOM | 1257 | C | MET | B | 185 | −3.343 | −72.796 | 19.651 | 1 | 52.74 | B | C |
| ATOM | 1258 | O | MET | B | 185 | −2.972 | −73.667 | 18.881 | 1 | 50.46 | B | O |
| ATOM | 1259 | N | LEU | B | 186 | −3.256 | −71.496 | 19.373 | 1 | 52.44 | B | N |
| ATOM | 1260 | CA | LEU | B | 186 | −2.648 | −71.022 | 18.148 | 1 | 50.77 | B | C |
| ATOM | 1261 | CB | LEU | B | 186 | −2.898 | −69.522 | 17.972 | 1 | 51.34 | B | C |
| ATOM | 1262 | CG | LEU | B | 186 | −4.333 | −69.131 | 17.619 | 1 | 57.24 | B | C |
| ATOM | 1263 | CD1 | LEU | B | 186 | −4.702 | −67.788 | 18.209 | 1 | 57.57 | B | C |
| ATOM | 1264 | CD2 | LEU | B | 186 | −4.535 | −69.1 | 16.11 | 1 | 60.68 | B | C |
| ATOM | 1265 | C | LEU | B | 186 | −1.149 | −71.282 | 18.14 | 1 | 52.74 | B | C |
| ATOM | 1266 | O | LEU | B | 186 | −0.594 | −71.713 | 17.148 | 1 | 50.08 | B | O |
| ATOM | 1267 | N | ALA | B | 187 | −0.479 | −70.953 | 19.233 | 1 | 54.61 | B | N |
| ATOM | 1268 | CA | ALA | B | 187 | 0.981 | −71.006 | 19.257 | 1 | 54.45 | B | C |
| ATOM | 1269 | CB | ALA | B | 187 | 1.525 | −70.386 | 20.538 | 1 | 59.9 | B | C |
| ATOM | 1270 | C | ALA | B | 187 | 1.488 | −72.407 | 19.091 | 1 | 47.38 | B | C |
| ATOM | 1271 | O | ALA | B | 187 | 2.471 | −72.618 | 18.411 | 1 | 53.43 | B | O |
| ATOM | 1272 | N | PHE | B | 188 | 0.787 | −73.366 | 19.672 | 1 | 53.03 | B | N |
| ATOM | 1273 | CA | PHE | B | 188 | 1.255 | −74.749 | 19.715 | 1 | 53.46 | B | C |
| ATOM | 1274 | CB | PHE | B | 188 | 1.254 | −75.265 | 21.179 | 1 | 53.65 | B | C |
| ATOM | 1275 | CG | PHE | B | 188 | 2.08 | −74.401 | 22.098 | 1 | 53.95 | B | C |
| ATOM | 1276 | CD1 | PHE | B | 188 | 3.438 | −74.276 | 21.895 | 1 | 57.34 | B | C |
| ATOM | 1277 | CE1 | PHE | B | 188 | 4.201 | −73.439 | 22.685 | 1 | 60.31 | B | C |
| ATOM | 1278 | CZ | PHE | B | 188 | 3.603 | −72.704 | 23.687 | 1 | 57.25 | B | C |
| ATOM | 1279 | CE2 | PHE | B | 188 | 2.249 | −72.811 | 23.897 | 1 | 55.85 | B | C |
| ATOM | 1280 | CD2 | PHE | B | 188 | 1.496 | −73.647 | 23.099 | 1 | 58.14 | B | C |
| ATOM | 1281 | C | PHE | B | 188 | 0.53 | −75.683 | 18.787 | 1 | 55.48 | B | C |
| ATOM | 1282 | O | PHE | B | 188 | 0.866 | −76.862 | 18.755 | 1 | 55.92 | B | O |
| ATOM | 1283 | N | CYS | B | 189 | −0.412 | −75.182 | 17.985 | 1 | 55.03 | B | N |
| ATOM | 1284 | CA | CYS | B | 189 | −1.143 | −76.056 | 17.026 | 1 | 53.99 | B | C |
| ATOM | 1285 | CB | CYS | B | 189 | −2.223 | −75.259 | 16.209 | 1 | 53.92 | B | C |
| ATOM | 1286 | SG | CYS | B | 189 | −1.575 | −74.201 | 14.852 | 1 | 52.05 | B | S |
| ATOM | 1287 | C | CYS | B | 189 | −0.134 | −76.737 | 16.069 | 1 | 47.38 | B | C |
| ATOM | 1288 | O | CYS | B | 189 | 0.885 | −76.149 | 15.684 | 1 | 43.82 | B | O |
| ATOM | 1289 | N | ASP | B | 190 | −0.432 | −77.958 | 15.661 | 1 | 51.77 | B | N |
| ATOM | 1290 | CA | ASP | B | 190 | 0.402 | −78.647 | 14.683 | 1 | 57.63 | B | C |
| ATOM | 1291 | CB | ASP | B | 190 | 1.408 | −79.561 | 15.403 | 1 | 63.18 | B | C |
| ATOM | 1292 | CG | ASP | B | 190 | 2.592 | −79.963 | 14.522 | 1 | 67.21 | B | C |
| ATOM | 1293 | OD1 | ASP | B | 190 | 2.941 | −79.209 | 13.574 | 1 | 58.51 | B | O |
| ATOM | 1294 | OD2 | ASP | B | 190 | 3.152 | −81.056 | 14.763 | 1 | 79.87 | B | O |
| ATOM | 1295 | C | ASP | B | 190 | −0.47 | −79.445 | 13.725 | 1 | 58.05 | B | C |
| ATOM | 1296 | O | ASP | B | 190 | −1.675 | −79.531 | 13.909 | 1 | 62.51 | B | O |
| ATOM | 1297 | N | CYS | B | 191 | 0.14 | −80.003 | 12.689 | 1 | 64.27 | B | N |
| ATOM | 1298 | CA | CYS | B | 191 | −0.58 | −80.768 | 11.682 | 1 | 71.75 | B | C |
| ATOM | 1299 | CB | CYS | B | 191 | −0.587 | −79.99 | 10.362 | 1 | 69.9 | B | C |
| ATOM | 1300 | SG | CYS | B | 191 | −1.43 | −78.411 | 10.435 | 1 | 73.19 | B | S |
| ATOM | 1301 | C | CYS | B | 191 | 0.084 | −82.104 | 11.399 | 1 | 73.29 | B | C |
| ATOM | 1302 | O | CYS | B | 191 | 1.324 | −82.162 | 11.275 | 1 | 75.73 | B | O |
| ATOM | 1303 | N | ALA | B | 192 | −0.746 | −83.146 | 11.242 | 1 | 73.48 | B | N |
| ATOM | 1304 | CA | ALA | B | 192 | −0.37 | −84.37 | 10.506 | 1 | 74.57 | B | C |
| ATOM | 1305 | CB | ALA | B | 192 | −1.611 | −85.186 | 10.186 | 1 | 67.42 | B | C |
| ATOM | 1306 | C | ALA | B | 192 | 0.371 | −83.993 | 9.215 | 1 | 78.28 | B | C |
| ATOM | 1307 | O | ALA | B | 192 | 0.022 | −82.997 | 8.579 | 1 | 86.12 | B | O |
| ATOM | 1308 | N | GLN | B | 193 | 1.406 | −84.748 | 8.847 | 1 | 86.15 | B | N |
| ATOM | 1309 | CA | GLN | B | 193 | 2.189 | −84.45 | 7.62 | 1 | 95.78 | B | C |
| ATOM | 1310 | CB | GLN | B | 193 | 3.411 | −85.382 | 7.514 | 1 | 98.28 | B | C |
| ATOM | 1311 | CG | GLN | B | 193 | 4.273 | −85.198 | 6.263 | 1 | 103.53 | B | C |
| ATOM | 1312 | CD | GLN | B | 193 | 4.754 | −83.764 | 6.07 | 1 | 112.66 | B | C |
| ATOM | 1313 | OE1 | GLN | B | 193 | 4.985 | −83.034 | 7.04 | 1 | 121.3 | B | O |
| ATOM | 1314 | NE2 | GLN | B | 193 | 4.917 | −83.356 | 4.815 | 1 | 112.47 | B | N |
| ATOM | 1315 | C | GLN | B | 193 | 1.311 | −84.547 | 6.349 | 1 | 95.46 | B | C |
| ATOM | 1316 | O | GLN | B | 193 | 1.425 | −83.73 | 5.427 | 1 | 80.54 | B | O |
| ATOM | 1317 | N | SER | B | 194 | 0.461 | −85.574 | 6.333 | 1 | 96.23 | B | N |
| ATOM | 1318 | CA | SER | B | 194 | −0.644 | −85.72 | 5.38 | 1 | 93.44 | B | C |
| ATOM | 1319 | CB | SER | B | 194 | −1.542 | −86.916 | 5.818 | 1 | 90.15 | B | C |
| ATOM | 1320 | OG | SER | B | 194 | −2.93 | −86.615 | 5.837 | 1 | 88.17 | B | O |
| ATOM | 1321 | C | SER | B | 194 | −1.472 | −84.419 | 5.159 | 1 | 87.76 | B | C |
| ATOM | 1322 | O | SER | B | 194 | −1.531 | −83.905 | 4.046 | 1 | 95.38 | B | O |
| ATOM | 1323 | N | ASP | B | 195 | −2.03 | −83.868 | 6.235 | 1 | 78.51 | B | N |
| ATOM | 1324 | CA | ASP | B | 195 | −3.197 | −82.954 | 6.184 | 1 | 75.66 | B | C |
| ATOM | 1325 | CB | ASP | B | 195 | −3.774 | −82.827 | 7.592 | 1 | 71.8 | B | C |
| ATOM | 1326 | CG | ASP | B | 195 | −5.114 | −82.209 | 7.606 | 1 | 75.31 | B | C |
| ATOM | 1327 | OD1 | ASP | B | 195 | −5.516 | −81.587 | 6.596 | 1 | 82.32 | B | O |
| ATOM | 1328 | OD2 | ASP | B | 195 | −5.773 | −82.338 | 8.648 | 1 | 77.88 | B | O |
| ATOM | 1329 | C | ASP | B | 195 | −2.97 | −81.544 | 5.553 | 1 | 73 | B | C |
| ATOM | 1330 | O | ASP | B | 195 | −2.546 | −80.586 | 6.228 | 1 | 63.09 | B | O |
| ATOM | 1331 | N | ILE | B | 196 | −3.297 | −81.438 | 4.262 | 1 | 67.49 | B | N |
| ATOM | 1332 | CA | ILE | B | 196 | −2.993 | −80.247 | 3.47 | 1 | 65.54 | B | C |
| ATOM | 1333 | CB | ILE | B | 196 | −3.176 | −80.496 | 1.932 | 1 | 66.74 | B | C |
| ATOM | 1334 | CG1 | ILE | B | 196 | −2.214 | −81.591 | 1.41 | 1 | 69.06 | B | C |
| ATOM | 1335 | CD1 | ILE | B | 196 | −0.744 | −81.247 | 1.455 | 1 | 74.54 | B | C |
| ATOM | 1336 | CG2 | ILE | B | 196 | −2.989 | −79.218 | 1.119 | 1 | 65.37 | B | C |
| ATOM | 1337 | C | ILE | B | 196 | −3.789 | −79.029 | 3.968 | 1 | 58.53 | B | C |
| ATOM | 1338 | O | ILE | B | 196 | −3.216 | −77.93 | 4.055 | 1 | 54.61 | B | O |
| ATOM | 1339 | N | PRO | B | 197 | −5.095 | −79.208 | 4.274 | 1 | 54.87 | B | N |
| ATOM | 1340 | CA | PRO | B | 197 | −5.834 | −78.068 | 4.828 | 1 | 57.6 | B | C |
| ATOM | 1341 | CB | PRO | B | 197 | −7.275 | −78.595 | 4.971 | 1 | 58.82 | B | C |
| ATOM | 1342 | CG | PRO | B | 197 | −7.35 | −79.886 | 4.202 | 1 | 56.94 | B | C |
| ATOM | 1343 | CD | PRO | B | 197 | −6.008 | −80.154 | 3.6 | 1 | 54.63 | B | C |
| ATOM | 1344 | C | PRO | B | 197 | −5.296 | −77.59 | 6.168 | 1 | 57.88 | B | C |
| ATOM | 1345 | O | PRO | B | 197 | −5.23 | −76.353 | 6.409 | 1 | 48.36 | B | O |
| ATOM | 1346 | N | CYS | B | 198 | −4.91 | −78.54 | 7.03 | 1 | 54.2 | B | N |
| ATOM | 1347 | CA | CYS | B | 198 | −4.291 | −78.174 | 8.304 | 1 | 53.53 | B | C |
| ATOM | 1348 | CB | CYS | B | 198 | −4.029 | −79.381 | 9.184 | 1 | 56.05 | B | C |
| ATOM | 1349 | SG | CYS | B | 198 | −3.412 | −78.923 | 10.822 | 1 | 58.84 | B | S |
| ATOM | 1350 | C | CYS | B | 198 | −3.001 | −77.406 | 8.026 | 1 | 51.97 | B | C |
| ATOM | 1351 | O | CYS | B | 198 | −2.771 | −76.334 | 8.571 | 1 | 52.83 | B | O |
| ATOM | 1352 | N | GLN | B | 199 | −2.209 | −77.937 | 7.119 | 1 | 47.79 | B | N |
| ATOM | 1353 | CA | GLN | B | 199 | −0.902 | −77.385 | 6.78 | 1 | 52.43 | B | C |
| ATOM | 1354 | CB | GLN | B | 199 | −0.185 | −78.37 | 5.793 | 1 | 58.44 | B | C |
| ATOM | 1355 | CG | GLN | B | 199 | 1.33 | −78.546 | 5.935 | 1 | 73.01 | B | C |
| ATOM | 1356 | CD | GLN | B | 199 | 1.782 | −79.426 | 7.115 | 1 | 74.68 | B | C |
| ATOM | 1357 | OE1 | GLN | B | 199 | 1.185 | −80.477 | 7.425 | 1 | 75.27 | B | O |
| ATOM | 1358 | NE2 | GLN | B | 199 | 2.86 | −79 | 7.77 | 1 | 74.04 | B | N |
| ATOM | 1359 | C | GLN | B | 199 | −1.058 | −75.967 | 6.193 | 1 | 49.15 | B | C |
| ATOM | 1360 | O | GLN | B | 199 | −0.213 | −75.071 | 6.419 | 1 | 47.93 | B | O |
| ATOM | 1361 | N | GLN | B | 200 | −2.122 | −75.765 | 5.412 | 1 | 48.73 | B | N |
| ATOM | 1362 | CA | GLN | B | 200 | −2.41 | −74.437 | 4.827 | 1 | 46.88 | B | C |
| ATOM | 1363 | CB | GLN | B | 200 | −3.532 | −74.512 | 3.797 | 1 | 43.45 | B | C |
| ATOM | 1364 | CG | GLN | B | 200 | −3.065 | −75.186 | 2.504 | 1 | 50.1 | B | C |
| ATOM | 1365 | CD | GLN | B | 200 | −4.157 | −75.825 | 1.653 | 1 | 49.03 | B | C |
| ATOM | 1366 | OE1 | GLN | B | 200 | −5.169 | −76.353 | 2.14 | 1 | 52.56 | B | O |
| ATOM | 1367 | NE2 | GLN | B | 200 | −3.932 | −75.82 | 0.372 | 1 | 53.41 | B | N |
| ATOM | 1368 | C | GLN | B | 200 | −2.739 | −73.445 | 5.943 | 1 | 46.96 | B | C |
| ATOM | 1369 | O | GLN | B | 200 | −2.29 | −72.306 | 5.913 | 1 | 51.15 | B | O |
| ATOM | 1370 | N | SER | B | 201 | −3.495 | −73.894 | 6.929 | 1 | 43.94 | B | N |
| ATOM | 1371 | CA | SER | B | 201 | −3.8 | −73.07 | 8.102 | 1 | 46.83 | B | C |
| ATOM | 1372 | CB | SER | B | 201 | −4.78 | −73.797 | 9.003 | 1 | 45.32 | B | C |
| ATOM | 1373 | OG | SER | B | 201 | −5.879 | −74.221 | 8.232 | 1 | 44.17 | B | O |
| ATOM | 1374 | C | SER | B | 201 | −2.559 | −72.712 | 8.904 | 1 | 46.79 | B | C |
| ATOM | 1375 | O | SER | B | 201 | −2.353 | −71.532 | 9.271 | 1 | 42.69 | B | O |
| ATOM | 1376 | N | LYS | B | 202 | −1.709 | −73.718 | 9.12 | 1 | 49.4 | B | N |
| ATOM | 1377 | CA | LYS | B | 202 | −0.425 | −73.512 | 9.793 | 1 | 47.05 | B | C |
| ATOM | 1378 | CB | LYS | B | 202 | 0.42 | −74.784 | 9.857 | 1 | 51.18 | B | C |
| ATOM | 1379 | CG | LYS | B | 202 | 1.588 | −74.627 | 10.861 | 1 | 55.38 | B | C |
| ATOM | 1380 | CD | LYS | B | 202 | 2.777 | −75.542 | 10.59 | 1 | 63 | B | C |
| ATOM | 1381 | CE | LYS | B | 202 | 2.715 | −76.857 | 11.374 | 1 | 67.06 | B | C |
| ATOM | 1382 | NZ | LYS | B | 202 | 3.977 | −77.067 | 12.16 | 1 | 73.1 | B | N |
| ATOM | 1383 | C | LYS | B | 202 | 0.379 | −72.414 | 9.141 | 1 | 45.09 | B | C |
| ATOM | 1384 | O | LYS | B | 202 | 0.823 | −71.466 | 9.799 | 1 | 49.56 | B | O |
| ATOM | 1385 | N | GLU | B | 203 | 0.572 | −72.533 | 7.844 | 1 | 48.11 | B | N |
| ATOM | 1386 | CA | GLU | B | 203 | 1.378 | −71.561 | 7.163 | 1 | 52.95 | B | C |
| ATOM | 1387 | CB | GLU | B | 203 | 1.463 | −71.848 | 5.658 | 1 | 67.8 | B | C |
| ATOM | 1388 | CG | GLU | B | 203 | 2.39 | −70.851 | 4.936 | 1 | 83.09 | B | C |
| ATOM | 1389 | CD | GLU | B | 203 | 2.568 | −71.13 | 3.456 | 1 | 91.01 | B | C |
| ATOM | 1390 | OE1 | GLU | B | 203 | 3.221 | −70.29 | 2.802 | 1 | 89.48 | B | O |
| ATOM | 1391 | OE2 | GLU | B | 203 | 2.067 | −72.171 | 2.957 | 1 | 90.34 | B | O |
| ATOM | 1392 | C | GLU | B | 203 | 0.853 | −70.155 | 7.397 | 1 | 47.85 | B | C |
| ATOM | 1393 | O | GLU | B | 203 | 1.633 | −69.259 | 7.611 | 1 | 46.98 | B | O |
| ATOM | 1394 | N | ALA | B | 204 | −0.467 | −69.983 | 7.357 | 1 | 48.71 | B | N |
| ATOM | 1395 | CA | ALA | B | 204 | −1.107 | −68.684 | 7.518 | 1 | 46.92 | B | C |
| ATOM | 1396 | CB | ALA | B | 204 | −2.604 | −68.797 | 7.174 | 1 | 49.41 | B | C |
| ATOM | 1397 | C | ALA | B | 204 | −0.945 | −68.221 | 8.935 | 1 | 47.26 | B | C |
| ATOM | 1398 | O | ALA | B | 204 | −0.606 | −67.079 | 9.202 | 1 | 46.67 | B | O |
| ATOM | 1399 | N | LEU | B | 205 | −1.23 | −69.136 | 9.855 | 1 | 44.98 | B | N |
| ATOM | 1400 | CA | LEU | B | 205 | −1.37 | −68.777 | 11.234 | 1 | 47.1 | B | C |
| ATOM | 1401 | CB | LEU | B | 205 | −2.06 | −69.881 | 12.042 | 1 | 45.17 | B | C |
| ATOM | 1402 | CG | LEU | B | 205 | −3.575 | −70.024 | 11.944 | 1 | 42.73 | B | C |
| ATOM | 1403 | CD1 | LEU | B | 205 | −4.038 | −71.106 | 12.904 | 1 | 45.26 | B | C |
| ATOM | 1404 | CD2 | LEU | B | 205 | −4.283 | −68.704 | 12.236 | 1 | 43.41 | B | C |
| ATOM | 1405 | C | LEU | B | 205 | −0.034 | −68.471 | 11.846 | 1 | 53.3 | B | C |
| ATOM | 1406 | O | LEU | B | 205 | 0.007 | −67.777 | 12.844 | 1 | 61.02 | B | O |
| ATOM | 1407 | N | HIS | B | 206 | 1.051 | −68.998 | 11.293 | 1 | 54.96 | B | N |
| ATOM | 1408 | CA | HIS | B | 206 | 2.375 | −68.74 | 11.886 | 1 | 61.58 | B | C |
| ATOM | 1409 | CB | HIS | B | 206 | 3.056 | −70.067 | 12.226 | 1 | 57.82 | B | C |
| ATOM | 1410 | CG | HIS | B | 206 | 2.336 | −70.838 | 13.296 | 1 | 59.97 | B | C |
| ATOM | 1411 | ND1 | HIS | B | 206 | 2.543 | −72.186 | 13.517 | 1 | 60.06 | B | N |
| ATOM | 1412 | CE1 | HIS | B | 206 | 1.762 | −72.591 | 14.503 | 1 | 60.33 | B | C |
| ATOM | 1413 | NE2 | HIS | B | 206 | 1.058 | −71.555 | 14.931 | 1 | 59.19 | B | N |
| ATOM | 1414 | CD2 | HIS | B | 206 | 1.399 | −70.447 | 14.198 | 1 | 56.57 | B | C |
| ATOM | 1415 | C | HIS | B | 206 | 3.273 | −67.77 | 11.088 | 1 | 61.5 | B | C |
| ATOM | 1416 | O | HIS | B | 206 | 4.45 | −67.606 | 11.416 | 1 | 72.4 | B | O |
| ATOM | 1417 | N | SER | B | 207 | 2.678 | −67.083 | 10.105 | 1 | 64.07 | B | N |
| ATOM | 1418 | CA | SER | B | 207 | 3.314 | −66.013 | 9.326 | 1 | 68.12 | B | C |
| ATOM | 1419 | CB | SER | B | 207 | 3.451 | −64.755 | 10.191 | 1 | 79.2 | B | C |
| ATOM | 1420 | OG | SER | B | 207 | 2.496 | −63.801 | 9.775 | 1 | 90.22 | B | O |
| ATOM | 1421 | C | SER | B | 207 | 4.643 | −66.423 | 8.707 | 1 | 64.74 | B | C |
| ATOM | 1422 | O | SER | B | 207 | 5.654 | −65.71 | 8.762 | 1 | 59.99 | B | O |
| ATOM | 1423 | N | LYS | B | 208 | 4.632 | −67.598 | 8.118 | 1 | 55.13 | B | N |
| ATOM | 1424 | CA | LYS | B | 208 | 5.874 | −68.231 | 7.785 | 1 | 58.17 | B | C |
| ATOM | 1425 | CB | LYS | B | 208 | 5.637 | −69.626 | 7.206 | 1 | 55.91 | B | C |
| ATOM | 1426 | CG | LYS | B | 208 | 4.904 | −70.554 | 8.15 | 1 | 59.38 | B | C |
| ATOM | 1427 | CD | LYS | B | 208 | 5.667 | −70.885 | 9.43 | 1 | 68.94 | B | C |
| ATOM | 1428 | CE | LYS | B | 208 | 6.486 | −72.171 | 9.327 | 1 | 69.92 | B | C |
| ATOM | 1429 | NZ | LYS | B | 208 | 5.643 | −73.323 | 8.862 | 1 | 76.72 | B | N |
| ATOM | 1430 | C | LYS | B | 208 | 6.713 | −67.349 | 6.846 | 1 | 54.5 | B | C |
| ATOM | 1431 | O | LYS | B | 208 | 7.799 | −66.88 | 7.214 | 1 | 60.6 | B | O |
| ATOM | 1432 | N | THR | B | 209 | 6.193 | −67.055 | 5.676 | 1 | 48.52 | B | N |
| ATOM | 1433 | CA | THR | B | 209 | 7.018 | −66.411 | 4.657 | 1 | 55.6 | B | C |
| ATOM | 1434 | CB | THR | B | 209 | 6.317 | −66.434 | 3.279 | 1 | 58.26 | B | C |
| ATOM | 1435 | OG1 | THR | B | 209 | 4.984 | −65.915 | 3.403 | 1 | 57.27 | B | O |
| ATOM | 1436 | CG2 | THR | B | 209 | 6.236 | −67.869 | 2.768 | 1 | 60.72 | B | C |
| ATOM | 1437 | C | THR | B | 209 | 7.419 | −64.964 | 5.016 | 1 | 54.63 | B | C |
| ATOM | 1438 | O | THR | B | 209 | 8.321 | −64.421 | 4.409 | 1 | 63.72 | B | O |
| ATOM | 1439 | N | CYS | B | 210 | 6.756 | −64.332 | 5.971 | 1 | 43.07 | B | N |
| ATOM | 1440 | CA | CYS | B | 210 | 7.209 | −63.03 | 6.414 | 1 | 48.49 | B | C |
| ATOM | 1441 | CB | CYS | B | 210 | 6.039 | −62.094 | 6.679 | 1 | 49.92 | B | C |
| ATOM | 1442 | SG | CYS | B | 210 | 6.613 | −60.434 | 7.149 | 1 | 66.9 | B | S |
| ATOM | 1443 | C | CYS | B | 210 | 8.065 | −63.107 | 7.666 | 1 | 46.66 | B | C |
| ATOM | 1444 | O | CYS | B | 210 | 9.156 | −62.569 | 7.7 | 1 | 48.31 | B | O |
| ATOM | 1445 | N | ALA | B | 211 | 7.552 | −63.752 | 8.711 | 1 | 52.73 | B | N |
| ATOM | 1446 | CA | ALA | B | 211 | 8.148 | −63.671 | 10.058 | 1 | 49.93 | B | C |
| ATOM | 1447 | CB | ALA | B | 211 | 7.033 | −63.804 | 11.092 | 1 | 55.41 | B | C |
| ATOM | 1448 | C | ALA | B | 211 | 9.245 | −64.705 | 10.356 | 1 | 47.06 | B | C |
| ATOM | 1449 | O | ALA | B | 211 | 10.049 | −64.513 | 11.272 | 1 | 46.57 | B | O |
| ATOM | 1450 | N | VAL | B | 212 | 9.255 | −65.796 | 9.605 | 1 | 41.81 | B | N |
| ATOM | 1451 | CA | VAL | B | 212 | 10.039 | −66.943 | 9.931 | 1 | 46.06 | B | C |
| ATOM | 1452 | CB | VAL | B | 212 | 9.123 | −68.176 | 10.156 | 1 | 46.19 | B | C |
| ATOM | 1453 | CG1 | VAL | B | 212 | 9.953 | −69.361 | 10.62 | 1 | 50.47 | B | C |
| ATOM | 1454 | CG2 | VAL | B | 212 | 8.09 | −67.873 | 11.236 | 1 | 48.47 | B | C |
| ATOM | 1455 | C | VAL | B | 212 | 11.145 | −67.221 | 8.894 | 1 | 46.16 | B | C |
| ATOM | 1456 | O | VAL | B | 212 | 12.287 | −67.473 | 9.259 | 1 | 46.25 | B | O |
| ATOM | 1457 | N | ASN | B | 213 | 10.808 | −67.222 | 7.623 | 1 | 43.22 | B | N |
| ATOM | 1458 | CA | ASN | B | 213 | 11.761 | −67.621 | 6.581 | 1 | 47.45 | B | C |
| ATOM | 1459 | CB | ASN | B | 213 | 11.023 | −68.281 | 5.415 | 1 | 51.03 | B | C |
| ATOM | 1460 | CG | ASN | B | 213 | 10.238 | −69.509 | 5.862 | 1 | 54.07 | B | C |
| ATOM | 1461 | OD1 | ASN | B | 213 | 10.593 | −70.151 | 6.849 | 1 | 71.04 | B | O |
| ATOM | 1462 | ND2 | ASN | B | 213 | 9.15 | −69.8 | 5.182 | 1 | 59.22 | B | N |
| ATOM | 1463 | C | ASN | B | 213 | 12.493 | −66.4 | 6.108 | 1 | 44.58 | B | C |
| ATOM | 1464 | O | ASN | B | 213 | 11.878 | −65.372 | 5.933 | 1 | 52.29 | B | O |
| ATOM | 1465 | N | MET | B | 214 | 13.804 | −66.515 | 5.942 | 1 | 44.41 | B | N |
| ATOM | 1466 | CA | MET | B | 214 | 14.648 | −65.436 | 5.451 | 1 | 47.37 | B | C |
| ATOM | 1467 | CB | MET | B | 214 | 15.526 | −64.867 | 6.569 | 1 | 54.35 | B | C |
| ATOM | 1468 | CG | MET | B | 214 | 14.767 | −64.394 | 7.773 | 1 | 61.23 | B | C |
| ATOM | 1469 | SD | MET | B | 214 | 13.69 | −63.064 | 7.235 | 1 | 71.27 | B | S |
| ATOM | 1470 | CE | MET | B | 214 | 12.336 | −63.241 | 8.402 | 1 | 76.54 | B | C |
| ATOM | 1471 | C | MET | B | 214 | 15.559 | −66.004 | 4.409 | 1 | 41.8 | B | C |
| ATOM | 1472 | O | MET | B | 214 | 15.947 | −67.185 | 4.505 | 1 | 42.11 | B | O |
| ATOM | 1473 | N | VAL | B | 215 | 15.952 | −65.16 | 3.455 | 1 | 38.97 | B | N |
| ATOM | 1474 | CA | VAL | B | 215 | 17.019 | −65.527 | 2.509 | 1 | 39.26 | B | C |
| ATOM | 1475 | CB | VAL | B | 215 | 16.545 | −65.327 | 1.033 | 1 | 37.45 | B | C |
| ATOM | 1476 | CG1 | VAL | B | 215 | 17.626 | −65.773 | 0.049 | 1 | 35.85 | B | C |
| ATOM | 1477 | CG2 | VAL | B | 215 | 15.246 | −66.088 | 0.784 | 1 | 38.92 | B | C |
| ATOM | 1478 | C | VAL | B | 215 | 18.299 | −64.714 | 2.783 | 1 | 39.17 | B | C |
| ATOM | 1479 | O | VAL | B | 215 | 18.308 | −63.499 | 2.56 | 1 | 40.63 | B | O |
| ATOM | 1480 | N | PRO | B | 216 | 19.374 | −65.329 | 3.261 | 1 | 40.45 | B | N |
| ATOM | 1481 | CA | PRO | B | 216 | 19.405 | −66.651 | 3.881 | 1 | 39.85 | B | C |
| ATOM | 1482 | CB | PRO | B | 216 | 20.894 | −66.996 | 3.9 | 1 | 39.29 | B | C |
| ATOM | 1483 | CG | PRO | B | 216 | 21.562 | −65.661 | 4.029 | 1 | 38.91 | B | C |
| ATOM | 1484 | CD | PRO | B | 216 | 20.696 | −64.665 | 3.305 | 1 | 38.69 | B | C |
| ATOM | 1485 | C | PRO | B | 216 | 18.943 | −66.55 | 5.312 | 1 | 38.35 | B | C |
| ATOM | 1486 | O | PRO | B | 216 | 18.862 | −65.42 | 5.843 | 1 | 35.46 | B | O |
| ATOM | 1487 | N | PRO | B | 217 | 18.659 | −67.707 | 5.936 | 1 | 37.06 | B | N |
| ATOM | 1488 | CA | PRO | B | 217 | 18.389 | −67.725 | 7.346 | 1 | 41.55 | B | C |
| ATOM | 1489 | CB | PRO | B | 217 | 18.232 | −69.217 | 7.678 | 1 | 40.56 | B | C |
| ATOM | 1490 | CG | PRO | B | 217 | 18.018 | −69.895 | 6.383 | 1 | 37.84 | B | C |
| ATOM | 1491 | CD | PRO | B | 217 | 18.758 | −69.073 | 5.388 | 1 | 39.17 | B | C |
| ATOM | 1492 | C | PRO | B | 217 | 19.576 | −67.195 | 8.101 | 1 | 41.29 | B | C |
| ATOM | 1493 | O | PRO | B | 217 | 20.685 | −67.537 | 7.786 | 1 | 42.44 | B | O |
| ATOM | 1494 | N | PRO | B | 218 | 19.352 | −66.357 | 9.101 | 1 | 42.2 | B | N |
| ATOM | 1495 | CA | PRO | B | 218 | 20.5 | −66.017 | 9.964 | 1 | 42.35 | B | C |
| ATOM | 1496 | CB | PRO | B | 218 | 19.912 | −65.029 | 10.934 | 1 | 39.93 | B | C |
| ATOM | 1497 | CG | PRO | B | 218 | 18.498 | −65.42 | 11.034 | 1 | 43.97 | B | C |
| ATOM | 1498 | CD | PRO | B | 218 | 18.084 | −65.897 | 9.662 | 1 | 43.63 | B | C |
| ATOM | 1499 | C | PRO | B | 218 | 21.066 | −67.226 | 10.733 | 1 | 38.6 | B | C |
| ATOM | 1500 | O | PRO | B | 218 | 20.371 | −68.194 | 10.957 | 1 | 38.82 | B | O |
| ATOM | 1501 | N | THR | B | 219 | 22.315 | −67.153 | 11.155 | 1 | 41.75 | B | N |
| ATOM | 1502 | CA | THR | B | 219 | 22.804 | −68.159 | 12.132 | 1 | 48.26 | B | C |
| ATOM | 1503 | CB | THR | B | 219 | 24.3 | −68.045 | 12.45 | 1 | 47.5 | B | C |
| ATOM | 1504 | OG1 | THR | B | 219 | 24.478 | −66.901 | 13.295 | 1 | 53.94 | B | O |
| ATOM | 1505 | CG2 | THR | B | 219 | 25.161 | −67.94 | 11.171 | 1 | 49.45 | B | C |
| ATOM | 1506 | C | THR | B | 219 | 22.073 | −67.969 | 13.491 | 1 | 44.27 | B | C |
| ATOM | 1507 | O | THR | B | 219 | 21.701 | −66.849 | 13.879 | 1 | 40.81 | B | O |
| ATOM | 1508 | N | CYS | B | 220 | 21.874 | −69.057 | 14.204 | 1 | 43.12 | B | N |
| ATOM | 1509 | CA | CYS | B | 220 | 21.302 | −68.984 | 15.564 | 1 | 49.09 | B | C |
| ATOM | 1510 | CB | CYS | B | 220 | 21.106 | −70.392 | 16.123 | 1 | 49.08 | B | C |
| ATOM | 1511 | SG | CYS | B | 220 | 20.04 | −71.439 | 15.065 | 1 | 52.72 | B | S |
| ATOM | 1512 | C | CYS | B | 220 | 22.135 | −68.088 | 16.528 | 1 | 49.31 | B | C |
| ATOM | 1513 | O | CYS | B | 220 | 21.556 | −67.36 | 17.341 | 1 | 46.31 | B | O |
| ATOM | 1514 | N | LEU | B | 221 | 23.464 | −68.076 | 16.382 | 1 | 47.69 | B | N |
| ATOM | 1515 | CA | LEU | B | 221 | 24.304 | −67.158 | 17.153 | 1 | 51.5 | B | C |
| ATOM | 1516 | CB | LEU | B | 221 | 25.778 | −67.328 | 16.787 | 1 | 54.75 | B | C |
| ATOM | 1517 | CG | LEU | B | 221 | 26.424 | −68.698 | 17.048 | 1 | 61.07 | B | C |
| ATOM | 1518 | CD1 | LEU | B | 221 | 27.93 | −68.535 | 17.101 | 1 | 67.76 | B | C |
| ATOM | 1519 | CD2 | LEU | B | 221 | 25.975 | −69.387 | 18.32 | 1 | 62.43 | B | C |
| ATOM | 1520 | C | LEU | B | 221 | 23.922 | −65.703 | 16.896 | 1 | 53.46 | B | C |
| ATOM | 1521 | O | LEU | B | 221 | 23.772 | −64.88 | 17.819 | 1 | 51.1 | B | O |
| ATOM | 1522 | N | SER | B | 222 | 23.766 | −65.399 | 15.619 | 1 | 47.09 | B | N |
| ATOM | 1523 | CA | SER | B | 222 | 23.421 | −64.074 | 15.204 | 1 | 49.79 | B | C |
| ATOM | 1524 | CB | SER | B | 222 | 23.438 | −64.03 | 13.687 | 1 | 51.28 | B | C |
| ATOM | 1525 | OG | SER | B | 222 | 23.087 | −62.738 | 13.252 | 1 | 62.16 | B | O |
| ATOM | 1526 | C | SER | B | 222 | 22.04 | −63.682 | 15.757 | 1 | 55.49 | B | C |
| ATOM | 1527 | O | SER | B | 222 | 21.797 | −62.517 | 16.099 | 1 | 48.23 | B | O |
| ATOM | 1528 | N | VAL | B | 223 | 21.151 | −64.675 | 15.854 | 1 | 51.98 | B | N |
| ATOM | 1529 | CA | VAL | B | 223 | 19.821 | −64.478 | 16.411 | 1 | 55.43 | B | C |
| ATOM | 1530 | CB | VAL | B | 223 | 18.917 | −65.72 | 16.133 | 1 | 54.4 | B | C |
| ATOM | 1531 | CG1 | VAL | B | 223 | 17.837 | −65.912 | 17.204 | 1 | 52.12 | B | C |
| ATOM | 1532 | CG2 | VAL | B | 223 | 18.313 | −65.608 | 14.726 | 1 | 54.49 | B | C |
| ATOM | 1533 | C | VAL | B | 223 | 19.863 | −64.13 | 17.909 | 1 | 51.19 | B | C |
| ATOM | 1534 | O | VAL | B | 223 | 19.207 | −63.179 | 18.359 | 1 | 50.51 | B | O |
| ATOM | 1535 | N | ILE | B | 224 | 20.586 | −64.918 | 18.679 | 1 | 50.34 | B | N |
| ATOM | 1536 | CA | ILE | B | 224 | 20.663 | −64.651 | 20.118 | 1 | 57.69 | B | C |
| ATOM | 1537 | CB | ILE | B | 224 | 21.334 | −65.782 | 20.905 | 1 | 57.66 | B | C |
| ATOM | 1538 | CG1 | ILE | B | 224 | 20.28 | −66.826 | 21.232 | 1 | 58.47 | B | C |
| ATOM | 1539 | CD1 | ILE | B | 224 | 20.151 | −67.791 | 20.122 | 1 | 62.49 | B | C |
| ATOM | 1540 | CG2 | ILE | B | 224 | 21.931 | −65.295 | 22.222 | 1 | 60.62 | B | C |
| ATOM | 1541 | C | ILE | B | 224 | 21.321 | −63.305 | 20.391 | 1 | 55.25 | B | C |
| ATOM | 1542 | O | ILE | B | 224 | 20.786 | −62.52 | 21.148 | 1 | 52.45 | B | O |
| ATOM | 1543 | N | ARG | B | 225 | 22.43 | −63.037 | 19.727 | 1 | 57.04 | B | N |
| ATOM | 1544 | CA | ARG | B | 225 | 23.126 | −61.775 | 19.926 | 1 | 61.8 | B | C |
| ATOM | 1545 | CB | ARG | B | 225 | 24.487 | −61.757 | 19.218 | 1 | 62.73 | B | C |
| ATOM | 1546 | CG | ARG | B | 225 | 25.489 | −62.614 | 20.002 | 1 | 66.98 | B | C |
| ATOM | 1547 | CD | ARG | B | 225 | 26.751 | −62.946 | 19.243 | 1 | 69.61 | B | C |
| ATOM | 1548 | NE | ARG | B | 225 | 27.349 | −64.187 | 19.728 | 1 | 73.27 | B | N |
| ATOM | 1549 | CZ | ARG | B | 225 | 28.253 | −64.909 | 19.061 | 1 | 76.17 | B | C |
| ATOM | 1550 | NH1 | ARG | B | 225 | 28.733 | −66.015 | 19.606 | 1 | 75.26 | B | N |
| ATOM | 1551 | NH2 | ARG | B | 225 | 28.682 | −64.545 | 17.854 | 1 | 79.05 | B | N |
| ATOM | 1552 | C | ARG | B | 225 | 22.275 | −60.593 | 19.578 | 1 | 58.51 | B | C |
| ATOM | 1553 | O | ARG | B | 225 | 22.108 | −59.697 | 20.406 | 1 | 62.26 | B | O |
| ATOM | 1554 | N | SER | B | 226 | 21.676 | −60.616 | 18.397 | 1 | 60.94 | B | N |
| ATOM | 1555 | CA | SER | B | 226 | 20.847 | −59.488 | 17.941 | 1 | 57.22 | B | C |
| ATOM | 1556 | CB | SER | B | 226 | 20.294 | −59.778 | 16.534 | 1 | 57.35 | B | C |
| ATOM | 1557 | OG | SER | B | 226 | 19.201 | −58.933 | 16.231 | 1 | 63.61 | B | O |
| ATOM | 1558 | C | SER | B | 226 | 19.731 | −59.193 | 18.971 | 1 | 55.74 | B | C |
| ATOM | 1559 | O | SER | B | 226 | 19.383 | −58.041 | 19.226 | 1 | 56.31 | B | O |
| ATOM | 1560 | N | CYS | B | 227 | 19.217 | −60.25 | 19.587 | 1 | 50.68 | B | N |
| ATOM | 1561 | CA | CYS | B | 227 | 18.222 | −60.127 | 20.634 | 1 | 57.7 | B | C |
| ATOM | 1562 | CB | CYS | B | 227 | 17.735 | −61.513 | 21.054 | 1 | 55 | B | C |
| ATOM | 1563 | SG | CYS | B | 227 | 16.422 | −61.442 | 22.266 | 1 | 59.65 | B | S |
| ATOM | 1564 | C | CYS | B | 227 | 18.763 | −59.421 | 21.872 | 1 | 60.4 | B | C |
| ATOM | 1565 | O | CYS | B | 227 | 18.096 | −58.535 | 22.419 | 1 | 59.19 | B | O |
| ATOM | 1566 | N | GLN | B | 228 | 19.96 | −59.834 | 22.303 | 1 | 59.75 | B | N |
| ATOM | 1567 | CA | GLN | B | 228 | 20.642 | −59.216 | 23.466 | 1 | 63.98 | B | C |
| ATOM | 1568 | CB | GLN | B | 228 | 22.051 | −59.764 | 23.75 | 1 | 65.86 | B | C |
| ATOM | 1569 | CG | GLN | B | 228 | 22.346 | −61.271 | 23.698 | 1 | 71.33 | B | C |
| ATOM | 1570 | CD | GLN | B | 228 | 21.696 | −62.106 | 24.79 | 1 | 72.08 | B | C |
| ATOM | 1571 | OE1 | GLN | B | 228 | 20.603 | −61.783 | 25.265 | 1 | 75.94 | B | O |
| ATOM | 1572 | NE2 | GLN | B | 228 | 22.363 | −63.217 | 25.177 | 1 | 68.73 | B | N |
| ATOM | 1573 | C | GLN | B | 228 | 20.788 | −57.699 | 23.296 | 1 | 65.31 | B | C |
| ATOM | 1574 | O | GLN | B | 228 | 20.733 | −56.984 | 24.288 | 1 | 63.24 | B | O |
| ATOM | 1575 | N | ASN | B | 229 | 20.96 | −57.217 | 22.057 | 1 | 64.32 | B | N |
| ATOM | 1576 | CA | ASN | B | 229 | 21.12 | −55.763 | 21.785 | 1 | 66.24 | B | C |
| ATOM | 1577 | CB | ASN | B | 229 | 21.998 | −55.507 | 20.547 | 1 | 66.43 | B | C |
| ATOM | 1578 | CG | ASN | B | 229 | 23.294 | −56.284 | 20.574 | 1 | 72.5 | B | C |
| ATOM | 1579 | OD1 | ASN | B | 229 | 23.814 | −56.62 | 21.643 | 1 | 78.79 | B | O |
| ATOM | 1580 | ND2 | ASN | B | 229 | 23.82 | −56.59 | 19.39 | 1 | 76.78 | B | N |
| ATOM | 1581 | C | ASN | B | 229 | 19.832 | −54.981 | 21.595 | 1 | 60.27 | B | C |
| ATOM | 1582 | O | ASN | B | 229 | 19.895 | −53.845 | 21.181 | 1 | 61.72 | B | O |
| ATOM | 1583 | N | ASP | B | 230 | 18.678 | −55.58 | 21.851 | 1 | 64.23 | B | N |
| ATOM | 1584 | CA | ASP | B | 230 | 17.404 | −54.854 | 21.818 | 1 | 69.88 | B | C |
| ATOM | 1585 | CB | ASP | B | 230 | 16.466 | −55.5 | 20.789 | 1 | 70.79 | B | C |
| ATOM | 1586 | CG | ASP | B | 230 | 15.059 | −54.94 | 20.855 | 1 | 69.9 | B | C |
| ATOM | 1587 | OD1 | ASP | B | 230 | 14.886 | −53.817 | 20.378 | 1 | 75.3 | B | O |
| ATOM | 1588 | OD2 | ASP | B | 230 | 14.131 | −55.598 | 21.395 | 1 | 68.56 | B | O |
| ATOM | 1589 | C | ASP | B | 230 | 16.794 | −54.926 | 23.215 | 1 | 73.46 | B | C |
| ATOM | 1590 | O | ASP | B | 230 | 16.878 | −55.983 | 23.856 | 1 | 72.37 | B | O |
| ATOM | 1591 | N | GLU | B | 231 | 16.172 | −53.826 | 23.672 | 1 | 77.53 | B | N |
| ATOM | 1592 | CA | GLU | B | 231 | 15.547 | −53.759 | 25.023 | 1 | 78.12 | B | C |
| ATOM | 1593 | CB | GLU | B | 231 | 14.932 | −52.38 | 25.282 | 1 | 82.98 | B | C |
| ATOM | 1594 | CG | GLU | B | 231 | 15.89 | −51.354 | 25.879 | 1 | 98.09 | B | C |
| ATOM | 1595 | CD | GLU | B | 231 | 15.236 | −50.505 | 26.963 | 1 | 101.71 | B | C |
| ATOM | 1596 | OE1 | GLU | B | 231 | 14.782 | −51.09 | 27.967 | 1 | 96.89 | B | O |
| ATOM | 1597 | OE2 | GLU | B | 231 | 15.179 | −49.261 | 26.817 | 1 | 101.61 | B | O |
| ATOM | 1598 | C | GLU | B | 231 | 14.465 | −54.831 | 25.304 | 1 | 74.28 | B | C |
| ATOM | 1599 | O | GLU | B | 231 | 14.634 | −55.702 | 26.184 | 1 | 63.5 | B | O |
| ATOM | 1600 | N | LEU | B | 232 | 13.371 | −54.755 | 24.549 | 1 | 63.39 | B | N |
| ATOM | 1601 | CA | LEU | B | 232 | 12.25 | −55.673 | 24.695 | 1 | 61.1 | B | C |
| ATOM | 1602 | CB | LEU | B | 232 | 11.137 | −55.247 | 23.727 | 1 | 65.21 | B | C |
| ATOM | 1603 | CG | LEU | B | 232 | 9.772 | −55.957 | 23.686 | 1 | 71.8 | B | C |
| ATOM | 1604 | CD1 | LEU | B | 232 | 8.72 | −54.963 | 23.21 | 1 | 72.13 | B | C |
| ATOM | 1605 | CD2 | LEU | B | 232 | 9.758 | −57.201 | 22.785 | 1 | 71.7 | B | C |
| ATOM | 1606 | C | LEU | B | 232 | 12.673 | −57.145 | 24.473 | 1 | 59.97 | B | C |
| ATOM | 1607 | O | LEU | B | 232 | 12.348 | −58.022 | 25.288 | 1 | 63.34 | B | O |
| ATOM | 1608 | N | CYS | B | 233 | 13.414 | −57.418 | 23.398 | 1 | 56.46 | B | N |
| ATOM | 1609 | CA | CYS | B | 233 | 13.854 | −58.79 | 23.132 | 1 | 56.16 | B | C |
| ATOM | 1610 | CB | CYS | B | 233 | 14.601 | −58.933 | 21.793 | 1 | 52.64 | B | C |
| ATOM | 1611 | SG | CYS | B | 233 | 14.738 | −60.677 | 21.271 | 1 | 55.85 | B | S |
| ATOM | 1612 | C | CYS | B | 233 | 14.697 | −59.373 | 24.294 | 1 | 58.07 | B | C |
| ATOM | 1613 | O | CYS | B | 233 | 14.414 | −60.498 | 24.75 | 1 | 52.08 | B | O |
| ATOM | 1614 | N | ARG | B | 234 | 15.705 | −58.618 | 24.76 | 1 | 58.38 | B | N |
| ATOM | 1615 | CA | ARG | B | 234 | 16.612 | −59.075 | 25.847 | 1 | 59.04 | B | C |
| ATOM | 1616 | CB | ARG | B | 234 | 17.627 | −57.989 | 26.223 | 1 | 63.8 | B | C |
| ATOM | 1617 | CG | ARG | B | 234 | 18.612 | −58.327 | 27.364 | 1 | 69.14 | B | C |
| ATOM | 1618 | CD | ARG | B | 234 | 19.167 | −57.049 | 27.998 | 1 | 72.12 | B | C |
| ATOM | 1619 | NE | ARG | B | 234 | 19.563 | −56.118 | 26.928 | 1 | 79.05 | B | N |
| ATOM | 1620 | CZ | ARG | B | 234 | 19.202 | −54.837 | 26.781 | 1 | 75.36 | B | C |
| ATOM | 1621 | NH1 | ARG | B | 234 | 19.661 | −54.16 | 25.727 | 1 | 73.44 | B | N |
| ATOM | 1622 | NH2 | ARG | B | 234 | 18.423 | −54.206 | 27.663 | 1 | 76.39 | B | N |
| ATOM | 1623 | C | ARG | B | 234 | 15.806 | −59.484 | 27.071 | 1 | 60.41 | B | C |
| ATOM | 1624 | O | ARG | B | 234 | 15.963 | −60.61 | 27.582 | 1 | 57.03 | B | O |
| ATOM | 1625 | N | ARG | B | 235 | 14.9 | −58.612 | 27.505 | 1 | 57.02 | B | N |
| ATOM | 1626 | CA | ARG | B | 235 | 14.072 | −58.951 | 28.659 | 1 | 66.3 | B | C |
| ATOM | 1627 | CB | ARG | B | 235 | 13.117 | −57.813 | 29.008 | 1 | 79.61 | B | C |
| ATOM | 1628 | CG | ARG | B | 235 | 13.789 | −56.674 | 29.763 | 1 | 89.89 | B | C |
| ATOM | 1629 | CD | ARG | B | 235 | 13.247 | −55.328 | 29.318 | 1 | 94.35 | B | C |
| ATOM | 1630 | NE | ARG | B | 235 | 13.547 | −54.263 | 30.267 | 1 | 102.33 | B | N |
| ATOM | 1631 | CZ | ARG | B | 235 | 13.286 | −52.969 | 30.069 | 1 | 103.92 | B | C |
| ATOM | 1632 | NH1 | ARG | B | 235 | 12.727 | −52.54 | 28.929 | 1 | 97.07 | B | N |
| ATOM | 1633 | NH2 | ARG | B | 235 | 13.594 | −52.088 | 31.022 | 1 | 97.99 | B | N |
| ATOM | 1634 | C | ARG | B | 235 | 13.284 | −60.221 | 28.416 | 1 | 61.7 | B | C |
| ATOM | 1635 | O | ARG | B | 235 | 13.244 | −61.102 | 29.271 | 1 | 57.45 | B | O |
| ATOM | 1636 | N | HIS | B | 236 | 12.674 | −60.325 | 27.235 | 1 | 57.83 | B | N |
| ATOM | 1637 | CA | HIS | B | 236 | 11.803 | −61.466 | 26.965 | 1 | 52.2 | B | C |
| ATOM | 1638 | CB | HIS | B | 236 | 10.859 | −61.186 | 25.776 | 1 | 57.98 | B | C |
| ATOM | 1639 | CG | HIS | B | 236 | 9.747 | −60.261 | 26.139 | 1 | 63.41 | B | C |
| ATOM | 1640 | ND1 | HIS | B | 236 | 9.722 | −58.938 | 25.761 | 1 | 71.46 | B | N |
| ATOM | 1641 | CE1 | HIS | B | 236 | 8.648 | −58.357 | 26.265 | 1 | 69.43 | B | C |
| ATOM | 1642 | NE2 | HIS | B | 236 | 8 | −59.246 | 26.994 | 1 | 76.52 | B | N |
| ATOM | 1643 | CD2 | HIS | B | 236 | 8.676 | −60.441 | 26.943 | 1 | 73.54 | B | C |
| ATOM | 1644 | C | HIS | B | 236 | 12.594 | −62.747 | 26.817 | 1 | 43.46 | B | C |
| ATOM | 1645 | O | HIS | B | 236 | 12.14 | −63.785 | 27.237 | 1 | 39.42 | B | O |
| ATOM | 1646 | N | TYR | B | 237 | 13.791 | −62.666 | 26.24 | 1 | 44.69 | B | N |
| ATOM | 1647 | CA | TYR | B | 237 | 14.661 | −63.827 | 26.188 | 1 | 48.16 | B | C |
| ATOM | 1648 | CB | TYR | B | 237 | 15.941 | −63.544 | 25.389 | 1 | 43.81 | B | C |
| ATOM | 1649 | CG | TYR | B | 237 | 16.795 | −64.789 | 25.305 | 1 | 50.24 | B | C |
| ATOM | 1650 | CD1 | TYR | B | 237 | 16.295 | −65.953 | 24.695 | 1 | 48.61 | B | C |
| ATOM | 1651 | CE1 | TYR | B | 237 | 17.06 | −67.116 | 24.635 | 1 | 51.92 | B | C |
| ATOM | 1652 | CZ | TYR | B | 237 | 18.34 | −67.14 | 25.21 | 1 | 53.26 | B | C |
| ATOM | 1653 | OH | TYR | B | 237 | 19.075 | −68.316 | 25.154 | 1 | 60.59 | B | O |
| ATOM | 1654 | CE2 | TYR | B | 237 | 18.846 | −66.011 | 25.841 | 1 | 50.02 | B | C |
| ATOM | 1655 | CD2 | TYR | B | 237 | 18.084 | −64.84 | 25.887 | 1 | 50.95 | B | C |
| ATOM | 1656 | C | TYR | B | 237 | 14.993 | −64.3 | 27.623 | 1 | 50.61 | B | C |
| ATOM | 1657 | O | TYR | B | 237 | 14.859 | −65.503 | 27.948 | 1 | 45.82 | B | O |
| ATOM | 1658 | N | ARG | B | 238 | 15.373 | −63.34 | 28.476 | 1 | 52.59 | B | N |
| ATOM | 1659 | CA | ARG | B | 238 | 15.795 | −63.625 | 29.866 | 1 | 52.11 | B | C |
| ATOM | 1660 | CB | ARG | B | 238 | 16.094 | −62.309 | 30.606 | 1 | 57.58 | B | C |
| ATOM | 1661 | CG | ARG | B | 238 | 16.975 | −62.4 | 31.867 | 1 | 70.39 | B | C |
| ATOM | 1662 | CD | ARG | B | 238 | 18.068 | −61.316 | 31.891 | 1 | 76.46 | B | C |
| ATOM | 1663 | NE | ARG | B | 238 | 17.553 | −59.937 | 31.755 | 1 | 84.04 | B | N |
| ATOM | 1664 | CZ | ARG | B | 238 | 18.248 | −58.876 | 31.302 | 1 | 92.92 | B | C |
| ATOM | 1665 | NH1 | ARG | B | 238 | 17.66 | −57.673 | 31.238 | 1 | 89.77 | B | N |
| ATOM | 1666 | NH2 | ARG | B | 238 | 19.522 | −58.989 | 30.898 | 1 | 92.96 | B | N |
| ATOM | 1667 | C | ARG | B | 238 | 14.697 | −64.428 | 30.55 | 1 | 50.23 | B | C |
| ATOM | 1668 | O | ARG | B | 238 | 14.95 | −65.515 | 31.085 | 1 | 50.31 | B | O |
| ATOM | 1669 | N | THR | B | 239 | 13.462 | −63.924 | 30.48 | 1 | 45.71 | B | N |
| ATOM | 1670 | CA | THR | B | 239 | 12.336 | −64.608 | 31.089 | 1 | 44.91 | B | C |
| ATOM | 1671 | CB | THR | B | 239 | 11.055 | −63.823 | 30.876 | 1 | 48.93 | B | C |
| ATOM | 1672 | OG1 | THR | B | 239 | 11.294 | −62.457 | 31.197 | 1 | 54.27 | B | O |
| ATOM | 1673 | CG2 | THR | B | 239 | 9.904 | −64.371 | 31.712 | 1 | 47.59 | B | C |
| ATOM | 1674 | C | THR | B | 239 | 12.138 | −65.986 | 30.498 | 1 | 54.66 | B | C |
| ATOM | 1675 | O | THR | B | 239 | 11.867 | −66.967 | 31.233 | 1 | 56.96 | B | O |
| ATOM | 1676 | N | PHE | B | 240 | 12.242 | −66.046 | 29.165 | 1 | 48.48 | B | N |
| ATOM | 1677 | CA | PHE | B | 240 | 12.064 | −67.27 | 28.451 | 1 | 47.12 | B | C |
| ATOM | 1678 | CB | PHE | B | 240 | 12.255 | −67.039 | 26.945 | 1 | 47.04 | B | C |
| ATOM | 1679 | CG | PHE | B | 240 | 12.375 | −68.308 | 26.141 | 1 | 44.93 | B | C |
| ATOM | 1680 | CD1 | PHE | B | 240 | 11.29 | −69.162 | 26.013 | 1 | 43.59 | B | C |
| ATOM | 1681 | CE1 | PHE | B | 240 | 11.376 | −70.317 | 25.235 | 1 | 46.67 | B | C |
| ATOM | 1682 | CZ | PHE | B | 240 | 12.555 | −70.63 | 24.592 | 1 | 47.72 | B | C |
| ATOM | 1683 | CE2 | PHE | B | 240 | 13.649 | −69.78 | 24.717 | 1 | 46.71 | B | C |
| ATOM | 1684 | CD2 | PHE | B | 240 | 13.559 | −68.634 | 25.493 | 1 | 44.77 | B | C |
| ATOM | 1685 | C | PHE | B | 240 | 13.02 | −68.333 | 28.935 | 1 | 46.87 | B | C |
| ATOM | 1686 | O | PHE | B | 240 | 12.609 | −69.47 | 29.203 | 1 | 47.31 | B | O |
| ATOM | 1687 | N | GLN | B | 241 | 14.296 | −67.959 | 28.957 | 1 | 49.9 | B | N |
| ATOM | 1688 | CA | GLN | B | 241 | 15.378 | −68.788 | 29.52 | 1 | 56.37 | B | C |
| ATOM | 1689 | CB | GLN | B | 241 | 16.703 | −68.026 | 29.52 | 1 | 58.66 | B | C |
| ATOM | 1690 | CG | GLN | B | 241 | 17.413 | −68.01 | 28.183 | 1 | 70.29 | B | C |
| ATOM | 1691 | CD | GLN | B | 241 | 17.812 | −69.391 | 27.723 | 1 | 80.28 | B | C |
| ATOM | 1692 | OE1 | GLN | B | 241 | 17.539 | −69.808 | 26.565 | 1 | 79.86 | B | O |
| ATOM | 1693 | NE2 | GLN | B | 241 | 18.451 | −70.126 | 28.624 | 1 | 82.86 | B | N |
| ATOM | 1694 | C | GLN | B | 241 | 15.107 | −69.272 | 30.937 | 1 | 52.32 | B | C |
| ATOM | 1695 | O | GLN | B | 241 | 15.297 | −70.445 | 31.234 | 1 | 50.25 | B | O |
| ATOM | 1696 | N | SER | B | 242 | 14.686 | −68.358 | 31.799 | 1 | 55.28 | B | N |
| ATOM | 1697 | CA | SER | B | 242 | 14.368 | −68.708 | 33.173 | 1 | 65.14 | B | C |
| ATOM | 1698 | CB | SER | B | 242 | 13.923 | −67.476 | 33.962 | 1 | 65.22 | B | C |
| ATOM | 1699 | OG | SER | B | 242 | 14.894 | −66.471 | 33.845 | 1 | 65.15 | B | O |
| ATOM | 1700 | C | SER | B | 242 | 13.295 | −69.799 | 33.256 | 1 | 69.35 | B | C |
| ATOM | 1701 | O | SER | B | 242 | 13.487 | −70.799 | 33.947 | 1 | 77.41 | B | O |
| ATOM | 1702 | N | LYS | B | 243 | 12.194 | −69.634 | 32.527 | 1 | 64.41 | B | N |
| ATOM | 1703 | CA | LYS | B | 243 | 11.041 | −70.506 | 32.725 | 1 | 59.29 | B | C |
| ATOM | 1704 | CB | LYS | B | 243 | 9.74 | −69.744 | 32.433 | 1 | 66.22 | B | C |
| ATOM | 1705 | CG | LYS | B | 243 | 9.51 | −68.509 | 33.308 | 1 | 73.5 | B | C |
| ATOM | 1706 | CD | LYS | B | 243 | 9.562 | −68.863 | 34.808 | 1 | 78.23 | B | C |
| ATOM | 1707 | CE | LYS | B | 243 | 9.345 | −67.666 | 35.722 | 1 | 86.66 | B | C |
| ATOM | 1708 | NZ | LYS | B | 243 | 8.068 | −67.796 | 36.483 | 1 | 96.67 | B | N |
| ATOM | 1709 | C | LYS | B | 243 | 11.114 | −71.799 | 31.946 | 1 | 56.26 | B | C |
| ATOM | 1710 | O | LYS | B | 243 | 10.514 | −72.787 | 32.332 | 1 | 62.26 | B | O |
| ATOM | 1711 | N | CYS | B | 244 | 11.844 | −71.806 | 30.847 | 1 | 55.61 | B | N |
| ATOM | 1712 | CA | CYS | B | 244 | 11.888 | −72.974 | 29.975 | 1 | 56.53 | B | C |
| ATOM | 1713 | CB | CYS | B | 244 | 11.679 | −72.529 | 28.494 | 1 | 59.59 | B | C |
| ATOM | 1714 | SG | CYS | B | 244 | 10.05 | −71.723 | 28.183 | 1 | 59.79 | B | S |
| ATOM | 1715 | C | CYS | B | 244 | 13.183 | −73.757 | 30.195 | 1 | 53.86 | B | C |
| ATOM | 1716 | O | CYS | B | 244 | 13.188 | −74.98 | 30.138 | 1 | 56.54 | B | O |
| ATOM | 1717 | N | TRP | B | 245 | 14.281 | −73.042 | 30.441 | 1 | 54.71 | B | N |
| ATOM | 1718 | CA | TRP | B | 245 | 15.603 | −73.621 | 30.514 | 1 | 55.17 | B | C |
| ATOM | 1719 | CB | TRP | B | 245 | 16.394 | −73.139 | 29.301 | 1 | 54.2 | B | C |
| ATOM | 1720 | CG | TRP | B | 245 | 15.687 | −73.481 | 28.011 | 1 | 52.88 | B | C |
| ATOM | 1721 | CD1 | TRP | B | 245 | 14.96 | −72.621 | 27.223 | 1 | 53.79 | B | C |
| ATOM | 1722 | NE1 | TRP | B | 245 | 14.446 | −73.292 | 26.136 | 1 | 52.15 | B | N |
| ATOM | 1723 | CE2 | TRP | B | 245 | 14.826 | −74.607 | 26.194 | 1 | 50.77 | B | C |
| ATOM | 1724 | CD2 | TRP | B | 245 | 15.61 | −74.772 | 27.366 | 1 | 51.74 | B | C |
| ATOM | 1725 | CE3 | TRP | B | 245 | 16.121 | −76.051 | 27.666 | 1 | 52.06 | B | C |
| ATOM | 1726 | CZ3 | TRP | B | 245 | 15.842 | −77.114 | 26.79 | 1 | 50.59 | B | C |
| ATOM | 1727 | CH2 | TRP | B | 245 | 15.051 | −76.917 | 25.647 | 1 | 53.71 | B | C |
| ATOM | 1728 | CZ2 | TRP | B | 245 | 14.524 | −75.671 | 25.338 | 1 | 51.5 | B | C |
| ATOM | 1729 | C | TRP | B | 245 | 16.235 | −73.242 | 31.881 | 1 | 57.41 | B | C |
| ATOM | 1730 | O | TRP | B | 245 | 17.279 | −72.583 | 31.951 | 1 | 60.76 | B | O |
| ATOM | 1731 | N | GLN | B | 246 | 15.547 | −73.651 | 32.96 | 1 | 61.29 | B | N |
| ATOM | 1732 | CA | GLN | B | 246 | 15.959 | −73.382 | 34.354 | 1 | 64.61 | B | C |
| ATOM | 1733 | CB | GLN | B | 246 | 15.007 | −74.083 | 35.34 | 1 | 73.7 | B | C |
| ATOM | 1734 | CG | GLN | B | 246 | 15.251 | −73.768 | 36.824 | 1 | 87.61 | B | C |
| ATOM | 1735 | CD | GLN | B | 246 | 15.382 | −75.022 | 37.712 | 1 | 99.98 | B | C |
| ATOM | 1736 | OE1 | GLN | B | 246 | 16.305 | −75.844 | 37.543 | 1 | 96.3 | B | O |
| ATOM | 1737 | NE2 | GLN | B | 246 | 14.468 | −75.163 | 38.679 | 1 | 101.16 | B | N |
| ATOM | 1738 | C | GLN | B | 246 | 17.408 | −73.829 | 34.617 | 1 | 56.51 | B | C |
| ATOM | 1739 | O | GLN | B | 246 | 18.2 | −73.047 | 35.145 | 1 | 57.89 | B | O |
| ATOM | 1740 | N | ARG | B | 247 | 17.748 | −75.057 | 34.207 | 1 | 50.96 | B | N |
| ATOM | 1741 | CA | ARG | B | 247 | 19.061 | −75.622 | 34.458 | 1 | 53.72 | B | C |
| ATOM | 1742 | CB | ARG | B | 247 | 19.175 | −77.065 | 34.011 | 1 | 61.5 | B | C |
| ATOM | 1743 | CG | ARG | B | 247 | 18.144 | −78.036 | 34.586 | 1 | 72.17 | B | C |
| ATOM | 1744 | CD | ARG | B | 247 | 18.741 | −79.447 | 34.651 | 1 | 82.68 | B | C |
| ATOM | 1745 | NE | ARG | B | 247 | 17.838 | −80.547 | 34.304 | 1 | 85.52 | B | N |
| ATOM | 1746 | CZ | ARG | B | 247 | 16.83 | −80.995 | 35.059 | 1 | 92.61 | B | C |
| ATOM | 1747 | NH1 | ARG | B | 247 | 16.511 | −80.401 | 36.216 | 1 | 91.54 | B | N |
| ATOM | 1748 | NH2 | ARG | B | 247 | 16.108 | −82.037 | 34.634 | 1 | 88.8 | B | N |
| ATOM | 1749 | C | ARG | B | 247 | 20.126 | −74.852 | 33.739 | 1 | 57.77 | B | C |
| ATOM | 1750 | O | ARG | B | 247 | 21.212 | −74.678 | 34.26 | 1 | 58.43 | B | O |
| ATOM | 1751 | N | VAL | B | 248 | 19.819 | −74.4 | 32.526 | 1 | 57.32 | B | N |
| ATOM | 1752 | CA | VAL | B | 248 | 20.779 | −73.626 | 31.736 | 1 | 55.31 | B | C |
| ATOM | 1753 | CB | VAL | B | 248 | 20.286 | −73.388 | 30.276 | 1 | 55.38 | B | C |
| ATOM | 1754 | CG1 | VAL | B | 248 | 21.155 | −72.369 | 29.537 | 1 | 52.27 | B | C |
| ATOM | 1755 | CG2 | VAL | B | 248 | 20.237 | −74.698 | 29.507 | 1 | 55.65 | B | C |
| ATOM | 1756 | C | VAL | B | 248 | 21.006 | −72.292 | 32.409 | 1 | 51.1 | B | C |
| ATOM | 1757 | O | VAL | B | 248 | 22.143 | −71.813 | 32.502 | 1 | 49.02 | B | O |
| ATOM | 1758 | N | THR | B | 249 | 19.923 | −71.669 | 32.843 | 1 | 48.56 | B | N |
| ATOM | 1759 | CA | THR | B | 249 | 20.045 | −70.334 | 33.44 | 1 | 54.69 | B | C |
| ATOM | 1760 | CB | THR | B | 249 | 18.664 | −69.713 | 33.67 | 1 | 52.47 | B | C |
| ATOM | 1761 | OG1 | THR | B | 249 | 18.017 | −69.582 | 32.397 | 1 | 56.62 | B | O |
| ATOM | 1762 | CG2 | THR | B | 249 | 18.779 | −68.352 | 34.334 | 1 | 55.42 | B | C |
| ATOM | 1763 | C | THR | B | 249 | 20.832 | −70.395 | 34.767 | 1 | 61.51 | B | C |
| ATOM | 1764 | O | THR | B | 249 | 21.72 | −69.568 | 34.994 | 1 | 63.58 | B | O |
| ATOM | 1765 | N | ARG | B | 250 | 20.519 | −71.382 | 35.613 | 1 | 61.06 | B | N |
| ATOM | 1766 | CA | ARG | B | 250 | 21.153 | −71.466 | 36.916 | 1 | 64.92 | B | C |
| ATOM | 1767 | CB | ARG | B | 250 | 20.343 | −72.35 | 37.872 | 1 | 69.17 | B | C |
| ATOM | 1768 | CG | ARG | B | 250 | 20.491 | −73.879 | 37.81 | 1 | 75.94 | B | C |
| ATOM | 1769 | CD | ARG | B | 250 | 19.602 | −74.516 | 38.902 | 1 | 85.03 | B | C |
| ATOM | 1770 | NE | ARG | B | 250 | 18.44 | −73.638 | 39.171 | 1 | 90.33 | B | N |
| ATOM | 1771 | CZ | ARG | B | 250 | 17.775 | −73.5 | 40.321 | 1 | 86.23 | B | C |
| ATOM | 1772 | NH1 | ARG | B | 250 | 18.041 | −74.247 | 41.389 | 1 | 83.37 | B | N |
| ATOM | 1773 | NH2 | ARG | B | 250 | 16.784 | −72.605 | 40.379 | 1 | 85.47 | B | N |
| ATOM | 1774 | C | ARG | B | 250 | 22.633 | −71.859 | 36.799 | 1 | 63.15 | B | C |
| ATOM | 1775 | O | ARG | B | 250 | 23.457 | −71.359 | 37.548 | 1 | 58.93 | B | O |
| ATOM | 1776 | N | LYS | B | 251 | 22.97 | −72.693 | 35.817 | 1 | 64.02 | B | N |
| ATOM | 1777 | CA | LYS | B | 251 | 24.343 | −73.147 | 35.637 | 1 | 58.66 | B | C |
| ATOM | 1778 | CB | LYS | B | 251 | 24.391 | −74.411 | 34.797 | 1 | 65.94 | B | C |
| ATOM | 1779 | CG | LYS | B | 251 | 25.216 | −75.515 | 35.403 | 1 | 79.06 | B | C |
| ATOM | 1780 | CD | LYS | B | 251 | 24.376 | −76.332 | 36.386 | 1 | 82.06 | B | C |
| ATOM | 1781 | CE | LYS | B | 251 | 23.651 | −77.463 | 35.676 | 1 | 86 | B | C |
| ATOM | 1782 | NZ | LYS | B | 251 | 24.621 | −78.449 | 35.127 | 1 | 84.52 | B | N |
| ATOM | 1783 | C | LYS | B | 251 | 25.187 | −72.068 | 34.963 | 1 | 58.16 | B | C |
| ATOM | 1784 | O | LYS | B | 251 | 26.35 | −71.885 | 35.308 | 1 | 52.69 | B | O |
| ATOM | 1785 | N | CYS | B | 252 | 24.618 | −71.351 | 33.988 | 1 | 61.02 | B | N |
| ATOM | 1786 | CA | CYS | B | 252 | 25.448 | −70.532 | 33.101 | 1 | 59.85 | B | C |
| ATOM | 1787 | CB | CYS | B | 252 | 25.4 | −71.051 | 31.67 | 1 | 59.08 | B | C |
| ATOM | 1788 | SG | CYS | B | 252 | 26.086 | −72.703 | 31.512 | 1 | 65.84 | B | S |
| ATOM | 1789 | C | CYS | B | 252 | 25.167 | −69.073 | 33.086 | 1 | 56.69 | B | C |
| ATOM | 1790 | O | CYS | B | 252 | 26.07 | −68.335 | 32.766 | 1 | 57.83 | B | O |
| ATOM | 1791 | N | HIS | B | 253 | 23.952 | −68.665 | 33.446 | 1 | 62.28 | B | N |
| ATOM | 1792 | CA | HIS | B | 253 | 23.429 | −67.329 | 33.121 | 1 | 69.15 | B | C |
| ATOM | 1793 | CB | HIS | B | 253 | 24.04 | −66.277 | 34.053 | 1 | 70.3 | B | C |
| ATOM | 1794 | CG | HIS | B | 253 | 23.658 | −66.509 | 35.482 | 1 | 83.02 | B | C |
| ATOM | 1795 | ND1 | HIS | B | 253 | 22.354 | −66.386 | 35.927 | 1 | 87.63 | B | N |
| ATOM | 1796 | CE1 | HIS | B | 253 | 22.299 | −66.707 | 37.206 | 1 | 85.12 | B | C |
| ATOM | 1797 | NE2 | HIS | B | 253 | 23.509 | −67.068 | 37.597 | 1 | 80.07 | B | N |
| ATOM | 1798 | CD2 | HIS | B | 253 | 24.375 | −66.965 | 36.537 | 1 | 77.72 | B | C |
| ATOM | 1799 | C | HIS | B | 253 | 23.501 | −67.031 | 31.603 | 1 | 71.88 | B | C |
| ATOM | 1800 | O | HIS | B | 253 | 22.916 | −67.79 | 30.817 | 1 | 66.3 | B | O |
| ATOM | 1801 | N | GLU | B | 254 | 24.207 | −65.985 | 31.185 | 1 | 77.82 | B | N |
| ATOM | 1802 | CA | GLU | B | 254 | 24.321 | −65.654 | 29.764 | 1 | 79 | B | C |
| ATOM | 1803 | CB | GLU | B | 254 | 23.894 | −64.189 | 29.569 | 1 | 85.87 | B | C |
| ATOM | 1804 | CG | GLU | B | 254 | 22.391 | −63.98 | 29.826 | 1 | 92.05 | B | C |
| ATOM | 1805 | CD | GLU | B | 254 | 21.966 | −62.524 | 30.038 | 1 | 102.75 | B | C |
| ATOM | 1806 | OE1 | GLU | B | 254 | 22.759 | −61.716 | 30.569 | 1 | 101.46 | B | O |
| ATOM | 1807 | OE2 | GLU | B | 254 | 20.808 | −62.184 | 29.7 | 1 | 102.92 | B | O |
| ATOM | 1808 | C | GLU | B | 254 | 25.734 | −65.96 | 29.217 | 1 | 78.28 | B | C |
| ATOM | 1809 | O | GLU | B | 254 | 26.071 | −65.593 | 28.1 | 1 | 80.04 | B | O |
| ATOM | 1810 | N | ASP | B | 255 | 26.541 | −66.689 | 29.989 | 1 | 79.32 | B | N |
| ATOM | 1811 | CA | ASP | B | 255 | 27.914 | −67.011 | 29.604 | 1 | 77.89 | B | C |
| ATOM | 1812 | CB | ASP | B | 255 | 28.718 | −67.492 | 30.817 | 1 | 80.03 | B | C |
| ATOM | 1813 | CG | ASP | B | 255 | 30.196 | −67.675 | 30.514 | 1 | 78.48 | B | C |
| ATOM | 1814 | OD1 | ASP | B | 255 | 30.56 | −68.574 | 29.724 | 1 | 70.82 | B | O |
| ATOM | 1815 | OD2 | ASP | B | 255 | 31.004 | −66.93 | 31.092 | 1 | 86.82 | B | O |
| ATOM | 1816 | C | ASP | B | 255 | 27.917 | −68.073 | 28.501 | 1 | 71.22 | B | C |
| ATOM | 1817 | O | ASP | B | 255 | 27.624 | −69.284 | 28.743 | 1 | 58.47 | B | O |
| ATOM | 1818 | N | GLU | B | 256 | 28.286 | −67.61 | 27.307 | 1 | 64.05 | B | N |
| ATOM | 1819 | CA | GLU | B | 256 | 28.216 | −68.428 | 26.093 | 1 | 63.27 | B | C |
| ATOM | 1820 | CB | GLU | B | 256 | 28.523 | −67.588 | 24.849 | 1 | 66.56 | B | C |
| ATOM | 1821 | CG | GLU | B | 256 | 27.55 | −66.427 | 24.619 | 1 | 66.96 | B | C |
| ATOM | 1822 | CD | GLU | B | 256 | 27.536 | −65.894 | 23.181 | 1 | 72.02 | B | C |
| ATOM | 1823 | OE1 | GLU | B | 256 | 26.817 | −64.901 | 22.892 | 1 | 69.46 | B | O |
| ATOM | 1824 | OE2 | GLU | B | 256 | 28.229 | −66.468 | 22.323 | 1 | 66.55 | B | O |
| ATOM | 1825 | C | GLU | B | 256 | 29.127 | −69.649 | 26.187 | 1 | 61.08 | B | C |
| ATOM | 1826 | O | GLU | B | 256 | 28.695 | −70.767 | 25.874 | 1 | 54.96 | B | O |
| ATOM | 1827 | N | ASN | B | 257 | 30.337 | −69.441 | 26.715 | 1 | 60.82 | B | N |
| ATOM | 1828 | CA | ASN | B | 257 | 31.34 | −70.526 | 26.936 | 1 | 67.28 | B | C |
| ATOM | 1829 | CB | ASN | B | 257 | 32.646 | −69.949 | 27.501 | 1 | 74.03 | B | C |
| ATOM | 1830 | CG | ASN | B | 257 | 32.968 | −68.566 | 26.94 | 1 | 83.48 | B | C |
| ATOM | 1831 | OD1 | ASN | B | 257 | 32.145 | −67.637 | 27.037 | 1 | 84.17 | B | O |
| ATOM | 1832 | ND2 | ASN | B | 257 | 34.146 | −68.419 | 26.349 | 1 | 85.42 | B | N |
| ATOM | 1833 | C | ASN | B | 257 | 30.844 | −71.617 | 27.877 | 1 | 63.6 | B | C |
| ATOM | 1834 | O | ASN | B | 257 | 30.998 | −72.837 | 27.632 | 1 | 59.11 | B | O |
| ATOM | 1835 | N | CYS | B | 258 | 30.235 | −71.161 | 28.966 | 1 | 62.33 | B | N |
| ATOM | 1836 | CA | CYS | B | 258 | 29.546 | −72.054 | 29.863 | 1 | 61.37 | B | C |
| ATOM | 1837 | CB | CYS | B | 258 | 28.931 | −71.33 | 31.073 | 1 | 63.61 | B | C |
| ATOM | 1838 | SG | CYS | B | 258 | 28.067 | −72.514 | 32.15 | 1 | 67.39 | B | S |
| ATOM | 1839 | C | CYS | B | 258 | 28.469 | −72.756 | 29.073 | 1 | 59.91 | B | C |
| ATOM | 1840 | O | CYS | B | 258 | 28.398 | −73.98 | 29.125 | 1 | 64.46 | B | O |
| ATOM | 1841 | N | ILE | B | 259 | 27.627 | −72 | 28.346 | 1 | 59.37 | B | N |
| ATOM | 1842 | CA | ILE | B | 259 | 26.481 | −72.636 | 27.633 | 1 | 59.51 | B | C |
| ATOM | 1843 | CB | ILE | B | 259 | 25.542 | −71.616 | 26.926 | 1 | 63.22 | B | C |
| ATOM | 1844 | CG1 | ILE | B | 259 | 24.783 | −70.796 | 27.958 | 1 | 57.51 | B | C |
| ATOM | 1845 | CD1 | ILE | B | 259 | 24.292 | −69.491 | 27.392 | 1 | 57.91 | B | C |
| ATOM | 1846 | CG2 | ILE | B | 259 | 24.504 | −72.317 | 26.027 | 1 | 58.36 | B | C |
| ATOM | 1847 | C | ILE | B | 259 | 26.999 | −73.665 | 26.642 | 1 | 52.47 | B | C |
| ATOM | 1848 | O | ILE | B | 259 | 26.458 | −74.758 | 26.515 | 1 | 50.11 | B | O |
| ATOM | 1849 | N | SER | B | 260 | 28.083 | −73.294 | 25.986 | 1 | 56.13 | B | N |
| ATOM | 1850 | CA | SER | B | 260 | 28.778 | −74.135 | 25.005 | 1 | 60.21 | B | C |
| ATOM | 1851 | CB | SER | B | 260 | 30.068 | −73.442 | 24.564 | 1 | 65.3 | B | C |
| ATOM | 1852 | OG | SER | B | 260 | 31.171 | −74.322 | 24.625 | 1 | 74.91 | B | O |
| ATOM | 1853 | C | SER | B | 260 | 29.128 | −75.537 | 25.428 | 1 | 61.1 | B | C |
| ATOM | 1854 | O | SER | B | 260 | 29.314 | −76.38 | 24.571 | 1 | 63.41 | B | O |
| ATOM | 1855 | N | THR | B | 261 | 29.294 | −75.779 | 26.726 | 1 | 71.34 | B | N |
| ATOM | 1856 | CA | THR | B | 261 | 29.716 | −77.103 | 27.223 | 1 | 70.79 | B | C |
| ATOM | 1857 | CB | THR | B | 261 | 31 | −76.973 | 28.069 | 1 | 67.73 | B | C |
| ATOM | 1858 | OG1 | THR | B | 261 | 30.73 | −76.145 | 29.207 | 1 | 56.84 | B | O |
| ATOM | 1859 | CG2 | THR | B | 261 | 32.147 | −76.384 | 27.229 | 1 | 66.27 | B | C |
| ATOM | 1860 | C | THR | B | 261 | 28.667 | −77.802 | 28.071 | 1 | 71.66 | B | C |
| ATOM | 1861 | O | THR | B | 261 | 28.992 | −78.731 | 28.805 | 1 | 73.46 | B | O |
| ATOM | 1862 | N | LEU | B | 262 | 27.412 | −77.384 | 27.961 | 1 | 71.62 | B | N |
| ATOM | 1863 | CA | LEU | B | 262 | 26.361 | −77.962 | 28.781 | 1 | 71.15 | B | C |
| ATOM | 1864 | CB | LEU | B | 262 | 25.095 | −77.117 | 28.702 | 1 | 71.6 | B | C |
| ATOM | 1865 | CG | LEU | B | 262 | 25.131 | −75.853 | 29.557 | 1 | 73.22 | B | C |
| ATOM | 1866 | CD1 | LEU | B | 262 | 23.891 | −75.052 | 29.227 | 1 | 74.93 | B | C |
| ATOM | 1867 | CD2 | LEU | B | 262 | 25.197 | −76.16 | 31.054 | 1 | 74.32 | B | C |
| ATOM | 1868 | C | LEU | B | 262 | 26.058 | −79.391 | 28.369 | 1 | 78.93 | B | C |
| ATOM | 1869 | O | LEU | B | 262 | 26.269 | −79.754 | 27.208 | 1 | 75.4 | B | O |
| ATOM | 1870 | N | SER | B | 263 | 25.557 | −80.181 | 29.329 | 1 | 82.51 | B | N |
| ATOM | 1871 | CA | SER | B | 263 | 25.221 | −81.602 | 29.114 | 1 | 86.95 | B | C |
| ATOM | 1872 | CB | SER | B | 263 | 25.238 | −82.391 | 30.446 | 1 | 86.43 | B | C |
| ATOM | 1873 | OG | SER | B | 263 | 23.973 | −82.392 | 31.116 | 1 | 84.17 | B | O |
| ATOM | 1874 | C | SER | B | 263 | 23.865 | −81.765 | 28.417 | 1 | 87.28 | B | C |
| ATOM | 1875 | O | SER | B | 263 | 23.132 | −80.804 | 28.246 | 1 | 78.91 | B | O |
| ATOM | 1876 | N | LYS | B | 264 | 23.534 | −82.995 | 28.041 | 1 | 87.4 | B | N |
| ATOM | 1877 | CA | LYS | B | 264 | 22.251 | −83.281 | 27.41 | 1 | 91.84 | B | C |
| ATOM | 1878 | CB | LYS | B | 264 | 22.236 | −84.697 | 26.82 | 1 | 98.53 | B | C |
| ATOM | 1879 | CG | LYS | B | 264 | 21.344 | −84.849 | 25.6 | 1 | 107.35 | B | C |
| ATOM | 1880 | CD | LYS | B | 264 | 22.037 | −84.35 | 24.334 | 1 | 108.82 | B | C |
| ATOM | 1881 | CE | LYS | B | 264 | 21.174 | −84.597 | 23.102 | 1 | 113.01 | B | C |
| ATOM | 1882 | NZ | LYS | B | 264 | 21.958 | −84.575 | 21.835 | 1 | 111.46 | B | N |
| ATOM | 1883 | C | LYS | B | 264 | 21.103 | −83.121 | 28.413 | 1 | 92.53 | B | C |
| ATOM | 1884 | O | LYS | B | 264 | 19.987 | −82.773 | 28.025 | 1 | 97.03 | B | O |
| ATOM | 1885 | N | GLN | B | 265 | 21.372 | −83.373 | 29.695 | 1 | 87.5 | B | N |
| ATOM | 1886 | CA | GLN | B | 265 | 20.319 | −83.328 | 30.716 | 1 | 91.93 | B | C |
| ATOM | 1887 | CB | GLN | B | 265 | 20.567 | −84.372 | 31.811 | 1 | 103.9 | B | C |
| ATOM | 1888 | CG | GLN | B | 265 | 20.047 | −85.769 | 31.461 | 1 | 111.32 | B | C |
| ATOM | 1889 | CD | GLN | B | 265 | 20.77 | −86.413 | 30.274 | 1 | 115.42 | B | C |
| ATOM | 1890 | OE1 | GLN | B | 265 | 21.914 | −86.87 | 30.403 | 1 | 107.03 | B | O |
| ATOM | 1891 | NE2 | GLN | B | 265 | 20.1 | −86.46 | 29.112 | 1 | 116.77 | B | N |
| ATOM | 1892 | C | GLN | B | 265 | 20.162 | −81.929 | 31.298 | 1 | 89.22 | B | C |
| ATOM | 1893 | O | GLN | B | 265 | 19.119 | −81.605 | 31.884 | 1 | 85.25 | B | O |
| ATOM | 1894 | N | ASP | B | 266 | 21.2 | −81.108 | 31.142 | 1 | 86.51 | B | N |
| ATOM | 1895 | CA | ASP | B | 266 | 21.077 | −79.654 | 31.324 | 1 | 86.54 | B | C |
| ATOM | 1896 | CB | ASP | B | 266 | 22.446 | −78.958 | 31.213 | 1 | 88.39 | B | C |
| ATOM | 1897 | CG | ASP | B | 266 | 23.438 | −79.41 | 32.294 | 1 | 91.97 | B | C |
| ATOM | 1898 | OD1 | ASP | B | 266 | 23.021 | −79.658 | 33.458 | 1 | 87.29 | B | O |
| ATOM | 1899 | OD2 | ASP | B | 266 | 24.646 | −79.508 | 31.972 | 1 | 85.33 | B | O |
| ATOM | 1900 | C | ASP | B | 266 | 20.098 | −79.046 | 30.304 | 1 | 81.55 | B | C |
| ATOM | 1901 | O | ASP | B | 266 | 19.343 | −78.13 | 30.649 | 1 | 75.33 | B | O |
| ATOM | 1902 | N | LEU | B | 267 | 20.124 | −79.564 | 29.067 | 1 | 75.82 | B | N |
| ATOM | 1903 | CA | LEU | B | 267 | 19.202 | −79.16 | 27.992 | 1 | 79.45 | B | C |
| ATOM | 1904 | CB | LEU | B | 267 | 19.846 | −79.346 | 26.62 | 1 | 77.92 | B | C |
| ATOM | 1905 | CG | LEU | B | 267 | 21.151 | −78.609 | 26.322 | 1 | 80.4 | B | C |
| ATOM | 1906 | CD1 | LEU | B | 267 | 21.433 | −78.726 | 24.822 | 1 | 80.52 | B | C |
| ATOM | 1907 | CD2 | LEU | B | 267 | 21.137 | −77.151 | 26.771 | 1 | 77.32 | B | C |
| ATOM | 1908 | C | LEU | B | 267 | 17.908 | −79.951 | 28.027 | 1 | 74.86 | B | C |
| ATOM | 1909 | O | LEU | B | 267 | 17.657 | −80.773 | 27.17 | 1 | 80.78 | B | O |
| ATOM | 1910 | N | THR | B | 268 | 17.098 | −79.669 | 29.035 | 1 | 74.15 | B | N |
| ATOM | 1911 | CA | THR | B | 268 | 15.797 | −80.288 | 29.243 | 1 | 73.89 | B | C |
| ATOM | 1912 | CB | THR | B | 268 | 15.883 | −81.355 | 30.361 | 1 | 80.45 | B | C |
| ATOM | 1913 | OG1 | THR | B | 268 | 16.199 | −82.619 | 29.764 | 1 | 84.66 | B | O |
| ATOM | 1914 | CG2 | THR | B | 268 | 14.579 | −81.49 | 31.179 | 1 | 85.35 | B | C |
| ATOM | 1915 | C | THR | B | 268 | 14.892 | −79.124 | 29.595 | 1 | 73.15 | B | C |
| ATOM | 1916 | O | THR | B | 268 | 15.291 | −78.238 | 30.366 | 1 | 70.7 | B | O |
| ATOM | 1917 | N | CYS | B | 269 | 13.701 | −79.107 | 29.005 | 1 | 74.77 | B | N |
| ATOM | 1918 | CA | CYS | B | 269 | 12.799 | −77.959 | 29.106 | 1 | 69.68 | B | C |
| ATOM | 1919 | CB | CYS | B | 269 | 12.028 | −77.717 | 27.796 | 1 | 64 | B | C |
| ATOM | 1920 | SG | CYS | B | 269 | 11.038 | −76.182 | 27.837 | 1 | 70.38 | B | S |
| ATOM | 1921 | C | CYS | B | 269 | 11.785 | −78.211 | 30.196 | 1 | 63.51 | B | C |
| ATOM | 1922 | O | CYS | B | 269 | 11.292 | −79.327 | 30.313 | 1 | 56.37 | B | O |
| ATOM | 1923 | N | SER | B | 270 | 11.446 | −77.165 | 30.949 | 1 | 55.48 | B | N |
| ATOM | 1924 | CA | SER | B | 270 | 10.246 | −77.19 | 31.781 | 1 | 65.92 | B | C |
| ATOM | 1925 | CB | SER | B | 270 | 10.205 | −75.966 | 32.715 | 1 | 66.81 | B | C |
| ATOM | 1926 | OG | SER | B | 270 | 8.994 | −75.232 | 32.577 | 1 | 72.63 | B | O |
| ATOM | 1927 | C | SER | B | 270 | 8.993 | −77.237 | 30.892 | 1 | 78.26 | B | C |
| ATOM | 1928 | O | SER | B | 270 | 8.793 | −76.366 | 30.035 | 1 | 92.88 | B | O |
| ATOM | 1929 | N | GLY | B | 271 | 8.117 | −78.211 | 31.116 | 1 | 83.93 | B | N |
| ATOM | 1930 | CA | GLY | B | 271 | 6.841 | −78.252 | 30.387 | 1 | 80.99 | B | C |
| ATOM | 1931 | C | GLY | B | 271 | 5.795 | −77.252 | 30.867 | 1 | 78 | B | C |
| ATOM | 1932 | O | GLY | B | 271 | 4.633 | −77.365 | 30.492 | 1 | 79.34 | B | O |
| ATOM | 1933 | N | SER | B | 272 | 6.189 | −76.243 | 31.649 | 1 | 78.71 | B | N |
| ATOM | 1934 | CA | SER | B | 272 | 5.237 | −75.512 | 32.488 | 1 | 72.94 | B | C |
| ATOM | 1935 | CB | SER | B | 272 | 5.956 | −74.897 | 33.677 | 1 | 69.32 | B | C |
| ATOM | 1936 | OG | SER | B | 272 | 6.457 | −73.632 | 33.335 | 1 | 66.77 | B | O |
| ATOM | 1937 | C | SER | B | 272 | 4.488 | −74.419 | 31.73 | 1 | 75.15 | B | C |
| ATOM | 1938 | O | SER | B | 272 | 4.789 | −74.119 | 30.576 | 1 | 72.98 | B | O |
| ATOM | 1939 | N | ASP | B | 273 | 3.509 | −73.837 | 32.409 | 1 | 76.08 | B | N |
| ATOM | 1940 | CA | ASP | B | 273 | 2.65 | −72.804 | 31.847 | 1 | 78.69 | B | C |
| ATOM | 1941 | CB | ASP | B | 273 | 1.37 | −72.664 | 32.675 | 1 | 82.12 | B | C |
| ATOM | 1942 | CG | ASP | B | 273 | 0.475 | −73.856 | 32.54 | 1 | 87.81 | B | C |
| ATOM | 1943 | OD1 | ASP | B | 273 | −0.169 | −73.979 | 31.476 | 1 | 96.56 | B | O |
| ATOM | 1944 | OD2 | ASP | B | 273 | 0.428 | −74.671 | 33.486 | 1 | 90.35 | B | O |
| ATOM | 1945 | C | ASP | B | 273 | 3.337 | −71.469 | 31.808 | 1 | 74.03 | B | C |
| ATOM | 1946 | O | ASP | B | 273 | 3.012 | −70.633 | 30.971 | 1 | 73.33 | B | O |
| ATOM | 1947 | N | ASP | B | 274 | 4.244 | −71.243 | 32.748 | 1 | 72.88 | B | N |
| ATOM | 1948 | CA | ASP | B | 274 | 5.054 | −70.033 | 32.728 | 1 | 70.28 | B | C |
| ATOM | 1949 | CB | ASP | B | 274 | 5.845 | −69.898 | 34.027 | 1 | 72.68 | B | C |
| ATOM | 1950 | CG | ASP | B | 274 | 4.986 | −69.494 | 35.199 | 1 | 75.17 | B | C |
| ATOM | 1951 | OD1 | ASP | B | 274 | 3.746 | −69.642 | 35.143 | 1 | 75.62 | B | O |
| ATOM | 1952 | OD2 | ASP | B | 274 | 5.575 | −69.032 | 36.2 | 1 | 82.45 | B | O |
| ATOM | 1953 | C | ASP | B | 274 | 6.008 | −70.057 | 31.53 | 1 | 62.69 | B | C |
| ATOM | 1954 | O | ASP | B | 274 | 6.292 | −69.012 | 30.945 | 1 | 54.56 | B | O |
| ATOM | 1955 | N | CYS | B | 275 | 6.528 | −71.249 | 31.212 | 1 | 55.4 | B | N |
| ATOM | 1956 | CA | CYS | B | 275 | 7.294 | −71.479 | 29.992 | 1 | 61.46 | B | C |
| ATOM | 1957 | CB | CYS | B | 275 | 7.834 | −72.912 | 29.946 | 1 | 58.81 | B | C |
| ATOM | 1958 | SG | CYS | B | 275 | 8.77 | −73.324 | 28.451 | 1 | 65.73 | B | S |
| ATOM | 1959 | C | CYS | B | 275 | 6.465 | −71.202 | 28.714 | 1 | 61.34 | B | C |
| ATOM | 1960 | O | CYS | B | 275 | 6.953 | −70.525 | 27.796 | 1 | 63.2 | B | O |
| ATOM | 1961 | N | LYS | B | 276 | 5.237 | −71.72 | 28.67 | 1 | 55.59 | B | N |
| ATOM | 1962 | CA | LYS | B | 276 | 4.328 | −71.501 | 27.536 | 1 | 60.46 | B | C |
| ATOM | 1963 | CB | LYS | B | 276 | 2.953 | −72.129 | 27.769 | 1 | 63.33 | B | C |
| ATOM | 1964 | CG | LYS | B | 276 | 2.929 | −73.639 | 27.622 | 1 | 72.11 | B | C |
| ATOM | 1965 | CD | LYS | B | 276 | 1.606 | −74.238 | 28.085 | 1 | 84.68 | B | C |
| ATOM | 1966 | CE | LYS | B | 276 | 1.567 | −75.757 | 27.935 | 1 | 91.28 | B | C |
| ATOM | 1967 | NZ | LYS | B | 276 | 0.79 | −76.144 | 26.724 | 1 | 99.33 | B | N |
| ATOM | 1968 | C | LYS | B | 276 | 4.151 | −70.021 | 27.291 | 1 | 58.34 | B | C |
| ATOM | 1969 | O | LYS | B | 276 | 4.341 | −69.542 | 26.161 | 1 | 51.53 | B | O |
| ATOM | 1970 | N | ALA | B | 277 | 3.835 | −69.299 | 28.36 | 1 | 51.27 | B | N |
| ATOM | 1971 | CA | ALA | B | 277 | 3.647 | −67.86 | 28.269 | 1 | 51.23 | B | C |
| ATOM | 1972 | CB | ALA | B | 277 | 3.109 | −67.305 | 29.572 | 1 | 51.4 | B | C |
| ATOM | 1973 | C | ALA | B | 277 | 4.899 | −67.101 | 27.83 | 1 | 54.22 | B | C |
| ATOM | 1974 | O | ALA | B | 277 | 4.793 | −66.134 | 27.068 | 1 | 53.3 | B | O |
| ATOM | 1975 | N | ALA | B | 278 | 6.084 | −67.522 | 28.288 | 1 | 54.48 | B | N |
| ATOM | 1976 | CA | ALA | B | 278 | 7.318 | −66.78 | 27.944 | 1 | 49.47 | B | C |
| ATOM | 1977 | CB | ALA | B | 278 | 8.452 | −67.119 | 28.899 | 1 | 46.82 | B | C |
| ATOM | 1978 | C | ALA | B | 278 | 7.749 | −67.053 | 26.507 | 1 | 45.05 | B | C |
| ATOM | 1979 | O | ALA | B | 278 | 8.44 | −66.231 | 25.901 | 1 | 46.65 | B | O |
| ATOM | 1980 | N | TYR | B | 279 | 7.397 | −68.237 | 26.02 | 1 | 40.37 | B | N |
| ATOM | 1981 | CA | TYR | B | 279 | 7.563 | −68.614 | 24.631 | 1 | 47.33 | B | C |
| ATOM | 1982 | CB | TYR | B | 279 | 7.216 | −70.093 | 24.387 | 1 | 47.12 | B | C |
| ATOM | 1983 | CG | TYR | B | 279 | 7.209 | −70.377 | 22.923 | 1 | 53.68 | B | C |
| ATOM | 1984 | CD1 | TYR | B | 279 | 8.403 | −70.367 | 22.17 | 1 | 58.31 | B | C |
| ATOM | 1985 | CE1 | TYR | B | 279 | 8.393 | −70.607 | 20.785 | 1 | 54.68 | B | C |
| ATOM | 1986 | CZ | TYR | B | 279 | 7.159 | −70.816 | 20.139 | 1 | 62.06 | B | C |
| ATOM | 1987 | OH | TYR | B | 279 | 7.043 | −71.03 | 18.771 | 1 | 56.98 | B | O |
| ATOM | 1988 | CE2 | TYR | B | 279 | 5.983 | −70.796 | 20.875 | 1 | 62.25 | B | C |
| ATOM | 1989 | CD2 | TYR | B | 279 | 6.013 | −70.567 | 22.249 | 1 | 58.9 | B | C |
| ATOM | 1990 | C | TYR | B | 279 | 6.703 | −67.726 | 23.728 | 1 | 42.07 | B | C |
| ATOM | 1991 | O | TYR | B | 279 | 7.196 | −67.115 | 22.762 | 1 | 46.8 | B | O |
| ATOM | 1992 | N | ILE | B | 280 | 5.432 | −67.631 | 24.074 | 1 | 41.62 | B | N |
| ATOM | 1993 | CA | ILE | B | 280 | 4.485 | −66.769 | 23.353 | 1 | 44.35 | B | C |
| ATOM | 1994 | CB | ILE | B | 280 | 3.071 | −66.869 | 23.98 | 1 | 45.22 | B | C |
| ATOM | 1995 | CG1 | ILE | B | 280 | 2.494 | −68.245 | 23.602 | 1 | 46.68 | B | C |
| ATOM | 1996 | CD1 | ILE | B | 280 | 1.283 | −68.703 | 24.374 | 1 | 46.49 | B | C |
| ATOM | 1997 | CG2 | ILE | B | 280 | 2.159 | −65.747 | 23.505 | 1 | 46.41 | B | C |
| ATOM | 1998 | C | ILE | B | 280 | 4.998 | −65.337 | 23.294 | 1 | 48.53 | B | C |
| ATOM | 1999 | O | ILE | B | 280 | 4.835 | −64.662 | 22.27 | 1 | 47.98 | B | O |
| ATOM | 2000 | N | ASP | B | 281 | 5.667 | −64.898 | 24.365 | 1 | 50.22 | B | N |
| ATOM | 2001 | CA | ASP | B | 281 | 6.192 | −63.539 | 24.441 | 1 | 51.71 | B | C |
| ATOM | 2002 | CB | ASP | B | 281 | 6.28 | −63.073 | 25.899 | 1 | 61.01 | B | C |
| ATOM | 2003 | CG | ASP | B | 281 | 4.922 | −63.109 | 26.605 | 1 | 72.44 | B | C |
| ATOM | 2004 | OD1 | ASP | B | 281 | 3.89 | −63.365 | 25.943 | 1 | 88.81 | B | O |
| ATOM | 2005 | OD2 | ASP | B | 281 | 4.869 | −62.902 | 27.833 | 1 | 82.45 | B | O |
| ATOM | 2006 | C | ASP | B | 281 | 7.51 | −63.336 | 23.712 | 1 | 47.62 | B | C |
| ATOM | 2007 | O | ASP | B | 281 | 7.969 | −62.188 | 23.571 | 1 | 50.43 | B | O |
| ATOM | 2008 | N | ILE | B | 282 | 8.12 | −64.401 | 23.201 | 1 | 44.51 | B | N |
| ATOM | 2009 | CA | ILE | B | 282 | 9.223 | −64.179 | 22.253 | 1 | 46.31 | B | C |
| ATOM | 2010 | CB | ILE | B | 282 | 10.44 | −65.109 | 22.473 | 1 | 48.05 | B | C |
| ATOM | 2011 | CG1 | ILE | B | 282 | 10.074 | −66.58 | 22.36 | 1 | 51.06 | B | C |
| ATOM | 2012 | CD1 | ILE | B | 282 | 11.273 | −67.509 | 22.296 | 1 | 48.95 | B | C |
| ATOM | 2013 | CG2 | ILE | B | 282 | 11.058 | −64.81 | 23.828 | 1 | 57.56 | B | C |
| ATOM | 2014 | C | ILE | B | 282 | 8.769 | −64.177 | 20.771 | 1 | 45.2 | B | C |
| ATOM | 2015 | O | ILE | B | 282 | 9.583 | −63.872 | 19.862 | 1 | 47.2 | B | O |
| ATOM | 2016 | N | LEU | B | 283 | 7.499 | −64.484 | 20.528 | 1 | 40.33 | B | N |
| ATOM | 2017 | CA | LEU | B | 283 | 6.983 | −64.445 | 19.175 | 1 | 43.21 | B | C |
| ATOM | 2018 | CB | LEU | B | 283 | 5.689 | −65.172 | 19.079 | 1 | 42.67 | B | C |
| ATOM | 2019 | CG | LEU | B | 283 | 5.762 | −66.657 | 19.392 | 1 | 43.36 | B | C |
| ATOM | 2020 | CD1 | LEU | B | 283 | 4.341 | −67.186 | 19.438 | 1 | 45.13 | B | C |
| ATOM | 2021 | CD2 | LEU | B | 283 | 6.595 | −67.449 | 18.404 | 1 | 44.09 | B | C |
| ATOM | 2022 | C | LEU | B | 283 | 6.831 | −62.98 | 18.772 | 1 | 44.95 | B | C |
| ATOM | 2023 | O | LEU | B | 283 | 6.53 | −62.133 | 19.577 | 1 | 47.39 | B | O |
| ATOM | 2024 | N | GLY | B | 284 | 7.133 | −62.682 | 17.527 | 1 | 46.39 | B | N |
| ATOM | 2025 | CA | GLY | B | 284 | 7.137 | −61.311 | 17.055 | 1 | 44.37 | B | C |
| ATOM | 2026 | C | GLY | B | 284 | 8.436 | −60.638 | 17.357 | 1 | 44.3 | B | C |
| ATOM | 2027 | O | GLY | B | 284 | 8.481 | −59.39 | 17.41 | 1 | 47.29 | B | O |
| ATOM | 2028 | N | THR | B | 285 | 9.491 | −61.451 | 17.541 | 1 | 44.46 | B | N |
| ATOM | 2029 | CA | THR | B | 285 | 10.867 | −60.963 | 17.763 | 1 | 41.33 | B | C |
| ATOM | 2030 | CB | THR | B | 285 | 11.384 | −61.215 | 19.238 | 1 | 43.83 | B | C |
| ATOM | 2031 | OG1 | THR | B | 285 | 11.737 | −62.609 | 19.474 | 1 | 41.17 | B | O |
| ATOM | 2032 | CG2 | THR | B | 285 | 10.329 | −60.763 | 20.271 | 1 | 45.7 | B | C |
| ATOM | 2033 | C | THR | B | 285 | 11.78 | −61.701 | 16.821 | 1 | 43.02 | B | C |
| ATOM | 2034 | O | THR | B | 285 | 11.364 | −62.698 | 16.239 | 1 | 45.28 | B | O |
| ATOM | 2035 | N | VAL | B | 286 | 13.049 | −61.273 | 16.77 | 1 | 45.07 | B | N |
| ATOM | 2036 | CA | VAL | B | 286 | 14.097 | −61.946 | 16.021 | 1 | 44.16 | B | C |
| ATOM | 2037 | CB | VAL | B | 286 | 15.459 | −61.176 | 16.16 | 1 | 44.39 | B | C |
| ATOM | 2038 | CG1 | VAL | B | 286 | 16.117 | −61.41 | 17.521 | 1 | 44.15 | B | C |
| ATOM | 2039 | CG2 | VAL | B | 286 | 16.43 | −61.58 | 15.076 | 1 | 41.98 | B | C |
| ATOM | 2040 | C | VAL | B | 286 | 14.213 | −63.455 | 16.384 | 1 | 43.33 | B | C |
| ATOM | 2041 | O | VAL | B | 286 | 14.614 | −64.291 | 15.54 | 1 | 45.19 | B | O |
| ATOM | 2042 | N | LEU | B | 287 | 13.803 | −63.811 | 17.597 | 1 | 43.65 | B | N |
| ATOM | 2043 | CA | LEU | B | 287 | 13.904 | −65.209 | 18.078 | 1 | 44.96 | B | C |
| ATOM | 2044 | CB | LEU | B | 287 | 13.717 | −65.287 | 19.617 | 1 | 47.77 | B | C |
| ATOM | 2045 | CG | LEU | B | 287 | 14.653 | −64.383 | 20.455 | 1 | 48.53 | B | C |
| ATOM | 2046 | CD1 | LEU | B | 287 | 14.159 | −64.209 | 21.878 | 1 | 54.36 | B | C |
| ATOM | 2047 | CD2 | LEU | B | 287 | 16.096 | −64.876 | 20.474 | 1 | 50.54 | B | C |
| ATOM | 2048 | C | LEU | B | 287 | 12.965 | −66.172 | 17.366 | 1 | 42.36 | B | C |
| ATOM | 2049 | O | LEU | B | 287 | 13.177 | −67.401 | 17.4 | 1 | 46.16 | B | O |
| ATOM | 2050 | N | GLN | B | 288 | 11.91 | −65.633 | 16.763 | 1 | 38.71 | B | N |
| ATOM | 2051 | CA | GLN | B | 288 | 10.988 | −66.402 | 15.919 | 1 | 43.11 | B | C |
| ATOM | 2052 | CB | GLN | B | 288 | 9.752 | −65.53 | 15.685 | 1 | 46.91 | B | C |
| ATOM | 2053 | CG | GLN | B | 288 | 8.634 | −66.188 | 14.89 | 1 | 51.62 | B | C |
| ATOM | 2054 | CD | GLN | B | 288 | 7.331 | −65.448 | 15.016 | 1 | 53.17 | B | C |
| ATOM | 2055 | OE1 | GLN | B | 288 | 7.306 | −64.26 | 15.349 | 1 | 55.32 | B | O |
| ATOM | 2056 | NE2 | GLN | B | 288 | 6.245 | −66.135 | 14.738 | 1 | 57.04 | B | N |
| ATOM | 2057 | C | GLN | B | 288 | 11.532 | −66.799 | 14.52 | 1 | 47.79 | B | C |
| ATOM | 2058 | O | GLN | B | 288 | 10.978 | −67.669 | 13.849 | 1 | 53.98 | B | O |
| ATOM | 2059 | N | VAL | B | 289 | 12.59 | −66.143 | 14.07 | 1 | 47.6 | B | N |
| ATOM | 2060 | CA | VAL | B | 289 | 13.104 | −66.322 | 12.712 | 1 | 49.86 | B | C |
| ATOM | 2061 | CB | VAL | B | 289 | 14.057 | −65.176 | 12.346 | 1 | 49.81 | B | C |
| ATOM | 2062 | CG1 | VAL | B | 289 | 14.729 | −65.433 | 10.999 | 1 | 51.02 | B | C |
| ATOM | 2063 | CG2 | VAL | B | 289 | 13.322 | −63.84 | 12.392 | 1 | 44.39 | B | C |
| ATOM | 2064 | C | VAL | B | 289 | 13.902 | −67.593 | 12.668 | 1 | 47.18 | B | C |
| ATOM | 2065 | O | VAL | B | 289 | 14.82 | −67.763 | 13.476 | 1 | 43.82 | B | O |
| ATOM | 2066 | N | GLN | B | 290 | 13.61 | −68.448 | 11.703 | 1 | 48.4 | B | N |
| ATOM | 2067 | CA | GLN | B | 290 | 14.293 | −69.752 | 11.623 | 1 | 57.86 | B | C |
| ATOM | 2068 | CB | GLN | B | 290 | 13.734 | −70.611 | 10.505 | 1 | 66.85 | B | C |
| ATOM | 2069 | CG | GLN | B | 290 | 13.869 | −72.104 | 10.756 | 1 | 78.97 | B | C |
| ATOM | 2070 | CD | GLN | B | 290 | 12.848 | −72.896 | 9.947 | 1 | 85.48 | B | C |
| ATOM | 2071 | OE1 | GLN | B | 290 | 12.878 | −72.891 | 8.714 | 1 | 92.17 | B | O |
| ATOM | 2072 | NE2 | GLN | B | 290 | 11.919 | −73.54 | 10.633 | 1 | 84.51 | B | N |
| ATOM | 2073 | C | GLN | B | 290 | 15.779 | −69.537 | 11.395 | 1 | 54.93 | B | C |
| ATOM | 2074 | O | GLN | B | 290 | 16.174 | −68.762 | 10.535 | 1 | 47.22 | B | O |
| ATOM | 2075 | N | CYS | B | 291 | 16.602 | −70.189 | 12.199 | 1 | 51.11 | B | N |
| ATOM | 2076 | CA | CYS | B | 291 | 18.03 | −69.936 | 12.16 | 1 | 45.32 | B | C |
| ATOM | 2077 | CB | CYS | B | 291 | 18.487 | −69.339 | 13.489 | 1 | 50.61 | B | C |
| ATOM | 2078 | SG | CYS | B | 291 | 18.262 | −70.423 | 14.915 | 1 | 50.63 | B | S |
| ATOM | 2079 | C | CYS | B | 291 | 18.742 | −71.229 | 11.87 | 1 | 47.34 | B | C |
| ATOM | 2080 | O | CYS | B | 291 | 18.217 | −72.318 | 12.134 | 1 | 44.04 | B | O |
| ATOM | 2081 | N | THR | B | 292 | 19.961 | −71.092 | 11.358 | 1 | 47.54 | B | N |
| ATOM | 2082 | CA | THR | B | 292 | 20.756 | −72.22 | 10.891 | 1 | 47.23 | B | C |
| ATOM | 2083 | CB | THR | B | 292 | 21.021 | −72.081 | 9.368 | 1 | 45.55 | B | C |
| ATOM | 2084 | OG1 | THR | B | 292 | 21.755 | −73.206 | 8.9 | 1 | 52.8 | B | O |
| ATOM | 2085 | CG2 | THR | B | 292 | 21.786 | −70.811 | 9.017 | 1 | 39.81 | B | C |
| ATOM | 2086 | C | THR | B | 292 | 22.085 | −72.324 | 11.664 | 1 | 50.44 | B | C |
| ATOM | 2087 | O | THR | B | 292 | 22.593 | −71.3 | 12.188 | 1 | 41.49 | B | O |
| ATOM | 2088 | N | CYS | B | 293 | 22.644 | −73.543 | 11.74 | 1 | 45.73 | B | N |
| ATOM | 2089 | CA | CYS | B | 293 | 24.027 | −73.711 | 12.224 | 1 | 49.18 | B | C |
| ATOM | 2090 | CB | CYS | B | 293 | 24.049 | −74.681 | 13.411 | 1 | 52.62 | B | C |
| ATOM | 2091 | SG | CYS | B | 293 | 23.219 | −73.935 | 14.859 | 1 | 52.05 | B | S |
| ATOM | 2092 | C | CYS | B | 293 | 25.108 | −74.037 | 11.16 | 1 | 55.13 | B | C |
| ATOM | 2093 | O | CYS | B | 293 | 26.287 | −74.085 | 11.48 | 1 | 57.44 | B | O |
| ATOM | 2094 | N | ARG | B | 294 | 24.728 | −74.182 | 9.895 | 1 | 57.21 | B | N |
| ATOM | 2095 | CA | ARG | B | 294 | 25.7 | −74.118 | 8.789 | 1 | 61.43 | B | C |
| ATOM | 2096 | CB | ARG | B | 294 | 24.996 | −74.318 | 7.457 | 1 | 61.02 | B | C |
| ATOM | 2097 | CG | ARG | B | 294 | 24.203 | −75.588 | 7.349 | 1 | 57.21 | B | C |
| ATOM | 2098 | CD | ARG | B | 294 | 23.287 | −75.537 | 6.147 | 1 | 55.49 | B | C |
| ATOM | 2099 | NE | ARG | B | 294 | 22.003 | −76.163 | 6.442 | 1 | 60.25 | B | N |
| ATOM | 2100 | CZ | ARG | B | 294 | 21.111 | −76.536 | 5.524 | 1 | 71.23 | B | C |
| ATOM | 2101 | NH1 | ARG | B | 294 | 19.953 | −77.072 | 5.89 | 1 | 73.42 | B | N |
| ATOM | 2102 | NH2 | ARG | B | 294 | 21.37 | −76.393 | 4.237 | 1 | 77.11 | B | N |
| ATOM | 2103 | C | ARG | B | 294 | 26.235 | −72.693 | 8.781 | 1 | 67.09 | B | C |
| ATOM | 2104 | O | ARG | B | 294 | 25.586 | −71.798 | 9.342 | 1 | 79.76 | B | O |
| ATOM | 2105 | N | THR | B | 295 | 27.359 | −72.464 | 8.119 | 1 | 61.68 | B | N |
| ATOM | 2106 | CA | THR | B | 295 | 28.039 | −71.149 | 8.086 | 1 | 70.86 | B | C |
| ATOM | 2107 | CB | THR | B | 295 | 27.126 | −69.922 | 8.366 | 1 | 71.01 | B | C |
| ATOM | 2108 | OG1 | THR | B | 295 | 25.879 | −70.065 | 7.686 | 1 | 90.81 | B | O |
| ATOM | 2109 | CG2 | THR | B | 295 | 27.793 | −68.595 | 7.922 | 1 | 76.24 | B | C |
| ATOM | 2110 | C | THR | B | 295 | 29.139 | −71.037 | 9.138 | 1 | 79.69 | B | C |
| ATOM | 2111 | O | THR | B | 295 | 30.075 | −70.245 | 8.98 | 1 | 82.47 | B | O |
| ATOM | 2112 | N | ILE | B | 296 | 29.024 | −71.827 | 10.203 | 1 | 77.89 | B | N |
| ATOM | 2113 | CA | ILE | B | 296 | 29.57 | −71.458 | 11.501 | 1 | 74.35 | B | C |
| ATOM | 2114 | CB | ILE | B | 296 | 28.526 | −71.832 | 12.602 | 1 | 71.68 | B | C |
| ATOM | 2115 | CG1 | ILE | B | 296 | 28.293 | −70.663 | 13.54 | 1 | 72.96 | B | C |
| ATOM | 2116 | CD1 | ILE | B | 296 | 27.492 | −69.55 | 12.921 | 1 | 70.06 | B | C |
| ATOM | 2117 | CG2 | ILE | B | 296 | 28.889 | −73.09 | 13.404 | 1 | 74.06 | B | C |
| ATOM | 2118 | C | ILE | B | 296 | 30.944 | −72.114 | 11.698 | 1 | 75.95 | B | C |
| ATOM | 2119 | O | ILE | B | 296 | 31.163 | −73.235 | 11.245 | 1 | 78.99 | B | O |
| ATOM | 2120 | N | THR | B | 297 | 31.866 | −71.433 | 12.377 | 1 | 81.17 | B | N |
| ATOM | 2121 | CA | THR | B | 297 | 33.196 | −72.024 | 12.645 | 1 | 84.16 | B | C |
| ATOM | 2122 | CB | THR | B | 297 | 34.152 | −71.035 | 13.313 | 1 | 83 | B | C |
| ATOM | 2123 | OG1 | THR | B | 297 | 33.546 | −70.575 | 14.524 | 1 | 80.52 | B | O |
| ATOM | 2124 | CG2 | THR | B | 297 | 34.478 | −69.86 | 12.382 | 1 | 78.19 | B | C |
| ATOM | 2125 | C | THR | B | 297 | 33.103 | −73.256 | 13.556 | 1 | 82.52 | B | C |
| ATOM | 2126 | O | THR | B | 297 | 32.245 | −73.326 | 14.435 | 1 | 84.61 | B | O |
| ATOM | 2127 | N | GLN | B | 298 | 33.981 | −74.223 | 13.309 | 1 | 83.17 | B | N |
| ATOM | 2128 | CA | GLN | B | 298 | 34.059 | −75.464 | 14.079 | 1 | 88.99 | B | C |
| ATOM | 2129 | CB | GLN | B | 298 | 35.404 | −76.159 | 13.777 | 1 | 97.97 | B | C |
| ATOM | 2130 | CG | GLN | B | 298 | 35.922 | −77.181 | 14.79 | 1 | 103.12 | B | C |
| ATOM | 2131 | CD | GLN | B | 298 | 35.061 | −78.423 | 14.894 | 1 | 104.5 | B | C |
| ATOM | 2132 | OE1 | GLN | B | 298 | 35.404 | −79.469 | 14.345 | 1 | 108.32 | B | O |
| ATOM | 2133 | NE2 | GLN | B | 298 | 33.948 | −78.322 | 15.607 | 1 | 101.78 | B | N |
| ATOM | 2134 | C | GLN | B | 298 | 33.89 | −75.203 | 15.579 | 1 | 94.1 | B | C |
| ATOM | 2135 | O | GLN | B | 298 | 33.079 | −75.878 | 16.24 | 1 | 88.07 | B | O |
| ATOM | 2136 | N | SER | B | 299 | 34.647 | −74.222 | 16.094 | 1 | 92.42 | B | N |
| ATOM | 2137 | CA | SER | B | 299 | 34.642 | −73.872 | 17.528 | 1 | 85.58 | B | C |
| ATOM | 2138 | CB | SER | B | 299 | 35.562 | −72.671 | 17.824 | 1 | 79.87 | B | C |
| ATOM | 2139 | OG | SER | B | 299 | 35.504 | −71.686 | 16.809 | 1 | 77.13 | B | O |
| ATOM | 2140 | C | SER | B | 299 | 33.221 | −73.63 | 18.071 | 1 | 88.52 | B | C |
| ATOM | 2141 | O | SER | B | 299 | 32.744 | −74.416 | 18.903 | 1 | 80.35 | B | O |
| ATOM | 2142 | N | GLU | B | 300 | 32.534 | −72.608 | 17.538 | 1 | 82.61 | B | N |
| ATOM | 2143 | CA | GLU | B | 300 | 31.222 | −72.152 | 18.069 | 1 | 78.03 | B | C |
| ATOM | 2144 | CB | GLU | B | 300 | 31.087 | −70.628 | 17.915 | 1 | 77.53 | B | C |
| ATOM | 2145 | CG | GLU | B | 300 | 31.04 | −70.088 | 16.495 | 1 | 80.94 | B | C |
| ATOM | 2146 | CD | GLU | B | 300 | 31.387 | −68.595 | 16.384 | 1 | 79.64 | B | C |
| ATOM | 2147 | OE1 | GLU | B | 300 | 31.581 | −68.109 | 15.245 | 1 | 83.2 | B | O |
| ATOM | 2148 | OE2 | GLU | B | 300 | 31.469 | −67.894 | 17.415 | 1 | 79.64 | B | O |
| ATOM | 2149 | C | GLU | B | 300 | 29.967 | −72.91 | 17.558 | 1 | 74.01 | B | C |
| ATOM | 2150 | O | GLU | B | 300 | 28.85 | −72.383 | 17.572 | 1 | 75.67 | B | O |
| ATOM | 2151 | N | GLU | B | 301 | 30.154 | −74.187 | 17.228 | 1 | 71.82 | B | N |
| ATOM | 2152 | CA | GLU | B | 301 | 29.174 | −74.997 | 16.539 | 1 | 68.92 | B | C |
| ATOM | 2153 | CB | GLU | B | 301 | 29.874 | −76.082 | 15.722 | 1 | 73.12 | B | C |
| ATOM | 2154 | CG | GLU | B | 301 | 28.99 | −76.788 | 14.711 | 1 | 80.11 | B | C |
| ATOM | 2155 | CD | GLU | B | 301 | 29.569 | −78.117 | 14.25 | 1 | 85.28 | B | C |
| ATOM | 2156 | OE1 | GLU | B | 301 | 30.755 | −78.136 | 13.866 | 1 | 92.5 | B | O |
| ATOM | 2157 | OE2 | GLU | B | 301 | 28.841 | −79.141 | 14.263 | 1 | 82.96 | B | O |
| ATOM | 2158 | C | GLU | B | 301 | 28.21 | −75.62 | 17.511 | 1 | 66.28 | B | C |
| ATOM | 2159 | O | GLU | B | 301 | 27.01 | −75.549 | 17.293 | 1 | 73.59 | B | O |
| ATOM | 2160 | N | SER | B | 302 | 28.711 | −76.249 | 18.571 | 1 | 67.68 | B | N |
| ATOM | 2161 | CA | SER | B | 302 | 27.822 | −76.77 | 19.644 | 1 | 69.12 | B | C |
| ATOM | 2162 | CB | SER | B | 302 | 28.573 | −77.507 | 20.78 | 1 | 69.64 | B | C |
| ATOM | 2163 | OG | SER | B | 302 | 29.948 | −77.656 | 20.501 | 1 | 75.15 | B | O |
| ATOM | 2164 | C | SER | B | 302 | 27.023 | −75.63 | 20.261 | 1 | 56.28 | B | C |
| ATOM | 2165 | O | SER | B | 302 | 25.845 | −75.827 | 20.549 | 1 | 63.02 | B | O |
| ATOM | 2166 | N | LEU | B | 303 | 27.669 | −74.466 | 20.472 | 1 | 49.66 | B | N |
| ATOM | 2167 | CA | LEU | B | 303 | 26.989 | −73.289 | 21.017 | 1 | 50.12 | B | C |
| ATOM | 2168 | CB | LEU | B | 303 | 27.901 | −72.075 | 21.13 | 1 | 46.8 | B | C |
| ATOM | 2169 | CG | LEU | B | 303 | 27.163 | −70.823 | 21.666 | 1 | 51.82 | B | C |
| ATOM | 2170 | CD1 | LEU | B | 303 | 26.509 | −71.065 | 23.018 | 1 | 51.97 | B | C |
| ATOM | 2171 | CD2 | LEU | B | 303 | 28.039 | −69.569 | 21.733 | 1 | 50.71 | B | C |
| ATOM | 2172 | C | LEU | B | 303 | 25.785 | −72.881 | 20.158 | 1 | 56.02 | B | C |
| ATOM | 2173 | O | LEU | B | 303 | 24.71 | −72.562 | 20.708 | 1 | 54.67 | B | O |
| ATOM | 2174 | N | CYS | B | 304 | 26 | −72.844 | 18.834 | 1 | 56.11 | B | N |
| ATOM | 2175 | CA | CYS | B | 304 | 24.921 | −72.617 | 17.852 | 1 | 50.82 | B | C |
| ATOM | 2176 | CB | CYS | B | 304 | 25.475 | −72.535 | 16.431 | 1 | 53.5 | B | C |
| ATOM | 2177 | SG | CYS | B | 304 | 24.226 | −72.068 | 15.19 | 1 | 53.28 | B | S |
| ATOM | 2178 | C | CYS | B | 304 | 23.82 | −73.675 | 17.963 | 1 | 50.65 | B | C |
| ATOM | 2179 | O | CYS | B | 304 | 22.647 | −73.323 | 18.168 | 1 | 49.96 | B | O |
| ATOM | 2180 | N | LYS | B | 305 | 24.177 | −74.958 | 17.927 | 1 | 49.41 | B | N |
| ATOM | 2181 | CA | LYS | B | 305 | 23.149 | −76.025 | 18.019 | 1 | 51.89 | B | C |
| ATOM | 2182 | CB | LYS | B | 305 | 23.752 | −77.427 | 17.865 | 1 | 62.33 | B | C |
| ATOM | 2183 | CG | LYS | B | 305 | 24.75 | −77.556 | 16.739 | 1 | 69.39 | B | C |
| ATOM | 2184 | CD | LYS | B | 305 | 24.608 | −78.857 | 16 | 1 | 80.74 | B | C |
| ATOM | 2185 | CE | LYS | B | 305 | 25.712 | −78.975 | 14.963 | 1 | 91.04 | B | C |
| ATOM | 2186 | NZ | LYS | B | 305 | 25.248 | −79.724 | 13.763 | 1 | 97.43 | B | N |
| ATOM | 2187 | C | LYS | B | 305 | 22.398 | −75.994 | 19.332 | 1 | 55.17 | B | C |
| ATOM | 2188 | O | LYS | B | 305 | 21.237 | −76.438 | 19.415 | 1 | 52.21 | B | O |
| ATOM | 2189 | N | ILE | B | 306 | 23.062 | −75.514 | 20.381 | 1 | 53.43 | B | N |
| ATOM | 2190 | CA | ILE | B | 306 | 22.405 | −75.461 | 21.674 | 1 | 52.66 | B | C |
| ATOM | 2191 | CB | ILE | B | 306 | 23.429 | −75.31 | 22.823 | 1 | 55.19 | B | C |
| ATOM | 2192 | CG1 | ILE | B | 306 | 24.067 | −76.68 | 23.082 | 1 | 57.15 | B | C |
| ATOM | 2193 | CD1 | ILE | B | 306 | 25.407 | −76.628 | 23.78 | 1 | 58.54 | B | C |
| ATOM | 2194 | CG2 | ILE | B | 306 | 22.767 | −74.788 | 24.1 | 1 | 58.3 | B | C |
| ATOM | 2195 | C | ILE | B | 306 | 21.353 | −74.368 | 21.657 | 1 | 45.36 | B | C |
| ATOM | 2196 | O | ILE | B | 306 | 20.221 | −74.593 | 22.086 | 1 | 44.63 | B | O |
| ATOM | 2197 | N | PHE | B | 307 | 21.735 | −73.179 | 21.197 | 1 | 45.64 | B | N |
| ATOM | 2198 | CA | PHE | B | 307 | 20.761 | −72.127 | 20.987 | 1 | 48.61 | B | C |
| ATOM | 2199 | CB | PHE | B | 307 | 21.416 | −70.861 | 20.456 | 1 | 48.72 | B | C |
| ATOM | 2200 | CG | PHE | B | 307 | 22.184 | −70.078 | 21.499 | 1 | 55.29 | B | C |
| ATOM | 2201 | CD1 | PHE | B | 307 | 21.676 | −69.882 | 22.783 | 1 | 55.79 | B | C |
| ATOM | 2202 | CE1 | PHE | B | 307 | 22.39 | −69.169 | 23.718 | 1 | 59 | B | C |
| ATOM | 2203 | CZ | PHE | B | 307 | 23.617 | −68.62 | 23.395 | 1 | 56.7 | B | C |
| ATOM | 2204 | CE2 | PHE | B | 307 | 24.129 | −68.793 | 22.122 | 1 | 57.01 | B | C |
| ATOM | 2205 | CD2 | PHE | B | 307 | 23.413 | −69.515 | 21.186 | 1 | 56.62 | B | C |
| ATOM | 2206 | C | PHE | B | 307 | 19.628 | −72.555 | 20.038 | 1 | 52.58 | B | C |
| ATOM | 2207 | O | PHE | B | 307 | 18.45 | −72.32 | 20.332 | 1 | 54.04 | B | O |
| ATOM | 2208 | N | GLN | B | 308 | 19.975 | −73.185 | 18.923 | 1 | 50.59 | B | N |
| ATOM | 2209 | CA | GLN | B | 308 | 18.951 | −73.687 | 17.998 | 1 | 54.01 | B | C |
| ATOM | 2210 | CB | GLN | B | 308 | 19.588 | −74.434 | 16.827 | 1 | 52.68 | B | C |
| ATOM | 2211 | CG | GLN | B | 308 | 18.66 | −74.684 | 15.654 | 1 | 53.08 | B | C |
| ATOM | 2212 | CD | GLN | B | 308 | 19.378 | −75.36 | 14.499 | 1 | 52.94 | B | C |
| ATOM | 2213 | OE1 | GLN | B | 308 | 19.394 | −74.863 | 13.364 | 1 | 56.88 | B | O |
| ATOM | 2214 | NE2 | GLN | B | 308 | 19.985 | −76.495 | 14.786 | 1 | 52.02 | B | N |
| ATOM | 2215 | C | GLN | B | 308 | 17.946 | −74.602 | 18.705 | 1 | 48.27 | B | C |
| ATOM | 2216 | O | GLN | B | 308 | 16.74 | −74.51 | 18.486 | 1 | 51.19 | B | O |
| ATOM | 2217 | N | HIS | B | 309 | 18.447 | −75.474 | 19.563 | 1 | 49.44 | B | N |
| ATOM | 2218 | CA | HIS | B | 309 | 17.601 | −76.426 | 20.286 | 1 | 49.58 | B | C |
| ATOM | 2219 | CB | HIS | B | 309 | 18.477 | −77.442 | 21.035 | 1 | 54.27 | B | C |
| ATOM | 2220 | CG | HIS | B | 309 | 17.705 | −78.433 | 21.85 | 1 | 58.26 | B | C |
| ATOM | 2221 | ND1 | HIS | B | 309 | 17.225 | −79.614 | 21.326 | 1 | 62.63 | B | N |
| ATOM | 2222 | CE1 | HIS | B | 309 | 16.604 | −80.292 | 22.273 | 1 | 62.63 | B | C |
| ATOM | 2223 | NE2 | HIS | B | 309 | 16.666 | −79.593 | 23.393 | 1 | 63.56 | B | N |
| ATOM | 2224 | CD2 | HIS | B | 309 | 17.338 | −78.42 | 23.153 | 1 | 55.98 | B | C |
| ATOM | 2225 | C | HIS | B | 309 | 16.692 | −75.694 | 21.25 | 1 | 49.81 | B | C |
| ATOM | 2226 | O | HIS | B | 309 | 15.528 | −76.04 | 21.373 | 1 | 54.66 | B | O |
| ATOM | 2227 | N | MET | B | 310 | 17.222 | −74.662 | 21.907 | 1 | 51.13 | B | N |
| ATOM | 2228 | CA | MET | B | 310 | 16.459 | −73.91 | 22.908 | 1 | 52.94 | B | C |
| ATOM | 2229 | CB | MET | B | 310 | 17.411 | −73.054 | 23.735 | 1 | 60.02 | B | C |
| ATOM | 2230 | CG | MET | B | 310 | 18.335 | −73.876 | 24.66 | 1 | 65.7 | B | C |
| ATOM | 2231 | SD | MET | B | 310 | 19.03 | −72.877 | 25.999 | 1 | 60.12 | B | S |
| ATOM | 2232 | CE | MET | B | 310 | 19.918 | −71.637 | 25.081 | 1 | 62.38 | B | C |
| ATOM | 2233 | C | MET | B | 310 | 15.358 | −73.028 | 22.317 | 1 | 58.43 | B | C |
| ATOM | 2234 | O | MET | B | 310 | 14.299 | −72.828 | 22.931 | 1 | 49.43 | B | O |
| ATOM | 2235 | N | LEU | B | 311 | 15.626 | −72.495 | 21.124 | 1 | 55.3 | B | N |
| ATOM | 2236 | CA | LEU | B | 311 | 14.663 | −71.701 | 20.379 | 1 | 54.76 | B | C |
| ATOM | 2237 | CB | LEU | B | 311 | 15.425 | −70.837 | 19.365 | 1 | 52.9 | B | C |
| ATOM | 2238 | CG | LEU | B | 311 | 16.244 | −69.77 | 20.085 | 1 | 51.8 | B | C |
| ATOM | 2239 | CD1 | LEU | B | 311 | 17.048 | −68.986 | 19.068 | 1 | 46.27 | B | C |
| ATOM | 2240 | CD2 | LEU | B | 311 | 15.346 | −68.873 | 20.96 | 1 | 48.62 | B | C |
| ATOM | 2241 | C | LEU | B | 311 | 13.538 | −72.515 | 19.702 | 1 | 53.84 | B | C |
| ATOM | 2242 | O | LEU | B | 311 | 12.5 | −71.952 | 19.309 | 1 | 51.52 | B | O |
| ATOM | 2243 | N | HIS | B | 312 | 13.713 | −73.828 | 19.61 | 1 | 56.51 | B | N |
| ATOM | 2244 | CA | HIS | B | 312 | 12.74 | −74.667 | 18.924 | 1 | 62.13 | B | C |
| ATOM | 2245 | CB | HIS | B | 312 | 13.404 | −75.873 | 18.282 | 1 | 62.52 | B | C |
| ATOM | 2246 | CG | HIS | B | 312 | 12.669 | −76.337 | 17.079 | 1 | 75.27 | B | C |
| ATOM | 2247 | ND1 | HIS | B | 312 | 11.639 | −77.245 | 17.156 | 1 | 81.3 | B | N |
| ATOM | 2248 | CE1 | HIS | B | 312 | 11.13 | −77.436 | 15.956 | 1 | 72.39 | B | C |
| ATOM | 2249 | NE2 | HIS | B | 312 | 11.77 | −76.652 | 15.108 | 1 | 81.34 | B | N |
| ATOM | 2250 | CD2 | HIS | B | 312 | 12.724 | −75.936 | 15.789 | 1 | 75.21 | B | C |
| ATOM | 2251 | C | HIS | B | 312 | 11.576 | −75.096 | 19.82 | 1 | 65.6 | B | C |
| ATOM | 2252 | O | HIS | B | 312 | 11.793 | −75.572 | 20.947 | 1 | 66.91 | B | O |
| ATOM | 2253 | N | ARG | B | 313 | 10.343 | −74.916 | 19.319 | 1 | 64.63 | B | N |
| ATOM | 2254 | CA | ARG | B | 313 | 9.137 | −75.116 | 20.137 | 1 | 68.65 | B | C |
| ATOM | 2255 | CB | ARG | B | 313 | 7.884 | −74.453 | 19.52 | 1 | 71.7 | B | C |
| ATOM | 2256 | CG | ARG | B | 313 | 7.382 | −75.039 | 18.208 | 1 | 70.13 | B | C |
| ATOM | 2257 | CD | ARG | B | 313 | 5.972 | −74.574 | 17.816 | 1 | 74.5 | B | C |
| ATOM | 2258 | NE | ARG | B | 313 | 5.468 | −75.415 | 16.713 | 1 | 75.53 | B | N |
| ATOM | 2259 | CZ | ARG | B | 313 | 4.196 | −75.538 | 16.31 | 1 | 76.08 | B | C |
| ATOM | 2260 | NH1 | ARG | B | 313 | 3.925 | −76.373 | 15.308 | 1 | 78.33 | B | N |
| ATOM | 2261 | NH2 | ARG | B | 313 | 3.19 | −74.852 | 16.878 | 1 | 74.93 | B | N |
| ATOM | 2262 | C | ARG | B | 313 | 8.869 | −76.584 | 20.44 | 1 | 70.81 | B | C |
| ATOM | 2263 | O | ARG | B | 313 | 8.305 | −76.893 | 21.486 | 1 | 70.23 | B | O |
| ATOM | 2264 | N | LYS | B | 314 | 9.22 | −77.465 | 19.498 | 1 | 76.73 | B | N |
| ATOM | 2265 | CA | LYS | B | 314 | 9.17 | −78.935 | 19.703 | 1 | 86.87 | B | C |
| ATOM | 2266 | CB | LYS | B | 314 | 9.552 | −79.717 | 18.416 | 1 | 95.74 | B | C |
| ATOM | 2267 | CG | LYS | B | 314 | 10.965 | −80.337 | 18.358 | 1 | 103.07 | B | C |
| ATOM | 2268 | CD | LYS | B | 314 | 11.272 | −80.941 | 16.982 | 1 | 110.25 | B | C |
| ATOM | 2269 | CE | LYS | B | 314 | 12.645 | −80.559 | 16.438 | 1 | 113.63 | B | C |
| ATOM | 2270 | NZ | LYS | B | 314 | 12.678 | −80.71 | 14.952 | 1 | 117.87 | B | N |
| ATOM | 2271 | C | LYS | B | 314 | 10.023 | −79.415 | 20.889 | 1 | 82.22 | B | C |
| ATOM | 2272 | O | LYS | B | 314 | 9.645 | −80.367 | 21.575 | 1 | 83.12 | B | O |
| ATOM | 2273 | N | SER | B | 315 | 11.167 | −78.76 | 21.105 | 1 | 71.79 | B | N |
| ATOM | 2274 | CA | SER | B | 315 | 12.013 | −79.017 | 22.273 | 1 | 69.76 | B | C |
| ATOM | 2275 | CB | SER | B | 315 | 13.304 | −78.194 | 22.192 | 1 | 64.2 | B | C |
| ATOM | 2276 | OG | SER | B | 315 | 13.918 | −78.345 | 20.929 | 1 | 66.4 | B | O |
| ATOM | 2277 | C | SER | B | 315 | 11.336 | −78.765 | 23.636 | 1 | 68.25 | B | C |
| ATOM | 2278 | O | SER | B | 315 | 11.833 | −79.263 | 24.656 | 1 | 74.38 | B | O |
| ATOM | 2279 | N | CYS | B | 316 | 10.261 | −77.966 | 23.67 | 1 | 62.65 | B | N |
| ATOM | 2280 | CA | CYS | B | 316 | 9.476 | −77.748 | 24.902 | 1 | 68.81 | B | C |
| ATOM | 2281 | CB | CYS | B | 316 | 9.439 | −76.256 | 25.246 | 1 | 68.41 | B | C |
| ATOM | 2282 | SG | CYS | B | 316 | 11.011 | −75.579 | 25.848 | 1 | 73.83 | B | S |
| ATOM | 2283 | C | CYS | B | 316 | 8.037 | −78.32 | 24.885 | 1 | 72.28 | B | C |
| ATOM | 2284 | O | CYS | B | 316 | 7.46 | −78.528 | 25.955 | 1 | 70.57 | B | O |
| ATOM | 2285 | N | PHE | B | 317 | 7.477 | −78.591 | 23.698 | 1 | 77.56 | B | N |
| ATOM | 2286 | CA | PHE | B | 317 | 6.055 | −78.931 | 23.552 | 1 | 78.82 | B | C |
| ATOM | 2287 | CB | PHE | B | 317 | 5.239 | −77.647 | 23.356 | 1 | 79.14 | B | C |
| ATOM | 2288 | CG | PHE | B | 317 | 5.56 | −76.562 | 24.345 | 1 | 83.93 | B | C |
| ATOM | 2289 | CD1 | PHE | B | 317 | 5.092 | −76.635 | 25.654 | 1 | 88.9 | B | C |
| ATOM | 2290 | CE1 | PHE | B | 317 | 5.394 | −75.632 | 26.574 | 1 | 88.22 | B | C |
| ATOM | 2291 | CZ | PHE | B | 317 | 6.164 | −74.536 | 26.192 | 1 | 82.39 | B | C |
| ATOM | 2292 | CE2 | PHE | B | 317 | 6.644 | −74.45 | 24.893 | 1 | 86.67 | B | C |
| ATOM | 2293 | CD2 | PHE | B | 317 | 6.345 | −75.465 | 23.975 | 1 | 90 | B | C |
| ATOM | 2294 | C | PHE | B | 317 | 5.768 | −79.865 | 22.381 | 1 | 79.06 | B | C |
| ATOM | 2295 | O | PHE | B | 317 | 5.958 | −79.477 | 21.233 | 1 | 78.14 | B | O |
| ATOM | 2296 | N | ASN | B | 318 | 5.335 | −81.092 | 22.675 | 1 | 86.74 | B | N |
| ATOM | 2297 | CA | ASN | B | 318 | 4.603 | −81.937 | 21.69 | 1 | 97.22 | B | C |
| ATOM | 2298 | CB | ASN | B | 318 | 5.472 | −82.382 | 20.488 | 1 | 96.55 | B | C |
| ATOM | 2299 | CG | ASN | B | 318 | 6.789 | −82.994 | 20.914 | 1 | 102.52 | B | C |
| ATOM | 2300 | OD1 | ASN | B | 318 | 6.81 | −83.983 | 21.644 | 1 | 108.78 | B | O |
| ATOM | 2301 | ND2 | ASN | B | 318 | 7.895 | −82.416 | 20.461 | 1 | 101.47 | B | N |
| ATOM | 2302 | C | ASN | B | 318 | 3.928 | −83.147 | 22.336 | 1 | 90.47 | B | C |
| ATOM | 2303 | O | ASN | B | 318 | 2.727 | −83.35 | 22.159 | 1 | 87.94 | B | O |

Column 1- record name;
Column 2 - atom serial number;
Column 3 - atom name;
Column 4 - amino acid residue name;
Column 5 - chain identifier;
Column 6 - amino acid residue sequence number;
Column 7 - orthoganal coordinates for X in Angstromes;
Column 8 - orthoganal coordinates for Y in Angstromes;
Column 9 - orthoganal coordinates for Z in Angstromes;
Column 10 - occupancy;
Column 11 - temperature factor;
Column 12 - Segment identifier;
Column 13 - element symbol.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for identifying an agent that binds to an extracellular domain of a GFRAL protein, the method comprising:
a) constructing a three-dimensional structure of a complex with a GDF15 protein defined by the atomic coordinates of Table 6;
b) employing the three-dimensional structure and a modeling method to identify a candidate agent that binds to the GFRAL protein;
c) assaying the candidate agent for binding to the extracellular domain of the GFRAL protein; and
d) comparing the binding of the candidate agent to the binding of the GDF15 protein to the extracellular domain of the GFRAL protein,
wherein the candidate agent is identified as an agent that binds to the extracellular domain of the GFRAL protein when the candidate agent binds with an affinity similar to the GDF15 protein.

2-8. (canceled)

9. A method for identifying an agent that modulates binding of a GDF15 protein to a GFRAL protein, the method comprising:
a) contacting a candidate agent with a recombinant cell genetically modified to express the GFRAL protein, wherein the extracellular domain of the GFRAL protein comprises one or more amino acid residues of a GFRAL domain associated with the interface between a GFRAL protein and a GDF15 protein, wherein the one or more amino acid residues of the GFRAL domain correspond to the amino acid residues at the positions selected from the group consisting of GLY140, LEU148, ALA149, ALA146, VAL142, ASN145, VAL139, ALA135, GLU136, LEU152, LEU132, SER201, ALA204, LEU205, LYS153, ILE196, PRO197, and GLN200 of SEQ ID NO: 9, and
wherein the contacting is in the presence of the GDF15 protein; and
b) assaying a level of binding of the GDF15 protein to the GFRAL protein;
wherein a change in the level of binding of the GDF15 protein to the GFRAL protein in the presence of the candidate agent as compared to a level of binding of the GDF15 protein to the GFRAL protein in absence of the candidate agent identifies the candidate agent as an agent that modulates binding of the GDF15 protein to the GFRAL protein, or alternatively,
a) constructing a three-dimensional structure of a complex with a GDF15 protein defined by the atomic coordinates of Table 6;
b) employing the three-dimensional structure and a modeling method to identify a candidate agent that modulates binding of a GDF15 protein to a GFRAL protein;
c) contacting the candidate agent with a recombinant cell genetically modified to express the GFRAL protein, wherein the contacting is in the presence of the GDF15 protein; and
d) assaying a level of binding of the GDF15 protein to the GFRAL protein,
wherein a change in the level of binding of the GDF15 protein to the GFRAL protein in the presence of the candidate agent as compared to a level of binding of the GDF15 protein to the GFRAL protein in absence of the candidate agent identifies the candidate agent as an agent that modulates binding of the GDF15 protein to the GFRAL protein.

10-22. (canceled)

23. A method for identifying an agent that modulates binding of a GFRAL protein comprising an extracellular domain to a RET protein, the method comprising:
a) contacting a candidate agent with a recombinant cell genetically modified to express the GFRAL protein and the RET protein wherein the extracellular domain of the GFRAL protein comprises one or more amino acid residues of a GFRAL domain associated with the interface between a GFRAL protein and a RET protein, wherein the one or more amino acid residues of the GFRAL domain correspond to the amino acid residues at the positions selected from the group consisting of GLN246, ARG247, ARG250, LYS251, CYS252, ASP255, GLU256, ASN257, CYS258, ILE259, SER260, THR261, LEU262, THR297, and GLN298 of SEQ ID NO: 9; and
b) assaying a level of binding of the GFRAL protein and the RET protein;
wherein a change in the level of binding of the GFRAL protein and the RET protein in the presence of the candidate agent as compared to a level of binding of the GFRAL protein and the RET protein in absence of the candidate agent identifies the candidate agent as an agent that modulates binding of the GFRAL protein to the RET protein, or alternatively,
a) constructing a three-dimensional structure of a complex with a GDF15 protein defined by the atomic coordinates of Table 6;
b) employing the three-dimensional structure and a modeling method to identify a candidate agent that modulates binding of the GFRAL protein to the RET protein;
c) contacting the candidate agent with a recombinant cell genetically modified to express the GFRAL protein and the RET protein; and
d) assaying a level of binding of the GFRAL protein and the RET protein;
wherein a change in the level of binding of the GFRAL protein and the RET protein in the presence of the candidate agent as compared to a level of binding of the GFRAL protein and the RET protein in absence of the candidate agent identifies the candidate agent as an agent that modulates binding of the GFRAL protein to the RET protein.

24-26. (canceled)

27. A method of reducing GDF15 protein activity in a subject, treating involuntary body weight loss in a subject, or preventing involuntary body weight loss in a subject at risk of involuntary body weight loss, the method comprising:
administering to the subject at least one of:
a) an agent that binds an extracellular domain of a GFRAL protein wherein the GFRAL-ECD comprises (i) one or more amino acid residues of a GFRAL domain associated with the interface between a GFRAL protein and a GDF15 protein, wherein the one or more amino acid residues of the GFRAL domain correspond to the amino acid residues at the positions selected from the group consisting of GLY140, LEU148, ALA149, ALA146, VAL142, ASN145, VAL139, ALA135, GLU136, LEU152, LEU132, SER201, ALA204, LEU205, LYS153, ILE196, PRO197, and GLN200 of SEQ ID NO: 9, and/or (ii) one or more amino acid residues of a GFRAL domain associated with the interface between a GFRAL protein and a RET protein, wherein the one or more amino acid residues of the GFRAL domain correspond to the amino acid residues at the positions selected from the group consisting of GLN246, ARG247, ARG250, LYS251, CYS252, ASP255, GLU256, ASN257, CYS258, ILE259, SER260, THR261, LEU262, THR297, and GLN298 of SEQ ID NO: 9; and
b) an extracellular domain of a GFRAL protein (GFRAL-ECD),
wherein the agent or GFRAL-ECD is administered in an amount effective to reduce the GDF15 protein activity, treat involuntary body weight loss, or prevent onset of involuntary body weight loss, in the subject.

28-42. (canceled)

43. A crystal comprising a GFRAL protein and a GDF15 protein.

44. The crystal of claim 43, wherein the crystal diffracts x-ray radiation to produce a diffraction pattern representing the three-dimensional structure of the complex having approximately the following cell constants: a=75.4 Å, b=88.8 Å, c=121.3 Å, and space group P21.

45. The crystal of claim 43, which diffracts x-ray radiations at a resolution of about 2.20 Å.

46. The crystal of claim 43, wherein the GFRAL protein comprises the amino acid sequence of SEQ ID NO: 23.

47. The crystal of claim 43, wherein the GDF15 protein is a homodimer.

48. The crystal of claim 43, having the atomic coordinates of Table 6.

49. The crystal of claim 43 for use in a screening assay for the identification of an antagonist of a GDF15 protein.

50. A composition comprising a crystal of any one of claims 43 to 48.

51. A method for identifying a variant GFRAL protein with the ability to bind a GDF15 protein or the ability to bind a RET protein, the method comprising:
a) constructing a three-dimensional structure of a complex comprising a GFRAL protein and a GDF15 protein defined by the atomic coordinates of Table 6;
b) employing the three-dimensional structure and a modeling method to identify a site for mutating the GFRAL protein and mutating the site to generate the variant GFRAL protein;
c) producing the variant GFRAL protein; and
d) assaying the variant GFRAL protein to determine its ability to bind the GDF15 protein or the RET protein.

52-56. (canceled)

57. A method for identifying a variant GDF15 protein with the ability to bind a GFRAL protein, the method comprising:
a) constructing a three-dimensional structure of a complex comprising a GFRAL protein and a GDF15 protein defined by the atomic coordinates of Table 6;
b) employing the three-dimensional structure and a modeling method to identify a site for mutating the GDF15 protein and mutating the site to generate the variant GDF15 protein;
c) producing the variant GDF15 protein; and
d) assaying the variant GDF15 protein to determine its ability to bind the GFRAL protein.

58-59. (canceled)

60. A method for producing an agent that inhibits formation of a complex comprising a GFRAL protein and a GDF15 protein (GFRAL/GDF15 complex) or a complex comprising a GFRAL protein and a RET protein (GFRAL/RET complex), comprising:
a) obtaining two or more 3-dimensional structures of a complex comprising a GFRAL protein and one of two or more agents (GFRAL/agent complex);
b) comparing each of the 3-dimensional GFRAL/agent complex structures with a 3-dimensional structure of the GFRAL/GDF15 complex or with a 3-dimensional structure of a GFRAL/RET complex;
c) selecting at least one of the two or more agents based on the structural similarity of the GFRAL/agent complex with the 3-dimensional structure of a GFRAL/GDF15 complex or with a 3-dimensional structure of a GFRAL/RET complex; and
d) producing the agent.

61-68. (canceled)


SEQUENCE LISTING (67 sequences)

SEQ ID NO: 1 - protein, 375 aa, Homo sapiens
  mature human GFRAL polypeptide
QTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEVIYAAMCMTVTCGILLLVMVKLRTSRISSKARDPSSIQIPGEL

SEQ ID NO: 2 - protein, 332 aa, Homo sapiens
  GFRAL ECD
QTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGE

SEQ ID NO: 3 - protein, 1049 aa, Homo sapiens
  mature human RET9
KVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 4 - protein, 351 aa, Artificial Sequence
  GFRAL ECD fragment
MIVFIFLAMGLSLENEYTSQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGE

SEQ ID NO: 5 - protein, 332 aa, Artificial Sequence; identical to SEQ ID NO: 2
  GFRAL ECD

SEQ ID NO: 6 - protein, 112 aa, Homo sapiens
  mature human GDF15 polypeptide
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 7 - protein, 367 aa, Homo sapiens
  Fc-hGDF15 protein sequence
MEWSWVFLFFLSVTTGVHSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGGGGGSGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 8 - protein, 274 aa, Homo sapiens
  Fc protein sequence
MEWSWVFLFFLSVTTGVHSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGKLTVDKSRWQQGNVFSCSVMHEALHNHYTQKSLSLSPGK

SEQ ID NO: 9 - protein, 394 aa, Homo sapiens
  full-length precursor human GFRAL with signal peptide sequence
MIVFIFLAMGLSLENEYTSQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEVIYAAMCMTVTCGILLLVMVKLRTSRISSKARDPSSIQIPGEL

SEQ ID NO: 10 - protein, 393 aa, Mus musculus
  Mouse GFRAL protein sequence
MLVFIFLAVTLSSENESSSQTNDCAHLIQKCLIDANGCEQSWRSMEDTCLTPGDSCKINNSLHCNLSIQALVEKNFQFKECLCMDDLHCTVNKLFGKKCTNKTDNMEKDNKDKWNLTTTPFYHGFKQMQSCLEVTEACVGDVVCNAQLALYLKACSANGNLCDVKHCQAAIRFFYQNMPFNTAQMLAFCDCAQSDIPCQQSKETLHSKPCALNIVPPPTCLSVIHTCRNDELCRTHYRTFQTECWPHITGKCHEDETCISMLGKQDLTCSGSESCRAAFLGTFGTVLQVPCACRGVTQAEEHVCMIFQHMLHSKSCFNYPTPNVKDISSYEKKNSKEITLTGFNSFFNGELLYVVVCMAVTCGILFLVMLKLRIQSEKRDPSSIEIAGGVIIQ

SEQ ID NO: 11 - protein, 394 aa, Rattus norvegicus
  Rat GFRAL protein sequence
MLVFIFLAVRLSSENESSSQTNDCAYFMRQCLTDTDGCKQSWRSMEDACLVSGDSCKINNPLPCNLSIQSLVEKHFQFKGCLCTDDLHCTVNKIFGKKCTNKTDSMKKDNKYKRNLTTPLYHDTGFKQMQSCLEVTEACVGDVVCNAQLALYLKACTANGNLCDVKHCQAAIRFFYQNMPFNTAQMLAFCDCAQSDIPCQQSKETLHSKPCALNVVPPPTCLSVIHTCRNDELCRTYYRTFQTECWPHVAGKCREDETCISMLGKQDLTCSGSDSCRAAYLGTFGTVLQVPCACRSITQGEEPLCMAFQHMLHSKSCFNYPTPNVKDISSYERKHSKEITLTGFNSPFSGELIYVVVCMVVTSGILSLVMLKLRIPSKKRDPAPIEIAGAVIIQ

SEQ ID NO: 12 - protein, 394 aa, Cynomolgus monkey
  Cynomolgus monkey GFRAL protein sequence
FVVVVIQALGLSLENEYTSQTNNCTYLREQCLHDANGCKHAWRIMEDACNDSDPGDPCKMNNSSYCNLSIQYLVESNFRFKECLCTDDFYCTVNKLLGKECVNKSDNMREDKFKWNLTTHSHHGFKGMWSCLEVAEACVGDVVCNAQLASYLKACSANGNPCDVKHCQAAIRFFYQNIPFNIAQMLAFCDCSQSDIPCQQSKEALHSKPCALNMVPPPTCLNVIRSCQNDELCRRHYRTFQSKCWQRVTRKCHEDENCISALSKQDLTCSGSDDCKAAYIDILGTVLQVQCNCRTITQSEESLCKIFQHMLHRKSCFNYPTLSNVKSMALYTRKHTNKITLTGFQSPFNGEVIYAAMCMTVTCGILLLVMVKLRTSRISSKARDPSLSQVPGEL

SEQ ID NO: 13 - protein, 1114 aa, Homo sapiens
  Human RET51 protein sequence
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 14 - protein, 1072 aa, Homo sapiens
  Human RET9 protein sequence
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 15 - protein, 1115 aa, Mus musculus
  Mouse RET51 protein sequence
MAKATSGAAGLGLKLILLLPLLGEAPLGLYFSRDAYWERLYVDQPAGTPLLYVHALRDAPGEVPSFRLGQHLYGVYRTRLHENDWIRINETTGLLYLNQSLDHSSWEQLSIRNGGFPLLTIFLQVFLGSTAQREGECHWPGCTRVYFSFINDTFPNCSSFKAQDLCIPETAVSFRVRENRPPGTFYHFHMLPVQFLCPNISVKYSLLGGDSLPFRCDPDCLEVSTRWALDRELREKYVLEALCIVAGPGANKETVTLSFPVTVYDEDDSAPTFSGGVGTASAVVEFKRKEGTVVATLQVFDADVVPASGELVRRYTNTLLSGDSWAQQTFRVEHSPIETLVQVNNNSVRATMHNYKLILNRSLSISESRVLQLAVLVNDSDFQGPGAGGILVLHFNVSVLPVTLNLPRAYSFPVNKRARRYAQIGKVCVENCQEFSGVSIQYKLQPSSINCTALGVVTSPEDTSGTLFVNDTEALRRPECTKLQYTVVATDRQTRRQTQASLVVTVEGTSITEEVGCPKSCAVNKRRPECEECGGLGSPTGRCEWRQGDGKGITRNFSTCSPSTRTCPDGHCDAVESRDANICPQDCLRADIVGGHERGERQGIKAGYGICNCFPDEKKCFCEPEDSQGPLCDALCRTIITAALFSLIISILLSIFCVCHHHKHGHKPPIASAEMTFCRPAQGFPISYSSSGTRRPSLDSTENQVPVDSFKIPEDPKWEFPRKNLVLGKTLGEGEFGKVVKATAFRLKGRAGYTTVAVKMLKENASQSELRDLLSEFNLLKQVNHPHVIKLYGACSQDGPLLLIVEYAKYGSLRGFLRDSRKIGPAYVSGGGSRNSSSLDHPDERVLTMGDLISFAWQISRGMQYLAEMKLVHRDLAARNILVAEGRKMKISDFGLSRDVYEEDSYVKKSKGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKSRDYLDLAASTPSDSLLYDDGLSEEETPLVDCNNAPLPRSLPSTWIENKLYGMSDPNWPGESPVPLTRADGTSTGFPRYANDSVYANWMVSPSAAKLMDTFDS

SEQ ID NO: 16 - protein, 1073 aa, Mus musculus
  Mouse RET9 protein sequence
MAKATSGAAGLGLKLILLLPLLGEAPLGLYFSRDAYWERLYVDQPAGTPLLYVHALRDAPGEVPSFRLGQHLYGVYRTRLHENDWIRINETTGLLYLNQSLDHSSWEQLSIRNGGFPLLTIFLQVFLGSTAQREGECHWPGCTRVYFSFINDTFPNCSSFKAQDLCIPETAVSFRVRENRPPGTFYHFHMLPVQFLCPNISVKYSLLGGDSLPFRCDPDCLEVSTRWALDRELREKYVLEALCIVAGPGANKETVTLSFPVTVYDEDDSAPTFSGGVGTASAVVEFKRKEGTVVATLQVFDADVVPASGELVRRYTNTLLSGDSWAQQTFRVEHSPIETLVQVNNNSVRATMHNYKLILNRSLSISESRVLQLAVLVNDSDFQGPGAGGILVLHFNVSVLPVTLNLPRAYSFPVNKRARRYAQIGKVCVENCQEFSGVSIQYKLQPSSINCTALGVVTSPEDTSGTLFVNDTEALRRPECTKLQYTVVATDRQTRRQTQASLVVTVEGTSITEEVGCPKSCAVNKRRPECEECGGLGSPTGRCEWRQGDGKGITRNFSTCSPSTRTCPDGHCDAVESRDANICPQDCLRADIVGGHERGERQGIKAGYGICNCFPDEKKCFCEPEDSQGPLCDALCRTIITAALFSLIISILLSIFCVCHHHKHGHKPPIASAEMTFCRPAQGFPISYSSSGTRRPSLDSTENQVPVDSFKIPEDPKWEFPRKNLVLGKTLGEGEFGKVVKATAFRLKGRAGYTTVAVKMLKENASQSELRDLLSEFNLLKQVNHPHVIKLYGACSQDGPLLLIVEYAKYGSLRGFLRDSRKIGPAYVSGGGSRNSSSLDHPDERVLTMGDLISFAWQISRGMQYLAEMKLVHRDLAARNILVAEGRKMKISDFGLSRDVYEEDSYVKKSKGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKSRDYLDLAASTPSDSLLYDDGLSEEETPLVDCNNAPLPRSLPSTWIENKLYGRISHAFTRF

SEQ ID NO: 17 - protein, 1114 aa, Rattus norvegicus
  IgK signal peptide-Rat RET51 protein sequence
MDMRVPAQLLGLLLLWLRGARCEAPLGLYFSRDAYWERLYVDQPAGTPLLYVHALRDAPGEVPSFRLGQYLYGVYRTRLHENDWIHIDSGTGLLYLNQSLDHSSWEQLSIRNGGFPLLTVFLQVFLGSTAQREGECHWPGCARVYFSFINDTFPNCSSFKARDLCTPETGVSFRIRENRPPGTFYQFRMLPVQFLCPNISVKYKLLEGDGLPFRCDPDCLEVSTRWALDRELQEKYVLEAECAVAGPGANKEKVAVSFPVTVYDEDDSPPTFSGGVGTASAVVEFKRKEGTVVATLQVFDADVVPASGELVRRYTSTLLSGDSWAQQTFRVEHTPNETLVQSNNNSVRATMHNYKLVLNRSLSISESRVLQLVVLVNDSDFQGPGSGVLFLHFNVSVLPVTLNLPMAYSFPVNRRARRYAQIGKVCVENCQEFSGVSIQYKLQPSSTNCSALGVVTSTEDTSGTLYVNDTEALRRPECTELQYTVVATDRQTRRQTQASLVVTVEGTYIAEEVGCPKSCAVNKRRPECEECGGLGSPTGRCEWRQGDGKGITRNFSTCSPSTRTCPDGHCDALESRDINICPQDCLRGPIVGGHERGERQGIKAGYGICNCFPDEKKCFCEPEDSQGPLCDELCRTVITAAVLFSFIISVLLSTFCIHRYHKHAHKPPIASAEMTFCRPAQGFPISYSSSGTRRPSLDSMENQVSVDSFKIPEDPKWEFPRKNLVLGKTLGEGEFGKVVKATAFRLKGRAGYTTVAVKMLKENASQSELRDLLSEFNLLKQVNHPHVIKLYGACSQDGPLLLIVEYAKYGSLRGFLRDSRKIGPAYVSSGGSRNSSSLDHPDERVLTMGDLISFAWQISRGMQYLAEMKLVHRDLAARNILVAEGRKMKISDFGLSRDVYEEDSYVKKSKGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKSRDYLDLAASTPSDSLLYDDGLSEEETPLVDCNSAPLPRSLPSTWIENKLYGMSDPNWPGESPVPLTRADGTSTGFPRYANDSVYANWMVSPSAAKLMDTFDS

SEQ ID NO: 18 - protein, 1072 aa, Rattus norvegicus
  IgK signal peptide-Rat RET9 protein sequence
MDMRVPAQLLGLLLLWLRGARCEAPLGLYFSRDAYWERLYVDQPAGTPLLYVHALRDAPGEVPSFRLGQYLYGVYRTRLHENDWIHIDSGTGLLYLNQSLDHSSWEQLSIRNGGFPLLTVFLQVFLGSTAQREGECHWPGCARVYFSFINDTFPNCSSFKARDLCTPETGVSFRIRENRPPGTFYQFRMLPVQFLCPNISVKYKLLEGDGLPFRCDPDCLEVSTRWALDRELQEKYVLEAECAVAGPGANKEKVAVSFPVTVYDEDDSPPTFSGGVGTASAVVEFKRKEGTVVATLQVFDADVVPASGELVRRYTSTLLSGDSWAQQTFRVEHTPNETLVQSNNNSVRATMHNYKLVLNRSLSISESRVLQLVVLVNDSDFQGPGSGVLFLHFNVSVLPVTLNLPMAYSFPVNRRARRYAQIGKVCVENCQEFSGVSIQYKLQPSSTNCSALGVVTSTEDTSGTLYVNDTEALRRPECTELQYTVVATDRQTRRQTQASLVVTVEGTYIAEEVGCPKSCAVNKRRPECEECGGLGSPTGRCEWRQGDGKGITRNFSTCSPSTRTCPDGHCDALESRDINICPQDCLRGPIVGGHERGERQGIKAGYGICNCFPDEKKCFCEPEDSQGPLCDELCRTVITAAVLFSFIISVLLSTFCIHRYHKHAHKPPIASAEMTFCRPAQGFPISYSSSGTRRPSLDSMENQVSVDSFKIPEDPKWEFPRKNLVLGKTLGEGEFGKVVKATAFRLKGRAGYTTVAVKMLKENASQSELRDLLSEFNLLKQVNHPHVIKLYGACSQDGPLLLIVEYAKYGSLRGFLRDSRKIGPAYVSSGGSRNSSSLDHPDERVLTMGDLISFAWQISRGMQYLAEMKLVHRDLAARNILVAEGRKMKISDFGLSRDVYEEDSYVKKSKGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKSRDYLDLAASTPSDSLLYDDGLSEEETPLVDCNSAPLPRSLPSTWIENKLYGRISHAFTRF

SEQ ID NO: 19 - protein, 1108 aa, Macaca fascicularis
  IgK signal peptide-Cynomolgus monkey RET51 protein sequence
MDMRVPAQLLGLLLLWLRGARCLYFSRDAYWEKLYVDQPAGTPLLYVHALRDAPEEVPSFRLGQHLYGTYRTRLHENNWICIQEDTGLLYLNRSLDRSSWEKLSGRNRGFPLLTVYLKVFLSPTSLREGECQWPGCARVYFSFFNTSFPACTSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWTQQTFRVEHWPNETSVQANGSFVRATVHDYRLVLNRNLSISENRTMQLAVLVNDSDFQGPGAGVLLLHFNVSVLPVSLHLPSSYSLSVSRRARRFAQIGKVCVENCQAFSGINVQYELHSSGANCSTLGVVTSAEDTSGILFVNDTKALRRPKCAELHYMVVATNHQTSRQAQAQLLVTVEGLYVAEEAGCPLSCAVSKRRPECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHRYHKFAHKPPIPSAEMTFRRPAQAFPVSYSSSGARRPSLDSMENQVSVDAFKIPEDPKWEFPRKNLVLGKTLGEGEFGKVVKATAFRLKGRAGYTTVAVKMLKENASPSELRDLLSEFNLLKQVNHPHVIKLYGACSQDGPLLLIVEYAKYGSLRGFLRESRKVGPGYLGSGGSRNSSSLDHPDERALTMGDLISFAWQISRGMQYLAEMKLVHRDLAARNILVAEGRKMKISDFGLSRDVYEEDSYVKRSKGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKSRDYLDLAASTPSDSLLYDDGLSEEETPLVDCNNAPLPRALPSTWIENKLYGMSDPNWPGESPVPLTRADGTNTGFPRYANDSVYANWMLSPSAAKLMDTFDS

SEQ ID NO: 20 - protein, 1066 aa, Cynomolgus monkey
  Cynomolgus monkey IgK signal peptide-RET9 protein sequence
MDMRVPAQLLGLLLLWLRGARCLYFSRDAYWEKLYVDQPAGTPLLYVHALRDAPEEVPSFRLGQHLYGTYRTRLHENNWICIQEDTGLLYLNRSLDRSSWEKLSGRNRGFPLLTVYLKVFLSPTSLREGECQWPGCARVYFSFFNTSFPACTSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWTQQTFRVEHWPNETSVQANGSFVRATVHDYRLVLNRNLSISENRTMQLAVLVNDSDFQGPGAGVLLLHFNVSVLPVSLHLPSSYSLSVSRRARRFAQIGKVCVENCQAFSGINVQYELHSSGANCSTLGVVTSAEDTSGILFVNDTKALRRPKCAELHYMVVATNHQTSRQAQAQLLVTVEGLYVAEEAGCPLSCAVSKRRPECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHRYHKFAHKPPIPSAEMTFRRPAQAFPVSYSSSGARRPSLDSMENQVSVDAFKIPEDPKWEFPRKNLVLGKTLGEGEFGKVVKATAFRLKGRAGYTTVAVKMLKENASPSELRDLLSEFNLLKQVNHPHVIKLYGACSQDGPLLLIVEYAKYGSLRGFLRESRKVGPGYLGSGGSRNSSSLDHPDERALTMGDLISFAWQISRGMQYLAEMKLVHRDLAARNILVAEGRKMKISDFGLSRDVYEEDSYVKRSKGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKSRDYLDLAASTPSDSLLYDDGLSEEETPLVDCNNAPLPRALPSTWIENKLYGRISHAFTRF

SEQ ID NO: 21 - protein, 586 aa, Artificial Sequence
  Gfral-Fc sequence
MDMRVPAQLLGLLLLWLRGARCQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 297 aa, Homo sapiens
  GFRAL ECD fragment
QTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSC

SEQ ID NO: 23 - protein, 237 aa, Homo sapiens
  GFRAL ECD fragment
WNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGE

SEQ ID NO: 24 - protein, 733 aa, Artificial Sequence
  HSA-hGDF15 protein sequence
MDMRVPAQLLGLLLLWLRGARCDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSIEGRARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 25 - protein, 308 aa, Homo sapiens
  precursor human GDF15 polypeptide
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 26 - protein, 1072 aa, Homo sapiens; identical to SEQ ID NO: 14
  full-length precursor human RET protein

SEQ ID NO: 27 - protein, 612 aa, Artificial Sequence
  RET-ECD
KVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCR

SEQ ID NO: 28 - protein, 607 aa, Artificial Sequence
  RET-ECD
LYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCR

SEQ ID NO: 29 - protein, 607 aa, Artificial Sequence; identical to SEQ ID NO: 28
  RET-ECD fragment

SEQ ID NO: 30 - protein, 394 aa, Pan troglodytes
  Chimpanzee GFRAL protein
MIVFIFLAMGLSLENEYTSQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDVKQCQAAIRFFYQNIPFNIAQMLAFCDCAQSDIPCQQSKEALHSKTCAVNLVPPPTCLSVIRSCQNDELCRRHYRTFQSKCWQRVTRKCHEDENCISALSKQDLTCSGSDDCKAAYIDILGTVLQVQCTCRTITQSEESLCKIFQHMLHRKSCFNYPTLSNVKGMALYTRKHANKITLTGFHSPFNGEVIYAAMCMTVTCGILLLVMVKLRTSRISSKARDPSPIQIPGEL

SEQ ID NO: 31 - protein, 394 aa, Macaca fascicularis
  Cynomolgus GFRAL protein
FVVVVIQALGLSLENEYTSQTNNCTYLREQCLHDANGCKHAWRIMEDACNDSDPGDPCKMNNSSYCNLSIQYLVESNFRFKECLCTDDFYCTVNKLLGKECVNKSDNMREDKFKWNLTTHSHHGFKGMWSCLEVAEACVGDVVCNAQLASYLKACSANGNPCDVKHCQAAIRFFYQNIPFNIAQMLAFCDCSQSDIPCQQSKEALHSKPCALNMVPPPTCLNVIRSCQNDELCRRHYRTFQTKCWQRVTRKCHEDENCISALSKQDLTCSGSDDCKAAYIDILGTVLQVQCNCRTITQSEESLCKIFQHMLHRKSCFNYPTLSNVKSMALYTRKHTNKITLTGFQSPFNGEVIYAAMCMTVTCGILLLVMVKLRTSRISSKARDPSLSQVPGEL

SEQ ID NO: 32 - protein, 395 aa, Ailuropoda melanoleuca
  Giant Panda GFRAL protein
MVVFIFLAVALCLENESTSETIDCTYLREQCLRDANGCKHAWRIMEDSCNVSEPGNPCKMKDSSKCNLSIQSLVESNFQFEDCLCTDNLYCTINKLLGEQCINESGNVKEDNQSKWNLTTLPYQGIKGIQSCLEVAEACVGDVVCNAQLAPYLKACSANGNLCDVKHCQAAIRFFYQNMPFNIAQMLAFCDCAQSDIPCQQSREALHSKPCAVNVVPPPTCLDVIHSCRNDELCRRRYRTFQSKCWQHVTRKCHEDELCISTLSQQDLICSGSDDCKAAYIGTFGTVLQVQCTCRTITQSEESLCKIFQHMLHRRSCFDYLTLSNVKGMALHKRKPAKEITLSGFHSPFNGEVIYAVMCMTVTCGILLLVMLKLRTSRISSQTRDHSPIRIPGGL

SEQ ID NO: 33 - protein, 395 aa, Canis familiaris
  Dog GFRAL protein
MIVFIFLAMVLCLENESTSQTIDCTHLREQCLSDADGCKHAWRIMEYSCNVSVPGNPCKMKDSSNCNLSIQSLVENNFQFEDCLCTDNIYCTINKLLGEQCMNESDNMKEDNQYKWNLTTLPYHGIKGIQSCLEVAEACVGDVVCNTQLAAYLKACSANGNLCDVKHCQAAIRFFYQNMPFNIAQMLAFCDCAPSDIPCQQSKEALHSKPCAVNVVPPPSCLDVIHSCRNDELCRRRYRTFQSKCWQHMIRKCHEDELCISTLSQQDLTCSGSDDCKAAYIGTFGTVLQVQCTCRTTTQNEESLCKIFQHMLHGRSCFDYPTLSNVKGMALHERKHAKEITLSGFHSPFNGEVIYAVMCMTVTCGILLLVMLKLRTSRISSQTRDHSPIQIPGGL

SEQ ID NO: 34 - protein, 395 aa, Felis catus
  Cat GFRAL protein
SVLIVISAMVLCLENESTSQTIDCTYLREQCLSDTNGCKLAWRKMEDSCNVSDPGNPCKMKDSSNCNLSIQSLVENNFQFEDCLCTGNLHCTINKLLGKKCINESDNMKEDNQSKWNLTTLPYNGIKGIQSCLEVAEACVGDVVCNAQLAPYLKACSANGNLCDVKHCQAAIRFFYQSMPFNIAQMLAFCDCAPSDIPCQQSKEALHSKPCAVNIVPPPTCLNVIHSCRNDELCRRRYRTFQWKCWQQVTRKCHEDEFCISTLSKQDLTCSGSDDCKAAYIGILGTVLQAQCTCRTITQGEESLCKIFQHMLHRRSCFNYPTLSNVKGIALHKRKHAKEITLSGFHSPFNGEVIYAVMCMTVTCGILLLVMLKLRTSRISSQTRDHSPVQILEEL

SEQ ID NO: 35 - protein, 395 aa, Sus scrofa
  Pig GFRAL protein
SVIAVLQAVGLYLENESTSQTTDCTYLRELCLNDTDGCKQAWRIMEDACNVSDPGNTCQMKDSSSCNQSIQSLAESNFQFKDCLCSDDLYCTVNNLIGKKCTNESDNMKEDGVFKRNLTAPSYHGIRGIQSCLEVAEACVRDTVCNAQLALYLKACSANGNLCDVKHCQAAIRFFYQNMPFNVAQMLAFCDCAPSDIPCQQSKEALHSKPCALNRVPAPTCLDVIRSCQNDELCRRRYRIFQAKCWQHVTAKCHDDETCISTLNKQDFTCSRSDDCKAAYMGTLGTALQGQCTCRTTTQSEESLCKIFQHMLHGKSCFNYPTLSNVKGIALHKRKHAKEISLSGCQSPFNGEVIYAVMCMTVTCGILLLVMFKLRNFRISSQTRDPSPIQIPGEP

SEQ ID NO: 36 - protein, 324 aa, Bos taurus
  Bovine GFRAL protein
PLIVVIQAVGLCLNKSASQTTDCTYLRELCLSDADGCKHAWRIMEDACNVSGNTCQMKNSSSCGLSIQSLVESNLQFKDCLCFDDLYCTFNKLLGKKCINESDNIKEDNKYKWNLTTLSYHGGRGVQSCLEVAEVCIGDALCNAQLALYLKACSADGKLCDVKHCQAAIRFFYQNMPFNIAQMLAFCDCAPADEPCQQSREALHSRPCAVNRVPTPTCLDVIHSCQDDELCRRRYRIFQAKCWQHVMRKCHEDETCIGTLSKQDLACSGSDDCKAAYIGTLGTVLQGQCTCRTITQSEESLCKTFQHMLHRKSCFNNKPTQNLK

SEQ ID NO: 37 - protein, 393 aa, Mus musculus; identical to SEQ ID NO: 10
  Mouse GFRAL protein

SEQ ID NO: 38 - protein, 394 aa, Rattus norvegicus; identical to SEQ ID NO: 11
  Rat GFRAL protein

SEQ ID NO: 39 - protein, 388 aa, Cricetulus griseus
  Chinese Hamster GFRAL protein
MDVLLNHSFTSFIEESTLPSDLVPRSNVTQGFLREDTFLTAERKDDCVAAAEACQESVHCSLLHENFKKACGKGTAQCHTLSGQQFCVALRESLRETVLWDCQCGFPLEGDCIRIWKGLFEDICIEDTQINQIATFSQDNEDRLKEDIASDNLKKDNKFKWDLTTLHHACSANGNLCDVKHCQAAIRFFYQNMPFNTAQMLAFCDCAQSDIPCQQSKETLHSKPCALNVVPPPTCLSVIHTCRNDELCRTRYQTFQSECWFHVMGKCHEDETCIGTLGKQDLICSGSDSCRAAYLGTFGTVLQVPCACRSITQDEEPLCMTFQHMLQSKSCFNYPTPNVKDISSYKGKHSKEITLTGFESPFNGELVYAVLCMVVTCGILFLVMLKLR

SEQ ID NO: 40 - protein, 319 aa, Ornithorhynchus anatinus
  Platypus GFRAL protein
MKHYFLFVVLMLGFKCESASLTTGCLHLRKQCVSAMDGCESAWAVIEDVCNVSGHNCTMKESLNCNLSIQLLADRYPAFKDCLCAEDISCSATNFLGRKCIIKTENEHKDKNIKSLWNSTSLLQHGFKGTRSCLEVTVACVGDTVCNKQLARFLKDCSTHGSLCNMNQCQAAIRFFYQNIPFNIAQMLAFCDCAQSDIPCQQSKEALHSKPCAVNVVPTPSCLNVIRSCRDDELCRQRFETFQSKCWQDMDKCSDDETCIVTLNKENITCSRNEECREAYIGTLGTYLHVQCTCSTLSLTEEYLCKIFHHILHSRSCFS

SEQ ID NO: 41 - protein, 308 aa, Macaca mulatta
  Rhesus monkey GDF15 protein
MPGQELKTLNGSQMLLVLLVLLWPPHGGAMSLAEASRASFPGPSDLHSEDSRFRELRKRYEDLLTRLRANQSWEDSNTDLIQAPEVRILTPEVRLGSGGHLHLRISRAVLPEGLPEASRIHRALFRLSPTASRSWDVTRPLRRQLRLARPQAPALHLRLSPPPSQSDQLLVKSSSSRPQLELHLRLRAARGRRRARARNGDRCPLGPGRCCRLHTVHASLEDLGWADWVLSPREVQVTMCIGACPSQFREANMHAQIKMNLHRLKPDTVPAPCCVPASYNPMVLIQKTDTGVSLQTYDDLLAKDCHCV

SEQ ID NO: 42 - protein, 308 aa, Hylobates sp.
  Gibbon GDF15 protein
MPRQEPRMLNGSQMLLVLLVLSWPPHGGALSLAEASPASFPGPSELHSKDSRFRELRKRYEDLLTRLRVNQSWEDLNTDLVPAPAVRILTPEVRLGSGGHLHLRISRAALPEGLPEASRLHRALFRLSPTASRSWDVTRPLRRQLSLARPQAPALHLRLSPPRSRSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 43 - protein, 308 aa, Pan troglodytes
  Chimpanzee GDF15 protein
MPRQELRTLNGSQMLLVLLVLSWPPHGGALSLAEASRASFSGPSELHSEDARFRELRKRYEDLLTRLRANQSWEDSNTDLVPAPAVRILTPEVRLGSGGHLHLRISRAALPEGLPEASRLHRALFRLSPTAARSWDVTRPLRRQLSLARPQAPALHLRLSPPPSQSDQLLAESSSARPQLELHLRSRAARGLRRARARNGDHCPLGPGRCCRLHTVRASLEDLGWSDWVLSPREVQVTMCIGACPSQFRAANMHAQIKTSLHRLKPDTVPAPCCVPASYNPMVLIQKTDTGVSLQTYDDLLAKDCHCI

SEQ ID NO: 44 - protein, 308 aa, Pongo sp.
  Orangutan GDF15 protein
MPRQELRTLNGFQMLLVLLVLSWPPHGGALSLAEASRASFPGPSDLHSEDSRFRELRKRYEDLLTRLRANQSWEDSNTDLVPAPAVRILTPEVRLGSGGHLHLRISRAALPEGLPEASRLHRALFRLSPTASRSWDVTRLLRRQLSLARPQAPALHLRLSPPPSQSDQLLAESSSARPQLELHLRPRAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 45 - protein, 308 aa, Bos taurus
  Cow GDF15 protein
MPGQRPAAPHRSRMLLMLLMFSWLRSGGALSLTQEHLQTFQGPSNVHSSPDISRFRELRKRYEDLLTRLRANQTWEDSNPDLIPPPQVRIVTPKLRLGPGGHLHLRIPRVNLTEGLPAASRLHRALLRLSPKAWSSWDVTRPLRRQLRLGGSRGPSIRLRLLARPDQLQEALPSAPPQLELHWRPSATRGRRHAHAHTRDGCPLGEGRCCHLQSLRASLEDLGWADWVLAPRELDVRMCIGACPSHFRSANTHAQMQARLHGLNPDAAPAPCCVPASYEPVVLMHQDSDGRVSLTPFDDLVAKDCHCV

SEQ ID NO: 46 - protein, 308 aa, Sus scrofa
  Pig GDF15 protein
MPGQLPSPPRRSPTLLMLLMLSWLPSGGALSLAQEHLPALPGPSDVHSSSDVSRFRELRKRYEDLLTRLRANQTWEDSNPNLIPAPQVRIVTPKFRLGPGGHLHLRIPRVNLTEGLPAASRLHRALLRLSPTARSSWDVTRPLRRQLSVGGSRGSAIHLRLLQRPDQLLEALPAGQPQLELHWRPSSTRGRRNTHAHTRDGCPLGTGRCCRLQSLRASLEDLGWADWVLAPRELDVRMCVGACPSHFRSANTHAQMQARLHGLNPDAAPAPCCVPASYEPVVLMHQDSDGRVSLTPFDDLVAKDCHCV

SEQ ID NO: 47 - protein, 307 aa, Canis familiaris
  Dog GDF15 protein
MPGQGPAPAHCSPMLVILVMLSWLPSGGALSLAQEHLPAFPGPSDPHSSTDVSRIQELRKRYEHLQTKLRLNQGWADSNPDLVPATRVRILTPKLRLGPRGHLHLRIARADLTAGLPAASRLHRALLRLSPTEPSSWDVTRPLQRQLSRVGSRTPTLRLRLLPRWDRSRALPSARPQLELHWRPRAARGRRNAHAHARDGCPLGEGRCCRLQSLRASLQDLGWANWVVAPRELDVRMCVGACPSQFRSANTHAQMQARLHGLNPDAAPAPCCVPASYEPVVLMHQDSDGRVSLTPFDDLVAKDCHCV

SEQ ID NO: 48 - protein, 308 aa, Ailuropoda melanoleuca
  Giant panda GDF15 protein
MPGQGLTPPPGSPMLLMLLMFSWLPSGGALSLAQEHLPAFPGPSDTHSSTDVSRAQEFRKRYAHLQTRLWLNQSRADSNSDLIPAAQVRILTPKLRLGSGGHLHLRIARADLTEGLPAASRLHRALLRLSPTEPSSWDVTRPLQRQLSLGGSRAPVLRLRLLPQWDPLGATLPSARPQLELHWRPRAARGRRNAHAHGGDGCPLGEGRCCRLQSLRASLEDLGWADWVVAPRELDVRMCVGACPSQFRSANTHAQMQARLHGLNPEAAPAPCCVPASYEPVVLMHQDSDGRVSLTPFDDLVAKDCHCV

SEQ ID NO: 49 - protein, 298 aa, Mustela furo
  Ferret GDF15 protein
HSSPMLPVLLMFSWLPSGGALSLAQERLPAFPGPSDAHSSMDISRAQEFRKHYEHLQTRLRLNQSWGDSNSDLNLTAPVRILTPKLRLGPGGHLHLRIALADLTEGLPATSRLHQALLRLSPMEPSSWDVTRPLQRQLSLGGSRASALHLRLLPRLNRLRATLPSARPQLELHWRPSAARGRRDAHAHAGDGCPLGEGRCCRLQSLRASLEDLGWANWVVAPRELDVRMCIGACPSQFRSANTHAQMQARLHGLNPDAAPAPCCVPASYEPVVLLHQDSDGRVSLTPFDDLVAKDCHC

SEQ ID NO: 50 - protein, 303 aa, Mus musculus
  Mouse GDF15 protein
MAPPALQAQPPGGSQLRFLLFLLLLLLLLSWPSQGDALAMPEQRPSGPESQLNADELRGRFQDLLSRLHANQSREDSNSEPSPDPAVRILSPEVRLGSHGQLLLRVNRASLSQGLPEAYRVHRALLLLTPTARPWDITRPLKRALSLRGPRAPALRLRLTPPPDLAMLPSGGTQLELRLRVAAGRGRRSAHAHPRDSCPLGPGRCCHLETVQATLEDLGWSDWVLSPRQLQLSMCVGECPHLYRSANTHAQIKARLHGLQPDKVPAPCCVPSSYTPVVLMHRTDSGVSLQTYDDLVARGCHCA

SEQ ID NO: 51 - protein, 301 aa, Cavia porcellus
  Guinea pig GDF15 protein
MPALGFTLLLLAAVSRPPPGGASGPAEKLPAEPDPDLDPDRDPDPAAHPARELRRRFEDLLARLRANRSADALPAERSPAPVVHTLTPDVRRGHDGSLHLRIPRASLPPGVPRASRVQHALLRLSPETREPWDVTRPLQRQLRLLDPRAPALLLHLWPPSDRWRELSSAPPRLELHLRTRSARGRRSTRMRTRDDCPLGPGRCCRLHTVRASLQDLGWTDWVLSPRELHVGICMGECPSLYRSANTHAQVKARLHGLKPDSVPAPCCVPSSYDLVVLMHKTDGGIALHTYDDLIAKGCHCA

SEQ ID NO: 52 - protein, 5 aa, ARTIFICIAL SEQUENCE
  Flexible linker
  REPEAT (1)..(5): Repeat 1 - 20 times
GSGGS

SEQ ID NO: 53 - protein, 5 aa, ARTIFICIAL SEQUENCE
  Flexible linker
  REPEAT (1)..(1): Repeat 1-20 times
  REPEAT (2)..(2): Repeat 1-20 times
  REPEAT (3)..(3): Repeat 1-20 times
  REPEAT (4)..(4): Repeat 1-20 times
  REPEAT (5)..(5): Repeat 1-20 times
  REPEAT (1)..(5): Repeat 1-20 times
GSGSG

SEQ ID NO: 54 - protein, 5 aa, ARTIFICIAL SEQUENCE
  Flexible linker
  REPEAT (5)..(5): Repeat 1 - 20 times
  REPEAT (1)..(5): Repeat 1 - 20 times
GSGGS

SEQ ID NO: 55 - protein, 5 aa, ARTIFICIAL SEQUENCE
  Flexible linker
  REPEAT (4)..(4): Repeat 1 - 20 times
  REPEAT (1)..(5): Repeat 1 - 20 times
GSGSG

SEQ ID NO: 56 - protein, 4 aa, ARTIFICIAL SEQUENCE
  Flexible linker
  REPEAT (4)..(4): Repeat 1 - 20 times
  REPEAT (1)..(4): Repeat 1 - 20 times
GGGS

SEQ ID NO: 57 - protein, 4 aa, ARTIFICIAL SEQUENCE
  Flexible linker
GGSG

SEQ ID NO: 58 - protein, 5 aa, ARTIFICIAL SEQUENCE
  Flexible linker
GGSGG

SEQ ID NO: 59 - protein, 5 aa, ARTIFICIAL SEQUENCE
  Flexible linker
GSGSG

SEQ ID NO: 60 - protein, 5 aa, ARTIFICIAL SEQUENCE
  Flexible linker
GSGGG

SEQ ID NO: 61 - protein, 5 aa, ARTIFICIAL SEQUENCE
  Flexible linker
GGGSG

SEQ ID NO: 62 - protein, 5 aa, ARTIFICIAL SEQUENCE
  Flexible linker
GSSSG

SEQ ID NO: 63 - protein, 5 aa, ARTIFICIAL SEQUENCE
  Flexible linker
  REPEAT (1)..(5): Repeat 1 - 50 times
GSGGS

SEQ ID NO: 64 - protein, 4 aa, ARTIFICIAL SEQUENCE
  Flexible linker
  REPEAT (1)..(4): Repeat 1 - 50 times
GGGS

SEQ ID NO: 65 - protein, 5 aa, ARTIFICIAL SEQUENCE
  Flexible linker
  REPEAT (1)..(5): Repeat 1 - 50 times
GGGGS

SEQ ID NO: 66 - protein, 4 aa, ARTIFICIAL SEQUENCE
  Flexible linker
GGGS

SEQ ID NO: 67 - protein, 5 aa, ARTIFICIAL SEQUENCE
  Flexible linker
GGGGS